(19) United States
(12) Patent Application Publication
(10) Pub. No.: US 2023/0036481 A1
(43) Pub. Date: Feb. 2, 2023

(54) A NOVEL CD16+ NATURAL KILLER CELL AND A METHOD OF CULTURING CD16+ NATURAL KILLER CELL

(71) Applicant: Acepodia Biotechnologies Ltd., New Taipei City (TW)

(72) Inventors: SAI-WEN TANG, San Mateo, CA (US); ZIH-FEI CHENG, New Taipei City (TW); CHIA-YUN LEE, New Taipei City (TW); HAO-KANG LI, New Taipei City (TW); HSIU-PING YANG, New Taipei City (TW); CHING-WEN HSIAO, Castro Valley, CA (US); SEN HEN YANG, Vallejo, CA (US); TAI-SHENG WU, New Taipei City (TW); YAN-LIANG LIN, New Taipei City (TW); YAN-DA LAI, New Taipei City (TW); SHIH-CHIA HSIAO, New Taipei City (TW)

(73) Assignee: Acepodia Biotechnologies Ltd., New Taipei City (TW)

(21) Appl. No.: 17/792,993

(22) PCT Filed: Jan. 15, 2021
(86) PCT No.: PCT/US2021/013577

§ 371 Date: Jul. 14, 2022

Related U.S. Application Data
(60) Provisional application No. 63/052,068, filed on Jul. 15, 2020.

(30) Foreign Application Priority Data
Jan. 16, 2020 (US) PCT/US2020/013883

(51) Int. Cl. A61K 35/17; C12N 5/0783; C07K 14/725; C07K 16/28; A61P 35/00; C07K 16/32

(52) U.S. Cl. CPC A61K 35/17; C12N 5/0646; C07K 14/7051; C07K 16/283; A61P 35/00; C07K 16/32; C12N 2501/599; C12N 2510/00; C07K 2317/622; C07K 2319/03

(57) ABSTRACT

The present invention provides a human CD16⁺ natural killer cell line and a CAR-expressing human CD16⁺ natural killer cell line. These human CD16⁺ natural killer cell line and a CAR-expressing human CD16⁺ natural killer cell line does not include synthetic, genetically modified or purposely deliberately delivered polynucleotide encoding the CD16 receptor and are non-tumorigenic cell lines. Therefore, this human CD16⁺ natural killer cell line and a CAR-expressing human CD16⁺ natural killer cell line might provide considerable long-term safety for disease treatment.

### FIELD OF THE INVENTION

This present invention relates to a CD16⁺ nature killer cell and a method of culturing CD16⁺ nature killer cell; more particularly relates to a CD16⁺ killer cell line that does not include genetically modified polynucleotide encoding the CD16 receptor as well as a culture method capable of mass proliferating CD16⁺ natural killer cells and maintaining CD16 expression.

### BACKGROUND OF THE INVENTION

Natural killer (NK) cells are lymphocytes that constitute an important component of the innate immune system, and they are best appreciated for innate defense against viral infections and in tumor cell surveillance. In humans, NK cells are classically identified by the absence of the T cell receptor complex (CD3⁻) and presence of neural cell adhesion molecule (CD56⁺). There are two main NK cell subsets in human peripheral blood, wherein the majority (>90%) of peripheral blood NK cells are CD3⁻CD56^{dim}CD16⁺ NK cells and the minority (10%) of peripheral blood NK cells are CD3⁻CD56^{bright}CD16⁻ NK cells (Orange JS, 2013).

CD 16 receptor (FcγRIII; it is a receptor for the Fc region of IgG and can bind to the Fc portion of IgG antibodies) is necessary for Antibody-dependent cell cytotoxicity (ADCC) processes carried out by human NK cells. In human, the polynucleotide encoding the CD16 receptor is located on q arm of chromosome 1 at position 1q23.3 Human NK cells expressing CD16 receptor can kill various types of target cells such as cancer cells, tumor cells, and HIV-infected cells through ADCC processes (Rezvani K and Rouce RH, 2015; Littwitz-Salomon et al, 2016; Eileen Scully and Galit Alter, 2016). Take tumor cells as an instance, tumor cells expressing tumor-associated antigens (such as human epidermal growth factor receptor 2, refer to as HER2) can bind to endogenous IgG antibodies or clinically approved therapeutic IgG antibodies targeting the tumor-associated antigens (such as trastuzumab, rituximab, or cetuximab). Once the CD 16 receptors (IgG Fc receptor FcγRIII) expressed by a NK cell bind to the Fc region of the endogenous IgG antibodies or clinically approved therapeutic IgG antibodies, NK cell-mediated ADCC will be triggered and the NK cell will then release cytotoxic factors that cause the death of tumor cells (Rezvani K and Rouce RH, 2015).

There are two major cancer treatment methods by using CD16⁺ NK cells. The first method includes the following steps: (a) obtain autologous or allogeneic blood; (b) isolate autologous or allogeneic primary CD16⁺ natural killer cells (primary CD16⁺ NK cells) from autologous or allogeneic blood; (c) proliferate autologous or allogeneic primary CD16⁺ NK cells in vitro; and (d) inject proliferated autologous or allogeneic primary CD16⁺NK cell back to the veins of a cancer patient, so that there will be enough CD16⁺ NK cells in the cancer patient to release cytotoxic factors that cause the death of cancer cells through ADCC process. However, due to the fact that primary CD16⁺ NK cell will age and even die after several weeks of short-term culture, it is necessary to continuously obtain primary CD16⁺ NK cell from autologous or allogeneic blood for long-term treatment. Moreover, studies have demonstrated that in all of the cultured cells which are obtained from culturing the high-purity CD16⁺ NK cell population (i.e., the amount of CD16⁺ NK cells by number are equal to or more than 99%) with conventional method for 4 days, there are only 10% of cells still express CD16. In other words, the current method of culturing CD16⁺ NK cells in vitro cannot make NK cell stably express CD16 after proliferation. Therefore, the aforesaid method not only has difficulty in retaining the source of primary CD16⁺ NK cells, but also lack of method capable of stably proliferating CD16⁺ NK cells in vitro. These problems often make it difficult for cancer patients to acquire sufficient number of CD16⁺ NK cells and it is difficult to carry out cancer treatment smoothly each time. Moreover, the aforesaid method also needs to face the problem of difficulty in controlling the efficacy caused by individual cell differences.

The second method involves a NK-92 cell line (Deposit number ATCC CRL-2407). NK-92 cell line is a CD16⁻ natural killer cell line, isolated from blood of a fifty-year-old Caucasian male suffering from malignant non-Hodgkin's lymphoma. The NK-92 cell line can be continuously subcultured without aging and death problem, and this NK-92 cell line is not tumorigenic to immune compromised mice. After irradiated with γ-ray, it is also not carcinogenic to allogeneic human subjects, and thus there is a certain degree of applicability. However, since the NK-92 cell line does not express the CD16 receptor, it is unable to destroy cancer cells through ADCC process. Therefore, the aforesaid second method requires the genetic transfer of the CD16 receptor gene into the NK-92 cell line via transgenic technology in order to obtain a transgenic CD16 NK-92 cell line capable of expressing CD16 receptor and exerting ADCC. Then, the NK-92 cell line transfected with CD16 is injected into the vein of the cancer patient; therefore, there are enough CD16⁺ natural killer cells in the cancer patient to release cytotoxic factors that cause the death of cancer cells through ADCC process. Unfortunately, the medical community and the general public are concerned about the long-term safety of transgenic immune cells in the human circulatory system. Hence, the development of the aforesaid method is limited to a considerable extent.

Consequently, there is still an urgent need for a non-transgenic, and non-tumorigenic cell line that can be subcultured continuously as well as a culture method that is capable of mass proliferating CD16⁺ NK while maintaining the expression of CD16.

Moreover, the inventors of the present invention believe that NK cell line has the potential to be further applied in cell therapy that specifically target abnormal cell. Therefore, there is a need to develop an antigen-binding unit-NK cell line conjugation based on chemical conjugation technology or develop a NK cell genetically modified to express an antigen-binding complex for cell therapy that specifically target abnormal cell.

### SUMMARY OF THE INVENTION

The present invention provides a natural killer cell line that can be continuously subcultured without the issue of aging or dying.

The second purpose of the present invention is to provide a natural killer cell that can still stably express CD16⁺ receptor after at least 3 months of proliferation.

Another purpose of the present invention is to provide a CD16⁺ natural killer cell line that does not include genetically modified polynucleotide encoding the CD16 receptor.

Another purpose of the present invention is to provide a CD16⁺ natural killer cell line that is not tumorigenic to immune compromised mice.

Another purpose of the present invention is to provide a CD16⁺ natural killer cell line that is not carcinogenic to an allogeneic human subject after irradiation with γ-ray.

Another purpose of the present invention is to provide a culture method for mass proliferating of CD16⁺ natural killer cells.

Another purpose of the present invention is to provide a culture method that enables CD16⁺ natural killer to stably express CD16 receptor after proliferation.

Another purpose of the present invention is to provide a composition substantially enriched in human CD16⁺ natural killer cells, wherein the number of the human CD16⁺ natural killer cells in the composition is at least 5×10⁵ and the human CD16⁺ natural killer cells are in an amount equal to or more than 5% by number, based on the total number of the cells in the composition as 100%; the human CD16⁺ natural killer cell having the following characteristics: retaining its capability to proliferate after subculture for at least 3 months.

Another purpose of the present invention is to provide a natural killer cell line conjugated with antigen-binding unit or antibody based on chemical conjugation technology.

Another purpose of the present invention is to provide a NK cell genetically modified to express an antigen-binding complex.

Another purpose of the present invention is to provide an antigen-specific NK cell line for cell therapy that specifically target abnormal cell.

Another purpose of the present invention is to provide a method of obtaining an antigen-specific NK cell line that does not include genetically modified polynucleotide encoding the CD16 receptor.

Another purpose of the present invention is to provide a method of treating cancer, tumor, autoimmune disease, neuronal disease, human immunodeficiency virus (HIV) infection, hematopoietic cell-related diseases, metabolic syndrome, pathogenic disease, viral infection, or bacterial infection.

The present invention provides a human natural killer cell having the following characteristics: (i) expressing a CD16 receptor; (ii) retaining its capability to proliferate after subculture for at least 3 months; and (iii) comprising an expressed polynucleotide sequence encoding the CD16 receptor, wherein the expressed polynucleotide sequence encoding the CD16 receptor is not synthetic, not genetically modified and/or not deliberately delivered into the cells.

Preferably, the human natural killer cell is capable of proliferating after subculture for at least 1 week, 2 weeks, 3 weeks, 1 month, 2 months, 3 months, 4 months, 5 months, 6 months, 7 months, 8 months, 9 months, 10 months, or 11 months.

Preferably, the human natural killer cell is capable of proliferating after subculture for at least 1 year, 2 years, or 3 years.

Preferably, the human natural killer cell is non-tumorigenic in an immune compromised mouse.

Preferably, the immune compromised mouse is a SCID/Beige, a NOD/SCID, a NSG, or a nude mouse.

The present invention further provides a composition substantially enriched in human CD16⁺ natural killer cells, wherein the number of the human CD16⁺ natural killer cells in the composition is at least 5×10⁵ and the human CD16⁺ natural killer cells are in an amount equal to or more than 5% by number, based on the total number of the cells in the composition as 100%; the human CD16⁺ natural killer cell having the following characteristics: (i) expressing a CD16 receptor, (ii) retaining its capability to proliferate after subculture for at least 3 months, and (iii) comprising an expressed polynucleotide sequence encoding the CD16 receptor, wherein the polynucleotide sequence encoding the CD16 receptor is not synthetic, not genetically modified and/or not deliberately delivered into the cells.

Preferably, the number of the human CD16⁺ natural killer cells in the composition is 5×10⁵-5×10⁹.

Preferably, the number of the human CD16⁺ natural killer cells in the composition is 1×10⁶, 1.1×10⁶, 5×10⁶, 5.1×10⁶, 1×10⁷, 1.1×10⁷, 5×10⁷, 5.1×10⁷, 1×10⁸, 1.1×10⁸, 5×10⁸, 5.1×10⁸, 1×10⁹, 1.1×10⁹, or 5×10⁹.

Preferably, the total amount of the human CD16⁺ natural killer cells is 5%-100%, based on the total number of the cells in the composition as 100%.

Preferably, the human CD16⁺ natural killer cells are in an amount equal to or more than 5%, 7%, 9%, 10%, 12%, 15%, 19%, 20%, 22%, 25%, 29%, 30%, 32%, 35%, 39%, 40%, 42%, 45%, 49%, 50%, 52%, 55%, 59%, 60%, 62%, 65%, 69%, 70%, 72%, 75%, 79%, 80%, 82%, 85%, 89%, or 95% by number, based on the total number of the cells in the composition as 100%.

Preferably, the CD16 receptor is a CD16a receptor or a CD16b receptor.

Preferably, the expressed polynucleotide sequence encoding the CD16a receptor or the CD16b receptor is not synthetic, not genetically modified and/or not deliberately delivered into cells.

A method of obtaining a composition substantially enriched in human CD16⁺ natural killer cells; the method comprising: (a) obtaining a population of human peripheral blood natural killer cells derived from the cell population having the deposit number ATCC CRL-2047; (b) contacting the population of human peripheral blood natural killer cells with an antibody specific for a CD16 receptor; and (c) separating cells that are specifically bound by the antibody thereby obtaining the composition substantially enriched in human CD16⁺ natural killer cells; wherein the human CD16⁺ natural killer cell comprises an expressed polynucleotide encoding a CD16 receptor, and the expressed polynucleotide sequence encoding the CD16 receptor is not synthetic, not genetically modified and/or not deliberately delivered into the cells.

Preferably, the human CD16⁺ natural killer cells are capable of retaining their capability to proliferate after subculture for at least 3 months.

Preferably, the human CD16⁺ natural killer cells are capable of retaining their capability to proliferate after subculture for at least 1 week, 2 weeks, 3 weeks, 1 month, 2 months, 3 months, 4 months, 5 months, 6 months, 7 months, 8 months, 9 months, 10 months, or 11 months.

Preferably, the human CD16⁺ natural killer cells are capable of retaining their capability to proliferate after subculture for at least 1 year, 2 years, or 3 years.

Preferably, the expressed polynucleotide sequence encoding the CD16 receptor is not synthetic, not genetically modified and/or not deliberately delivered into the cells.

Preferably, in the composition, the human CD16⁺ natural killer cells are in an amount equal to or more than 80% by number, based on the total number of the cells in the composition as 100%.

Preferably, the human CD16⁺ natural killer cells are in an amount equal to or more than 5% by number, based on the total number of the cells in the composition as 100%.

Preferably, the human CD16⁺ natural killer cells are in an amount equal to or more than 50% by number, based on the total number of the cells in the composition as 100%.

Preferably, the concentration of the dissolved glucose in the culture medium is higher than 1500 mg/L.

Preferably, the culture medium is fully aerated, the concentration of the dissolved oxygen in the culture medium is maintained in a stable range, or the concentration of the dissolved glucose in the culture medium is 1500-5000 mg/L.

Preferably, the concentration of the dissolved glucose in the culture medium is 2500, 2501, 3500, 3501, 4000, or 4500 mg/L.

Preferably, the number of the human CD16⁺ natural killer cells in the composition is at least 5×10⁵, and the human CD16⁺ natural killer cells are in an amount equal to or more than 5% by number, based on the total number of the cells in the composition as 100%.

Preferably, the number of the human CD16⁺ natural killer cells in the composition is 5×10⁵-5×10⁹.

Preferably, the number of the human CD16⁺ natural killer cells in the composition is 1×10⁶, 1.1×10⁶, 5×10⁶, 1×10⁷, 1.1×10⁷, 5×10⁷, 5.1×10⁷, 1×10⁸, 1.1×10⁸, 5×10⁸, 5.1×10⁸, 1×10⁹, 1.1×10⁹, 5×10⁹, 1×10¹⁰, 1.1×10¹⁰, 5×10¹⁰, 1×10¹¹, 1.1×10¹¹, 5×10¹¹, 5.1×10¹¹, 1×10¹², 1.1×10¹², 5×10¹², 5.1×10¹², 1×10¹³, 1.1×10¹⁴, 5×10¹⁴, 1×10¹⁵, 1.1×10¹⁵, 5×10¹⁵, 1×10¹⁶, 1.1×10¹⁶, 5×10¹⁶, 5.1×10¹⁶, 1×10¹⁷, 1.1×10¹⁷, 5×10¹⁷, 5.1×10¹⁷, 1×10¹⁸, 1.1×10¹⁸, 5×10¹⁸, 1×10¹⁹, 1.1×10¹⁹, 5×10¹⁹, 1×10²⁰, 1.1×10²⁰, 5×10²⁰, 5.1×10²⁰, 1×10²¹, 1.1×10²¹, 5×10²¹, 5.1×10²¹, 1×10²², 1.1×10²², 5×10²², 1×10²³, 1.1×10²³, 5×10²³, 1×10²⁴, 1.1×10²⁴, 5×10²⁴, 5.1×10²⁴, 1×10²⁵, 1.1×10²⁵, 5×10²⁵, 5.1×10²⁵, 1×10²⁶, 1.1×10²⁶, 5×10²⁶, 1×10²⁷, 1.1×10²⁷, 5×10²⁷, 1×10²⁸, 1.1×10²⁸, 5×10²⁸, 5.1×10²⁸, 1×10²⁹, 1.1×10²⁹, 5×10²⁹, 5.1×10²⁹, 1×10³⁰, 1.1×10³⁰, 5×10³⁰, 1×10³¹, 1.1×10³¹, 5×10³¹, 1×10³², 1.1×10³², 5×10³², 5.1×10³², 1×10³³, 1.1×10³³, 5×10³³, 5.1×10³³, 1×10³⁴, 1.1×10³⁴, 5×10³⁴, 1×10³⁵, 1.1×10³⁵, 5×10³⁵, 1×10³⁶, 1.1×10³⁶, 5×10³⁶, 5.1×10³⁶, 1×10³⁷, 1.1×10³⁷, 5×10³⁷, 5.1×10³⁷, 1×10³⁸, 1.1×10³⁸, 5×10³⁸, 1×10³⁹, 1.1×10³⁹, 5×10³⁹, 5.1×10³⁹, 1×10⁴⁰, 1.1×10⁴⁰, 5×10⁴⁰. Preferably, the number of the human CD16⁺ natural killer cells in the composition is 1×10⁶-1×10⁴¹.

Preferably, the total number of the human CD16⁺ natural killer cells is 5%-100%, based on the total number of the cells in the composition as 100%.

Preferably, the human CD16⁺ natural killer cells are in an amount equal to or more than 5%, 7%, 9%, 10%, 12%, 15%, 19%, 20%, 22%, 25%, 29%, 30%, 32%, 35%, 39%, 40%, 42%, 45%, 49%, 50%, 52%, 55%, 59%, 60%, 62%, 65%, 69%, 70%, 72%, 75%, 79%, 80%, 82%, 85%, 89%, or 95% by number, based on the total number of the cells in the composition as 100%.

The present invention further provides a method of culturing and expanding human CD16⁺ natural killer cells; the method comprising: (x) in a container, contacting the human CD16⁺ natural killer cells with a culture medium comprising human platelet lysate and IL-2; and (y) culturing the cells for multiple days; wherein the human CD16⁺ natural killer cell comprises an expressed polynucleotide encoding a CD16 receptor and the expressed polynucleotide sequence encoding the CD16 receptor is not synthetic, not genetically modified and/or not deliberately delivered into the cells.

Preferably, the concentration of the dissolved glucose in the culture medium is higher than 1500 mg/L.

Preferably, the step (y) comprises substeps: (y1) culturing the cells for at least one day; and (y2) sub-culturing the cells for at least 3 months.

Preferably, wherein the step (y2) is to sub-culturing the cells for at least 1 week, 2 weeks, 3 weeks, 1 month, 2 months, 3 months, 4 months, 5 months, 6 months, 7 months, 8 months, 9 months, 10 months, or 11 months.

Preferably, wherein the step (y2) is to sub-culturing the cells for at least 1 year, 2 years, or 3 years.

Preferably, the human CD16⁺ natural killer cells are capable of retaining their capability to proliferate after subculture for at least 3 months.

Preferably, the human CD16⁺ natural killer cells are capable of retaining their capability to proliferate after subculture for at least 1 week, 2 weeks, 3 weeks, 1 month, 2 months, 3 months, 4 months, 5 months, 6 months, 7 months, 8 months, 9 months, 10 months, or 11 months.

Preferably, the human CD16⁺ natural killer cells are capable of retaining their capability to proliferate after subculture for at least 1 year, 2 years, or 3 years.

Preferably, the expressed polynucleotide sequence encoding the CD16 receptor is not synthetic, not genetically modified and/or not deliberately delivered in the cells.

Preferably, the human CD16⁺ natural killer cell expresses CD2 molecule (CD2⁺).

Preferably, the human CD16⁺ natural killer cell expresses NKp44, NKp46, NKG2D, or CD107a.

The present invention provides a human natural killer cell having the following characteristics: (i) expressing a CD16 receptor; (ii) retaining its capability to proliferate after subculture for at least 3 months; and (iii) comprising an expressed CD16A gene encoding the CD16 receptor, wherein the expressed CD16A gene is located on q arm of chromosome 1.

Preferably, the expressed CD16A gene is located on q arm of chromosome 1 at position 1q23.3.

The present invention provides a human natural killer cell having the following characteristics: (i) expressing a CD16 receptor; (ii) retaining its capability to proliferate after subculture for at least 3 months; and (iii) comprising an expressed CD16A gene encoding the CD16 receptor, wherein the expressed CD16A gene polynucleotide sequence is not synthetic, not genetically modified and/or not deliberately delivered into cells.

Preferably, the expressed CD16A gene polynucleotide sequence is not synthetic, not genetically modified and/or not deliberately delivered into the cells.

The present invention provides a human natural killer cell which is (A) deposited at NPMD having the deposit number NITE BP-03017; or (B) having the following characteristics:

i) expressing a CD16 receptor;

ii) retaining its capability to proliferate after subculture for at least 3 months; and

iii) x) not including synthetic, genetically modified and/or deliberately delivered polynucleotide encoding the CD16 receptor, or y) by using ddPCR system to analyze the genomic DNA of the cell, the ratio of CD16 F176F probe-detectable DNA molecule to CD16 F176V probe-detectable DNA molecule is equal to higher than 1, wherein the sequence of the CD16 F176F probe is SEQ ID NO: 11 and the sequence of the CD16 F176V probe is SEQ ID NO: 12.

Preferably, the CD16 receptor is a CD16a receptor or a CD16b receptor.

Preferably, an expressed polynucleotide encoding the CD16 receptor is located on q arm of chromosome 1 at position 1q23.3.

Preferably, the cell is non-tumorigenic in an immune compromised mouse.

Preferably, after being irradiated with γ-ray, the cell is non-tumorigenic in an allogeneic subject.

Preferably, a polynucleotide encoding the CD16 receptor comprising a nucleotide sequence of SEQ ID NO:1, SEQ ID NO:2, or SEQ ID NO:19.

Preferably, the CD16 receptor comprising an amino acid sequence of SEQ ID NO:3, SEQ ID NO:4, or SEQ ID NO:20.

Preferably, the cell further comprises an inactive tumor suppressor gene or a mutated and highly expressed oncogene.

Preferably, the cell is capable of mediating an antibody-dependent cell cytotoxicity (ADCC) response, and the cell is a male cell.

Preferably, the cell further comprises at least an exogenous targeting unit complexed to the cell, wherein the exogenous targeting unit comprises a targeting moiety that is characterized in that: (a) it exhibits specific binding to a biological marker on a target cell; (b) it is not a nucleic acid; and (c) it is not produced by the cell.

Preferably, the exogenous targeting unit is complexed to the cell via an interaction between a first linker conjugated to the targeting moiety and a second linker conjugated to the cell.

Preferably, the first linker is a first polynucleotide, or the second linker is a second polynucleotide.

Preferably, the targeting moiety comprises an antigen-binding unit.

Preferably, the first polynucleotide comprises a single-stranded region.

Preferably, the first linker is a first polynucleotide, and the second linker is a second polynucleotide.

Preferably, the first linker and the second linker are selected from the group consisting of: a DNA binding domain and a target DNA; a leucine zipper and a target DNA; biotin and avidin; biotin and streptavidin; calmodulin binding protein and calmodulin; a hormone and a hormone receptor; lectin and a carbohydrate; a cell membrane receptor and a receptor ligand; an enzyme and a substrate; an antigen and an antibody; an agonist and an antagonist; polynucleotide hybridizing sequences; an aptamer and a target; and a zinc finger and a target DNA.

Preferably, the first linker comprises a first reactive group, and the second linker comprises a second reactive group, and wherein the cell is complexed to the targeting moiety via a covalent bond formed by a reaction between the second reactive group and the first reactive group.

Preferably, the targeting moiety comprises an antigen-binding unit.

Preferably, the second linker comprises a PEG region.

Preferably, the targeting moiety and the cell are separated by a length of 1 nm to 400 nm, or the targeting moiety and the cell are separated by a length of 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 42, 44, 46, 48, 50, 60, 70, 80, 90, 100, 130, 170, 200, 230, 270, 300, 330, or 370 mm.

Preferably, the exogenous targeting unit comprises an antigen-binding unit, and the antigen-binding unit binds to a cancer antigen, glycolipid, glycoprotein, cluster of differentiation antigen present on cells of a hematopoietic lineage, gamma-glutamyltranspeptidase, adhesion protein, hormone, growth factor, cytokine, ligand receptor, ion channel, membrane-bound form of an immunoglobulin μ. chain, alfa-fetoprotein, C-reactive protein, chromogranin A, epithelial mucin antigen, human epithelium specific antigen, Lewis(a) antigen, multidrug resistance related protein, Neu oncogene protein, neuron specific enolase, P-glycoprotein, multidrug-resistance-related antigen, p170, multidrug-resistance-related antigen, prostate specific antigen, NCAM, ganglioside molecule, MART-1, heat shock protein, sialylTn, tyrosinase, MUC-1, HER-2/neu, KSA, PSMA, p53, RAS, EGF-R, VEGF, MAGE, or other target antigen (marker) expressed by a target cell.

Preferably, the antigen-binding unit is an antibody against a cancer antigen selected from HER2/neu (ERBB2), HER3 (ERBB3), EGFR, VEGF, VEGFR2, GD2, CTLA4, CD19, CD20, CD22, CD30, CD33 (Siglec-3), CD52 (CAMPATH-1 antigen), CD326 (EpCAM), CA-125 (MUC16), MMP9, DLL3, CD274 (PD-L1), CEA, MSLN (mesothelin), CA19-9, CD73, CD205 (DEC205), CD51, c-MET, TRAIL-R2, IGF-1R, CD3, MIF, folate receptor alpha (FOLR1), CSF1, OX-40, CD137, TfR, MUC1, CD25 (IL-2R), CD115 (CSF1R), IL1B, CD105 (Endoglin), KIR, CD47, CEA, IL-17A, DLL4, CD51, angiopoietin 2, neuropilin-1, CD37, CD223 (LAG-3), CD40, LIV-1 (SLC39A6), CD27 (TNFRSF7), CD276 (B7-H3), Trop2, Claudin1 (CLDN1), PSMA, TIM-1 (HAVcr-1), CEACAM5, CD70, LY6E, BCMA, CD135 (FLT3), APRIL, TF(F3), nectin-4, FAP, GPC3, FGFR3, a killer-cell immunoglobulin-like receptors (KIRs), a TNF receptor protein, an immunoglobulin protein, a cytokine receptor, an integrin, activating NK cell receptors, and combinations thereof.

Preferably, the targeting moiety is conjugated to the first polynucleotide using a coupling group, wherein the coupling group is an NHS ester, other activated ester, an alkyl or acyl halide, a bifunctional crosslinker, or maleimide group.

Preferably, the first polynucleotide or second polynucleotide comprise a sequence selected from 20-mer poly-CA, 20-mer poly-GGTT, SEQ ID NO: 5, SEQ ID NO: 6, SEQ ID NO: 8, SEQ ID NO: 9, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, 23-mer SEQ ID NO: 7, and SEQ ID NO:10.

Preferably, the binding affinity of the targeting moiety for the biological marker is less than 250 nM, or the binding affinity of the targeting moiety for the biological marker is 5 nM, 10 nM, 40 nM, 90 nM, 130 nM, or 170 nM.

Preferably, the length of the first polynucleotide or the length of the second polynucleotide are 4 nt to 500 nt. Preferably, the length of the first polynucleotide or the length of the second polynucleotide are 4, 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 42, 44, 46, 48, 50, 55, 60, 65, 70, 75, 80, 85, 90, 95, 100, 120, 160, 220, 300, 400, or 480 nt.

Preferably, the binding affinity between the first linker and the second linker is less than 250 nM. Preferably, the binding affinity between the first linker and the second linker is 5 nM, 10 nM, 40 nM, 90 nM, 130 nM, or 170 nM.

Preferably, the first linker or the second linker is conjugated to a native functional group of the targeting unit or a surface of the cell, wherein the native functional group is an amino acid, a sugar, or an amine.

Preferably, the targeting moiety is a peptide, protein, or aptamer.

The present invention provides a composition substantially enriched in human CD16⁺ natural killer cells, wherein the number of the human CD16⁺ natural killer cells in the composition is at least 5×10⁵ and the human CD16⁺ natural killer cells are in an amount equal to or more than 5% by number, based on the total number of the cells in the composition as 100%; the human CD16⁺ natural killer cell is (A) deposited at NPMD having the deposit number NITE BP-03017; or (B) having the following characteristics:

i) expressing a CD16 receptor,

ii) retaining its capability to proliferate after subculture for at least 3 months, and

iii) x) not including synthetic, genetically modified and/or deliberately delivered polynucleotide encoding the CD16 receptor, or y) by using ddPCR system to analyze the genomic DNA of the cell, the ratio of CD16 F176F probe-detectable DNA molecule to CD16 F176V probe-detectable DNA molecule is equal to or higher than 1, wherein the sequence of the CD16 F176F probe is SEQ ID NO: 11 and the sequence of the CD16 F176V probe is SEQ ID NO: 12.

Preferably, the CD16 receptor is a CD16a receptor or a CD16b receptor.

Preferably, a polynucleotide encoding the CD16 receptor is located on q arm of chromosome 1 at position 1q23.3.

Preferably, the human CD16⁺ natural killer cells are non-tumorigenic in an immune compromised mouse.

Preferably, after being irradiated with γ-ray, the human CD16⁺ natural killer cells are non-tumorigenic in an allogeneic subject.

Preferably, a polynucleotide encoding the CD16 receptor comprises a nucleotide sequence of SEQ ID NO:1, SEQ ID NO:2, or SEQ ID NO:19.

Preferably, the CD16 receptor comprising an amino acid sequence of SEQ ID NO:3, SEQ ID NO:4, or SEQ ID NO:20.

Preferably, the human CD16⁺ natural killer cell further comprises an inactive tumor suppressor gene or a mutated and highly expressed oncogene.

Preferably, the human CD16⁺ natural killer cell is capable of mediating an antibody-dependent cell cytotoxicity (ADCC) response, and the cell is a male cell.

Preferably, the human CD16⁺ natural killer cell further comprises at least an exogenous targeting unit complexed to the human CD16⁺ natural killer cell, wherein the exogenous targeting unit comprises a targeting moiety that is characterized in that: (a) it exhibits specific binding to a biological marker on a target cell; (b) it is not a nucleic acid; and (c) it is not produced by the human CD16⁺ natural killer cell.

Preferably, the exogenous targeting unit is complexed to the human CD16⁺ natural killer cell via an interaction between a first linker conjugated to the targeting moiety and a second linker conjugated to the human CD16⁺ natural killer cell.

Preferably, the first linker is a first polynucleotide, or the second linker is a second polynucleotide.

Preferably, the targeting moiety comprises an antigen-binding unit.

Preferably, the first polynucleotide comprises a single-stranded region.

Preferably, the first linker is a first polynucleotide, and the second linker is a second polynucleotide.

Preferably, the first linker and the second linker are selected from the group consisting of: a DNA binding domain and a target DNA; a leucine zipper and a target DNA; biotin and avidin; biotin and streptavidin; calmodulin binding protein and calmodulin; a hormone and a hormone receptor; lectin and a carbohydrate; a cell membrane receptor and a receptor ligand; an enzyme and a substrate; an antigen and an antibody; an agonist and an antagonist; polynucleotide hybridizing sequences; an aptamer and a target; and a zinc finger and a target DNA.

Preferably, the first linker comprises a first reactive group, and the second linker comprises a second reactive group, and wherein the human CD16⁺ natural killer cell is complexed to the targeting moiety via a covalent bond formed by a reaction between the second reactive group and the first reactive group.

Preferably, the targeting moiety comprises an antigen-binding unit.

Preferably, the second linker comprises a PEG region.

Preferably, the targeting moiety and the human CD16⁺ natural killer cells are separated by a length of 1 nm to 400 nm.

Preferably, the exogenous targeting unit comprises an antigen-binding unit, and the antigen-binding unit binds to a cancer antigen, glycolipid, glycoprotein, cluster of differentiation antigen present on cells of a hematopoietic lineage, gamma-glutamyltranspeptidase, adhesion protein, hormone, growth factor, cytokine, ligand receptor, ion channel, membrane-bound form of an immunoglobulin μ. chain, alfa-fetoprotein, C-reactive protein, chromogranin A, epithelial mucin antigen, human epithelium specific antigen, Lewis(a) antigen, multidrug resistance related protein, Neu oncogene protein, neuron specific enolase, P-glycoprotein, multidrug-resistance-related antigen, p170, multidrug-resistance-related antigen, prostate specific antigen, NCAM, ganglioside molecule, MART-1, heat shock protein, sialylTn, tyrosinase, MUC-1, HER-2/neu, KSA, PSMA, p53, RAS, EGF-R, VEGF, MAGE, or other target antigen (marker) expressed by a target cell.

Preferably, the antigen-binding unit is an antibody against a cancer antigen selected from HER2/neu (ERBB2), HER3 (ERBB3), EGFR, VEGF, VEGFR2, GD2, CTLA4, CD19, CD20, CD22, CD30, CD33 (Siglec-3), CD52 (CAMPATH-1 antigen), CD326 (EpCAM), CA-125 (MUC16), MMP9, DLL3, CD274 (PD-L1), CEA, MSLN (mesothelin), CA19-9, CD73, CD205 (DEC205), CD51, c-MET, TRAIL-R2, IGF-1R, CD3, MIF, folate receptor alpha (FOLR1), CSF1, OX-40, CD137, TfR, MUC1, CD25 (IL-2R), CD115 (CSF1R), IL1B, CD105 (Endoglin), KIR, CD47, CEA, IL-17A, DLL4, CD51, angiopoietin 2, neuropilin-1, CD37, CD223 (LAG-3), CD40, LIV-1 (SLC39A6), CD27 (TNFRSF7), CD276 (B7-H3), Trop2, Claudin1 (CLDN1), PSMA, TIM-1 (HAVcr-1), CEACAM5, CD70, LY6E, BCMA, CD135 (FLT3), APRIL, TF(F3), nectin-4, FAP, GPC3, FGFR3, ICAM-1 (CD54), ROBO1, NKG2D ligands, CD123, CS1/SLAMF7/CD319/CRACC, CD7, CD142 (platelet tissue factor, factor III, tissue factor), CD38, CD138, EGFR VIII, EGFR, EGFR806, EGFR family member, PD-1, ROR1, CSPG4, CLL-1 (CLEC12A), CD147, PSCA, EPHA2, GPRC5D, CD133, B7H6, DSC2, AE1 (SLC4A1), GUCY2C, CDH17, HPSE, CD24, MUC4, AFP-L3, SP17, DCLK1, CAIX (CA9), IL13RA2, IL13Ra, CD56, CD44v6, TCR beta-chain, ligands of chlorotoxin, claudin-6, claudin-18.2, EIIIB (fibronectin), Glypican-1 (GPC1), PLAP (Placental alkaline phosphatase), uPAR, HCMV glycoprotein B (gB), HLA-DR (Lym1 antibody target), tumor-associated αvβ6 integrin, LunX, integrin αvβ3, folate receptor beta (FRβ), LILRB4, MISIIR (Müllerian inhibiting substance type 2 receptor), 5T4, CD83 ligand, HBsAg, CD171 (L1-CAM), TAG72 (TAG72 (Tumour-associated glycoprotein 72)), B7-H4, CD166 (ALCAM), AC133 (PROM1), LeY, CD13 (TIM1), CD117, TEM8 (ANTXR1), CD26, IL13Ra2, IGF1R, Muc3a, IL1RAP, TSLPR (CRLF2), LMP1, Siglec7, Siglec9, Epstein-Barr Virus gp350, CD1a, CLEC14A, MAGE-A1, MAGE-A4, Neurofilament M (NEFM), HERV-K env protein, HLA-A*0201/NY-ESO-1(157-165) peptide, 2B4, TACI (TNFRSF13B), CD32A(131R), AXL, Lewis Y, CD80, CD86, ROR2, a killer-cell immunoglobulin-like receptors (KIRs), a TNF receptor protein, an immunoglobulin protein, a cytokine receptor, an integrin, activating NK cell receptors, and combinations thereof.

Preferably, the targeting moiety is conjugated to the first polynucleotide using a coupling group, wherein the coupling group is an NHS ester, other activated ester, an alkyl or acyl halide, a bifunctional crosslinker, or maleimide group.

Preferably, the first polynucleotide or second polynucleotide comprise a sequence selected from 20-mer poly-CA, 20-mer poly-GGTT, SEQ ID NO: 5, SEQ ID NO: 6, SEQ ID NO: 8, SEQ ID NO: 9, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, 23-mer SEQ ID NO: 7, and SEQ ID NO:10.

Preferably, the binding affinity of the targeting moiety for the biological marker is less than 250 nM.

Preferably, the length of the first polynucleotide or the length of the second polynucleotide are 4 nt to 500 nt.

Preferably, the binding affinity between the first linker and the second linker is less than 250 nM.

Preferably, the first linker or the second linker is conjugated to a native functional group of the targeting unit or a surface of the human CD16⁺ natural killer cell, wherein the native functional group is an amino acid, a sugar, or an amine.

Preferably, the targeting moiety is a peptide, protein, or aptamer.

The present invention provides a method of obtaining a composition substantially enriched in human CD16⁺ natural killer cells; the method comprising: (a) obtaining a population of human peripheral blood natural killer cells derived from the cell population having the deposit number ATCC CRL-2407; (b) contacting the population of human peripheral blood natural killer cells with an antibody specific for a CD16 receptor; and (c) separating cells that are specifically bound by the antibody thereby obtaining the composition substantially enriched in human CD16⁺ natural killer cells; wherein the human CD16⁺ natural killer cell is: (A) deposited at NPMD having the deposit number NITE BP-03017; or (B) having the following characteristics:

i) expressing a CD16 receptor, and

ii) x) not including synthetic, genetically modified and/or deliberately delivered polynucleotide encoding the CD16 receptor, or y) by using ddPCR system to analyze the genomic DNA of the cell, the ratio of CD16 F176F probe-detectable DNA molecule to CD16 F176V probe-detectable DNA molecule is equal to or higher than 1, wherein the sequence of the CD16 F176F probe is SEQ ID NO: 11 and the sequence of the CD16 F176V probe is SEQ ID NO: 12.

Preferably, the antibody is specific for at least one of a CD16a receptor and a CD16b receptor.

Preferably, the human CD16⁺ natural killer cells are capable of retaining their capability to proliferate after subculture for at least 1 week.

Preferably, an expressed polynucleotide encoding the CD16 receptor is located on q arm of chromosome 1 at position 1q23.3.

Preferably, the human CD16⁺ natural killer cells are non-tumorigenic in an immune compromised mouse.

Preferably, after being irradiated with γ-ray, the human CD16⁺ natural killer cells are non-tumorigenic in an allogeneic subject.

Preferably, a polynucleotide encoding the CD16 receptor comprising a nucleotide sequence of SEQ ID NO:1, SEQ ID NO:2, or SEQ ID NO:19.

Preferably, the CD16 receptor comprising an amino acid sequence of SEQ ID NO:3, SEQ ID NO:4, or SEQ ID NO:20.

Preferably, the human CD16⁺ natural killer cell further comprises an inactive tumor suppressor gene or a mutated and highly expressed oncogene.

Preferably, in the composition, the human CD16⁺ natural killer cells are in an amount equal to or more than 5% by number, based on the total number of the cells in the composition as 100%.

Preferably, the human CD16⁺ natural killer cells are capable of mediating an antibody-dependent cell cytotoxicity (ADCC) response, and the human CD16⁺ natural killer cells are male cells.

Preferably, the step (c) comprises substeps: (c1) separating cells that are specifically bound by the antibody; (c2) in a container, contacting the cells that are specifically bound by the antibody with a culture medium comprising human platelet lysate and IL-2; and (c3) culturing the cells for multiple days thereby obtaining the composition substantially enriched in human CD16⁺ natural killer cells.

Preferably, the container is G-Rex culture devices.

Preferably, the container comprises a bottom for seeding cells, and the bottom is air-permeable and water-impermeable.

Preferably, the concentration of the dissolved glucose in the culture medium is 1500-5000 mg/L.

Preferably, the number of the human CD16⁺ natural killer cells in the composition is at least 5×10⁵, and the human CD16⁺ natural killer cells are in an amount equal to or more than 5% by number, based on the total number of the cells in the composition as 100%.

The present invention provides a method of culturing and expanding human CD16⁺ natural killer cells; the method comprising (x) in a container, contacting the human CD16⁺ natural killer cells with a culture medium comprising 0.5-10 vol % human platelet lysate and 100-3000 IU/mLIL-2; and (y) culturing the cells for multiple days. Preferably, the culture medium comprised 1 vol %, 2 vol %, 3 vol %, 4 vol %, 5 vol %, 6 vol %, 7 vol %, 8 vol %, 9 vol %, 10 vol %, 11 vol %, 12 vol %, 13 vol %, 14 vol %, or 15 vol % human platelet lysate. Preferably, the culture medium comprised 0.5-20 vol % human platelet lysate. Preferably, the culture medium comprised 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, or 3000 IU/mL IL-2.

Preferably, the container is G-Rex culture devices.

Preferably, the container comprises a bottom for seeding cells, and the bottom is air-permeable and water-impermeable.

Preferably, the concentration of the dissolved glucose in the culture medium is 1500-5000 mg/L.

Preferably, the step (y) comprises substeps: (y1) culturing the cells for at least one day; and (y2) sub-culturing the cells for at least 1 months.

Preferably, the human CD16⁺ natural killer cells are capable of retaining their capability to proliferate after subculture for at least 3 months.

Preferably the human CD16⁺ natural killer cell is: (A) deposited at NPMD having the deposit number NITE BP-03017; or (B) having the following characteristics:

i) expressing a CD16 receptor, and

ii) x) not including synthetic, genetically modified and/or deliberately delivered polynucleotide encoding the CD16 receptor, or y) by using ddPCR system to analyze the genomic DNA of the cell, the ratio of CD16 F176F probe-detectable DNA molecule to CD16 F176V probe-detectable DNA molecule is equal to or higher than 1, wherein the sequence of the CD16 F176F probe is SEQ ID NO: 11 and the sequence of the CD16 F176V probe is SEQ ID NO: 12

Preferably, the human CD16⁺ natural killer cells are non-tumorigenic in an immune compromised mouse.

Preferably, after being irradiated with γ-ray, the human CD16⁺ natural killer cells are non-tumorigenic in an allogeneic subject.

Preferably, a polynucleotide encoding the CD16 receptor comprising a nucleotide sequence of SEQ ID NO:1, SEQ ID NO:2, or SEQ ID NO:19.

Preferably, the CD16 receptor comprising an amino acid sequence of SEQ ID NO:3, SEQ ID NO:4, or SEQ ID NO:20.

Preferably, the human CD16⁺ natural killer cell further comprises an inactive tumor suppressor gene or a mutated and highly expressed oncogene.

The present invention provides a method of treating cancer, autoimmune disease, neuronal disease, human immunodeficiency virus (HIV) infection, hematopoietic cell-related diseases, metabolic syndrome, pathogenic disease, viral infection, or bacterial infection, comprising administering a composition comprising an effective amount of a human natural killer cell to a subject in need thereof; the human natural killer cell is (A) deposited at NPMD having the deposit number NITE BP-03017; or (B) having the following characteristics:

i) expressing a CD16 receptor,

ii) retaining its capability to proliferate after subculture for at least 3 months, and iii) x) not including synthetic, genetically modified and/or deliberately delivered polynucleotide encoding the CD16 receptor, or y) by using ddPCR system to analyze the genomic DNA of the human natural killer cell, the ratio of CD16 F176F probe-detectable DNA molecule to CD16 F176V probe-detectable DNA molecule is higher than 1, wherein the sequence of the CD16 F176F probe is SEQ ID NO: 11 and the sequence of the CD16 F176V probe is SEQ ID NO: 12.

Preferably, the human natural killer cell further comprises at least an exogenous targeting unit complexed to the human natural killer cell, wherein the exogenous targeting unit comprises a targeting moiety that is characterized in that: (a) it exhibits specific binding to a biological marker on a target cell; (b) it is not a nucleic acid; and (c) it is not produced by the human natural killer cell.

Preferably, the exogenous targeting unit is complexed to the human natural killer cell via an interaction between a first linker conjugated to the targeting moiety and a second linker conjugated to the human natural killer cell.

Preferably, the first linker is a first polynucleotide, or the second linker is a second polynucleotide.

Preferably, the targeting moiety comprises an antigen-binding unit.

Preferably, the first polynucleotide comprises a single-stranded region.

Preferably, the first linker is a first polynucleotide, and the second linker is a second polynucleotide.

Preferably, the first linker and the second linker are selected from the group consisting of: a DNA binding domain and a target DNA; a leucine zipper and a target DNA; biotin and avidin; biotin and streptavidin; calmodulin binding protein and calmodulin; a hormone and a hormone receptor; lectin and a carbohydrate; a cell membrane receptor and a receptor ligand; an enzyme and a substrate; an antigen and an antibody; an agonist and an antagonist; polynucleotide hybridizing sequences; an aptamer and a target; and a zinc finger and a target DNA.

Preferably, the first linker comprises a first reactive group, and the second linker comprises a second reactive group, and wherein the human natural killer cell is complexed to the targeting moiety via a covalent bond formed by a reaction between the second reactive group and the first reactive group.

Preferably, the targeting moiety comprises an antigen-binding unit.

Preferably, the second linker comprises a PEG region.

Preferably, the targeting moiety and the human natural killer cell are separated by a length of 1 nm to 400 nm.

Preferably, the exogenous targeting unit comprises an antigen-binding unit, and the antigen-binding unit binds to a cancer antigen, glycolipid, glycoprotein, cluster of differentiation antigen present on cells of a hematopoietic lineage, gamma-glutamyltranspeptidase, adhesion protein, hormone, growth factor, cytokine, ligand receptor, ion channel, membrane-bound form of an immunoglobulin μ. chain, alfa-fetoprotein, C-reactive protein, chromogranin A, epithelial mucin antigen, human epithelium specific antigen, Lewis(a) antigen, multidrug resistance related protein, Neu oncogene protein, neuron specific enolase, P-glycoprotein, multidrug-resistance-related antigen, p170, multidrug-resistance-related antigen, prostate specific antigen, NCAM, ganglioside molecule, MART-1, heat shock protein, sialylTn, tyrosinase, MUC-1, HER-2/neu, KSA, PSMA, p53, RAS, EGF-R, VEGF, MAGE, or other target antigen (marker) expressed by a target cell.

Preferably, the antigen-binding unit is an antibody against a cancer antigen selected from HER2/neu (ERBB2), HER3 (ERBB3), EGFR, VEGF, VEGFR2, GD2, CTLA4, CD19, CD20, CD22, CD30, CD33 (Siglec-3), CD52 (CAMPATH-1 antigen), CD326 (EpCAM), CA-125 (MUC16), MMP9, DLL3, CD274 (PD-L1), CEA, MSLN (mesothelin), CA19-9, CD73, CD205 (DEC205), CD51, c-MET, TRAIL-R2, IGF-1R, CD3, MIF, folate receptor alpha (FOLR1), CSF1, OX-40, CD137, TfR, MUC1, CD25 (IL-2R), CD115 (CSF1R), IL1B, CD105 (Endoglin), KIR, CD47, CEA, IL-17A, DLL4, CD51, angiopoietin 2, neuropilin-1, CD37, CD223 (LAG-3), CD40, LIV-1 (SLC39A6), CD27 (TNFRSF7), CD276 (B7-H3), Trop2, Claudin1 (CLDN1), PSMA, TIM-1 (HAVcr-1), CEACAM5, CD70, LY6E, BCMA, CD135 (FLT3), APRIL, TF(F3), nectin-4, FAP, GPC3, FGFR3, ICAM-1 (CD54), ROBO1, NKG2D ligands, CD123, CS1/SLAMF7/CD319/CRACC, CD7, CD142 (platelet tissue factor, factor III, tissue factor), CD38, CD138, EGFR VIII, EGFR, EGFR806, EGFR family member, PD-1, ROR1, CSPG4, CLL-1 (CLEC12A), CD147, PSCA, EPHA2, GPRC5D, CD133, B7H6, DSC2, AE1 (SLC4A1), GUCY2C, CDH17, HPSE, CD24, MUC4, AFP-L3, SP17, DCLK1, CAIX (CA9), IL13RA2, IL13Ra, CD56, CD44v6, TCR beta-chain, ligands of chlorotoxin, claudin-6, claudin-18.2, EIIIB (fibronectin), Glypican-1 (GPC1), PLAP (Placental alkaline phosphatase), uPAR, HCMV glycoprotein B (gB), HLA-DR (Lym1 antibody target), tumor-associated αvβ6 integrin, LunX, integrin αvβ3, folate receptor beta (FRβ), LILRB4, MISIIR (Müllerian inhibiting substance type 2 receptor), 5T4, CD83 ligand, HBsAg, CD171 (L1-CAM), TAG72 (TAG72 (Tumour-associated glycoprotein 72)), B7-H4, CD166 (ALCAM), AC133 (PROM1), LeY, CD13 (TIM1), CD117, TEM8 (ANTXR1), CD26, IL13Ra2, IGF1R, Muc3a, IL1RAP, TSLPR (CRLF2), LMP1, Siglec7, Siglec9, Epstein-Barr Virus gp350, CD1a, CLEC14A, MAGE-A1, MAGE-A4, Neurofilament M (NEFM), HERV-K env protein, HLA-A*0201/NY-ESO-1(157-165) peptide, 2B4, TACI (TNFRSF13B), CD32A(131R), AXL, Lewis Y, CD80, CD86, ROR2, a killer-cell immunoglobulin-like receptors (KIRs), a TNF receptor protein, an immunoglobulin protein, a cytokine receptor, an integrin, activating NK cell receptors, and combinations thereof.

The cancer antigen HER2/neu (ERBB2) is an antigen encoded by HER2/neu (ERBB2) gene. HER2/neu (ERBB2) gene localized but not limited at chromosome 17 q arm 12 encodes a member of the epidermal growth factor (EGF) receptor family of receptor tyrosine kinases. This protein has no ligand binding domain of its own and therefore cannot bind growth factors. The Gene ID in NCBI is 2064 but not limited to. Please refer to https://www.ncbi.nlm.nih.gov/gene/2064.

The cancer antigen HER3 (ERBB3) is an antigen encoded by HER3 (ERBB3) gene. HER3 (ERBB3) gene localized but not limited at chromosome 12 q arm 13.2 encodes a member of the epidermal growth factor receptor (EGFR) family of receptor tyrosine kinases. This membrane-bound protein has a neuregulin binding domain but not an active kinase domain. It therefore can bind this ligand but not convey the signal into the cell through protein phosphorylation. This protein has no ligand binding domain of its own and therefore cannot bind growth factors. The Gene ID in NCBI is 2065 but not limited to. Please refer to https://www.ncbi.nlm.nih.gov/gene/2065.

The cancer antigen EGFR is an antigen encoded by EGFR gene. EGFR gene is localized but not limited at chromosome 7 p arm 11.2. The protein encoded by this gene is a transmembrane glycoprotein that is a member of the protein kinase superfamily. This protein is a receptor for members of the epidermal growth factor family. EGFR is a cell surface protein that binds to epidermal growth factor, thus inducing receptor dimerization and tyrosine autophosphorylation leading to cell proliferation. The Gene ID in NCBI is 1956 but not limited to. Please refer to https://www.ncbi.nlm.nih.gov/gene/1956.

The cancer antigen VEGF is an antigen encoded by VEGF gene. VEGF gene is localized but not limited at chromosome 6 p arm 21.1 is a member of the PDGF/VEGF growth factor family. It encodes a heparin-binding protein, which exists as a disulfide-linked homodimer. This growth factor induces proliferation and migration of vascular endothelial cells, and is essential for both physiological and pathological angiogenesis. Disruption of this gene in mice resulted in abnormal embryonic blood vessel formation. This gene is upregulated in many known tumors and its expression is correlated with tumor stage and progression. Elevated levels of this protein are found in patients with POEMS syndrome, also known as Crow-Fukase syndrome. The Gene ID in NCBI is 7422 but not limited to. Please refer to https://www.ncbi.nlm.nih.gov/gene/7422.

The cancer antigen VEGFR2 is an antigen encoded by VEGFR2 gene. VEGFR2 gene is localized but not limited at chromosome 4 q arm 12 encodes one of the two receptors of the VEGF. This receptor, known as kinase insert domain receptor, is a type III receptor tyrosine kinase. It functions as the main mediator of VEGF-induced endothelial proliferation, survival, migration, tubular morphogenesis and sprouting. The Gene ID in NCBI is 3791 but not limited to. Please refer to https://www.ncbi.nlm.nih.gov/gene/3791.

The cancer antigen GD2 is a disialoganglioside expressed on tumors of neuroectodermal origin, including human neuroblastoma and melanoma, with highly restricted expression on normal tissues, principally to the cerebellum and peripheral nerves in humans. The Gene ID in NCBI is 6644 but not limited to. Please refer to https://www.immunol.org/content/181/9/6644.

The cancer antigen CTLA4 is an antigen encoded by CTLA4 gene. CTLA4 gene localized but not limited at chromosome 2 q arm 33.2 is a member of the immunoglobulin superfamily and encodes a protein which transmits an inhibitory signal to T cells. The protein contains a V domain, a transmembrane domain, and a cytoplasmic tail. Alternate transcriptional splice variants, encoding different isoforms, have been characterized. The membrane-bound isoform functions as a homodimer interconnected by a disulfide bond, while the soluble isoform functions as a monomer. The Gene ID in NCBI is 1493 but not limited to. Please refer to https://www.ncbi.nlm.nih.gov/gene/1493.

The cancer antigen CD19 is an antigen encoded by CD19 gene. CD19 gene localized but not limited at chromosome 16 p arm 11.2 encodes a member of the immunoglobulin gene superfamily. Expression of this cell surface protein is restricted to B cell lymphocytes. This protein is a reliable marker for pre-B cells but its expression diminishes during terminal B cell differentiation in antibody secreting plasma cells. The protein has two N-terminal extracellular Ig-like domains separated by a non-Ig-like domain, a hydrophobic transmembrane domain, and a large C-terminal cytoplasmic domain. The Gene ID in NCBI is 930 but not limited to. Please refer to https://www.ncbi.nlm.nih.gov/gene/930.

The cancer antigen CD20 is an antigen encoded by CD20 gene. CD20 gene localized but not limited at chromosome 11 q arm 12.2 encodes a member of the membrane-spanning 4A gene family. Members of this nascent protein family are characterized by common structural features and similar intron/exon splice boundaries and display unique expression patterns among hematopoietic cells and nonlymphoid tissues. This gene encodes a B-lymphocyte surface molecule which plays a role in the development and differentiation of B-cells into plasma cells. The Gene ID in NCBI is 931 but not limited to. Please refer to https://www.ncbi.nlm.nih.gov/gene/931.

The cancer antigen CD22 is an antigen encoded by CD22 gene. CD22 gene localized but not limited at chromosome 19 q arm 13.12 is a molecule belonging to the SIGLEC family of lectins which specifically binds sialic acid with an immunoglobulin (Ig) domain located at its N-terminus. The presence of Ig domains makes CD22 a member of the immunoglobulin superfamily. CD22 functions as an inhibitory receptor for B cell receptor (BCR) signaling. The Gene ID in NCBI is 933 but not limited to. Please refer to https://www.ncbi.nlm.nih.gov/gene/933.

The cancer antigen CD30 is an antigen encoded by CD30 gene. CD30 gene localized but not limited at chromosome 1 p arm 36.22 encodes a member of the TNF-receptor superfamily. This receptor is expressed by activated, but not by resting, T and B cells. TRAF2 and TRAF5 can interact with this receptor, and mediate the signal transduction that leads to the activation of NF-kappaB. This receptor is a positive regulator of apoptosis, and also has been shown to limit the proliferative potential of autoreactive CD8 effector T cells and protect the body against autoimmunity. The Gene ID in NCBI is 943 but not limited to. Please refer to https://www.ncbi.nlm.nih.gov/gene/943.

The cancer antigen CD33 (Siglec-3) is an antigen encoded by CD33 (Siglec-3) gene. CD33 (Siglec-3) gene localized but not limited at chromosome 19 q arm 13.41 encodes a transmembrane receptor expressed on cells of myeloid lineage. It binds sialic acids, therefore is a member of the SIGLEC family of lectins. The Gene ID in NCBI is 945 but not limited to. Please refer to https://www.ncbi.nlm.nih.gov/gene/945.

The cancer antigen CD52 (CAMPATH-1 antigen) is an antigen encoded by CD52 (CAMPATH-1 antigen) gene. CD52 (CAMPATH-1 antigen) gene localized but not limited at chromosome 1 p arm 36.11 encodes a glycoprotein present on the surface of mature lymphocytes, but not on the stem cells from which these lymphocytes were derived. CD52 binds the ITIM (immunoreceptor tyrosine-based inhibitory motif)-bearing sialic acid-binding lectin SIGLEC10. The Gene ID is 1043 but not limited to. Please refer to https://www.ncbi.nlm.nih.gov/gene/1043.

The cancer antigen CD326 (EpCAM) is an antigen encoded by CD326 (EpCAM) gene. CD326 (EpCAM) gene localized but not limited at chromosome 2 p arm 21 encodes a carcinoma-associated antigen and is a member of a family that includes at least two type I membrane proteins. This antigen is expressed on most normal epithelial cells and gastrointestinal carcinomas and functions as a homotypic calcium-independent cell adhesion molecule. The antigen is being used as a target for immunotherapy treatment of human carcinomas. The Gene ID in NCBI is 4072 but not limited to. Please refer to https://www.ncbi.nlm.nih.gov/gene/4072.

The cancer antigen CA-125 (MUC16) is an antigen encoded by CA-125 (MUC16) gene. CA-125 (MUC16) gene localized but not limited at chromosome 19 p arm 13.2 encodes a protein that is a member of the mucin family. Mucins are high molecular weight, O-glycosylated proteins that play an important role in forming a protective mucous barrier, and are found on the apical surfaces of the epithelia. The encoded protein is a membrane-tethered mucin that contains an extracellular domain at its amino terminus, a large tandem repeat domain, and a transmembrane domain with a short cytoplasmic domain. The Gene ID in NCBI is 94025 but not limited to. Please refer to https://www.ncbi.nlm.nih.gov/gene/94025.

The cancer antigen MMP9 is an antigen encoded by MMP9 gene. MMP9 gene localized but not limited at chromosome 20 q arm 13.12 encodes a 92 kDa type IV collagenase, 92 kDa gelatinase or gelatinase B (GELB), is a matrixin, a class of enzymes that belong to the zinc-metalloproteinases family involved in the degradation of the extracellular matrix. The Gene ID in NCBI is 4318 but not limited to. Please refer to https://www.ncbi.nlm.nih.gov/gene/4318.

The cancer antigen DLL3 is an antigen encoded by DLL3 gene. DLL3 gene localized but not limited at chromosome 19 q arm 13.2 encodes a member of the delta protein ligand family. This family functions as Notch ligands that are characterized by a DSL domain, EGF repeats, and a transmembrane domain. The Gene ID in NCBI is 10683 but not limited to. Please refer to https://www.ncbi.nlm.nih.gov/gene/10683.

The cancer antigen CD274 (PD-L1) is an antigen encoded by CD274 (PD-L1) gene. CD274 (PD-L1) gene localized but not limited at chromosome 9 p arm 24.1 encodes an immune inhibitory receptor ligand that is expressed by hematopoietic and non-hematopoietic cells, such as T cells and B cells and various types of tumor cells. The encoded protein is a type I transmembrane protein that has immunoglobulin V-like and C-like domains Interaction of this ligand with its receptor inhibits T-cell activation and cytokine production. The Gene ID in NCBI is 29126 but not limited to. Please refer to https://www.ncbi.nlm.nih.gov/gene/29126.

The cancer antigen CEA is an antigen encoded by CEA gene. CEA gene localized but not limited at chromosome 19 q arm 13.2 encodes a cell surface glycoprotein that represents the founding member of the carcinoembryonic antigen (CEA) family of proteins. The encoded protein is used as a clinical biomarker for gastrointestinal cancers and may promote tumor development through its role as a cell adhesion molecule. Additionally, the encoded protein may regulate differentiation, apoptosis, and cell polarity. The Gene ID in NCBI is 1048 but not limited to. Please refer to https://www.ncbi.nlm.nih.gov/gene/1048.

The cancer antigen MSLN (mesothelin) is an antigen encoded by MSLN (mesothelin) gene. MSLN (mesothelin) gene localized but not limited at chromosome 16 p arm 13.3 encodes a preproprotein that is proteolytically processed to generate two protein products, megakaryocyte potentiating factor and mesothelin. Megakaryocyte potentiating factor functions as a cytokine that can stimulate colony formation of bone marrow megakaryocytes. Mesothelin is a glycosylphosphatidylinositol-anchored cell-surface protein that may function as a cell adhesion protein. This protein is overexpressed in epithelial mesotheliomas, ovarian cancers and in specific squamous cell carcinomas. The Gene ID in NCBI is 10232 but not limited to. Please refer to https://www.ncbi.nlm.nih.gov/gene/10232.

The cancer antigen CD73 is an antigen encoded by CD73 gene. CD73 gene localized but not limited at chromosome 6 q arm 14.3 encodes a plasma membrane protein that catalyzes the conversion of extracellular nucleotides to membrane-permeable nucleosides. The encoded protein is used as a determinant of lymphocyte differentiation. Defects in this gene can lead to the calcification of joints and arteries. The Gene ID in NCBI is 4907 but not limited to. Please refer to https://www.ncbi.nlm.nih.gov/gene/4907.

The cancer antigen CD205 (DEC205) is an antigen encoded by CD205 (DEC205) gene. CD205 (DEC205) gene LY75 localized but not limited at chromosome 2 q arm 24.2 encodes a protein CD205 or DEC-205. The Gene ID in NCBI is 4065 but not limited to. Please refer to https://www.ncbi.nlm.nih.gov/gene/4065.

The cancer antigen CD51 is an antigen encoded by CD51 gene. CD51 gene localized but not limited at chromosome 2 q arm 32.1 encodes the integrin alpha chain family. Integrins are heterodimeric integral membrane proteins composed of an alpha subunit and a beta subunit that function in cell surface adhesion and signaling. The encoded preproprotein is proteolytically processed to generate light and heavy chains that comprise the alpha V subunit. This subunit associates with beta 1, beta 3, beta 5, beta 6 and beta 8 subunits. The heterodimer consisting of alpha V and beta 3 subunits is also known as the vitronectin receptor. This integrin may regulate angiogenesis and cancer progression. The Gene ID in NCBI is 3685 but not limited to. Please refer to https://www.ncbi.nlm.nih.gov/gene/3685)

The cancer antigen c-MET is an antigen encoded by c-MET gene. c-MET gene localized but not limited at chromosome 7 q arm 31.2 encodes a member of the receptor tyrosine kinase family of proteins and the product of the proto-oncogene MET. The encoded preproprotein is proteolytically processed to generate alpha and beta subunits that are linked via disulfide bonds to form the mature receptor. Further processing of the beta subunit results in the formation of the M10 peptide, which has been shown to reduce lung fibrosis. Binding of its ligand, hepatocyte growth factor, induces dimerization and activation of the receptor, which plays a role in cellular survival, embryogenesis, and cellular migration and invasion. Mutations in this gene are associated with papillary renal cell carcinoma, hepatocellular carcinoma, and various head and neck cancers. Amplification and overexpression of this gene are also associated with multiple human cancers. The Gene ID in NCBI is 4233 but not limited to. Please refer to https://www.ncbi.nlm.nih.gov/gene/4233)

The cancer antigen TRAIL-R2 is an antigen encoded by TRAIL-R2 gene. TRAIL-R2 gene localized but not limited at chromosome 8 p arm 21.3 encodes a member of the TNF-receptor superfamily, and contains an intracellular death domain. This receptor can be activated by tumor necrosis factor-related apoptosis inducing ligand (TNFSF10/TRAIL/APO-2L), and transduces an apoptosis signal. Studies with FADD-deficient mice suggested that FADD, a death domain containing adaptor protein, is required for the apoptosis mediated by this protein. The Gene ID in NCBI is 8795 but not limited to. Please refer to https://www.ncbi.nlm.nih.gov/gene/8795.

The cancer antigen IGF-1R is an antigen encoded by IGF-1R gene. IGF-1R gene localized but not limited at chromosome 15 q arm 26.3 encodes a receptor binding insulin-like growth factor with a high affinity. It has tyrosine kinase activity. The insulin-like growth factor I receptor plays a critical role in transformation events. Cleavage of the precursor generates alpha and beta subunits. It is highly overexpressed in most malignant tissues where it functions as an anti-apoptotic agent by enhancing cell survival. The Gene ID in NCBI is 3480 but not limited to. Please refer to https://www.ncbi.nlm.nih.gov/gene/3480.

The cancer antigen MIF is an antigen encoded by MIF gene. MIF gene localized but not limited at chromosome 22 q arm 11.23 encodes a lymphokine involved in cell-mediated immunity, immunoregulation, and inflammation. It plays a role in the regulation of macrophage function in host defense through the suppression of anti-inflammatory effects of glucocorticoids. This lymphokine and the JAB1 protein form a complex in the cytosol near the peripheral plasma membrane, which may indicate an additional role in integrin signaling pathways. The Gene ID in NCBI is 4282 but not limited to. Please refer to https://www.ncbi.nlm.nih.gov/gene/4282)

The cancer antigen folate receptor alpha (FOLR1) is an antigen encoded by folate receptor alpha (FOLR1) gene. Folate receptor alpha (FOLR1) gene localized but not limited at chromosome 11 q arm 13.4 encodes a member of the folate receptor family Members of this gene family bind folic acid and its reduced derivatives, and transport 5-methyltetrahydrofolate into cells. This gene product is a secreted protein that either anchors to membranes via a glycosyl-phosphatidylinositol linkage or exists in a soluble form. Mutations in this gene have been associated with neurodegeneration due to cerebral folate transport deficiency. The Gene ID in NCBI is 2348 but not limited to. Please refer to https://www.ncbi.nlm.nih.gov/gene/2348.

The cancer antigen CSF1 is an antigen encoded by CSF1 gene. CSF1 gene localized but not limited at chromosome 1 p arm 13.3 encodes a cytokine that controls the production, differentiation, and function of macrophages. The active form of the protein is found extracellularly as a disulfide-linked homodimer, and is thought to be produced by proteolytic cleavage of membrane-bound precursors. The encoded protein may be involved in development of the placenta. The Gene ID in NCBI is 1435 but not limited to. Please refer to https://www.ncbi.nlm.nih.gov/gene/1435.

The cancer antigen CSF1 is an antigen encoded by CSF1 gene. OX-40 gene localized but not limited at chromosome 1 p arm 36.33 encodes a member of the TNF-receptor superfamily. This receptor has been shown to activate NF-kappaB through its interaction with adaptor proteins TRAF2 and TRAF5. Knockout studies in mice suggested that this receptor promotes the expression of apoptosis inhibitors BCL2 and BCL21L1/BCL2-XL, and thus suppresses apoptosis. The knockout studies also suggested the roles of this receptor in CD4+ T cell response, as well as in T cell-dependent B cell proliferation and differentiation. The Gene ID in NCBI is 7293 but not limited to. Please refer to https://www.ncbi.nlm.nih.gov/gene/7293.

The cancer antigen CD137 is an antigen encoded by CD137 gene. CD137 gene localized but not limited at chromosome 1 p arm 36.23 encodes a member of the TNF-receptor superfamily. This receptor contributes to the clonal expansion, survival, and development of T cells. It can also induce proliferation in peripheral monocytes, enhance T cell apoptosis induced by TCR/CD3 triggered activation, and regulate CD28 co-stimulation to promote Th1 cell responses. The expression of this receptor is induced by lymphocyte activation. TRAF adaptor proteins have been shown to bind to this receptor and transduce the signals leading to activation of NF-kappaB. The Gene ID in NCBI is 3604 but not limited to. Please refer to https://www.ncbi.nlm.nih.gov/gene/3604.

The cancer antigen TfR is an antigen encoded by TfR gene. TfR gene localized but not limited at chromosome 3 q arm 29 encodes a cell surface receptor necessary for cellular iron uptake by the process of receptor-mediated endocytosis. This receptor is required for erythropoiesis and neurologic development. The Gene ID in NCBI is 7037 but not limited to. Please refer to https://www.ncbi.nlm.nih.gov/gene/7037.

The cancer antigen MUC1 is an antigen encoded by MUC1 gene. MUC1 gene localized but not limited at chromosome 1 q arm 22 encodes a membrane-bound protein that is a member of the mucin family. Mucins are 0-glycosylated proteins that play an essential role in forming protective mucous barriers on epithelial surfaces. These proteins also play a role in intracellular signaling. This protein is expressed on the apical surface of epithelial cells that line the mucosal surfaces of many different tissues including lung, breast stomach and pancreas. This protein is proteolytically cleaved into alpha and beta subunits that form a heterodimeric complex. The N-terminal alpha subunit functions in cell-adhesion and the C-terminal beta subunit is involved in cell signaling. Overexpression, aberrant intracellular localization, and changes in glycosylation of this protein have been associated with carcinomas. The Gene ID in NCBI is 4582 but not limited to. Please refer to https://www.ncbi.nlm.nih.gov/gene/4582.

The cancer antigen CD25 (IL-2R) is an antigen encoded by CD25 (IL-2R) gene. CD25 (IL-2R) gene localized but not limited at chromosome 10 p arm 15.1 encodes IL-2 receptor alpha (IL2RA), together with the common beta (IL2RB) gamma chain (IL2RG), constitute the high-affinity IL2 receptor. Homodimeric alpha chains (IL2RA) result in low-affinity receptor, while homodimeric beta (IL2RB) chains produce a medium-affinity receptor. Normally an integral-membrane protein, soluble IL2RA has been isolated and determined to result from extracellular proteolysis. The Gene ID in NCBI is 3559 but not limited to. Please refer to https://www.ncbi.nlm.nih.gov/gene/3559.

The cancer antigen CD115 (CSF1R) is an antigen encoded by CD115 (CSF1R) gene. CD115 (CSF1R) gene localized but not limited at chromosome 5 q arm 32 encodes the receptor for colony stimulating factor 1, a cytokine which controls the production, differentiation, and function of macrophages. This receptor mediates most if not all of the biological effects of this cytokine. Ligand binding activates the receptor kinase through a process of oligomerization and transphosphorylation. The encoded protein is a tyrosine kinase transmembrane receptor and member of the CSF1/PDGF receptor family of tyrosine-protein kinases. The Gene ID in NCBI is 1436 but not limited to. Please refer to https://www.ncbi.nlm.nih.gov/gene/1436.

The cancer antigen IL1B is an antigen encoded by IL1B gene. IL1B gene localized but not limited at chromosome 2 q arm 14.1 encodes a member of the interleukin 1 cytokine family. This cytokine is produced by activated macrophages as a proprotein, which is proteolytically processed to its active form by caspase 1 (CASP1/ICE). This cytokine is an important mediator of the inflammatory response, and is involved in a variety of cellular activities, including cell proliferation, differentiation, and apoptosis. The induction of cyclooxygenase-2 (PTGS2/COX2) by this cytokine in the central nervous system (CNS) is found to contribute to inflammatory pain hypersensitivity. Similarly, IL-1B has been implicated in human osteoarthritis pathogenesis. The Gene ID in NCBI is 3553 but not limited to. Please refer to https://www.ncbi.nlm.nih.gov/gene/3553.

The cancer antigen CD105 (Endoglin) is an antigen encoded by CD105 (Endoglin) gene. CD105 (Endoglin) gene localized but not limited at chromosome 9 q arm 34.11 encodes a homodimeric transmembrane protein which is a major glycoprotein of the vascular endothelium. This protein is a component of the transforming growth factor beta receptor complex and it binds to the beta1 and beta3 peptides with high affinity. Mutations in this gene cause hereditary hemorrhagic telangiectasia, also known as Osler-Rendu-Weber syndrome 1, an autosomal dominant multisystemic vascular dysplasia. This gene may also be involved in preeclampsia and several types of cancer. The Gene ID in NCBI is 2022 but not limited to. Please refer to https://www.ncbi.nlm.nih.gov/gene/2022.

The cancer antigen CD47 is an antigen encoded by CD47 gene. CD47 gene localized but not limited at chromosome 3 q arm 13.12 encodes a membrane protein, which is involved in the increase in intracellular calcium concentration that occurs upon cell adhesion to extracellular matrix. The encoded protein is also a receptor for the C-terminal cell binding domain of thrombospondin, and it may play a role in membrane transport and signal transduction. This gene has broad tissue distribution, and is reduced in expression on Rh erythrocytes. The Gene ID in NCBI is 961 but not limited to. Please refer to https://www.ncbi.nlm.nih.gov/gene/961.

The cancer antigen CEA is an antigen encoded by CEA gene. CEA gene localized but not limited at chromosome 19 q arm 13.2 encodes a member of the family of carcinoembryonic antigen-related cell adhesion molecules (CEACAMs), which are used by several bacterial pathogens to bind and invade host cells. The encoded transmembrane protein directs phagocytosis of several bacterial species that is dependent on the small GTPase Rac. It is thought to serve an important role in controlling human-specific pathogens by the innate immune system. The Gene ID in NCBI is 1084 but not limited to. Please refer to https://www.ncbi.nlm.nih.gov/gene/1084.

The cancer antigen IL-17A is an antigen encoded by IL-17A gene. IL-17A gene localized but not limited at chromosome 6 p arm 12.2 encodes a member of the IL-17 receptor family which includes five members (IL-17RA-E) and the encoded protein is a proinflammatory cytokine produced by activated T cells. IL-17A-mediated downstream pathways induce the production of inflammatory molecules, chemokines, antimicrobial peptides, and remodeling proteins. The encoded protein elicits crucial impacts on host defense, cell trafficking, immune modulation, and tissue repair, with a key role in the induction of innate immune defenses. This cytokine stimulates non-hematopoietic cells and promotes chemokine production thereby attracting myeloid cells to inflammatory sites. This cytokine also regulates the activities of NF-kappaB and mitogen-activated protein kinases and can stimulate the expression of IL6 and cyclooxygenase-2 (PTGS2/COX-2), as well as enhance the production of nitric oxide (NO). IL-17A plays a pivotal role in various infectious diseases, inflammatory and autoimmune disorders, and cancer. High levels of this cytokine are associated with several chronic inflammatory diseases including rheumatoid arthritis, psoriasis and multiple sclerosis. The Gene ID in NCBI is 3605 but not limited to. Please refer to https://www.ncbi.nlm.nih.gov/gene/3605.

The cancer antigen DLL4 is an antigen encoded by DLL4 gene. DLL4 gene localized but not limited at chromosome 15 q arm 15.1 encodes a homolog of the Drosophila delta gene. The delta gene family encodes Notch ligands that are characterized by a DSL domain, EGF repeats, and a transmembrane domain. The Gene ID in NCBI is 54567 but not limited to. Please refer to https://www.ncbi.nlm.nih.gov/gene/54567.

The cancer antigen CD51 is an antigen encoded by CD51 gene. CD51 gene localized but not limited at chromosome 2 q arm 32.1 encodes the product belonging to the integrin alpha chain family. Integrins are heterodimeric integral membrane proteins composed of an alpha subunit and a beta subunit that function in cell surface adhesion and signaling. The encoded preproprotein is proteolytically processed to generate light and heavy chains that comprise the alpha V subunit. This subunit associates with beta 1, beta 3, beta 5, beta 6 and beta 8 subunits. The heterodimer consisting of alpha V and beta 3 subunits is also known as the vitronectin receptor. This integrin may regulate angiogenesis and cancer progression. The Gene ID in NCBI is 3685 but not limited to. Please refer to https://www.ncbi.nlm.nih.gov/gene/3685.

The cancer antigen angiopoietin 2 is an antigen encoded by angiopoietin 2 gene. angiopoietin 2 gene localized but not limited at chromosome 8 p arm 23.1 encodes the product belonging to the angiopoietin family of growth factors. The protein encoded by this gene is an antagonist of angiopoietin 1, and both angiopoietin 1 and angiopoietin 2 are ligands for the endothelial TEK receptor tyrosine kinase Angiopoietin 2 is upregulated in multiple inflammatory diseases and is implicated in the direct control of inflammation-related signaling pathways. The encoded protein affects angiogenesis during embryogenesis and tumorigenesis, disrupts the vascular remodeling ability of angiopoietin 1, and may induce endothelial cell apoptosis. The Gene ID in NCBI is 285 but not limited to. Please refer to https://www.ncbi.nlm.nih.gov/gene/285.

The cancer antigen neuropilin-1 is an antigen encoded by neuropilin-1 gene. neuropilin-1 gene localized but not limited at chromosome 10 p arm 11.22 encodes one of two neuropilins, which contain specific protein domains which allow them to participate in several different types of signaling pathways that control cell migration. Neuropilins contain a large N-terminal extracellular domain, made up of complement-binding, coagulation factor V/VIII, and meprin domains. These proteins also contain a short membrane-spanning domain and a small cytoplasmic domain Neuropilins bind many ligands and various types of co-receptors; they affect cell survival, migration, and attraction. Some of the ligands and co-receptors bound by neuropilins are vascular endothelial growth factor (VEGF) and semaphorin family members. The Gene ID in NCBI is 8829 but not limited to. Please refer to https://www.ncbi.nlm.nih.gov/gene/8829.

The cancer antigen CD37 is an antigen encoded by CD37 gene. CD37 gene localized but not limited at chromosome 19 q arm 13.33 encodes a member of the transmembrane 4 superfamily, also known as the tetraspanin family. Most of these members are cell-surface proteins that are characterized by the presence of four hydrophobic domains. The proteins mediate signal transduction events that play a role in the regulation of cell development, activation, growth and motility. This encoded protein is a cell surface glycoprotein that is known to complex with integrins and other transmembrane 4 superfamily proteins. It may play a role in T-cell-B-cell interactions. The Gene ID in NCBI is 951 but not limited to. Please refer to https://www.ncbi.nlm.nih.gov/gene/951.

The cancer antigen CD223 (LAG-3) is an antigen encoded by CD223 (LAG-3) gene. CD223 (LAG-3) gene localized but not limited at chromosome 12 p arm 13.31 encodes lymphocyte-activation protein 3 belonging to Ig superfamily and contains 4 extracellular Ig-like domains. The Gene ID in NCBI is 3902 but not limited to. Please refer to https://www.ncbi.nlm.nih.gov/gene/3902.

The cancer antigen CD40 is an antigen encoded by CD40 gene. CD40 gene localized but not limited at chromosome 20 q arm 13.12 encodes a receptor on antigen-presenting cells of the immune system and is essential for mediating a broad variety of immune and inflammatory responses including T cell-dependent immunoglobulin class switching, memory B cell development, and germinal center formation. The Gene ID in NCBI is 958 but not limited to. Please refer to https://www.ncbi.nlm.nih.gov/gene/958.

The cancer antigen LIV-1 (SLC39A6) is an antigen encoded by LIV-1 (SLC39A6) gene. LIV-1 (SLC39A6) gene localized but not limited at chromosome 18 q arm 12.2 encodes a protein belonging to a subfamily of proteins that show structural characteristics of zinc transporters. The Gene ID in NCBI is 25800 but not limited to. Please refer to https://www.ncbi.nlm.nih.gov/gene/25800.

The cancer antigen CD27 (TNFRSF7) is an antigen encoded by CD27 (TNFRSF7) gene. CD27 (TNFRSF7) gene localized but not limited at chromosome 12 p arm 13.31 encodes a member of the TNF-receptor superfamily. This receptor is required for generation and long-term maintenance of T cell immunity. It binds to ligand CD70, and plays a key role in regulating B-cell activation and immunoglobulin synthesis. This receptor transduces signals that lead to the activation of NF-kappaB and MAPK8/INK. Adaptor proteins TRAF2 and TRAF5 have been shown to mediate the signaling process of this receptor. The Gene ID in NCBI is 939 but not limited to. Please refer to https://www.ncbi.nlm.nih.gov/gene/939.

The cancer antigen CD276 (B7-H3) is an antigen encoded by CD276 (B7-H3) gene. CD276 (B7-H3) gene localized but not limited at chromosome 15 q arm 24.1 encodes a protein belonging to the immunoglobulin superfamily, and thought to participate in the regulation of T-cell-mediated immune response. Studies show that while the transcript of this gene is ubiquitously expressed in normal tissues and solid tumors, the protein is preferentially expressed only in tumor tissues. The Gene ID in NCBI is 80381 but not limited to. Please refer to https://www.ncbi.nlm.nih.gov/gene/80381.

The cancer antigen Trop2 is an antigen encoded by Trop2 gene. Trop2 gene localized but not limited at chromosome 1 p arm 32.1 encodes a carcinoma-associated antigen. This antigen is a cell surface receptor that transduces calcium signals. The Gene ID in NCBI is 4070 but not limited to. Please refer to https://www.ncbi.nlm.nih.gov/gene/4070.

The cancer antigen Claudin1 (CLDN1) is an antigen encoded by Claudin1 (CLDN1) gene. Claudin1 (CLDN1) gene localized but not limited at chromosome 17 p arm 13.1 encodes a member of the claudin family. Claudins are integral membrane proteins and components of tight junction strands. Tight junction strands serve as a physical barrier to prevent solutes and water from passing freely through the paracellular space between epithelial or endothelial cell sheets, and also play critical roles in maintaining cell polarity and signal transduction. Differential expression of this gene has been observed in different types of malignancies, including breast cancer, ovarian cancer, hepatocellular carcinomas, urinary tumors, prostate cancer, lung cancer, head and neck cancers, thyroid carcinomas, etc. The Gene ID in NCBI is 1366 but not limited to. Please refer to https://www.ncbi.nlm.nih.gov/gene/1366.

The cancer antigen PSMA is an antigen encoded by PSMA gene. PSMA gene localized but not limited at chromosome 11 p arm 11.12 encodes a type II transmembrane glycoprotein belonging to the M28 peptidase family. The protein acts as a glutamate carboxypeptidase on different alternative substrates, including the nutrient folate and the neuropeptide N-acetyl-1-aspartyl-1-glutamate and is expressed in a number of tissues such as prostate, central and peripheral nervous system and kidney. A mutation in this gene may be associated with impaired intestinal absorption of dietary folates, resulting in low blood folate levels and consequent hyperhomocysteinemia. Expression of this protein in the brain may be involved in a number of pathological conditions associated with glutamate excitotoxicity. In the prostate the protein is up-regulated in cancerous cells and is used as an effective diagnostic and prognostic indicator of prostate cancer. The Gene ID in NCBI is 2346 but not limited to. Please refer to https://www.ncbi.nlm.nih.gov/gene/2346.

The cancer antigen TIM-1 (HAVcr-1) is an antigen encoded by TIM-1 (HAVcr-1) gene. TIM-1 (HAVcr-1) gene localized but not limited at chromosome 5 q arm 33.3 encodes a membrane receptor for both human hepatitis A virus (HHAV) and TIMD4. The encoded protein may be involved in the moderation of asthma and allergic diseases. The Gene ID in NCBI is 26762 but not limited to. Please refer to https://www.ncbi.nlm.nih.gov/gene/26762.

The cancer antigen CEACAM5 is an antigen encoded by CEACAM5 gene. CEACAM5 gene localized but not limited at chromosome 19 q arm 13.2 encodes a cell surface glycoprotein that represents the founding member of the carcinoembryonic antigen (CEA) family of proteins. The encoded protein is used as a clinical biomarker for gastrointestinal cancers and may promote tumor development through its role as a cell adhesion molecule. Additionally, the encoded protein may regulate differentiation, apoptosis, and cell polarity. The Gene ID in NCBI is 1048 but not limited to. Please refer to https://www.ncbi.nlm.nih.gov/gene/1048.

The cancer antigen CD70 is an antigen encoded by CD70 gene. CD70 gene localized but not limited at chromosome 19 p arm 13.3 encodes a cytokine that belongs to the tumor necrosis factor (TNF) ligand family. This cytokine is a ligand for TNFRSF27/CD27. It is a surface antigen on activated, but not on resting, T and B lymphocytes. It induces proliferation of costimulated T cells, enhances the generation of cytolytic T cells, and contributes to T cell activation. This cytokine is also reported to play a role in regulating B-cell activation, cytotoxic function of natural killer cells, and immunoglobulin synthesis. The Gene ID in NCBI is 970 but not limited to. Please refer to https://www.ncbi.nlm.nih.gov/gene/970.

The cancer antigen LY6E is an antigen encoded by LY6E gene. LY6E gene localized but not limited at chromosome 8 q arm 24.3 encodes a protein whose increased expression is associated with poor survival outcome in multiple malignancies as determined by a survey of more than 130 published clinical studies of gene expression studies on cancer tissue samples and adjacent normal tissues. The Gene ID in NCBI is 4061 but not limited to. Please refer to https://www.ncbi.nlm.nih.gov/gene/4061.

The cancer antigen BCMA is an antigen encoded by BCMA gene. BCMA gene localized but not limited at chromosome 16 p arm 13.13 encodes a member of the TNF-receptor superfamily. This receptor is preferentially expressed in mature B lymphocytes, and may be important for B cell development and autoimmune response. This receptor has been shown to specifically bind to the tumor necrosis factor (ligand) superfamily, member 13b (TNFSF13B/TALL-1/BAFF), and to lead to NF-kappaB and MAPK8/INK activation. This receptor also binds to various TRAF family members, and thus may transduce signals for cell survival and proliferation. The Gene ID in NCBI is 608 but not limited to. Please refer to https://www.ncbi.nlm.nih.gov/gene/608.

The cancer antigen CD135 (FLT3) is an antigen encoded by CD135 (FLT3) gene. CD135 (FLT3) gene localized but not limited at chromosome 13 q arm 12.2 encodes a class III receptor tyrosine kinase that regulates hematopoiesis. This receptor is activated by binding of the fms-related tyrosine kinase 3 ligand to the extracellular domain, which induces homodimer formation in the plasma membrane leading to autophosphorylation of the receptor. The activated receptor kinase subsequently phosphorylates and activates multiple cytoplasmic effector molecules in pathways involved in apoptosis, proliferation, and differentiation of hematopoietic cells in bone marrow. Mutations that result in the constitutive activation of this receptor result in acute myeloid leukemia and acute lymphoblastic leukemia. The Gene ID in NCBI is 2322 but not limited to. Please refer to https://www.ncbi.nlm.nih.gov/gene/2322.

The cancer antigen APRIL is an antigen encoded by APRIL gene. APRIL gene localized but not limited at chromosome 9 q arm 22.33 encodes a member of the tumor necrosis factor superfamily whose alternative name, A PRoliferation Inducing Ligand, shares the same acronym as that for ANP32B. The Gene ID in NCBI is 10541 but not limited to. Please refer to https://www.ncbi.nlm.nih.gov/gene/10541.

The cancer antigen nectin-4 is an antigen encoded by nectin-4 gene. nectin-4 gene localized but not limited at chromosome 1 q arm 23.3 encodes a member of the nectin family. The encoded protein contains two immunoglobulin-like (Ig-like) C2-type domains and one Ig-like V-type domain. It is involved in cell adhesion through trans-homophilic and -heterophilic interactions. It is a single-pass type I membrane protein. The soluble form is produced by proteolytic cleavage at the cell surface by the metalloproteinase ADAM17/TACE. The secreted form is found in both breast tumor cell lines and breast tumor patients. Mutations in this gene are the cause of ectodermal dysplasia-syndactyly syndrome type 1, an autosomal recessive disorder. The Gene ID in NCBI is 81607 but not limited to. Please refer to https://www.ncbi.nlm.nih.gov/gene/81607.

The cancer antigen FAP is an antigen encoded by FAP gene. FAP gene localized but not limited at chromosome 2 q arm 24.2 encodes a homodimeric integral membrane gelatinase belonging to the serine protease family. It is selectively expressed in reactive stromal fibroblasts of epithelial cancers, granulation tissue of healing wounds, and malignant cells of bone and soft tissue sarcomas. This protein is thought to be involved in the control of fibroblast growth or epithelial-mesenchymal interactions during development, tissue repair, and epithelial carcinogenesis. The Gene ID in NCBI is 2191 but not limited to. Please refer to https://www.ncbi.nlm.nih.gov/gene/2191.

The cancer antigen GPC3 is an antigen encoded by GPC3 gene. GPC3 gene localized but not limited at chromosome X q arm 26.2 encodes a membrane-associated protein core substituted with a variable number of heparan sulfate chains Members of the glypican-related integral membrane proteoglycan family (GRIPS) contain a core protein anchored to the cytoplasmic membrane via a glycosyl phosphatidylinositol linkage. These proteins may play a role in the control of cell division and growth regulation. The protein encoded by this gene can bind to and inhibit the dipeptidyl peptidase activity of CD26, and it can induce apoptosis in certain cell types. Deletion mutations in this gene are associated with Simpson-Golabi-Behmel syndrome, also known as Simpson dysmorphia syndrome. The Gene ID in NCBI is 2719 but not limited to. Please refer to https://www.ncbi.nlm.nih.gov/gene/2719.

The cancer antigen FGFR3 is an antigen encoded by FGFR3 gene. FGFR3 gene localized but not limited at chromosome 4 p arm 16.3 encodes a member of the fibroblast growth factor receptor (FGFR) family, with its amino acid sequence being highly conserved between members and among divergent species. FGFR family members differ from one another in their ligand affinities and tissue distribution. A full-length representative protein would consist of an extracellular region, composed of three immunoglobulin-like domains, a single hydrophobic membrane-spanning segment and a cytoplasmic tyrosine kinase domain. The extracellular portion of the protein interacts with fibroblast growth factors, setting in motion a cascade of downstream signals, ultimately influencing mitogenesis and differentiation. This particular family member binds acidic and basic fibroblast growth hormone and plays a role in bone development and maintenance. Mutations in this gene lead to craniosynostosis and multiple types of skeletal dysplasia. The Gene ID in NCBI is 2261 but not limited to. Please refer to https://www.ncbi.nlm.nih.gov/gene/2261.

The cancer antigen ROBO1 is an antigen encoded by ROBO1 gene. ROBO1 gene localized but not limited at chromosome 3 p arm 12.3 encodes an integral membrane protein that functions in axon guidance and neuronal precursor cell migration. This receptor is activated by SLIT-family proteins, resulting in a repulsive effect on glioma cell guidance in the developing brain. The product of this gene is a member of the immunoglobulin gene superfamily. The Gene ID in NCBI is 6091 but not limited to. Please refer to https://www.ncbi.nlm.nih.gov/gene/6091.

NKG2D ligands include a diverse family of ligands that include MHC class I chain-related A and B proteins and UL-16 binding proteins, where ligand-receptor interactions can result in the activation of NK and T cells. The surface expression of these ligands is important for the recognition of stressed cells by the immune system, and thus this protein and its ligands are therapeutic targets for the treatment of immune diseases and cancers. The receptor is NKG2D encoded by KLRK1 gene localizaed but not limited at chromosome 12 p arm 13.2. The Gene ID of KLRK1 in NCBI is 22914 but not limited to. Please refer to https://www.ncbi.nlm.nih.gov/gene/22914.

The cancer antigen CD123 is an antigen encoded by CD123 gene. CD123 gene localized but not limited at chromosome X p arm 22.33 and chromosome Y p arm 11.2 encodes an interleukin 3 specific subunit of a heterodimeric cytokine receptor. The receptor is comprised of a ligand specific alpha subunit and a signal transducing beta subunit shared by the receptors for interleukin 3 (IL3), colony stimulating factor 2 (CSF2/GM-CSF), and interleukin 5 (IL5). The binding of this protein to IL3 depends on the beta subunit. The beta subunit is activated by the ligand binding, and is required for the biological activities of IL3. This gene and the gene encoding the colony stimulating factor 2 receptor alpha chain (CSF2RA) form a cytokine receptor gene cluster in a X-Y pseudoautosomal region on chromosomes X or Y. The Gene ID in NCBI is 3563 but not limited to. Please refer to https://www.ncbi.nlm.nih.gov/gene/3563.

The cancer antigen SLAMF7 (CS1/CS319) is an antigen encoded by SLAMF7 (CS1/CS319) gene. SLAMF7 (CS1/CS319) gene localized but not limited at chromosome 1 q arm 23.3 encodes a robust marker of normal plasma cells and malignant plasma cells in multiple myeloma. In contrast to CD138 (the traditional plasma cell marker), CD319/SLAMF7 is much more stable and allows robust isolation of malignant plasma cells from delayed or even cryopreserved samples. The Gene ID in NCBI is 57823 but not limited to. Please refer to https://www.ncbi.nlm.nih.gov/gene/57823.

The cancer antigen CD7 is an antigen encoded by CD7 gene. CD7 gene localized but not limited at chromosome 17 q arm 25.3 encodes a transmembrane protein which is a member of the immunoglobulin superfamily. This protein is found on thymocytes and mature T cells. It plays an essential role in T-cell interactions and also in T-cell/B-cell interaction during early lymphoid development. The Gene ID in NCBI is 924 but not limited to. Please refer to https://www.ncbi.nlm.nih.gov/gene/924.

The cancer antigen CD142 (F3 coagulation factor III) is an antigen encoded by CD142 (F3 coagulation factor III) gene. CD142 (F3 coagulation factor III) gene localized but not limited at chromosome 1 p arm 21.3 encodes coagulation factor III which is a cell surface glycoprotein. This factor enables cells to initiate the blood coagulation cascades, and it functions as the high-affinity receptor for the coagulation factor VII. The resulting complex provides a catalytic event that is responsible for initiation of the coagulation protease cascades by specific limited proteolysis. Unlike the other cofactors of these protease cascades, which circulate as nonfunctional precursors, this factor is a potent initiator that is fully functional when expressed on cell surfaces, for example, on monocytes. There are 3 distinct domains of this factor: extracellular, transmembrane, and cytoplasmic. Platelets and monocytes have been shown to express this coagulation factor under procoagulatory and proinflammatory stimuli, and a major role in HIV-associated coagulopathy has been described. The Gene ID in NCBI is 2152 but not limited to. Please refer to https://www.ncbi.nlm.nih.gov/gene/2152.

The cancer antigen CD38 is an antigen encoded by CD38 gene. CD38 gene localized but not limited at chromosome 4 p arm 15.32 encodes a non-lineage-restricted, type II transmembrane glycoprotein that synthesizes and hydrolyzes cyclic adenosine 5′-diphosphate-ribose, an intracellular calcium ion mobilizing messenger. The release of soluble protein and the ability of membrane-bound protein to become internalized indicate both extracellular and intracellular functions for the protein. This protein has an N-terminal cytoplasmic tail, a single membrane-spanning domain, and a C-terminal extracellular region with four N-glycosylation sites. Crystal structure analysis demonstrates that the functional molecule is a dimer, with the central portion containing the catalytic site. It is used as a prognostic marker for patients with chronic lymphocytic leukemia. The Gene ID in NCBI is 952 but not limited to. Please refer to https://www.ncbi.nlm.nih.gov/gene/952.

The cancer antigen CD138 (SDC1, syndecan) is an antigen encoded by CD138 (SDC1, syndecan) gene. CD138 (SDC1, syndecan) gene localized but not limited at chromosome 2 p arm 24.1 encodes a transmembrane (type I) heparan sulfate proteoglycan and is a member of the syndecan proteoglycan family. The syndecans mediate cell binding, cell signaling, and cytoskeletal organization and syndecan receptors are required for internalization of the HIV-1 tat protein. The syndecan-1 protein functions as an integral membrane protein and participates in cell proliferation, cell migration and cell-matrix interactions via its receptor for extracellular matrix proteins. Altered syndecan-1 expression has been detected in several different tumor types. While several transcript variants may exist for this gene, the full-length natures of only two have been described to date. The Gene ID in NCBI is 6382 but not limited to. Please refer to https://www.ncbi.nlm.nih.gov/gene/6382.

The cancer antigen EGFR is an antigen encoded by EGFR gene. EGFR gene localized but not limited at chromosome 7 p arm 11.2 encodes a transmembrane glycoprotein that is a member of the protein kinase superfamily. This protein is a receptor for members of the epidermal growth factor family. EGFR is a cell surface protein that binds to epidermal growth factor, thus inducing receptor dimerization and tyrosine autophosphorylation leading to cell proliferation. Mutations in this gene are associated with lung cancer. The Gene ID in NCBI is 1956 but not limited to. Please refer to https://www.ncbi.nlm.nih.gov/gene/1956.

The cancer antigen PD-1 (CD279, PDCD1, hPD-1) is an antigen encoded by PD-1 (CD279, PDCD1, hPD-1) gene. PD-1 (CD279, PDCD1, hPD-1) gene localized but not limited at chromosome 2 q arm 37.3 encodes an immune-inhibitory receptor expressed in activated T cells; it is involved in the regulation of T-cell functions, including those of effector CD8+ T cells. In addition, this protein can also promote the differentiation of CD4+ T cells into T regulatory cells. PDCD1 is expressed in many types of tumors including melanomas, and has demonstrated to play a role in anti-tumor immunity. Moreover, this protein has been shown to be involved in safeguarding against autoimmunity, however, it can also contribute to the inhibition of effective anti-tumor and anti-microbial immunity. The Gene ID in NCBI is 5133 but not limited to. Please refer to https://www.ncbi.nlm.nih.gov/gene/5133.

The cancer antigen ROR1 is an antigen encoded by ROR1 gene. ROR1 gene localized but not limited at chromosome 1 p arm 31.3 encodes a receptor tyrosine kinase-like orphan receptor that modulates neurite growth in the central nervous system. The encoded protein is a glycosylated type I membrane protein that belongs to the ROR subfamily of cell surface receptors. It is a pseudokinase that lacks catalytic activity and may interact with the non-canonical Wnt signalling pathway. This gene is highly expressed during early embryonic development but expressed at very low levels in adult tissues. Increased expression of this gene is associated with B-cell chronic lymphocytic leukaemia. The Gene ID in NCBI is 4919 but not limited to. Please refer to https://www.ncbi.nlm.nih.gov/gene/4919.

The cancer antigen CSPG4 is an antigen encoded by CSPG4 gene. CSPG4 gene localized but not limited at chromosome 15 q arm 24.2 encodes a human melanoma-associated chondroitin sulfate proteoglycan which plays a role in stabilizing cell-substratum interactions during early events of melanoma cell spreading on endothelial basement membranes. CSPG4 represents an integral membrane chondroitin sulfate proteoglycan expressed by human malignant melanoma cells. The Gene ID in NCBI is 1464 but not limited to. Please refer to https://www.ncbi.nlm.nih.gov/gene/1464.

The cancer antigen CLL-1 (CLEC12A) is an antigen encoded by CLL-1 (CLEC12A) gene. CLL-1 (CLEC12A) gene localized but not limited at chromosome 12 p arm 13.31 encodes a member of the C-type lectin/C-type lectin-like domain (CTL/CTLD) superfamily. Members of this family share a common protein fold and have diverse functions, such as cell adhesion, cell-cell signaling, glycoprotein turnover, and roles in inflammation and immune response. The protein encoded by this gene is a negative regulator of granulocyte and monocyte function. Several alternatively spliced transcript variants of this gene have been described, but the full-length nature of some of these variants has not been determined. This gene is closely linked to other CTL/CTLD superfamily members in the natural killer gene complex region on chromosome 12p13. The Gene ID in NCBI is 160364 but not limited to. Please refer to https://www.ncbi.nlm.nih.gov/gene/160364.

The cancer antigen CD147 (BSG basigin) is an antigen encoded by CD147(BSG basigin) gene. CD147 (BSG basigin) gene localized but not limited at chromosome 19 p arm 13.3 encodes a plasma membrane protein that is important in spermatogenesis, embryo implantation, neural network formation, and tumor progression. Basigin is also a member of the immunoglobulin superfamily, ubiquitously expressed in various tissues. The Gene ID in NCBI is 682 but not limited to. Please refer to https://www.ncbi.nlm.nih.gov/gene/682.

The cancer antigen PSCA is an antigen encoded by PSCA gene. PSCA gene localized but not limited at chromosome 8 q arm 24.3 encodes a glycosylphosphatidylinositol-anchored cell membrane glycoprotein. In addition to being highly expressed in the prostate it is also expressed in the bladder, placenta, colon, kidney, and stomach. This gene is up-regulated in a large proportion of prostate cancers and is also detected in cancers of the bladder and pancreas. This gene includes a polymorphism that results in an upstream start codon in some individuals; this polymorphism is thought to be associated with a risk for certain gastric and bladder cancers. The Gene ID in NCBI is 8000 but not limited to. Please refer to https://www.ncbi.nlm.nih.gov/gene/8000.

The cancer antigen EPHA2 is an antigen encoded by EPHA2 gene. EPHA2 gene localized but not limited at chromosome 1 p arm 36.13 encodes a protein that binds ephrin-A ligands. This gene belongs to the ephrin receptor subfamily of the protein-tyrosine kinase family. EPH and EPH-related receptors have been implicated in mediating developmental events, particularly in the nervous system. Receptors in the EPH subfamily typically have a single kinase domain and an extracellular region containing a Cys-rich domain and 2 fibronectin type III repeats. The ephrin receptors are divided into 2 groups based on the similarity of their extracellular domain sequences and their affinities for binding ephrin-A and ephrin-B ligands. Mutations in this gene are the cause of certain genetically-related cataract disorders. The Gene ID in NCBI is 1969 but not limited to. Please refer to https://www.ncbi.nlm.nih.gov/gene/1969.

The cancer antigen GPRC5D is an antigen encoded by GPRC5D gene. GPRC5D gene localized but not limited at chromosome 12 p arm 13.1 encodes a member of the G protein-coupled receptor family. The Gene ID in NCBI is 55507 but not limited to. Please refer to https://www.ncbi.nlm.nih.gov/gene/55507.

The cancer antigen CD133 (PROM1, AC133) is an antigen encoded by CD133 (PROM1, AC133) gene. CD133 (PROM1, AC133) gene localized but not limited at chromosome 4 p arm 15.32 encodes a pentaspan transmembrane glycoprotein. The protein localizes to membrane protrusions and is often expressed on adult stem cells, where it is thought to function in maintaining stem cell properties by suppressing differentiation. Mutations in this gene have been shown to result in retinitis pigmentosa and Stargardt disease. Expression of this gene is also associated with several types of cancer. This gene is expressed from at least five alternative promoters that are expressed in a tissue-dependent manner. The Gene ID in NCBI is 8842 but not limited to. Please refer to https://www.ncbi.nlm.nih.gov/gene/8842.

The cancer antigen B7H6 (NCR3LG1) is an antigen encoded by B7H6 (NCR3LG1) gene. B7H6 (NCR3LG1) gene localized but not limited at chromosome 11 p arm 15.1 encodes a natural killer cell cytotoxicity receptor 3. B7H6 belongs to the B7 family (see MIM 605402) and is selectively expressed on tumor cells. Interaction of B7H6 with NKp30 (NCR3; MIM 611550) results in natural killer (NK) cell activation and cytotoxicity. The Gene ID in NCBI is 374383 but not limited to. Please refer to https://www.ncbi.nlm.nih.gov/gene/374383.

The cancer antigen DSC2 (DG2, DSC3) is an antigen encoded by DSC2 (DG2, DSC3) gene. DSC2 (DG2, DSC3) gene localized but not limited at chromosome 18 q arm 12.1 encodes a member of the desmocollin protein subfamily. Desmocollins, along with desmogleins, are cadherin-like transmembrane glycoproteins that are major components of the desmosome. Desmosomes are cell-cell junctions that help resist shearing forces and are found in high concentrations in cells subject to mechanical stress. This gene is found in a cluster with other desmocollin family members on chromosome 18. Mutations in this gene are associated with arrhythmogenic right ventricular dysplasia-11, and reduced protein expression has been described in several types of cancer. The Gene ID in NCBI is 1824 but not limited to. Please refer to https://www.ncbi.nlm.nih.gov/gene/1824.

The cancer antigen AE1 (SLC4A1) is an antigen encoded by AE1 (SLC4A1) gene. AE1 (SLC4A1) gene localized but not limited at chromosome 17 q arm 21.31 encodes part of the anion exchanger (AE) family and is expressed in the erythrocyte plasma membrane, where it functions as a chloride/bicarbonate exchanger involved in carbon dioxide transport from tissues to lungs. The protein comprises two domains that are structurally and functionally distinct. The N-terminal 40 kDa domain is located in the cytoplasm and acts as an attachment site for the red cell skeleton by binding ankyrin. The glycosylated C-terminal membrane-associated domain contains 12-14 membrane spanning segments and carries out the stilbene disulphonate-sensitive exchange transport of anions. The cytoplasmic tail at the extreme C-terminus of the membrane domain binds carbonic anhydrase II. The encoded protein associates with the red cell membrane protein glycophorin A and this association promotes the correct folding and translocation of the exchanger. This protein is predominantly dimeric but forms tetramers in the presence of ankyrin. Many mutations in this gene are known in man, and these mutations can lead to two types of disease: destabilization of red cell membrane leading to hereditary spherocytosis, and defective kidney acid secretion leading to distal renal tubular acidosis. Other mutations that do not give rise to disease result in novel blood group antigens, which form the Diego blood group system. Southeast Asian ovalocytosis (SAO, Melanesian ovalocytosis) results from the heterozygous presence of a deletion in the encoded protein and is common in areas where Plasmodium falciparum malaria is endemic. One null mutation in this gene is known, resulting in very severe anemia and nephrocalcinosis. The Gene ID in NCBI is 6521 but not limited to. Please refer to https://www.ncbi.nlm.nih.gov/gene/6521.

The cancer antigen GUCY2C (GC-C, MUCIL) is an antigen encoded by GUCY2C (GC-C, MUCIL) gene. GUCY2C (GC-C, MUCIL) gene localized but not limited at chromosome 12p arm 12.3 encodes a transmembrane protein that functions as a receptor for endogenous peptides guanylin and uroguanylin, and the heat-stable E. coli enterotoxin. The encoded protein activates the cystic fibrosis transmembrane conductance regulator. Mutations in this gene are associated with familial diarrhea (autosomal dominant) and meconium ileus (autosomal recessive). The Gene ID in NCBI is 2984 but not limited to. Please refer to https://www.ncbi.nlm.nih.gov/gene/2984.

The cancer antigen CDH17 (HPT1) is an antigen encoded by CDH17 (HPT1) gene. CDH17 (HPT1) gene localized but not limited at chromosome 8 q arm 22.1 encodes a member of the cadherin superfamily, genes encoding calcium-dependent, membrane-associated glycoproteins. The encoded protein is cadherin-like, consisting of an extracellular region, containing 7 cadherin domains, and a transmembrane region but lacking the conserved cytoplasmic domain. The protein is a component of the gastrointestinal tract and pancreatic ducts, acting as an intestinal proton-dependent peptide transporter in the first step in oral absorption of many medically important peptide-based drugs. The protein may also play a role in the morphological organization of liver and intestine. The Gene ID in NCBI is 1015 but not limited to. Please refer to https://www.ncbi.nlm.nih.gov/gene/1015.

The cancer antigen HPSE is an antigen encoded by HPSE gene. HPSE gene localized but not limited at chromosome 4 q arm 21.23 encodes an enzyme that cleaves heparan sulfate proteoglycans to permit cell movement through remodeling of the extracellular matrix. In addition, this cleavage can release bioactive molecules from the extracellular matrix. Several transcript variants encoding different isoforms have been found for this gene. The Gene ID in NCBI is 10855 but not limited to. Please refer to https://www.ncbi.nlm.nih.gov/gene/10855.

The cancer antigen CD24 is an antigen encoded by CD24 gene. CD24 gene localized but not limited at chromosome 6 q arm 21 encodes a sialoglycoprotein that is expressed on mature granulocytes and B cells and modulates growth and differentiation signals to these cells. The precursor protein is cleaved to a short 32 amino acid mature peptide which is anchored via a glycosyl phosphatidylinositol (GPI) link to the cell surface. This gene was missing from previous genome assemblies, but is properly located on chromosome 6. Non-transcribed pseudogenes have been designated on chromosomes 1, 15, 20, and Y. The Gene ID in NCBI is 100133941 but not limited to. Please refer to https://www.ncbi.nlm.nih.gov/gene/100133941.

The cancer antigen MUC4(ASGP) is an antigen encoded by MUC4(ASGP) gene. MUC4(ASGP) gene localized but not limited at chromosome 3 q arm 29 encodes the major constituents of mucus, mucin, covers epithelial surfaces such as those in the trachea, colon, and cervix, that is expressed on mature granulocytes and B cells and modulates growth and differentiation signals to these cells. The precursor protein is cleaved to a short 32 amino acid mature peptide which is anchored via a glycosyl phosphatidylinositol (GPI) link to the cell surface. This gene was missing from previous genome assemblies, but is properly located on chromosome 6. Non-transcribed pseudogenes have been designated on chromosomes 1, 15, 20, and Y. The Gene ID in NCBI is 4585 but not limited to. Please refer to https://www.ncbi.nlm.nih.gov/gene/4585.

The cancer antigen AFP-L3 is an antigen encoded by AFP-L3 gene. AFP-L3 gene is Lens culinaris agglutinin (LCA)-bound isoform of alpha-fetoprotein (AFP), a substance typically used in the triple test during pregnancy and for screening chronic liver disease patients for hepatocellular carcinoma (HCC). AFP can be fractionated by affinity electrophoresis into 3 glycoforms: L1, L2, and L3 based on the reactivity with the lectin Lens culinaris agglutinin (LCA). AFP-L3 binds strongly to LCA via an additional a 1-6 fucose residue attached at the reducing terminus of N-acetylglucosamine; this is in contrast to the L1 isoform.

The cancer antigen SP17 (SPA17) is an antigen encoded by SP17 (SPA17) gene. SP17 (SPA17) gene localized but not limited at chromosome 11 q arm 24.2 encodes a protein present at the cell surface. The N-terminus has sequence similarity to human cAMP-dependent protein kinase A (PKA) type II alpha regulatory subunit (RIIa) while the C-terminus has an IQ calmodulin-binding motif. The central portion of the protein has carbohydrate binding motifs and likely functions in cell-cell adhesion. The protein was initially characterized by its involvement in the binding of sperm to the zona pellucida of the oocyte. Recent studies indicate that it is also involved in additional cell-cell adhesion functions such as immune cell migration and metastasis. A retrotransposed pseudogene is present on chromosome 10q22. The Gene ID in NCBI is 53340 but not limited to. Please refer to https://www.ncbi.nlm.nih.gov/gene/53340.

The cancer antigen DCLK1 is an antigen encoded by DCLK1 gene. DCLK1 gene localized but not limited at chromosome 13 q arm 13.3 encodes a member of the protein kinase superfamily and the doublecortin family. The protein encoded by this gene contains two N-terminal doublecortin domains, which bind microtubules and regulate microtubule polymerization, a C-terminal serine/threonine protein kinase domain, which shows substantial homology to Ca2+/calmodulin-dependent protein kinase, and a serine/proline-rich domain in between the doublecortin and the protein kinase domains, which mediates multiple protein-protein interactions. The microtubule-polymerizing activity of the encoded protein is independent of its protein kinase activity. The encoded protein is involved in several different cellular processes, including neuronal migration, retrograde transport, neuronal apoptosis and neurogenesis. This gene is up-regulated by brain-derived neurotrophic factor and associated with memory and general cognitive abilities. Multiple transcript variants generated by two alternative promoter usage and alternative splicing have been reported, but the full-length nature and biological validity of some variants have not been defined. These variants encode different isoforms, which are differentially expressed and have different kinase activities. The Gene ID in NCBI is 9201 but not limited to. Please refer to https://www.ncbi.nlm.nih.gov/gene/9201.

The cancer antigen CAIX (CA9) is an antigen encoded by CAIX (CA9) gene. CAIX (CA9) gene localized but not limited at chromosome 9 p arm 13.3 encodes a transmembrane protein and is one of only two tumor-associated carbonic anhydrase isoenzymes known. It is expressed in all clear-cell renal cell carcinoma, but is not detected in normal kidney or most other normal tissues. It may be involved in cell proliferation and transformation. The Gene ID in NCBI is 768 but not limited to. Please refer to https://www.ncbi.nlm.nih.gov/gene/768.

The cancer antigen IL13RA2 (IL13Ra2) gene localized but not limited at chromosome X q arm 23 encodes a subuint of the interleukin 13 receptor complex. This protein binds IL13 with high affinity, but lacks cytoplasmic domain, and does not appear to function as a signal mediator. It is reported to play a role in the internalization of IL13. The Gene ID in NCBI is 3598 but not limited to. Please refer to https://www.ncbi.nlm.nih.gov/gene/3598.

The cancer antigen CD56 (NCAM1) is an antigen encoded by CD56 (NCAM1) gene. CD56 (NCAM1) gene localized but not limited at chromosome 11 q arm 23.2 encodes a cell adhesion protein which is a member of the immunoglobulin superfamily. The encoded protein is involved in cell-to-cell interactions as well as cell-matrix interactions during development and differentiation. The encoded protein plays a role in the development of the nervous system by regulating neurogenesis, neurite outgrowth, and cell migration. This protein is also involved in the expansion of T lymphocytes, B lymphocytes and natural killer (NK) cells which play an important role in immune surveillance. This protein plays a role in signal transduction by interacting with fibroblast growth factor receptors, N-cadherin and other components of the extracellular matrix and by triggering signalling cascades involving FYN-focal adhesion kinase (FAK), mitogen-activated protein kinase (MAPK), and phosphatidylinositol 3-kinase (PI3K). One prominent isoform of this gene, cell surface molecule CD56, plays a role in several myeloproliferative disorders such as acute myeloid leukemia and differential expression of this gene is associated with differential disease progression. The Gene ID in NCBI is 4684 but not limited to. Please refer to https://www.ncbi.nlm.nih.gov/gene/4684.

The cancer antigen CD44v6 is an antigen encoded by CD44v6 gene. CD44v6 gene localized but not limited at chromosome 11 p arm 13 encodes a cell-surface glycoprotein involved in cell-cell interactions, cell adhesion and migration. It is a receptor for hyaluronic acid (HA) and can also interact with other ligands, such as osteopontin, collagens, and matrix metalloproteinases (MMPs). This protein participates in a wide variety of cellular functions including lymphocyte activation, recirculation and homing, hematopoiesis, and tumor metastasis. Transcripts for this gene undergo complex alternative splicing that results in many functionally distinct isoforms, however, the full length nature of some of these variants has not been determined. Alternative splicing is the basis for the structural and functional diversity of this protein, and may be related to tumor metastasis. The Gene ID in NCBI is 960 but not limited to. Please refer to https://www.ncbi.nlm.nih.gov/gene/960.

The cancer antigen Claudin-6 is an antigen encoded by Claudin-6 gene. Claudin-6 gene localized but not limited at chromosome 16 p arm 13.3 encodes a component of tight junction strands, which is a member of the claudin family. The protein is an integral membrane protein and is one of the entry cofactors for hepatitis C virus. The gene methylation may be involved in esophageal tumorigenesis. This gene is adjacent to another family member CLDN9 on chromosome 16. Tight junctions represent one mode of cell-to-cell adhesion in epithelial or endothelial cell sheets, forming continuous seals around cells and serving as a physical barrier to prevent solutes and water from passing freely through the paracellular space. These junctions are comprised of sets of continuous networking strands in the outwardly facing cytoplasmic leaflet, with complementary grooves in the inwardly facing extracytoplasmic leaflet. The Gene ID in NCBI is 9074 but not limited to. Please refer to https://www.ncbi.nlm.nih.gov/gene/9074.

The cancer antigen Glypican-1 (GPC1) is an antigen encoded by Glypican-1 (GPC1) gene. Glypican-1 (GPC1) gene localized but not limited at chromosome 2 q arm 37.3 encodes a component of cell surface heparan sulfate proteoglycans which are composed of a membrane-associated protein core substituted with a variable number of heparan sulfate chains Members of the glypican-related integral membrane proteoglycan family (GRIPS) contain a core protein anchored to the cytoplasmic membrane via a glycosyl phosphatidylinositol linkage. These proteins may play a role in the control of cell division and growth regulation. The Gene ID in NCBI is 2817 but not limited to. Please refer to https://www.ncbi.nlm.nih.gov/gene/2817.

The cancer antigen PLAP (ALPP) is an antigen encoded by PLAP (ALPP) gene. PLAP (ALPP) gene localized but not limited at chromosome 2 q arm 37.1 encodes an alkaline phosphatase, a metalloenzyme that catalyzes the hydrolysis of phosphoric acid monoesters. It belongs to a multigene family composed of four alkaline phosphatase isoenzymes. The enzyme functions as a homodimer and has a catalytic site containing one magnesium and two zinc ions, which are required for its enzymatic function. One of the main sources of this enzyme is the liver, and thus, its one of several indicators of liver injury in different clinical conditions. In pregnant women, this protein is primarily expressed in placental and endometrial tissue, however, strong ectopic expression has been detected in ovarian adenocarcinoma, serous cystadenocarcinoma, and other ovarian cancer cells. The Gene ID in NCBI is 250 but not limited to. Please refer to https://www.ncbi.nlm.nih.gov/gene/250.

The cancer antigen uPAR (PLAUR) is an antigen encoded by uPAR (PLAUR) gene. uPAR (PLAUR) gene localized but not limited at chromosome 19 q arm 13.31 encodes the receptor for urokinase plasminogen activator and, given its role in localizing and promoting plasmin formation, likely influences many normal and pathological processes related to cell-surface plasminogen activation and localized degradation of the extracellular matrix. It binds both the proprotein and mature forms of urokinase plasminogen activator and permits the activation of the receptor-bound pro-enzyme by plasmin. The protein lacks transmembrane or cytoplasmic domains and may be anchored to the plasma membrane by a glycosyl-phosphatidylinositol (GPI) moiety following cleavage of the nascent polypeptide near its carboxy-terminus. However, a soluble protein is also produced in some cell types. Alternative splicing results in multiple transcript variants encoding different isoforms. The Gene ID in NCBI is 5329 but not limited to. Please refer to https://www.ncbi.nlm.nih.gov/gene/5329.

The cancer antigen LunX (BPIFAI) is an antigen encoded by LunX (BPIFAI) gene. LunX (BPIFAI) gene localized but not limited at chromosome 20 q arm 11.21 encodes the human homolog of murine plunc, and like the mouse gene, is specifically expressed in the upper airways and nasopharyngeal regions. The encoded antimicrobial protein displays antibacterial activity against Gram-negative bacteria. It is thought to be involved in inflammatory responses to irritants in the upper airways and may also serve as a potential molecular marker for detection of micrometastasis in non-small-cell lung cancer. The Gene ID in NCBI is 51297 but not limited to. Please refer to https://www.ncbi.nlm.nih.gov/gene/51297.

The cancer antigen Folate receptor beta (FRβ, FOLR2) is an antigen encoded by Folate receptor beta (FRβ, FOLR2) gene. Folate receptor beta (FRβ, FOLR2) gene localized but not limited at chromosome 11 q arm 13.4 encodes a member of the folate receptor (FOLR) family, and these genes exist in a cluster on chromosome 11. Members of this gene family have a high affinity for folic acid and for several reduced folic acid derivatives, and they mediate delivery of 5-methyltetrahydrofolate to the interior of cells. This protein has a 68% and 79% sequence homology with the FOLR1 and FOLR3 proteins, respectively. Although this protein was originally thought to be specific to placenta, it can also exist in other tissues, and it may play a role in the transport of methotrexate in synovial macrophages in rheumatoid arthritis patients. The Gene ID in NCBI is 2350 but not limited to. Please refer to https://www.ncbi.nlm.nih.gov/gene/2350.

The cancer antigen LILRB4 (ILT3, CD85K) is an antigen encoded by LILRB4 (ILT3, CD85K) gene. LILRB4 (ILT3, CD85K) gene localized but not limited at chromosome 19 q arm 13.42 encodes a member of the leukocyte immunoglobulin-like receptor (LIR) family, which is found in a gene cluster at chromosomal region 19q13.4. The encoded protein belongs to the subfamily B class of LIR receptors which contain two or four extracellular immunoglobulin domains, a transmembrane domain, and two to four cytoplasmic immunoreceptor tyrosine-based inhibitory motifs (ITIMs). The receptor is expressed on immune cells where it binds to MHC class I molecules on antigen-presenting cells and transduces a negative signal that inhibits stimulation of an immune response. The receptor can also function in antigen capture and presentation. It is thought to control inflammatory responses and cytotoxicity to help focus the immune response and limit autoreactivity. The Gene ID in NCBI is 11006 but not limited to. Please refer to https://www.ncbi.nlm.nih.gov/gene/11006.

The cancer antigen MISIIR (Müllerian inhibiting substance type 2 receptor, AMHR2) is an antigen encoded by MISIIR (Müllerian inhibiting substance type 2 receptor, AMHR2) gene. MISIIR (Müllerian inhibiting substance type 2 receptor, AMHR2) gene localized but not limited at chromosome 12 q arm 13.13 encodes the receptor for the anti-Mullerian hormone (AMH) which, in addition to testosterone, results in male sex differentiation AMH and testosterone are produced in the testes by different cells and have different effects. Testosterone promotes the development of male genitalia while the binding of AMH to the encoded receptor prevents the development of the mullerian ducts into uterus and Fallopian tubes. Mutations in this gene are associated with persistent Mullerian duct syndrome type II. The Gene ID in NCBI is 269 but not limited to. Please refer to https://www.ncbi.nlm.nih.gov/gene/269.

The cancer antigen 5T4 (TPBG) is an antigen encoded by 5T4 (TPBG) gene. 5T4 (TPBG) gene localized but not limited at chromosome 6 q arm 14.1 encodes a leucine-rich transmembrane glycoprotein that may be involved in cell adhesion. The encoded protein is an oncofetal antigen that is specific to trophoblast cells. In adults this protein is highly expressed in many tumor cells and is associated with poor clinical outcome in numerous cancers. The Gene ID in NCBI is 7162 but not limited to. Please refer to https://www.ncbi.nlm.nih.gov/gene/7162.

The CD83 ligand is encoded by CD83 ligand gene. CD83 ligand gene localized but not limited at chromosome 6 p arm 23 encodes a single-pass type I membrane protein and member of the immunoglobulin superfamily of receptors. The encoded protein may be involved in the regulation of antigen presentation. A soluble form of this protein can bind to dendritic cells and inhibit their maturation. The Gene ID in NCBI is 9308 but not limited to. Please refer to https://www.ncbi.nlm.nih.gov/gene/9308.

The cancer antigen CD171 (L1-CAM) is an antigen encoded by CD171 (L1-CAM) gene. CD171 (L1-CAM) gene localized but not limited at chromosome X q arm 28 encodes an axonal glycoprotein belonging to the immunoglobulin supergene family. The ectodomain, consisting of several immunoglobulin-like domains and fibronectin-like repeats (type III), is linked via a single transmembrane sequence to a conserved cytoplasmic domain. This cell adhesion molecule plays an important role in nervous system development, including neuronal migration and differentiation. Mutations in the gene cause X-linked neurological syndromes known as CRASH (corpus callosum hypoplasia, retardation, aphasia, spastic paraplegia and hydrocephalus). The Gene ID in NCBI is 3897 but not limited to. Please refer to https://www.ncbi.nlm.nih.gov/gene/3897.

The cancer antigen B7-H4 (VTCN1) is an antigen encoded by B7-H4 (VTCN1) gene. B7-H4 (VTCN1) gene localized but not limited at chromosome 1 p arm 13.1-p12 encodes a protein belonging to the B7 costimulatory protein family. Proteins in this family are present on the surface of antigen-presenting cells and interact with ligand bound to receptors on the surface of T cells. Studies have shown that high levels of the encoded protein has been correlated with tumor progression. A pseudogene of this gene is located on chromosome 20. The Gene ID in NCBI is 79679 but not limited to. Please refer to https://www.ncbi.nlm.nih.gov/gene/79679.

The cancer antigen CD166 (ALCAM) is an antigen encoded by CD166 (ALCAM) gene. CD166(ALCAM) gene localized but not limited at chromosome 3 q arm 13.11 encodes activated leukocyte cell adhesion molecule (ALCAM), also known as CD166 (cluster of differentiation 166), which is a member of a subfamily of immunoglobulin receptors with five immunoglobulin-like domains (VVC2C2C2) in the extracellular domain. This protein binds to T-cell differentiation antigen CD6, and is implicated in the processes of cell adhesion and migration. The Gene ID in NCBI is 214 but not limited to. Please refer to https://www.ncbi.nlm.nih.gov/gene/214.

The cancer antigen CD13 (ANPEP) is an antigen encoded by CD13 (ANPEP) gene. CD13 (ANPEP) gene localized but not limited at chromosome 15 q arm 26.1 encodes known enzymes named aminopeptidase N. Aminopeptidase N is located in the small-intestinal and renal microvillar membrane, and also in other plasma membranes. In the small intestine aminopeptidase N plays a role in the final digestion of peptides generated from hydrolysis of proteins by gastric and pancreatic proteases. Its function in proximal tubular epithelial cells and other cell types is less clear. The large extracellular carboxyterminal domain contains a pentapeptide consensus sequence characteristic of members of the zinc-binding metalloproteinase superfamily. The enzyme was thought to be involved in the metabolism of regulatory peptides by diverse cell types, including small intestinal and renal tubular epithelial cells, macrophages, granulocytes, and synaptic membranes from the CNS. The Gene ID in NCBI is 290 but not limited to. Please refer to https://www.ncbi.nlm.nih.gov/gene/290.

The cancer antigen CD117 is an antigen encoded by CD117 gene. CD117 gene localized but not limited at chromosome 4 q arm 12 encodes a receptor tyrosine kinase. This gene was initially identified as a homolog of the feline sarcoma viral oncogene v-kit and is often referred to as proto-oncogene c-Kit. The canonical form of this glycosylated transmembrane protein has an N-terminal extracellular region with five immunoglobulin-like domains, a transmembrane region, and an intracellular tyrosine kinase domain at the C-terminus. Upon activation by its cytokine ligand, stem cell factor (SCF), this protein phosphorylates multiple intracellular proteins that play a role in in the proliferation, differentiation, migration and apoptosis of many cell types and thereby plays an important role in hematopoiesis, stem cell maintenance, gametogenesis, melanogenesis, and in mast cell development, migration and function. This protein can be a membrane-bound or soluble protein. Mutations in this gene are associated with gastrointestinal stromal tumors, mast cell disease, acute myelogenous leukemia, and piebaldism. The Gene ID in NCBI is 3815 but not limited to. Please refer to https://www.ncbi.nlm.nih.gov/gene/3815.

The cancer antigen TEM8 (ANTXR1) is an antigen encoded by TEM8 (ANTXR1) gene. TEM8 (ANTXR1) gene localized but not limited at chromosome 2 p arm 13.3 encodes a type I transmembrane protein and is a tumor-specific endothelial marker that has been implicated in colorectal cancer. The encoded protein has been shown to also be a docking protein or receptor for Bacillus anthracis toxin, the causative agent of the disease, anthrax. The binding of the protective antigen (PA) component, of the tripartite anthrax toxin, to this receptor protein mediates delivery of toxin components to the cytosol of cells. Once inside the cell, the other two components of anthrax toxin, edema factor (EF) and lethal factor (LF) disrupt normal cellular processes. The Gene ID in NCBI is 84168 but not limited to. Please refer to https://www.ncbi.nlm.nih.gov/gene/84168.

The cancer antigen CD26 (DPP4) is an antigen encoded by CD26 (DPP4) gene. CD26 (DPP4) gene localized but not limited at chromosome 2 q arm 24.2 encodes dipeptidyl peptidase 4, which is identical to adenosine deaminase complexing protein-2, and to the T-cell activation antigen CD26. It is an intrinsic type II transmembrane glycoprotein and a serine exopeptidase that cleaves X-proline dipeptides from the N-terminus of polypeptides. Dipeptidyl peptidase 4 is highly involved in glucose and insulin metabolism, as well as in immune regulation. The Gene ID in NCBI is 1803 but not limited to. Please refer to https://www.ncbi.nlm.nih.gov/gene/1803.

The cancer antigen IGF1R is an antigen encoded by IGF1R gene. IGF1R gene localized but not limited at chromosome 15 q arm 26.3 encodes a receptor binding insulin-like growth factor with a high affinity. It has tyrosine kinase activity. The insulin-like growth factor I receptor plays a critical role in transformation events. Cleavage of the precursor generates alpha and beta subunits. It is highly overexpressed in most malignant tissues where it functions as an anti-apoptotic agent by enhancing cell survival. The Gene ID in NCBI is 3480 but not limited to. Please refer to https://www.ncbi.nlm.nih.gov/gene/3480.

The cancer antigen Muc3a (MUC3A) is an antigen encoded by Muc3a (MUC3A) gene. Muc3a (MUC3A) gene localized but not limited at chromosome 7 q arm 22.1 encodes epithelial glycoproteins, some of which are secreted and some membrane bound. Each of the genes contains at least one large domain of tandemly repeated sequence that encodes the peptide sequence rich in serine and/or threonine residues, which carries most of the O-linked glycosylation. The Gene ID in NCBI is 4584 but not limited to. Please refer to https://www.ncbi.nlm.nih.gov/gene/4584.

The cancer antigen IL1RAP (IL1R3) is an antigen encoded by IL1RAP (IL1R3) gene. IL1RAP (IL1R3) gene localized but not limited at chromosome 3 q arm 28 encodes a component of the interleukin 1 receptor complex, which initiates signalling events that result in the activation of interleukin 1-responsive genes. Alternative splicing of this gene results in membrane-bound and soluble isoforms differing in their C-terminus. The ratio of soluble to membrane-bound forms increases during acute-phase induction or stress. The Gene ID in NCBI is 3556 but not limited to. Please refer to https://www.ncbi.nlm.nih.gov/gene/3556.

The cancer antigen TSLPR (CRLF2) is an antigen encoded by TSLPR (CRLF2) gene. TSLPR (CRLF2) gene localized but not limited at chromosome X p arm 22.33 and chromosome Y p arm 11.2 encodes a member of the type I cytokine receptor family. The encoded protein is a receptor for thymic stromal lymphopoietin (TSLP). Together with the interleukin 7 receptor (IL7R), the encoded protein and TSLP activate STAT3, STATS, and JAK2 pathways, which control processes such as cell proliferation and development of the hematopoietic system. Rearrangement of this gene with immunoglobulin heavy chain gene (IGH) on chromosome 14, or with P2Y purinoceptor 8 gene (P2RY8) on the same X or Y chromosomes is associated with B-progenitor acute lymphoblastic leukemia (ALL) and Down syndrome ALL. The Gene ID in NCBI is 64109 but not limited to. Please refer to https://www.ncbi.nlm.nih.gov/gene/64109.

The cancer antigen LMP1 (LMP3, PDLIM7) is an antigen encoded by LMP1 (LMP3, PDLIM7) gene. LMP1 (LMP3, PDLIM7) gene localized but not limited at chromosome 5 q arm 35.3 encodes one member of a family of proteins composed of conserved PDZ and LIM domains LIM domains are proposed to function in protein-protein recognition in a variety of contexts including gene transcription and development and in cytoskeletal interaction. The LIM domains of this protein bind to protein kinases, whereas the PDZ domain binds to actin filaments. The gene product is involved in the assembly of an actin filament-associated complex essential for transmission of ret/ptc2 mitogenic signaling. The biological function is likely to be that of an adapter, with the PDZ domain localizing the LIM-binding proteins to actin filaments of both skeletal muscle and nonmuscle tissues. The Gene ID in NCBI is 9260 but not limited to. Please refer to https://www.ncbi.nlm.nih.gov/gene/9260.

The cancer antigen Siglec7 (SIGLEC7, CD328) is an antigen encoded by Siglec7 (SIGLEC7, CD328) gene. Siglec7 (SIGLEC7, CD328) gene localized but not limited at chromosome 19 q arm 13.41 encodes a protein that in humans is encoded by the SIGLEC7 gene. SIGLEC7 has also been designated as CD328 (cluster of differentiation 328). The Gene ID in NCBI is 27036 but not limited to. Please refer to https://www.ncbi.nlm.nih.gov/gene/27036.

The cancer antigen Siglec9 (CD329) is an antigen encoded by Siglec9 (CD329) gene. Siglec9 (CD329) gene localized but not limited at chromosome 19 q arm 13.41 encodes a protein that in humans is encoded by the SIGLEC9 gene. The Gene ID in NCBI is 27180 but not limited to. Please refer to https://www.ncbi.nlm.nih.gov/gene/27180.

The cancer antigen CD1a (CD1, HTA1) is an antigen encoded by CD1a (CD1, HTA1) gene. CD1a (CD1, HTA1) gene localized but not limited at chromosome 1 q arm 23.1 encodes a member of the CD1 family of transmembrane glycoproteins, which are structurally related to the major histocompatibility complex (MHC) proteins and form heterodimers with beta-2-microglobulin. The CD1 proteins mediate the presentation of primarily lipid and glycolipid antigens of self or microbial origin to T cells. The human genome contains five CD1 family genes organized in a cluster on chromosome 1. The CD1 family members are thought to differ in their cellular localization and specificity for particular lipid ligands. The protein encoded by this gene localizes to the plasma membrane and to recycling vesicles of the early endocytic system. The Gene ID in NCBI is 909 but not limited to. Please refer to https://www.ncbi.nlm.nih.gov/gene/909.

The cancer antigen CLEC14A (C14orf27, CEG1) is an antigen encoded by CLEC14A (C14orf27, CEG1) gene. CLEC14A (C14orf27, CEG1) gene localized but not limited at chromosome 14 q arm 21.1 encodes a member of the C-type lectin/C-type lectin-like domain (CTL/CTLD) superfamily. Members of this family share a common protein fold and have diverse functions, such as cell adhesion, cell-cell signalling, glycoprotein turnover, and roles in inflammation and immune response. This family member plays a role in cell-cell adhesion and angiogenesis. It functions in filopodia formation, cell migration and tube formation. Due to its presence at higher levels in tumor endothelium than in normal tissue endothelium, it is considered to be a candidate for tumor vascular targeting. The Gene ID in NCBI is 161198 but not limited to. Please refer to https://www.ncbi.nlm.nih.gov/gene/161198.

The cancer antigen MAGE-A1 (MAGEA1) is an antigen encoded by MAGE-A1 (MAGEA1) gene. MAGE-A1 (MAGEA1) gene localized but not limited at chromosome X q arm 28 encodes a member of the MAGEA gene family. The members of this family encode proteins with 50 to 80% sequence identity to each other. The promoters and first exons of the MAGEA genes show considerable variability, suggesting that the existence of this gene family enables the same function to be expressed under different transcriptional controls. The MAGEA genes are clustered at chromosomal location Xq28. They have been implicated in some hereditary disorders, such as dyskeratosis congenita. The Gene ID in NCBI is 4100 but not limited to. Please refer to https://www.ncbi.nlm.nih.gov/gene/4100.

The cancer antigen MAGE-A4 (MAGEA4) is an antigen encoded by MAGE-A4 (MAGEA4) gene. MAGE-A4 (MAGEA4) gene localized but not limited at chromosome X q arm 28 encodes a member of the MAGEA gene family. The members of this family encode proteins with 50 to 80% sequence identity to each other. The promoters and first exons of the MAGEA genes show considerable variability, suggesting that the existence of this gene family enables the same function to be expressed under different transcriptional controls. The MAGEA genes are clustered at chromosomal location Xq28. They have been implicated in some hereditary disorders, such as dyskeratosis congenita. Several variants encoding the same protein have been found for this gene. The Gene ID in NCBI is 4103 but not limited to. Please refer to https://www.ncbi.nlm.nih.gov/gene/4103.

The cancer antigen Neurofilament M (NEFM) is an antigen encoded by Neurofilament M (NEFM) gene. Neurofilament M (NEFM) gene localized but not limited at chromosome 8 p arm 21.2 encodes the medium neurofilament protein. This protein is commonly used as a biomarker of neuronal damage. Neurofilaments are type IV intermediate filament heteropolymers composed of light, medium, and heavy chains Neurofilaments comprise the axoskeleton and functionally maintain neuronal caliber. They may also play a role in intracellular transport to axons and dendrites. The Gene ID in NCBI is 4741 but not limited to. Please refer to https://www.ncbi.nlm.nih.gov/gene/4741.

The cancer antigen 2B4 (CD244) is an antigen encoded by 2B4 (CD244) gene. 2B4 (CD244) gene localized but not limited at chromosome 1 q arm 23.3 encodes a cell surface receptor expressed on natural killer (NK) cells (and some T cells) that mediate non-major histocompatibility complex (MHC) restricted killing. The interaction between NK-cell and target cells via this receptor is thought to modulate NK-cell cytolytic activity. The Gene ID in NCBI is 51744 but not limited to. Please refer to https://www.ncbi.nlm.nih.gov/gene/51744.

The cancer antigen TACI (TNFRSF13B, CD267) is an antigen encoded by TACI (TNFRSF13B, CD267) gene. TACI (TNFRSF13B, CD267) gene localized but not limited at chromosome 17 p arm 11.2 encodes a lymphocyte-specific member of the tumor necrosis factor (TNF) receptor superfamily. It interacts with calcium-modulator and cyclophilin ligand (CAML). The protein induces activation of the transcription factors NFAT, AP1, and NF-kappa-B and plays a crucial role in humoral immunity by interacting with a TNF ligand. This gene is located within the Smith-Magenis syndrome region on chromosome 17. The Gene ID in NCBI is 23495 but not limited to. Please refer to https://www.ncbi.nlm.nih.gov/gene/23495.

The cancer antigen CD32A (FCGR2A, IGFR2, CD32) is an antigen encoded by CD32A (FCGR2A, IGFR2, CD32) gene. CD32A (FCGR2A, IGFR2, CD32) gene localized but not limited at chromosome 1 q arm 23.3 encodes one member of a family of immunoglobulin Fc receptor genes found on the surface of many immune response cells. The protein encoded by this gene is a cell surface receptor found on phagocytic cells such as macrophages and neutrophils, and is involved in the process of phagocytosis and clearing of immune complexes. The Gene ID in NCBI is 2212 but not limited to. Please refer to https://www.ncbi.nlm.nih.gov/gene/2212.

The cancer antigen AXL (ARK, UFO) is an antigen encoded by AXL (ARK, UFO) gene. AXL (ARK, UFO) gene localized but not limited at chromosome 19 q arm 13.2 encodes a member of the Tyro3-Axl-Mer (TAM) receptor tyrosine kinase subfamily. The encoded protein possesses an extracellular domain which is composed of two immunoglobulin-like motifs at the N-terminal, followed by two fibronectin type-III motifs. It transduces signals from the extracellular matrix into the cytoplasm by binding to the vitamin K-dependent protein growth arrest-specific 6 (Gas6). This gene may be involved in several cellular functions including growth, migration, aggregation and anti-inflammation in multiple cell types. The Gene ID in NCBI is 558 but not limited to. Please refer to https://www.ncbi.nlm.nih.gov/gene/558.

The cancer antigen CD80 (B7, BB1, CD28LG) is an antigen encoded by CD80 (B7, BB1, CD28LG) gene. CD80 (B7, BB1, CD28LG) gene localized but not limited at chromosome 3 q arm 13.33 encodes a membrane receptor that is activated by the binding of CD28 or CTLA-4. The activated protein induces T-cell proliferation and cytokine production. This protein can act as a receptor for adenovirus subgroup B and may play a role in lupus neuropathy. The Gene ID in NCBI is 941 but not limited to. Please refer to https://www.ncbi.nlm.nih.gov/gene/941.

The cancer antigen CD86 (B70, B&-2, CD28LG2) is an antigen encoded by CD86 (B70, B&-2, CD28LG2) gene. CD86 (B70, B&-2, CD28LG2) gene localized but not limited at chromosome 3 q arm 13.33 encodes a type I membrane protein that is a member of the immunoglobulin superfamily. This protein is expressed by antigen-presenting cells, and it is the ligand for two proteins at the cell surface of T cells, CD28 antigen and cytotoxic T-lymphocyte-associated protein 4. Binding of this protein with CD28 antigen is a costimulatory signal for activation of the T-cell. Binding of this protein with cytotoxic T-lymphocyte-associated protein 4 negatively regulates T-cell activation and diminishes the immune response. The Gene ID in NCBI is 942 but not limited to. Please refer to https://www.ncbi.nlm.nih.gov/gene/942.

The cancer antigen ROR2 (BDB, NTRKR2) is an antigen encoded by ROR2 (BDB, NTRKR2) gene. ROR2 (BDB, NTRKR2) gene localized but not limited at chromosome 9 q arm 22.31 encodes a receptor protein tyrosine kinase and type I transmembrane protein that belongs to the ROR subfamily of cell surface receptors. The protein may be involved in the early formation of the chondrocytes and may be required for cartilage and growth plate development. Mutations in this gene can cause brachydactyly type B, a skeletal disorder characterized by hypoplasia/aplasia of distal phalanges and nails. In addition, mutations in this gene can cause the autosomal recessive form of Robinow syndrome, which is characterized by skeletal dysplasia with generalized limb bone shortening, segmental defects of the spine, brachydactyly, and a dysmorphic facial appearance. The Gene ID in NCBI is 4920 but not limited to. Please refer to https://www.ncbi.nlm.nih.gov/gene/4920.

Preferably, the targeting moiety is conjugated to the first polynucleotide using a coupling group, wherein the coupling group is an NHS ester, other activated ester, an alkyl or acyl halide, a bifunctional crosslinker, or maleimide group.

Preferably, the first polynucleotide or second polynucleotide comprise a sequence selected from 20-mer poly-CA, 20-mer poly-GGTT, SEQ ID NO: 5, SEQ ID NO: 6, SEQ ID NO: 8, SEQ ID NO: 9, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, 23-mer SEQ ID NO: 7, and SEQ ID NO:10.

Preferably, the binding affinity of the targeting moiety for the biological marker is less than 250 nM. Preferably, the binding affinity of the targeting moiety for the biological marker is 5 nM, 10 nM, 40 nM, 90 nM, 130 nM or 170 nM.

Preferably, the length of the first polynucleotide or the length of the second polynucleotide are 4 nt to 500 nt. Preferably, the length of the first polynucleotide or the length of the second polynucleotide are 4, 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 42, 44, 46, 48, 50, 55, 60, 65, 70, 75, 80, 85, 90, 95, 100, 120, 160, 220, 300, 400, or 480 nt.

Preferably, the binding affinity between the first linker and the second linker is less than 250 nM. Preferably, the binding affinity between the first linker and the second linker is 2, 10, 25, 50, 62, 70, 85, 100, 102, 110, 125, 150, 162, 170, 185, 200, 202, 210, 225, or 250 nM.

Preferably, the first linker or the second linker is conjugated to a native functional group of the targeting unit or a surface of the cell, wherein the native functional group is an amino acid, a sugar, or an amine.

Preferably, the targeting moiety is a peptide, protein, or aptamer.

Preferably, the CD16 receptor is a CD16a receptor or a CD16b receptor.

Preferably, an expressed polynucleotide encoding the CD16 receptor is located on q arm of chromosome 1 at position 1q23.3.

Preferably, the cell is non-tumorigenic in an immune compromised mouse.

Preferably, after being irradiated with γ-ray, the cell is non-tumorigenic in an allogeneic subject.

Preferably, a polynucleotide encoding the CD16 receptor comprising a nucleotide sequence of SEQ ID NO:1, SEQ ID NO:2, or SEQ ID NO:19.

Preferably, the CD16 receptor comprising an amino acid sequence of SEQ ID NO:3, SEQ ID NO:4, or SEQ ID NO:20.

Preferably, the human natural killer cell further comprises an inactive tumor suppressor gene or a mutated and highly expressed oncogene.

Preferably, the human natural killer cell is capable of mediating an antibody-dependent cell cytotoxicity (ADCC) response, and the human natural killer cell is a male cell.

Preferably, the number of the human natural killer cells in the composition is at least 5×10⁵ and the human natural killer cells are in an amount equal to or more than 5% by number, based on the total number of the cells in the composition as 100%.

Preferably, the subject is a human.

Preferably, the method is for treating cancer selected form Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in feta, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma. Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, other cancer, and combinations thereof.

Preferably, the biological marker is selected form carbohydrates, glycolipids, glycoproteins, CD (cluster of differentiation) antigens present on cells of a hematopoietic lineage such as CD2, CD4, CD8, CD21, etc.), γ-glutamyltranspeptidase; an adhesion protein (e.g., ICAM-1, ICAM-2, ELAM-1, VCAM-1); hormone, growth factor, cytokine, and other ligand receptors; ion channels; and the membrane-bound form of an immunoglobulin μ. Chain.

Preferably, the CD (cluster of differentiation) antigens present on cells of a hematopoietic lineage is CD2, CD4, CD8, CD21, or other CD (cluster of differentiation) antigens.

Preferably, the adhesion protein is ICAM-1, ICAM-2, ELAM-1, VCAM-1, or other adhesion protein.

Preferably, the cell further comprising a synthetic, genetically modified and/or deliberately delivered polynucleotide encoding chimeric antigen receptor (CAR) comprising a target-binding single-chain variable fragment (scFv) against an antigen selected from CD2 (clone examples: AICD2.M1 and AICD2.M2), CD3 delta, CD3 epsilon (clone example: OKT3), CD3 gamma, CD4 (clone example: DP81), CD7 (clone example: T3-3A1), CD8a, CD8 (clone example: SK1), CD11a (ITGAL, clone example: S6F1), CD11b (ITGAM, clone example: 17aba), CD11c (ITGAX, clone example: Bu15), CD11d (ITGAD), CD 18 (ITGB2, clone example: 23F2G), CD 19 (B4, clone example: FMC63, SJ25C1), CD27 (TNFRSF7, clone example: M-T271), CD28 (clone example: KOL-2), CD29 (ITGB1, clone example: AJ2), CD30 (TNFRSF8, clone example: 5F11), CD40 (TNFRSF5, clone example: G28-5), CD48 (SLAMF2, clone example: MEM-102), CD49a (ITGA1, clone example: mAQC2), CD49d (ITGA4, clone example: P3E3), CD49f (ITGA6, clone example: FW14-14), CD66a (CEACAM1), CD66b (CEACAM8, clone example BW 250/183), CD66c (CEACAM6), CD66d (CEACAM3), CD66e (CEACAM5, clone example: CE25), CD69 (CLEC2, clone example: FN50), CD79A (B-cell antigen receptor complex-associated alpha chain, clone example: HM47), CD79B (B-cell antigen receptor complex-associated beta chain, clone example: CB3-1), CD84 (SLAMF5, clone example: CD84.1.21), CD96 (Tactile, clone example: TH-111), CD100 (SEMA4D, clone example: A8), CD103 (ITGAE, clone example: 2E7), CD134 (OX40, clone example: Ber-ACT35), CD137 (4-1BB, clone example: 4B4-1-1), CD150 (SLAMF1, clone example: A12), CD158A (KIR2DL1, clone example: HP-DM1), CD158B1 (KIR2DL2, clone example: DX27), CD158B2 (KIR2DL3, clone example: DX27), CD158C (KIR3DP1), CD158D (KIRDL4, clone example: mAb 33), CD158F1 (KIR2DL5A, clone example: UP-R1), CD158F2 (KIR2DL5B, clone example: UP-R1), CD158K (KIR3DL2), CD160 (clone example: BY55), CD162 (SELPLG, clone example: KPL-1), CD226 (DNAM1, clone example: 11A8), CD229 (SLAMF3, clone example: HLy-9.1.25), CD244 (SLAMF4, clone example: 2-69), CD247 (CD3-zeta, clone example: 6B10.2), CD258 (LIGHT, clone example: T5-39), CD268 (BAFFR, clone example: 11C1), CD270 (TNFSF14, clone example: 122), CD272 (BTLA, clone example: M1H26), CD276 (B7-H3, clone example: DCN.70), CD279 (PD-1, clone example: 4B9), CD314 (NKG2D, clone example: 1D11), CD319 (SLAMF7, clone example: 162.1), CD335 (NK-p46, clone example: 9E2), CD336 (NK-p44, clone example: P44-8), CD337 (NK-p30, clone example: P30-15), CD352 (SLAMF6, clone example: NT-7), CD353 (SLAMF8, clone example: Cr24.1), CD355 (CRTAM), CD357 (TNFRSF18, clone example: 108-17), inducible T cell co-stimulator (ICOS), LFA-1 (CD11a/CD18), NKG2C, DAP-10, ICAM-1 (clone example: 1A6), NKp80 (KLRF1, clone example: 5D12), IL-2R beta (clone example: TU27), IL-2R gamma (clone example: AE.C9), IL-7R alpha (clone example: A019D5), LFA-1, SLAMF9, LAT (clone example: W15102A), GADS Claudin-6 (GrpL), SLP-76 (LCP2, clone example: H76), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll-like receptor, HER2 (clone example: 4D5), BCMA (clone example: C11D5.3), PD-L1 (clone example: PD-L1.1), VEGFR2 (clone example: 2C6), TCR b-chain, and combinations thereof.

Preferably, a target-binding scFv against an antigen can be derived from monoclonal antibody generated in splenocytes of immunized mice, Fab phage display system, and the applications are not limited to the acquisition of scFv clones.

Preferably, proteins and DNA sequences of a target-binding scFv against an antigen can be obtained from public databases including GeneDatabase (https://www.ncbi.nlm.nih.gov/nuccore/), EMBL-EBI (https://www.ebi.ac.uk/),

ScFv against B cell maturation antigen (BCMA) derived from monoclonal antibody (clone example C11D5.3) is generated in female RBF mice immunized against BCMA-Fc/KLH conjugate protein. Clone C11D5.3 is a subclass 1 immunoglobulin G (IgG) specifically binding to naïve B cells, plasma cells, and/or memory B cells. The DNA sequence of C11D5.3 scFv is SEQ ID NO: 48 and protein sequence of C11D5.3 scFv is SEQ ID NO: 49.

ScFv against human epidermal growth factor receptor 2 (HER2) derived from monoclonal antibody (clone example 4D5) is generated in BALB/c mice immunized against intraperitoneally delivered human epidermoid carcinoma cell line A431 cells. The mice with the highest serume titer are then boosted with intravenously delivered purified A431 membrane extracts. Splenocyte derived from the immunized mice are fused with mouse myeloma line X63.Ag8.653, and clone 4D5 against HER2 is selected. The monoclonal antibody binds to extracellular domain of p185^{HER2} without cross-reactivity with other epidermal growth factor receptors. The DNA sequence of 4D5 scFv is SEQ ID NO: 50 and protein sequence of 4D5 scFv is SEQ ID NO: 51.

ScFv against intercellular adhesion molecule 1 (ICAM-1) derived from monoclonal antibody (clone example 1A6) is constructed by a structure-guided complementarity-determining region (CDR) grafting procedure. Through sequence matching of CDR region of crystal structure of antibodies fragment from PDB id. 1A3R and 1A14, the final structure of antibodies is determined by energy minimizing process provided by the software Swiss PDB. The clone-encoded plasmid is transformed into TOP10 cells, and the lysed cell pellets are passed through Ni+-chelating column for subsequent purification according to manufacturer's instruction. The DNA sequence of 1A6 scFv is SEQ ID NO: 52 and protein sequence of 1A6 scFv is SEQ ID NO: 53.

ScFv against programmed cell death protein 1 (PD-1) derived from monoclonal antibody (clone example 4B9) is generated in BALB/c mice immunized against mitomycin-treated L929-PD-1 cells. The splenocytes of immunized mice are fused with murine myeloma cell line SP2/0 cells, and clone 4B9 against PD-1 is selected by its strong reactivity against L929-PD-1 cells, not L929/mock. The DNA sequence of C11D5.3 scFv is SEQ ID NO: 54 and protein sequence of C11D5.3 scFv is SEQ ID NO: 55.

ScFv against vascular endothelial growth factor receptor 2 (VEGFR2) derived from monoclonal antibody (clone example 2C6) is isolated from a large human Fab phage display library (containing 3.7×10¹⁰ clones) by several rounds of selection against immobilized recombinant kinase inserting domain-containing receptor (KDR) protein. The DNA sequence of 2C6 scFv is SEQ ID NO: 56 and protein sequence of 2C6 scFv is SEQ ID NO: 57.

Preferably, sequences of scFv clones can be synthesized and constructed according to public databases, NCBI Nucleotide (https://www.ncbi.nlm.nih.gov/nuccore) for example.

Preferably, the cell is capable of mediating an antibody-dependent cell cytotoxicity (ADCC) response.

The present invention provides a method of treating cancer, autoimmune disease, neuronal disease, human immunodeficiency virus (HIV) infection, hematopoietic cell-related diseases, metabolic syndrome, pathogenic disease, viral infection, or bacterial infection, comprising administering a composition comprising an effective amount of a human natural killer cell to a subject in need thereof; the human natural killer cell comprises a synthetic, genetically modified and/or deliberately delivered polynucleotide encoding chimeric antigen receptor (CAR) comprising a target-binding single-chain variable fragment (scFv) against an antigen selected from CD2, CD3 delta, CD3 epsilon, CD3 gamma, CD4, CD7, CD8a, CD8, CD11a (ITGAL), CD11b (ITGAM), CD11c (ITGAX), CD11d (ITGAD), CD 18 (ITGB2), CD 19 (B4), CD27 (TNFRSF7), CD28, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll-like receptor, HER2, BCMA, PD-L1, and combinations thereof, and the human natural killer cell is (A) deposited at NPMD having the deposit number NITE BP-03017; or (B) having the following characteristics:

i) expressing a CD16 receptor,

ii) retaining its capability to proliferate after subculture for at least 3 months, and

iii) x) not including synthetic, genetically modified and/or deliberately delivered polynucleotide encoding the CD16 receptor, or y) by using ddPCR system to analyze the genomic DNA of the human natural killer cell, the ratio of CD16 F176F probe-detectable DNA molecule to CD16 F176V probe-detectable DNA molecule is equal to or higher than 1, wherein the sequence of the CD16 F176F probe is SEQ ID NO: 11 and the sequence of the CD16 F176V probe is SEQ ID NO: 12.

Preferably, the number of the human natural killer cells in the composition is at least 5×10⁵ and the human natural killer cells are in an amount equal to or more than 5% by number, based on the total number of the cells in the composition as 100%.

Preferably, the subject is a human.

Preferably, the method is for treating cancer selected form Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in feta, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma. Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, other cancer, and combinations thereof.

The present invention provides a human cell with cytotoxic capability which has the following characteristics:

i) carrying a phenotype of CD3⁻ CD56⁺ and expressing a CD16 receptor; and

ii) comprising at least an antigen-binding complex in the cell membrane, wherein the antigen-binding complex is a means for inducing the cytotoxic activity of the cell via being specifically bound by an antigen selected from cancer antigen, glycolipid, glycoprotein, cluster of differentiation antigen present on cells of a hematopoietic lineage, antigen peptide bound by major histocompatibility complex, gamma-glutamyltranspeptidase, adhesion protein, hormone, growth factor, cytokine, ligand receptor, ion channel, membrane-bound form of an immunoglobulin μ. chain, alfa-fetoprotein, C-reactive protein, chromogranin A, epithelial mucin antigen, human epithelium specific antigen, Lewis(a) antigen, multidrug resistance related protein, Neu oncogene protein, neuron specific enolase, P-glycoprotein, multidrug-resistance-related antigen, p170, multidrug-resistance-related antigen, prostate specific antigen, NCAM, ganglioside molecule, MART-1, heat shock protein, sialylTn, tyrosinase, MUC-1, HER-2/neu, KSA, PSMA, p53, RAS, EGF-R, VEGF, MAGE, or other target antigen (marker) expressed by a target cell; wherein the cell is not genetically modified from the natural killer cell having the deposit number ATCC CRL-2407.

Preferably, the CD16 receptor is a CD16a receptor or a CD16b receptor.

Preferably, an expressed polynucleotide encoding the CD16 receptor is located on q arm of chromosome 1 at position 1q23.3.

Preferably, the cell is non-tumorigenic in an immune compromised mouse.

Preferably, after being irradiated with γ-ray, the cell is non-tumorigenic in an allogeneic subject.

Preferably, an expressed polynucleotide encoding the CD16 receptor comprises a nucleotide sequence of SEQ ID NO:1, SEQ ID NO:2, or SEQ ID NO:19.

Preferably, the CD16 receptor comprises an amino acid sequence of SEQ ID NO:3, SEQ ID NO:4, or SEQ ID NO:20.

Preferably, the cell further comprises an inactive tumor suppressor gene or a mutated and highly expressed oncogene.

Preferably, the cell is capable of mediating an antibody-dependent cell cytotoxicity (ADCC) response, and the cell is a male cell.

Preferably, the cell is a natural killer cell genetically modified to express the antigen-binding complex.

Preferably, the antigen is a cancer antigen selected from HER2/neu (ERBB2), HER3 (ERBB3), EGFR, VEGF, VEGFR2, GD2, CTLA4, CD19, CD20, CD22, CD30, CD33 (Siglec-3), CD52 (CAMPATH-1 antigen), CD326 (EpCAM), CA-125 (MUC16), MMP9, DLL3, CD274 (PD-L1), CEA, MSLN (mesothelin), CA19-9, CD73, CD205 (DEC205), CD51, c-MET, TRAIL-R2, IGF-1R, CD3, MIF, folate receptor alpha (FOLR1), CSF1, OX-40, CD137, TfR, MUC1, CD25 (IL-2R), CD115 (CSF1R), IL1B, CD105 (Endoglin), KIR, CD47, CEA, IL-17A, DLL4, CD51, angiopoietin 2, neuropilin-1, CD37, CD223 (LAG-3), CD40, LIV-1 (SLC39A6), CD27 (TNFRSF7), CD276 (B7-H3), Trop2, Claudin1 (CLDN1), PSMA, TIM-1 (HAVcr-1), CEACAM5, CD70, LY6E, BCMA, CD135 (FLT3), APRIL, TF(F3), nectin-4, FAP, GPC3, FGFR3, ICAM-1 (CD54), ROBO1, NKG2D ligands, CD123, CS1/SLAMF7/CD319/CRACC, CD7, CD142 (platelet tissue factor, factor III, tissue factor), CD38, CD138, EGFR VIII, EGFR, EGFR806, EGFR family member, PD-1, ROR1, CSPG4, CLL-1 (CLEC12A), CD147, PSCA, EPHA2, GPRC5D, CD133, B7H6, DSC2, AE1 (SLC4A1), GUCY2C, CDH17, HPSE, CD24, MUC4, AFP-L3, SP17, DCLK1, CAIX (CA9), IL13RA2, IL13Ra, CD56, CD44v6, TCR beta-chain, ligands of chlorotoxin, claudin-6, claudin-18.2, EIIIB (fibronectin), Glypican-1 (GPC1), PLAP (Placental alkaline phosphatase), uPAR, HCMV glycoprotein B (gB), HLA-DR (Lym1 antibody target), tumor-associated αvβ6 integrin, LunX, integrin αvβ3, folate receptor beta (FRβ), LILRB4, MISIIR (Müllerian inhibiting substance type 2 receptor), 5T4, CD83 ligand, HBsAg, CD171 (L1-CAM), TAG72 (TAG72 (Tumour-associated glycoprotein 72)), B7-H4, CD166 (ALCAM), AC133 (PROM1), LeY, CD13 (TIM1), CD117, TEM8 (ANTXR1), CD26, IL13Ra2, IGF1R, Muc3a, IL1RAP, TSLPR (CRLF2), LMP1, Siglec7, Siglec9, Epstein-Barr Virus gp350, CD1a, CLEC14A, MAGE-A1, MAGE-A4, Neurofilament M (NEFM), HERV-K env protein, HLA-A*0201/NY-ESO-1(157-165) peptide, 2B4, TACI (TNFRSF13B), CD32A(131R), AXL, Lewis Y, CD80, CD86, ROR2, a killer-cell immunoglobulin-like receptors (KIRs), a T cell receptor, a major histocompatibility complex protein, a TNF receptor protein, an immunoglobulin protein, a cytokine receptor, an integrin, activating NK cell receptors, and combinations thereof, or the antigen-binding complex is a chimeric antigen receptor (CAR).

Preferably, the cell and the natural killer cell line NK3.3 are derived from different subjects.

Preferably, the cell is derived from a subject with a cancer.

Preferably, the cell is derived from a Caucasian male.

Preferably, the cell and the natural killer cell having the deposit number ATCC CRL-2407 are derived from the same subject.

Preferably, the cell retains its capability to proliferate after subculture for at least 1 month, 2 months, 3 months, 4 months, 5 months, 6 months, 7 months, 8 months, 9 months, 10 months, 11 months, 1 year, 2 years, 3 years, or 4 years.

Preferably, the antigen-binding complex is produced by the cell.

Preferably, the cell further exhibits IL-15 secretion capability, IL-18 secretion capability, IL-21 secretion capability, IL-2 secretion capability, or other proliferation-inducing cytokine secretion capability, or the combination thereof.

Preferably, the cell further carries a phenotype of CD2⁺.

Preferably, the cell further carries a phenotype of CD45⁺.

Preferably, the cell further carries a phenotype selected from CD4⁺, CD25⁺, NKp30⁺, NKG2D⁺, NKp44⁺, NKp46⁺, CD27⁺, OX40⁺, CD107a⁺, NKG2A⁺, PD-1⁺, TIGIT⁺, SIRPα⁺, CD158⁺ and the combination thereof.

Preferably, the antigen-binding complex comprises CD3 zeta (CD3) subunit.

Preferably, the antigen-binding complex further comprises CD28 subunit, ICOS (CD278) subunit, 4-1BB (CD137) subunit, OX40 (CD134) subunit, CD27 subunit, CD40 subunit, CD40L subunit, TLRs subunit, or other costimulatory molecule expressed by at least one of effector cells, or the combination thereof.

Preferably, the cell further comprising a synthetic, genetically modified and/or deliberately delivered polynucleotide encoding a target-binding single-chain variable fragment (scFv) against the antigen, and the target-binding single-chain variable fragment is at least a subunit of the antigen-binding complex.

Preferably, a chromosome DNA sequence of the cell is at least 90% or 95% similar to the corresponding chromosome DNA sequence of the natural killer cell deposited at NPMD having the deposit number NITE BP-03017. Preferably, a chromosome DNA sequence of the cell is at least 99%, 99.99%, or 99.995% similar to the corresponding chromosome DNA sequence of the natural killer cell deposited at NPMD having the deposit number NITE BP-03017.

Preferably, the chromosome DNA sequence is a DNA of chromosome 17, a DNA sequence of chromosome 19, a DNA sequence of chromosome 22, a DNA sequence of chromosome 4, a DNA sequence of chromosome 18, a DNA sequence of chromosome Y, or a DNA sequence of chromosome X. Preferably, the chromosome DNA sequence is a DNA sequence of chromosome 1, a DNA sequence of chromosome 2, a DNA sequence of chromosome 5, a DNA sequence of chromosome 6, a DNA sequence of chromosome 7, a DNA sequence of chromosome 8, a DNA sequence of chromosome 9, a DNA sequence of chromosome 10, a DNA sequence of chromosome 11, a DNA sequence of chromosome 12, a DNA sequence of chromosome 13, a DNA sequence of chromosome 14, a DNA sequence of chromosome 15, a DNA sequence of chromosome 16, a DNA sequence of chromosome 20, a DNA sequence of chromosome 21, or a DNA sequence of chromosome 3.

Preferably, a whole genome of the cell is at least 99.995% similar to the whole genome of the natural killer cell deposited at NPMD having the deposit number NITE BP-03017.

Preferably, the cell does not include synthetic, genetically modified and/or deliberately delivered polynucleotide encoding the CD16 receptor.

Preferably, by using ddPCR system to analyze the genomic DNA of the cell, the ratio of CD16 F176F probe-detectable DNA molecule to CD16 F176V probe-detectable DNA molecule is equal to or higher than 1, wherein the sequence of the CD16 F176F probe is SEQ ID NO: 11 and the sequence of the CD16 F176V probe is SEQ ID NO: 12.

The present invention provides a composition substantially enriched in human cells with cytotoxic capability, wherein the number of the human cells in the composition is at least 5×10⁵ and the human cells are in an amount equal to or more than 5% by number, based on the total number of the cells in the composition as 100%; the human cell has the following characteristics:

i) carrying a phenotype of CD3CD56⁺ and expressing a CD16 receptor, and

ii) comprising at least an antigen-binding complex in the cell membrane, wherein the antigen-binding complex is a means for inducing the cytotoxic activity of the cell via being specifically bound by an antigen selected from cancer antigen, glycolipid, glycoprotein, cluster of differentiation antigen present on cells of a hematopoietic lineage, antigen peptide bound by major histocompatibility complex, gamma-glutamyltranspeptidase, adhesion protein, hormone, growth factor, cytokine, ligand receptor, ion channel, membrane-bound form of an immunoglobulin μ. chain, alfa-fetoprotein, C-reactive protein, chromogranin A, epithelial mucin antigen, human epithelium specific antigen, Lewis(a) antigen, multidrug resistance related protein, Neu oncogene protein, neuron specific enolase, P-glycoprotein, multidrug-resistance-related antigen, p170, multidrug-resistance-related antigen, prostate specific antigen, NCAM, ganglioside molecule, MART-1, heat shock protein, sialylTn, tyrosinase, MUC-1, HER-2/neu, KSA, PSMA, p53, RAS, EGF-R, VEGF, MAGE, or other target antigen (marker) expressed by a target cell, wherein the cell is not genetically modified from the natural killer cell having the deposit number ATCC CRL-2407.

Preferably, the CD16 receptor is a CD16a receptor or a CD16b receptor.

Preferably, an expressed polynucleotide encoding the CD16 receptor is located on q arm of chromosome 1 at position 1q23.3.

Preferably, the human cells are non-tumorigenic in an immune compromised mouse.

Preferably, after being irradiated with γ-ray, the human cells are non-tumorigenic in an allogeneic subject.

Preferably, an expressed polynucleotide encoding the CD16 receptor comprises a nucleotide sequence of SEQ ID NO:1, SEQ ID NO:2, or SEQ ID NO:19.

Preferably, the CD16 receptor comprises an amino acid sequence of SEQ ID NO:3, SEQ ID NO:4, or SEQ ID NO:20.

Preferably, the human cell further comprises an inactive tumor suppressor gene or a mutated and highly expressed oncogene.

Preferably, the human cell is capable of mediating an antibody-dependent cell cytotoxicity (ADCC) response, and the cell is a male cell.

Preferably, the cell is a natural killer cell genetically modified to express the antigen-binding complex.

Preferably, the antigen is a cancer antigen selected from HER2/neu (ERBB2), HER3 (ERBB3), EGFR, VEGF, VEGFR2, GD2, CTLA4, CD19, CD20, CD22, CD30, CD33 (Siglec-3), CD52 (CAMPATH-1 antigen), CD326 (EpCAM), CA-125 (MUC16), MMP9, DLL3, CD274 (PD-L1), CEA, MSLN (mesothelin), CA19-9, CD73, CD205 (DEC205), CD51, c-MET, TRAIL-R2, IGF-1R, CD3, MIF, folate receptor alpha (FOLR1), CSF1, OX-40, CD137, TfR, MUC1, CD25 (IL-2R), CD115 (CSF1R), IL1B, CD105 (Endoglin), KIR, CD47, CEA, IL-17A, DLL4, CD51, angiopoietin 2, neuropilin-1, CD37, CD223 (LAG-3), CD40, LIV-1 (SLC39A6), CD27 (TNFRSF7), CD276 (B7-H3), Trop2, Claudin1 (CLDN1), PSMA, TIM-1 (HAVcr-1), CEACAM5, CD70, LY6E, BCMA, CD135 (FLT3), APRIL, TF(F3), nectin-4, FAP, GPC3, FGFR3, ICAM-1 (CD54), ROBO1, NKG2D ligands, CD123, CS1/SLAMF7/CD319/CRACC, CD7, CD142 (platelet tissue factor, factor III, tissue factor), CD38, CD138, EGFR VIII, EGFR, EGFR806, EGFR family member, PD-1, ROR1, CSPG4, CLL-1 (CLEC12A), CD147, PSCA, EPHA2, GPRC5D, CD133, B7H6, DSC2, AE1 (SLC4A1), GUCY2C, CDH17, HPSE, CD24, MUC4, AFP-L3, SP17, DCLK1, CAIX (CA9), IL13RA2, IL13Ra, CD56, CD44v6, TCR beta-chain, ligands of chlorotoxin, claudin-6, claudin-18.2, EIIIB (fibronectin), Glypican-1 (GPC1), PLAP (Placental alkaline phosphatase), uPAR, HCMV glycoprotein B (gB), HLA-DR (Lym1 antibody target), tumor-associated αvβ6 integrin, LunX, integrin αvβ3, folate receptor beta (FRβ), LILRB4, MISIIR (Müllerian inhibiting substance type 2 receptor), 5T4, CD83 ligand, HBsAg, CD171 (L1-CAM), TAG72 (TAG72 (Tumour-associated glycoprotein 72)), B7-H4, CD166 (ALCAM), AC133 (PROM1), LeY, CD13 (TIM1), CD117, TEM8 (ANTXR1), CD26, IL13Ra2, IGF1R, Muc3a, IL1RAP, TSLPR (CRLF2), LMP1, Siglec7, Siglec9, Epstein-Barr Virus gp350, CD1a, CLEC14A, MAGE-A1, MAGE-A4, Neurofilament M (NEFM), HERV-K env protein, HLA-A*0201/NY-ESO-1(157-165) peptide, 2B4, TACI (TNFRSF13B), CD32A(131R), AXL, Lewis Y, CD80, CD86, ROR2, a killer-cell immunoglobulin-like receptors (KIRs), a TNF receptor protein, an immunoglobulin protein, a cytokine receptor, an integrin, activating NK cell receptors, and combinations thereof, or the antigen-binding complex is a chimeric antigen receptor (CAR).

Preferably, the human cell and the natural killer cell line NK3.3 are derived from different subjects.

Preferably, the human cell is derived from a subject with a cancer.

Preferably, the human cell is derived from a Caucasian male.

Preferably, the human cell and the natural killer cell having the deposit number ATCC CRL-2407 are derived from the same subject.

Preferably, the human cell retains its capability to proliferate after subculture for at least 1 month, 2 months, 3 months, 4 months, 5 months, 6 months, 7 months, 8 months, 9 months, 10 months, 11 months, 1 year, 2 years, 3 years, or 4 years.

Preferably, the number of the human cells in the composition is 5×10⁵-5×10⁹.

Preferably, the number of the human cells in the composition is 1×10⁶, 1.1×10⁶, 5×10⁶, 1×10⁷, 1.1×10⁷, 5×10⁷, 5.1×10⁷, 1×10⁸, 1.1×10⁸, 5×10⁸, 5.1×10⁸, 1×10⁹, 1.1×10⁹, 5×10⁹, 1×10¹⁰, 1.1×10¹⁰, 5×10¹⁰, 1×10¹¹, 1.1×10¹¹, 5×10¹¹, 5.1×10¹¹, 1×10¹², 1.1×10¹², 5×10¹², 5.1×10¹², 1×10¹³, 1.1×10¹⁴, 5×10¹⁴, 1×10¹⁵, 1.1×10¹⁵, 5×10¹⁵, 1×10¹⁶, 1.1×10¹⁶, 5×10¹⁶, 5.1×10¹⁶, 1×10¹⁷, 1.1×10¹⁷, 5×10¹⁷, 5.1×10¹⁷, 1×10¹⁸, 1.1×10¹⁸, 5×10¹⁸, 1×10¹⁹, 1.1×10¹⁹, 5×10¹⁹, 1×10²⁰, 1.1×10²⁰, 5×10²⁰, 5.1×10²⁰, 1×10²¹, 1.1×10²¹, 5×10²¹, 5.1×10²¹, 1×10²², 1.1×10²², 5×10²², 1×10²³, 1.1×10²³, 5×10²³, 1×10²⁴, 1.1×10²⁴, 5×10²⁴, 5.1×10²⁴, 1×10²⁵, 1.1×10²⁵, 5×10²⁵, 5.1×10²⁵, 1×10²⁶, 1.1×10²⁶, 5×10²⁶, 1×10²⁷, 1.1×10²⁷, 5×10²⁷, 1×10²⁸, 1.1×10²⁸, 5×10²⁸, 5.1×10²⁸, 1×10²⁹, 1.1×10²⁹, 5×10²⁹, 5.1×10²⁹, 1×10³⁰, 1.1×10³⁰, 5×10³⁰, 1×10³¹, 1.1×10³¹, 5×10³¹, 1×10³², 1.1×10³², 5×10³², 5.1×10³², 1×10³³, 1.1×10³³, 5×10³³, 5.1×10³³, 1×10³⁴, 1.1×10³⁴, 5×10³⁴, 1×10³⁵, 1.1×10³⁵, 5×10³⁵, 1×10³⁶, 1.1×10³⁶, 5×10³⁶, 5.1×10³⁶, 1×10³⁷, 1.1×10³⁷, 5×10³⁷, 5.1×10³⁷, 1×10³⁸, 1.1×10³⁸, 5×10³⁸, 1×10³⁹, 1.1×10³⁹, 5×10³⁹, 5.1×10³⁹, 1×10⁴⁰, 1.1×10⁴⁰, 5×10⁴⁰. Preferably, the number of the human cells in the composition is 1×10⁶-1×10⁴¹.

Preferably, the total number of the human cells is 5%-100%, based on the total number of the cells in the composition as 100%. Preferably, the human cells are in an amount equal to or more than 5%, 7%, 9%, 10%, 12%, 15%, 19%, 20%, 22%, 25%, 29%, 30%, 32%, 35%, 39%, 40%, 42%, 45%, 49%, 50%, 52%, 55%, 59%, 60%, 62%, 65%, 69%, 70%, 72%, 75%, 79%, 80%, 82%, 85%, 89%, or 95% by number, based on the total number of the cells in the composition as 100%.

Preferably, the antigen-binding complex is produced by the human cell.

Preferably, the human cell further exhibits IL-15 secretion capability, IL-18 secretion capability, IL-21 secretion capability, IL-2 secretion capability, or other proliferation-inducing cytokine secretion capability, or the combination thereof.

Preferably, the human cell further carries a phenotype of CD2⁺.

Preferably, the human cell further carries a phenotype of CD45⁺.

Preferably, the human cell further carries a phenotype selected from CD4⁺, CD25⁺, NKp30⁺, NKG2D⁺, NKp44⁺, NKp46⁺, CD27⁺, OX40⁺, CD107a⁺, NKG2A⁺, PD-1⁺, TIGIT⁺, SIRPα⁺, CD 158⁺ and the combination thereof.

Preferably, the antigen-binding complex comprises CD3 zeta (CD3) subunit.

Preferably, the antigen-binding complex further comprises CD28 subunit, ICOS (CD278) subunit, 4-1BB (CD137) subunit, OX40 (CD134) subunit, CD27 subunit, CD40 subunit, CD40L subunit, TLRs subunit, or other costimulatory molecule expressed by at least one of effector cells, or the combination thereof.

Preferably, the cell further comprising a synthetic, genetically modified and/or deliberately delivered polynucleotide encoding a target-binding single-chain variable fragment (scFv) against the antigen, and the target-binding single-chain variable fragment is at least a subunit of the antigen-binding complex.

Preferably, a chromosome DNA sequence of the human cell is at least 90% or 95% similar to the corresponding chromosome DNA sequence of the natural killer cell deposited at NPMD having the deposit number NITE BP-03017.

Preferably, a chromosome DNA sequence of the cell is at least 99%, 99.99%, or 99.995% similar to the corresponding chromosome DNA sequence of the natural killer cell deposited at NPMD having the deposit number NITE BP-03017.

Preferably, the chromosome DNA sequence is a DNA of chromosome 17, a DNA sequence of chromosome 19, a DNA sequence of chromosome 22, a DNA sequence of chromosome 4, a DNA sequence of chromosome 18, a DNA sequence of chromosome Y, or a DNA sequence of chromosome X. Preferably, the chromosome DNA sequence is a DNA sequence of chromosome 1, a DNA sequence of chromosome 2, a DNA sequence of chromosome 5, a DNA sequence of chromosome 6, a DNA sequence of chromosome 7, a DNA sequence of chromosome 8, a DNA sequence of chromosome 9, a DNA sequence of chromosome 10, a DNA sequence of chromosome 11, a DNA sequence of chromosome 12, a DNA sequence of chromosome 13, a DNA sequence of chromosome 14, a DNA sequence of chromosome 15, a DNA sequence of chromosome 16, a DNA sequence of chromosome 20, a DNA sequence of chromosome 21, or a DNA sequence of chromosome 3.

Preferably, a whole genome of the cell is at least 99.995% similar to the whole genome of the natural killer cell deposited at NPMD having the deposit number NITE BP-03017.

Preferably, the cell does not include synthetic, genetically modified and/or deliberately delivered polynucleotide encoding the CD16 receptor.

Preferably, by using ddPCR system to analyze the genomic DNA of the cell, the ratio of CD16 F176F probe-detectable DNA molecule to CD16 F176V probe-detectable DNA molecule is equal to or higher than 1, wherein the sequence of the CD16 F176F probe is SEQ ID NO: 11 and the sequence of the CD16 F176V probe is SEQ ID NO: 12.

The present invention provides a method of obtaining a composition substantially enriched in human cells; the method comprising:

(a) obtaining a population of human CD16⁺ natural killer cells; and

(b) delivering a polynucleotide encoding the antigen-binding complex comprising a target-binding single-chain variable fragment (scFv) against the antigen into the human CD16⁺ natural killer cells thereby obtaining the composition substantially enriched in human cells;

wherein the human CD16⁺ natural killer cell has the following characteristics:

i) a chromosome DNA sequence of the human CD16⁺ natural killer cells is at least 90% or 95% similar to the corresponding chromosome DNA sequence of the natural killer cell deposited at NPMD having the deposit number NITE BP-03017, and

ii) not genetically modified from the natural killer cell having the deposit number ATCC CRL-2407.

Preferably, the antigen-binding complex comprises a CD3 zeta (CD3) peptide.

Preferably, the antigen-binding complex further comprises CD28 peptide, ICOS (CD278) peptide, 4-1BB (CD137) peptide, OX40 (CD134) peptide, CD27 peptide, CD40 peptide, CD40L peptide, TLRs peptide, or other peptide of costimulatory molecule expressed by at least one of effector cells, or the combination thereof.

Preferably, the number of the human cells in the composition is at least 5×10⁵, and the human cells are in an amount equal to or more than 5% by number, based on the total number of the cells in the composition as 100%.

Preferably, the number of the human cells in the composition is 5×10⁵-5×10⁹.

Preferably, the number of the human cells in the composition is 1×10⁶, 1.1×10⁶, 5×10⁶, 1×10⁷, 1.1×10⁷, 5×10⁷, 5.1×10⁷, 1×10⁸, 1.1×10⁸, 5×10⁸, 5.1×10⁸, 1×10⁹, 1.1×10⁹, 5×10⁹, 1×10¹⁰, 1.1×10¹⁰, 5×10¹⁰, 1×10¹¹, 1.1×10¹¹, 5×10¹¹, 5.1×10¹¹, 1×10¹², 1.1×10¹², 5×10¹², 5.1×10¹², 1×10¹³, 1.1×10¹⁴, 5×10¹⁴, 1×10¹⁵, 1.1×10¹⁵, 5×10¹⁵, 1×10¹⁶, 1.1×10¹⁶, 5×10¹⁶, 5.1×10¹⁶, 1×10¹⁷, 1.1×10¹⁷, 5×10¹⁷, 5.1×10¹⁷, 1×10¹⁸, 1.1×10¹⁸, 5×10¹⁸, 1×10¹⁹, 1.1×10¹⁹, 5×10¹⁹, 1×10²⁰, 1.1×10²⁰, 5×10²⁰, 5.1×10²⁰, 1×10²¹, 1.1×10²¹, 5×10²¹, 5.1×10²¹, 1×10²², 1.1×10²², 5×10²², 1×10²³, 1.1×10²³, 5×10²³, 1×10²⁴, 1.1×10²⁴, 5×10²⁴, 5.1×10²⁴, 1×10²⁵, 1.1×10²⁵, 5×10²⁵, 5.1×10²⁵, 1×10²⁶, 1.1×10²⁶, 5×10²⁶, 1×10²⁷, 1.1×10²⁷, 5×10²⁷, 1×10²⁸, 1.1×10²⁸, 5×10²⁸, 5.1×10²⁸, 1×10²⁹, 1.1×10²⁹, 5×10²⁹, 5.1×10²⁹, 1×10³⁰, 1.1×10³⁰, 5×10³⁰, 1×10³¹, 1.1×10³¹, 5×10³¹, 1×10³², 1.1×10³², 5×10³², 5.1×10³², 1×10³³, 1.1×10³³, 5×10³³, 5.1×10³³, 1×10³⁴, 1.1×10³⁴, 5×10³⁴, 1×10³⁵, 1.1×10³⁵, 5×10³⁵, 1×10³⁶, 1.1×10³⁶, 5×10³⁶, 5.1×10³⁶, 1×10³⁷, 1.1×10³⁷, 5×10³⁷, 5.1×10³⁷, 1×10³⁸, 1.1×10³⁸, 5×10³⁸, 1×10³⁹, 1.1×10³⁹, 5×10³⁹, 5.1×10³⁹, 1×10⁴⁰, 1.1×10⁴⁰, 5×10⁴⁰. Preferably, the number of the human cells in the composition is 1×10⁶-1×10⁴¹.

Preferably, the total number of the human cells is 5%-100%, based on the total number of the cells in the composition as 100%. Preferably, the human cells are in an amount equal to or more than 5%, 7%, 9%, 10%, 12%, 15%, 19%, 20%, 22%, 25%, 29%, 30%, 32%, 35%, 39%, 40%, 42%, 45%, 49%, 50%, 52%, 55%, 59%, 60%, 62%, 65%, 69%, 70%, 72%, 75%, 79%, 80%, 82%, 85%, 89%, or 95% by number, based on the total number of the cells in the composition as 100%.

Preferably, a chromosome DNA sequence of the cell is at least 99%, 99.99%, or 99.995% similar to the corresponding chromosome DNA sequence of the natural killer cell deposited at NPMD having the deposit number NITE BP-03017.

Preferably, the chromosome DNA sequence is a DNA of chromosome 17, a DNA sequence of chromosome 19, a DNA sequence of chromosome 22, a DNA sequence of chromosome 4, a DNA sequence of chromosome 18, a DNA sequence of chromosome Y, or a DNA sequence of chromosome X. Preferably, the chromosome DNA sequence is a DNA sequence of chromosome 1, a DNA sequence of chromosome 2, a DNA sequence of chromosome 5, a DNA sequence of chromosome 6, a DNA sequence of chromosome 7, a DNA sequence of chromosome 8, a DNA sequence of chromosome 9, a DNA sequence of chromosome 10, a DNA sequence of chromosome 11, a DNA sequence of chromosome 12, a DNA sequence of chromosome 13, a DNA sequence of chromosome 14, a DNA sequence of chromosome 15, a DNA sequence of chromosome 16, a DNA sequence of chromosome 20, a DNA sequence of chromosome 21, or a DNA sequence of chromosome 3.

Preferably, a whole genome of the cell is at least 99.995% similar to the whole genome of the natural killer cell deposited at NPMD having the deposit number NITE BP-03017.

The present invention provides a method of culturing and expanding the human cells; the method comprising

(x) in a container, contacting the human cells with a culture medium comprising 0.5-10 vol % human platelet lysate and 100-3000 IU/mLIL-2; and

(y) culturing the cells for multiple days;

Preferably, the container comprises a bottom for seeding cells, and the bottom is air-permeable and water-impermeable.

Preferably, the step (y) comprises substeps:

(y1) culturing the cells for at least one day; and

(y2) sub-culturing the cells for at least 1 months.

The present invention provides a method of treating cancer, tumor, autoimmune disease, neuronal disease, human immunodeficiency virus (HIV) infection, hematopoietic cell-related diseases, metabolic syndrome, pathogenic disease, viral infection, or bacterial infection, comprising administering a composition comprising an effective amount of the cell to a subject in need thereof.

Preferably, the antigen is a cancer antigen.

Preferably, the method is for treating cancer or tumor.

Preferably, the method is for treating Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in feta, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma. Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor.

Preferably, the method is for treating solid tumor.

Preferably, the method is for treating liquid tumor.

The term “oNK” refers to (a) the isolated non-transgenic human CD16⁺ natural killer cell line derived from the population of human peripheral blood natural killer cells having the deposit number ATCC CRL-2407; (b) the non-transgenic human CD16⁺ natural killer cell line obtained by culturing the cell of (a) for multiple days with the culture method disclosed in the embodiments 2.1; (c) the cell which is deposited at NPMID having the deposit number NITE BP-03017; or (d) a human natural killer cell having the following characteristics:

i) expressing a CD16 receptor;

ii) retaining its capability to proliferate after subculture for at least 3 months; and

iii) x) not including synthetic, genetically modified and/or deliberately delivered polynucleotide encoding the CD16 receptor, or y) by using ddPCR system to analyze the genomic DNA of the cell, the ratio of CD16 F176F probe-detectable DNA molecule to CD16 F176V probe-detectable DNA molecule is equal to or higher than 1, wherein the sequence of the CD16 F176F probe is SEQ ID NO: 11 and the sequence of the CD16 F176V probe is SEQ ID NO: 12

### BRIEF DESCRIPTION OF THE DRAWING

FIG. 1 is the flowchart of obtaining a CD16⁺ natural killer cell line that does not include genetically modified polynucleotide encoding the CD16 receptor.

FIG. 2A is the two-dimensional dot plot presenting the population of human peripheral blood natural killer cells without the labeling of CD16 fluorescent labeled antibody, wherein the population of human peripheral blood natural killer cells is derived from the cell population having the deposit number ATCC CRL-2407.

FIG. 2B is the two-dimensional dot plot presenting the population of human peripheral blood natural killer cells with the labeling of CD16 fluorescent labeled antibody, wherein the population of human peripheral blood natural killer cells is derived from the cell population having the deposit number ATCC CRL-2407.

FIG. 2C is the two-dimensional dot plot presenting CD16 receptor expressing cell isolated from the population of human peripheral blood natural killer cells by the labeling of CD16 fluorescent labeled antibody.

FIG. 3 is the flowchart of culturing human CD16⁺ natural killer cells.

FIG. 4 is the line graph presenting the cell viability of non-transgenic human CD16⁺ natural killer cell line after different days of culturing.

FIG. 5 is the bar chart presenting the cytotoxicity of the cultured non-transgenic human CD16⁺ natural killer cell line against different cancer cells.

FIG. 6 is the bar chart presenting the comparison of the cytotoxic function between the cultured non-transgenic human CD16⁺ natural killer cell line and the NK-92 cell line to kill cancer cells.

FIG. 7A is the bar chart presenting the comparison of the cytotoxic activity between different numbers of non-transgenic human CD16⁺ natural killer cell line to kill cancer cells.

FIG. 7B is the bar chart presenting the comparison of the cytotoxic activity between different numbers of anti-HER2 antibody-conjugated non-transgenic human CD16⁺ natural killer cell line to kill cancer cells through ADCC process.

FIG. 8 is the bar chart presenting the comparison of the cytotoxic function between the anti-HER2 antibody-conjugated non-transgenic human CD16⁺ natural killer cell line and the anti-HER2 antibody co-cultured non-transgenic human CD16⁺ natural killer cell line to kill cancer cells through ADCC process.

FIG. 9 is the bar chart presenting the comparison of genotype between the non-transgenic human CD16⁺ natural killer cell line and the CD16-transgenic NK-92 cell line.

FIG. 10A-10E illustrates the principle by which two-color FISH analysis with a CD16a receptor gene-specific test probe labeled in one color and a reference probe labeled in another color can be applied to detect transgenic, synthetic, genetically modified, or deliberately delivered DNA sequence encoding the CD16a receptor in human natural killer cells.

FIG. 11 is the bar chart presenting the cytotoxic function of non-transgenic human CD16⁺ natural killer cell line to kill cancer cells through ADCC process.

FIG. 12A is the bar chart presenting the comparison of the cytotoxic function between the non-transgenic human CD16⁺ natural killer cell line and the CD16-transgenic NK-92 cell line to kill cancer cells at different effetor (E) to target (T) ratio.

FIG. 12B is the bar chart presenting the comparison of the cytotoxic function between the non-transgenic human CD16⁺ natural killer cell line and the CD16-transgenic NK-92 cell line to kill cancer cells through ADCC process at different effetor (E) to target (T) ratio.

FIG. 13A is the line graph presenting the effect of human platelet lysate on total cell number after different days of culturing human CD16⁺ natural killer cell line.

FIG. 13B is the line graph presenting the effect of human platelet lysate on cell viability after different days of culturing human CD16⁺ natural killer cell line.

FIG. 13C is the line graph presenting the effect of human platelet lysate on maintaining the expression of CD16 after different days of culturing human CD16⁺ natural killer cell line.

FIG. 14A is the line graph presenting the effect of low concentration of IL-2 on total cell number after different days of culturing human CD16⁺ natural killer cell line.

FIG. 14B is the line graph presenting the effect of high concentration of IL-2 on total cell number after different days of culturing human CD16⁺ natural killer cell line.

FIG. 14C is the line graph presenting the effect of low concentration of IL-2 on cell viability after different days of culturing human CD16⁺ natural killer cell line.

FIG. 14D is the line graph presenting the effect of high concentration of IL-2 on cell viability after different days of culturing human CD16⁺ natural killer cell line.

FIG. 14E is the line graph presenting the effect of low concentration of IL-2 on maintaining the expression of CD16 after different days of culturing human CD16⁺ natural killer cell line.

FIG. 14F is the line graph presenting the effect of high concentration of IL-2 on maintaining the expression of CD16 after different days of culturing human CD16⁺ natural killer cell line.

FIG. 15A is the line graph presenting the effect of air-permeable container on total cell number after different days of culturing human CD16⁺ natural killer cell line.

FIG. 15B is the line graph presenting the effect of air-permeable container on cell viability after different days of culturing human CD16⁺ natural killer cell line.

FIG. 15C is the line graph presenting the effect of air-permeable container on maintaining the expression of CD16 after different days of culturing human CD16⁺ natural killer cell line.

FIG. 16A-16G demonstrate the constructions of CD19 CAR.

FIG. 17 illustrates the method of preparing oNK comprising a synthetic, genetically modified and/or purposely deliberately delivered polynucleotide encoding a chimeric antigen receptor (CAR) such as shown in FIG. 16A-16G.

FIG. 18A is the two-dimensional dot plot representing the Myc⁺ cell population with CD19 binding activity in the cultured oNK cell suspension without the transuded anti-CD19 CAR construct.

FIG. 18B is the two-dimensional dot plot representing the Myc⁺ cell population with CD19 binding activity in the cultured oNK cell suspension with the transuded anti-CD19 CAR construct.

FIG. 18C is the two-dimensional dot plot representing the isolated Myc⁺ cells with CD19 binding activity that are isolated from the cell suspension as shown in FIG. 18B by the labeling of tagged CD19 recombinant protein and fluorescence-conjugated anti-Myc antibody.

FIG. 19A is the histogram presenting the CD19 binding activity of the oNK and CAR19-oNK.

FIG. 19B is the bar chart presenting the comparison of the cytotoxic function between the oNK and CAR19-oNK to kill CD19⁺ B-cell lymphoma at different effector (E) to target (T) ratio.

FIG. 20 is the bar chart presenting the comparison of the cytotoxic function between the oNK and CAR19-oNK to kill CD19 cancer cell at different effector (E) to target (T) ratio.

FIG. 21A is the fluorescent images of tumor cells in mice on Day 4, 7, 11, 14, and 18.

FIG. 21B is the statistical analysis of luminescence shown in FIG. 21A.

FIG. 21C is the survival rate of mice shown in FIG. 21A.

FIG. 22A is the line graph presenting the cell viability, CD19 binding activity and cell surface markers of CAR19-oNK within 83 days of culturing.

FIG. 22B is the line graph presenting the proliferation of CAR19-oNK within 83 days of culturing.

FIG. 23 is the bar chart presenting the IL-15 secretion of CAR19-oNK.

FIG. 24 is the line graph presenting the effect of IL-2 on fold increase in total cell number after different days of culturing CAR19-oNK.

### DETAILED DESCRIPTION OF THE INVENTION

The following is a detailed description using the embodiments of the present invention as well as the techniques and features of the present invention, however, these embodiments are not intended to limit the invention, any changes and modifications made without departing from the spirit and scope of the invention by anyone who is familiar with this technology are intended to be included in the scope of the invention.

### Embodiment 1: Obtaining CD16⁺ Natural Killer Cell Line that does not Include Genetically Modified Polynucleotide Encoding the CD16 Receptor

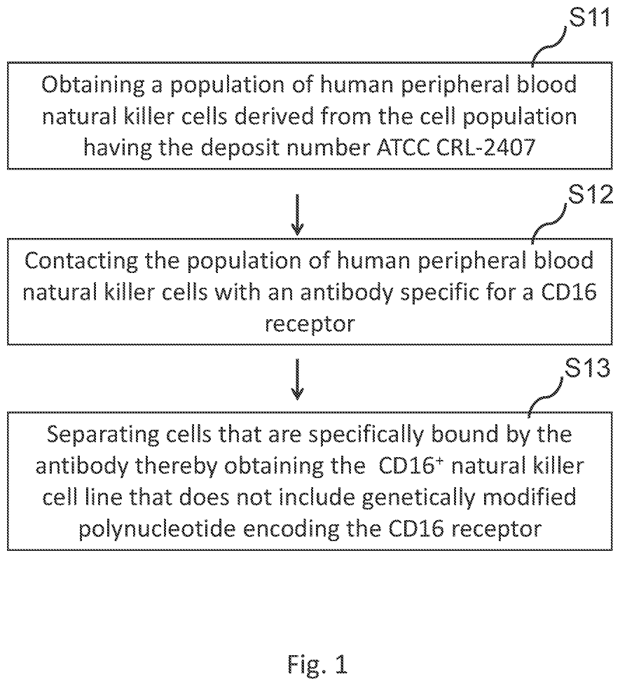

Please refer to FIG. 1. FIG. 1 is the flowchart of obtaining a CD16⁺ natural killer cell line that does not include genetically modified polynucleotide encoding the CD16 receptor. The method for obtaining a non-transgenic human CD16⁺ natural killer cell line in the present invention comprises at least the following steps:

Step S11: Obtaining a population of human peripheral blood natural killer cells derived from a cell population having the deposit number ATCC CRL-2407; Step S12: Contacting the population of human peripheral blood natural killer cells with an antibody specific for a CD16 receptor; Step S13: Separating cells that are specifically bound by the antibody thereby obtaining the CD16⁺ natural killer cell line that does not include genetically modified polynucleotide encoding the CD16 receptor.

Preferably, in Step S12, the CD16 receptor is a CD16a receptor.

Preferably, flow cytometry, bead, or any material with antibody-modified surface is used to separate the cells that are specifically bound by the antibody in Step S13.

Preferably, the term “CD16⁺ natural killer cell line that does not include genetically modified polynucleotide encoding the CD16 receptor” refers to non-genetically modified human CD16⁺ natural killer cell line and/or human CD16⁺ natural killer cell line without synthetic or exogenous polynucleotide sequence encoding the CD16 receptor.

Detailed description of preferred embodiment is elaborated below.

### Embodiment 1.1 Label and Sorting of CD16⁺ Natural Killer Cell Line that does not Include Genetically Modified Polynucleotide Encoding the CD16 Receptor

This embodiment consists of an experimental group and a control group. The population of human peripheral blood natural killer cells with the deposit number ATCC CRL-2407 was centrifuged at a speed of 100-1000×g for 3-5 minutes. The supernatant was removed, and the population of human peripheral blood natural killer cells was resuspended with a buffer. The population of human peripheral blood natural killer cells was evenly distributed into the cell culture dishes of the control group and the experimental group. The population of human peripheral blood natural killer cells of experimental group was cultured in said cell culture dishes containing cell culture medium (DMEM culture medium, alpha modification of Eagle's minimum essential medium, or XVIVO 10 culture medium), 0.5%-30% (Volume percent, vol %, v/v) Human platelet lysate, and 100-3000 IU/mL Interleukin 2 (IL-2), and then mixed with CD16 fluorescently labeled antibody (CD16-PE-Cy7, an antibody against CD16a receptor and CD16b receptor) to label the cells expressing CD16 receptor in the population of human peripheral blood natural killer cells; while the population of human peripheral blood natural killer cells of the control group was mixed with an equal volume of the buffer. The cells in the experimental group and control group were separately centrifuged, the supernatant was removed, and a sorting buffer was added to adjust the cell concentration to 1×10⁷ cells per mL. Finally, the cell population of the experimental and control groups were analyzed using a cell sorter.

Wherein, the buffer was Pre-Sort buffer, Flow cytometry sample preparation buffers, or Dulbecco's phosphate buffer saline (DPBS). The sorting buffer was Pre-Sort buffer, Flow cytometry sample preparation buffers, or Dulbecco's phosphate buffer saline (DPBS) supplemented with fetal bovine serum (FBS). The cell sorter was, for example, a flow cytometer of Becton Dickinson-FACSAria lllu model.

Preferably, the sorting buffer comprises 0.1˜10% (Volume percent, vol %, v/v) Fetal bovine serum (Fetal Bovine Serum, FBS).

Preferably, the sorting time is 1 hour, and the sorting speed is 50-70000 events/second.

After using the forward scatter (FSC) and side scatter (SSC) of the cell sorter to analyze 10,000 particles in the control group and the experimental group respectively, 6771 particles in the 10,000 particles in the control group were cells (that the amount of cells is 67.7% when the number of the total particles is 100%), and 6944 particles in the 10,000 particles in the experimental group were cells (when the number of the total particles is 100%, the amount of cells is 69.4%).

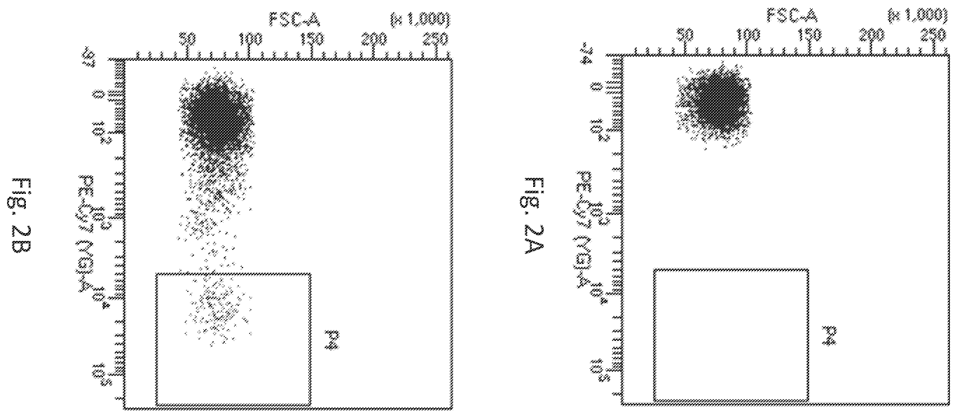

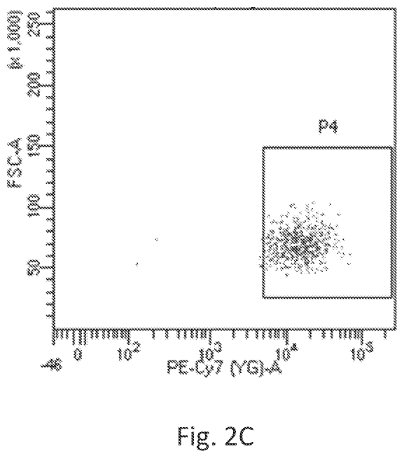

The results for fluorescent analysis of the control group cells are shown in FIG. 2A, FIG. 2A is the two-dimensional dot plot presenting the population of human peripheral blood natural killer cells without the labeling of CD16 fluorescent labeled antibody, wherein the population of human peripheral blood natural killer cells is derived from the cell population having the deposit number ATCC CRL-2407; The result for fluorescent analysis of experimental group cells are shown in FIG. 2B, FIG. 2B is the two-dimensional dot plot presenting the population of human peripheral blood natural killer cells with the labeling of CD16 fluorescent labeled antibody, wherein the population of human peripheral blood natural killer cells is derived from the cell population having the deposit number ATCC CRL-2407.

In FIG. 2A and FIG. 2B, the abscissa is the relative value of PE-Cy7 fluorescence intensity (the CD16 fluorescent labeled antibody used in this experiment emits PE-Cy7 fluorescence), and the ordinate is the relative value of forward scatter (FSC) intensity.

The results in FIG. 2A show that all of the 6771 cells analyzed in the control group did not emit PE-Cy7 fluorescence (0 cell in the rectangular region). Thus, in the absence of CD16-PE-cy7 fluorescent labeled antibody labeling, there were no other radiated light with similar wavelengths to PE-Cy7 fluorescent dye interfering the experimental result of control group cells.

The results in FIG. 2B show that most of the 6944 cells analyzed in the experimental group did not have PE-Cy7 fluorescence, and only a few cells had PE-Cy7 fluorescence (there are only 174 cells in the rectangular area). Thus, it is known that 6944 of 10,000 particles in the experimental group are cells of which 174 cells exhibit CD16 receptor, which means only 1.7% of the particles are cells expressing CD16 receptor (174÷10000=1.7%), and only 2.5%˜2.6% of the cells are cells expressing CD16 receptor (174÷6944≈2.6%). In the experimental group, based on the condition of the cell concentration is 1×10⁷ cells per mL, each mL of cell solution in experimental group contained roughly 2.6×10⁵ cells expressing the CD16 receptor.

Cells expressing the CD16 receptor were sorted from the experimental group cells in order to obtain high-purity CD16⁺ cells (hereinafter referred to as “purified CD16⁺ cell population”, “isolated oNK”, or “isolated non-transgenic human CD16⁺ natural killer cell line”.)

Please refer to FIG. 2C, FIG. 2C is the two-dimensional dot plot presenting CD16 receptor expressing cell isolated from the population of human peripheral blood natural killer cells by the labeling of CD16 fluorescent labeled antibody. The results in FIG. 2C are shown that most cells in the purified CD16⁺ cell population emit PE-Cy7 fluorescence, and the purity of the cells expressing CD16 receptor is as high as 99%.

The aforesaid cells expressing CD16 receptor in the purified CD16⁺ cell population are non-transgenic cells; all of the aforesaid cells expressing CD16 receptor in the purified CD16⁺ cell population have the feature of CD3⁻CD56⁺ after analysis, they can be continuously subcultured and are non-tumorigenic; therefore, aforesaid cell expressing CD16 receptor in the purified CD16⁺ cell population is a novel non-transgenic human CD16⁺ natural killer cell line.

### Embodiment 2: Culturing Human CD16⁺ Natural Killer Cells

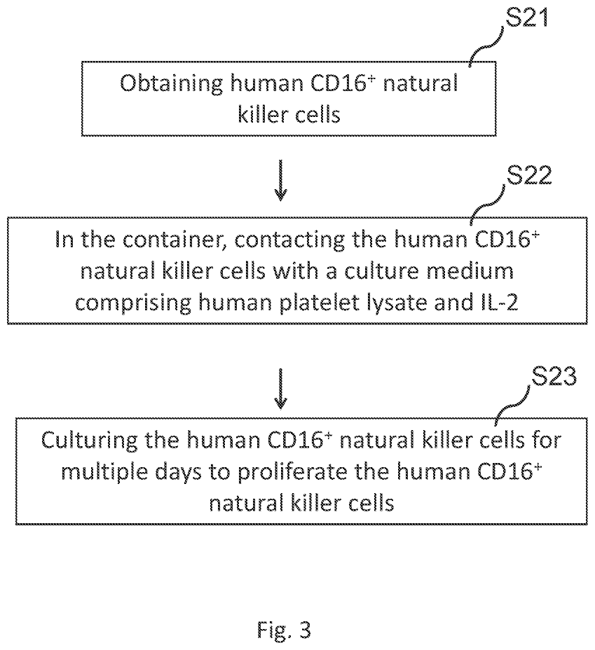

Please refer to FIG. 3. FIG. 3 is the flowchart of culturing human CD16⁺ natural killer cells. The method for culturing human CD16⁺ natural killer cells comprises at least the following steps:

Step S21: Obtaining human CD16⁺ natural killer cells;

Step S22: In the container, contacting the human CD16⁺ natural killer cells with a culture medium comprising human platelet lysate and IL-2; and

Step S23: Culturing the human CD16⁺ natural killer cells for multiple days to proliferate the human CD16⁺ natural killer cells.

The following describes a specific embodiment of culturing a non-transgenic human CD16⁺ natural killer cell line by the present invention, but the application of the invention is not limited thereto, which means the invention can also be used for culturing other human CD16⁺ natural killer cells. For example, primary CD16⁺ natural killer cell isolated from autologous or allogeneic blood, CD16-transgenic NK-92 cell line, or other human CD16⁺ natural killer cells.

### Embodiment 2.1 Culturing Non-Transgenic Human CD16⁺ Natural Killer Cell Line

Step S21′: The purified CD16⁺ cell population (the proportion of cell expressing CD16 receptor was as high as 99%) sorted by Embodiment 1 was centrifuged and the supernatant was removed.

Step S22′: after resuspending the cells with 1 mL of cell culture medium, the cell suspension was placed in a first container to make the first container contain 6 54×10⁵ non-transgenic human CD16⁺ natural killer cell line in 40 mL cell culture medium; the cell culture medium comprises: 0.5%-30% (Volume percent, vol %, v/v) Human platelet lysate; 100-3000 IU/mL Interleukin 2 (IL-2); and DMEM culture medium (Dulbecco's Modified Eagle Medium), alpha modification of Eagle's minimum essential medium, or XVIVO 10 culture medium.

Step S23′: After multiple days of culture, a composition substantially enriched in human CD16⁺ natural killer cells was obtained, and in the composition substantially enriched in human CD16⁺ natural killer cells, the number of non-transgenic human CD16⁺ natural killer cell line is at lease 5×10⁵; the multiple days are, for example, 1 day to 3 years.

Preferably, the multiple days are 1 week, 2 weeks, 3 weeks, 1 month, 2 months, 3 months, 4 months, 5 months, 6 months, 7 months, 8 months, 9 months, 10 months, 11 months, 1 year, 2 years, or 3 years.

Preferably, the cell culture medium comprises 0.5%, 1%, 1.5%, 1.6%, 2%, 2.5%, 2.6%, 3%, 3.5%, 3.6%, 4%, 4.5%, 4.6%, 5.0%, 5.1%, 5.5%, 5.6%, 6%, 6.1%, 6.5%, 6.6%, 7%, 7.1%, 7.5%, 7.6%, 8%, 8.1%, 8.5%, 8.6%, 9%, 9.1%, 9.5%, 9.6%, or 10% (Volume percent, vol %, v/v) human platelet lysate.

Preferably, the cell culture medium comprises 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, or 3000 IU/mL IL-2.

Preferably, Step S23′ further comprise substeps:

Step S231′: after multiple days of culture, the number of the cells in the cell culture medium reached the first cell number, and the first cell number was 1.25×10⁶˜5×10⁶;

Step S232′: The cell suspension was placed in a second container to make the number of cells in the second container be the first cell number; after multiple days of culture, the number of the cells reached the second cell number, and the second cell number was 5×10⁷˜1×10⁹; and

Step S233′: The cell suspension was placed in a third container to make the number of cells in the third container be the second cell number; after multiple days of culture, the number of the cells reached the third cell number in order to obtain a composition substantially enriched in human CD16⁺ natural killer cells; the third cell number was, for example, 5×10⁹, or 1×10⁴⁰.

Wherein, the first container was, for example, a T25 cell culture flask (T25 flask), or G-Rex 6-well cell culture plate. The second and third containers comprised a gas permeable but water impermeable membrane, or, the second and third container can make concentration of the dissolved oxygen fully aerated or make the concentration of the dissolved glucose in the culture medium maintain in the 1500-5000 mg/L. Preferably, the second container was, for example, G-Rex 100M bottle (Product number 81100, WILSON WOLF, USA), the third container was, for example, G-Rex-500M (Product number 85500S, WILSON WOLF, USA). Please refer to the product manual of these containers for the instruction of using G-Rex 6M 6-well cell culture plate-, G-Rex 100M bottle, and G-Rex-500M.

In the steps S23′ and S231′˜S233′, 0.5%-30% (Volume percent, vol %, v/v) Human platelet lysate and 100-3000 IU/mL Interleukin 2 (IL-2) were added to a medium for culturing the cells. And the medium is for example, DMEM culture medium (Dulbecco's Modified Eagle Medium), alpha modification of Eagle's minimum essential medium, XVIVO 10 culture medium, or X-VIVO 10 Serum-free Hemapoietic Cell Medium.

In the steps S23′ and S231′˜S233′, the cells were incubated under the condition of 37° C. and 5% carbon dioxide.

### Embodiment 2.2 Detecting Cell Viability of the Cultured Cells Obtained from Embodiment 2.1

Each sample of the cell suspensions, which were obtained by culturing for different days with the culture method disclosed in the embodiments 2.1, was mixed with an equal volume of Trypan blue, and the number of cells and the cell survival rate were observed.

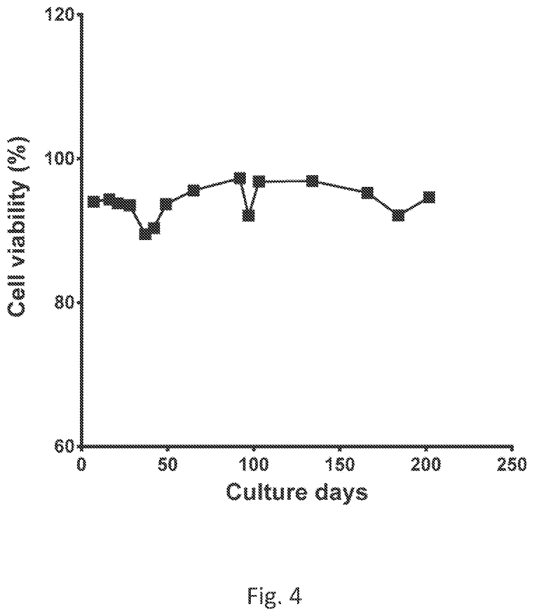

The experimental results showed that after culturing for 7, 16, 21, 28, 37, 42, 49, 65, 92, 97, 103, 134, 166, 184, and 202 days, the number of cells respectively reached 1.61×10⁶, 1.01×10⁹, 2.53×10⁹, 5.06×10⁹, 1.01×10¹⁰, 1.62×10¹⁰, 3.24×10¹⁰, 1.13×10¹¹, 1.81×10′⁵, 3.25×10¹⁶, 6.50×10¹⁷, 1.35×10²², 3.24×10²⁷, 1.30×10³³, and 1.04×10³⁹. Please refer to FIG. 4. FIG. 4 shows that cell viability was maintained at 84-97% after 7, 16, 21, 28, 37, 42, 49, 65, 92, 97, 103, 134, 166, 184, and 202 days of culture of non-transgenic human CD16⁺ natural killer cell line. Thus, culturing the non-transgenic human CD16⁺ natural killer cell line with the culture method of the present invention can make the cells number expand at lease 1.59×10³³ folds [(1.04×10³⁹)÷(6.54×10⁵)≈1.59×10³³], while effectively maintaining the cell viability rate after the proliferation.

### Embodiment 3: Detection of Cell Condition and Cell Surface Markers

### Embodiment 3.1 Long-Term Culture of Non-Transgenic Human CD16⁺ Natural Killer Cell Line by the Culture Method of the Present Invention

There are two experimental trials in this embodiment. The first batch of the purified CD16⁺ cell population and the second batch of the purified CD16⁺ cell population (the proportions of cells expressing the CD16 receptor in both of the batches were as high as 99%) were sorted by the method of Embodiment 1.1, then the first batch of the purified CD16⁺ cell population and the second batch of the purified CD16⁺ cell population were cultured respectively by the culture method of Embodiment 2.1 to obtain the cell suspensions of the first experimental trial and the cell suspensions of the second experimental trial. The first batch of the purified CD16⁺ cell population was cultured for 35 days in total, while the second batch of the purified CD16⁺ cell population was cultured for at least a long period of time until day 202.

### Embodiment 3.2 Detecting the Condition of the Cultured Cells

Each sample of the cell suspensions, which were obtained at different time points in Embodiment 3.1, was centrifuged; the supernatant was removed, the cells were resuspended in the buffer, then mix with 1 μL of propidium iodide (PI). The cell sorter or flow cytometer was used to detect whether the cells were stained with propidium iodide to determine the percentage of cells that were undergoing apoptosis or have died.

### Embodiment 3.3 Detection of CD56 CD3 and CD2 Surface Markers of the Cultured Cells

Each sample of the cell suspensions, which were obtained at different time points in Embodiment 3.1, was centrifuged; the supernatant was removed, the cells were resuspended in the buffer, then mixed with 1 μL of CD56 fluorescent labeled antibody (Cat. No. 318304, Biolegend, USA), 14, of CD3 fluorescent labeled antibody (Cat. No. 300410, Biolegend, USA), and 1 μL of CD2 fluorescent labeled antibody (Cat. No. 300222, Biolegend, USA) to simultaneously label cells expressing CD56 molecule, CD3 molecule, and/or CD2 molecule. Finally, the cell sorter or flow cytometer was used to analyze whether the cells exhibited CD56 molecules, CD3 molecules, and/or CD2 molecules, and the percentage of cells with various cell surface makers was calculated.

### Embodiment 3.4 Detection of CD16 Surface Markers of the Cultured Cells

Each sample of the cell suspensions, which were obtained at different time points in Embodiment 3.1, was centrifuged; the supernatant was removed, the cells were resuspended in the buffer, then mixed with 1 μL of CD16 fluorescent labeled antibody (Cat. No. 302016, Biolegend, USA) to label cells expressing the CD16 receptor. Finally, the cell sorter was used to analyze whether the cells exhibited CD16 receptor, and the percentage of cells with CD16 receptor was calculated.

### Embodiment 3.5 Detection of Cytotoxic Function of the Cultured Cells

xCELLigence Real Time Cell Analysis System (xCELLigence RTCA system, ACEA Biosciences Inc., USA) was used in this embodiment to detect the cytotoxic ability of the cultured cell toward target cells. This embodiment comprised a 96well xCELLigence E-Plate to carry out cytotoxicity test, and the wells in xCELLigence E-Plate were divided into control wells, experimental wells, and target cell maximum lysis control well. The effector cells used in this embodiment were the cell suspensions obtained by culturing at different time points in Embodiment 3.1, and the target cells were SK-OV-3 cell line (HTB-77, purchased from ATCC), which is an adherent ovarian cancer cell line. SK-OV-3 cells were seeded in control well, experimental well, and target cell maximum lysis control well, so that each well-contained 20000 SK-OV-3 cells, and then allowed it to sit 30 minutes.

A sample of the cell suspension obtained in Embodiment 3.1 was added to the experimental well, and the ratio of the number of effector cell to the number of SK-OV-3 cells (target cells) was 2, 5 and 10; added a tenth equal volume of lysis buffer to the sample of cell suspension into target cell maximum lysis control well; any sample or lysis buffer was not added to control well. The xCELLigence E-Plate was placed in the xCELLigence Real Time Cell Analysis System to detect real time change in the cell index (CI) under the condition of 37° C. and 5% carbon dioxide.

Wherein, the greater the number of target cells attached to the bottom of the xCELLigence E-Plate, the higher the cell index detected by the xCELLigence Real Time Cell Analysis System. Therefore, the cell index can be used to convert the percentage of target cells that are lysed in the experimental well. The formula used to convert the cell index to the percentage of target cells that are lysed in the experimental well is:

Percentage of lysed target cell (%)=1−[(cell index of experimental well−cell index of target cell maximum lysis control well)−(cell index of control well−cell index of target cell maximum lysis control well)]×100%

Please refer to Table 1 and Table 2. Table 1 shows the results of the cell suspensions obtained from the first experimental trial, and Table 2 shows the results of the cell suspension obtained from the second experimental trial.

In Table 1, the first column “day” indicates the number of culture days; the second column “PI” indicates the percentage of cells undergoing apoptosis or have died, based on the total number of the cells in the cell suspension as 100%; since natural killer cells, CD4⁺ T cells, and CD8⁺ T cells all exhibit CD56+(Pernick, N, 2018), so the third column “CD56⁺” indicates the percentage of the total number of natural killer cells, CD4⁺ T cells, and CD8⁺ T cells, based on the total number of the cells in the cell suspension as 100%; since T cells all exhibit CD3+(Pernick, N, 2018), the fourth column“CD3⁻” indicates the percentage of cells that are not T cells, based on the total number of the cells in the cell suspension as 100%; since natural killer cells, peripheral blood T cells, and most thymocytes all exhibit CD2⁺ (Pernick, N, 2018) and the cells to be bested in Embodiment 3 are derived from peripheral blood, so the fifth column “CD2⁺” indicates the percentage of the total number of natural killer cells and T cells, based on the total number of the cells in the cell suspension as 100%; the sixth column “CD56⁺CD3⁻” indicates the percentage of natural killer cells, based on the total number of the cells in the cell suspension as 100%; the seventh column “CD56⁺CD2⁺” indicates percentage of the total number of natural killer cells and T cells, based on the total number of the cells in the cell suspension as 100%; since natural killer cell and macrophage exhibit CD16⁺ (Pernick, N, 2018), and CD16 is involved in Antibody-dependent cell cytotoxicity (ADCC), the eighth column “CD16⁺” indicates the percentage of the total number of natural killer cells and macrophages with ADCC function, based on the total number of the cells in the cell suspension as 100%; the ninth column “CD56⁺CD16⁺” indicates the percentage of natural killer cells with ADCC function, based on the total number of natural killer cells (i.e., CD56⁺CD3⁻ cells) as 100%.

The indication of the first to eighth columns in Table 2 is the same as in Table 1; when the ninth column “killing test” marks “✓” symbol, this indicates that the cytotoxic function of the cells in the cell suspension at certain time point was simultaneously tested and confirmed that the cells have cytotoxic function.

Table 1 shows that (1) in the cell suspension that was obtained after the first batch of the purified CD16⁺ cell population (wherein the proportion of human CD16⁺ natural killer cell line is as high as 99%) was cultured for 7˜35 days, the percentage of cells undergoing apoptosis or have died is 5.65%˜7.34%, thus, the percentage of cell survival during culture is 92.66%−94.35%; (2) in the cell suspension that was obtained after the first batch of the purified CD16⁺ cell population was cultured for 7˜35 days, the percentage of total number of natural killer cell, CD4⁺T cell, and CD8⁺T cell is 99.08˜99.56%, based on the total number of the cells in the cell suspension as 100%; (3) in the cell suspension that was obtained after the first batch of the purified CD16⁺ cell population was cultured for 7˜35 days, the percentage of cells that are not T cells is 99.88˜100%, based on the total number of the cells in the cell suspension as 100%; (4) in the cell suspension that was obtained after the first batch of the purified CD16⁺ cell population was cultured for 7˜35 days, the percentage of total number of natural killer cell and T cell is 98.08˜99.22%, based on the total number of the cells in the cell suspension as 100%; (5) in the cell suspension that was obtained after the first batch of the purified CD16⁺ cell population was cultured for 7˜35 days, the percentage of natural killer cells is 98.21˜98.76%, based on the total number of the cells in the cell suspension as 100%; (6) in the cell suspension that was obtained after the first batch of the purified CD16⁺ cell population was cultured for 7˜35 days, the percentage of total number of natural killer cell and T cell is 98.78˜99.33%, based on the total number of the cells in the cell suspension as 100%; (7) in the cell suspension that was obtained after the first batch of the purified CD16⁺ cell population was cultured for 7˜35 days, the percentage of the total number of natural killer cells and macrophages with ADCC function is 90.17˜92.36%, based on the total number of the cells in the cell suspension as 100%; (8) in the cell suspension that was obtained after the first batch of the purified CD16⁺ cell population was cultured for 7˜35 days, the percentage of natural killer cell with ADCC function is 88.79˜92.11%, based on the total number of natural killer cell (i.e., CD56⁺CD3⁻ cell) as 100%.

TABLE 1
the test result of cell condition and cell surface marker of the cell suspension
obtained by culturing the first batch of the purified CD16⁺ cell population.

| | PI⁺ | CD56⁺ | CD3⁻ | CD2⁺ | CD56⁺CD3⁻ | CD56⁺CD2⁺ | CD16⁺ | CD56⁺/CD16⁺ |
|---|---|---|---|---|---|---|---|---|
| | (% of | (% of | (% of | (% of | (% of | (% of | (% of | (% of |
| | total | total | total | total | total | total | total | CD56⁺CD3⁻ |
| Day | cells) | cells) | cells) | cells) | cells) | cells) | cells) | cells) |
| 7 | 6.54 | 99.45 | 100 | 98.08 | 98.76 | 99.01 | 90.17 | — |
| 16 | 5.65 | 99.08 | 99.96 | 98.86 | 98.21 | 98.78 | 90.35 | — |
| 21 | 7.34 | 99.56 | 99.9 | 98.75 | 98.71 | 99.33 | 90.67 | — |
| 23 | — | — | — | — | — | — | — | 88.79 |
| 26 | — | — | — | — | — | — | — | 90.51 |
| 28 | 7.18 | 99.33 | 99.88 | 99.22 | 98.29 | 99.15 | 92.36 | — |
| 30 | — | — | — | — | — | — | — | 92.11 |
| 35 | — | — | — | — | — | — | — | 91.37 |

Table 2 shows that (1) in the cell suspension that was obtained after the second batch of the purified CD16⁺ cell population (wherein the proportion of human CD16⁺ natural killer cell line is as high as 99%) was cultured for 7-202 days, the percentage of cells undergoing apoptosis or have died is 2.7%-10.5%, thus, the percentage of cell survival during culture is 89.5%-97.3%; (2) in the cell suspension that was obtained after the second batch of the purified CD16⁺ cell population was cultured for 7-202 days, the percentage of total number of natural killer cell, CD4⁺ T cell, and CD8⁺ T cell is 98.85%-99.65%, based on the total number of the cells in the cell suspension as 100%; (3) in the cell suspension that was obtained after the second batch of the purified CD16⁺ cell population was cultured for 7-202 days, the percentage of cells that are not T cells is 99.82%-100%, based on the total number of the cells in the cell suspension as 100%; (4) in the cell suspension that was obtained after the second batch of the purified CD16⁺ cell population was cultured for 7-202 days, the percentage of total number of natural killer cell and T cell is 94.5%-99.68%, based on the total number of the cells in the cell suspension as 100%; (5) in the cell suspension that was obtained after the second batch of the purified CD16⁺ cell population was cultured for 7-202 days, the percentage of natural killer cells is 97.65%-99.05%, based on the total number of the cells in the cell suspension as 100%; (6) in the cell suspension that was obtained after the second batch of the purified CD16⁺ cell population was cultured for 7-202 days, the percentage of total number of natural killer cell and T cell is 97.83%-99.61%, based on the total number of the cells in the cell suspension as 100%; (7) in the cell suspension that was obtained after the second batch of the purified CD16⁺ cell population was cultured for 7-202 days, the percentage of the total number of natural killer cells and macrophages with ADCC function is 83.88%-94.04%%, based on the total number of the cells in the cell suspension as 100%; (8) The cell in the cell suspension that was obtained after the second batch of the purified CD16⁺ cell population was cultured for 7-202 days was confirmed to have cytotoxic function.

The cell suspension obtained by culturing for 28 days with the culture method disclosed in the embodiment 2.1 has been deposited at NPMD with the deposit number NITE BP-03017. The results disclosed in this invention indicate that the oNK cell line could retain its capability to proliferate after subculture for at least 3 months and thus may comprised deregulated genes responsible for cell growth control (e.g. the oNK cell line may comprised an inactive tumor suppressor gene, or a mutated and highly expressed oncogene).

TABLE 2
the test results of cell condition, cell surface marker and cytotoxicity of the cell suspension
obtained by culturing the second batch of the purified CD16⁺ cell population.

| | | PI⁺ | CD56⁺ | CD3⁻ | CD2⁺ | CD56⁺CD3⁻ | CD56⁺CD2⁺ | CD16⁺ | |
|---|---|---|---|---|---|---|---|---|---|
| | | (% of | (% of | (% of | (% of | (% of | (% of | (% of | |
| | Total cell | total | total | total | total | total | total | total | Killing |
| Day | number | cells) | cells) | cells) | cells) | cells) | cells) | cells) | test |
| 7 | 1.61 × 10⁶ | 5.97 | 99.45 | 100.00 | 98.08 | 98.76 | 99.01 | 90.17 | |
| 16 | 1.01 × 10⁹ | 5.65 | 99.09 | 99.96 | 98.86 | 98.21 | 98.8 | 90.36 | |
| 21 | 2.53 × 10⁹ | 6.2 | 99.56 | 99.91 | 98.75 | 98.72 | 99.33 | 90.7 | |
| 28 | 5.06 × 10⁹ | 6.46 | 99.33 | 99.88 | 99.22 | 98.29 | 99.15 | 92.36 | |
| 37 | 1.01 × 10¹⁰ | 10.5 | 98.85 | 99.99 | 98.48 | 97.65 | 98.66 | 91.96 | |
| 42 | 1.62 × 10¹⁰ | 9.63 | 99.15 | 100.00 | 98.24 | 98.06 | 98.8 | 93.09 | |
| 49 | 3.24 × 10¹⁰ | 6.31 | 98.99 | 100.00 | 94.5 | 97.71 | 97.83 | 94.04 | |
| 65 | 1.13 × 10¹¹ | 4.41 | 99.15 | 99.99 | 98.55 | 97.81 | 98.85 | 90.35 | |
| 92 | 1.81 × 10¹⁵ | 2.7 | 99.62 | 99.99 | 99.43 | 98.58 | 99.42 | 85.99 | ✓ |
| 97 | 3.25 × 10¹⁶ | 7.91 | 99.23 | 99.90 | 99.58 | 98.3 | 99.05 | 86.98 | ✓ |
| 103 | 6.50 × 10¹⁷ | 3.17 | 99.65 | 99.82 | 99.5 | 98.71 | 99.45 | 83.88 | ✓ |
| 134 | 1.35 × 10²² | 3.09 | 99.62 | 99.99 | 99.68 | 98.75 | 99.42 | 86.18 | ✓ |
| 166 | 3.24 × 10²⁷ | 4.74 | 99.17 | 100.00 | 99.06 | 99.05 | 99.61 | 89.93 | ✓ |
| 184 | 1.30 × 10³³ | 7.87 | 99.61 | 99.99 | 98.23 | 98.77 | 99.37 | 92.38 | ✓ |
| 202 | 1.04 × 10³⁹ | 5.36 | 99.59 | 99.96 | 97.52 | 98.94 | 99.33 | 93.02 | ✓ |

### Embodiment 3.6 Detection of Activation Markers, Inhibitory Markers, and Other NK Cell Markers of the Cultured Cells

Cell suspensions obtained by culturing for 93 days with the culture method disclosed in the embodiments 2.1 (refer to as 93-day cultured oNK suspension) were used in this embodiment. Cells in the Cell suspensions were evenly assigned into 19 groups. Cells in the first group were centrifuged; the supernatant was removed, the cells were resuspended in the buffer, then mixed with 1 μL of CD56 fluorescent labeled antibody (Cat. No. 318304, Biolegend, USA), 1 L of CD3 fluorescent labeled antibody (Cat. No. 300410, Biolegend, USA), and 1 μL of CD2 fluorescent labeled antibody (Cat. No. 300222, Biolegend, USA) to simultaneously label cells expressing CD56 molecule, CD3 molecule, and/or CD2 molecule. Finally, the cell sorter or flow cytometer was used to analyze whether the cells exhibited CD56 molecules, CD3 molecules, and/or CD2 molecules, and the percentage of cells with various cell surface makers was calculated.

Cells in the other 18 groups were centrifuged; the supernatant was removed, the cells were resuspended in the buffer, then respectively mixed with 1 μL of CD16 fluorescent labeled antibody (Cat. No. 302016, Biolegend, USA), CD45 fluorescent labeled antibody (Cat. No. 368512, Biolegend, USA), CD4 fluorescent labeled antibody (Cat. No. 300514, Biolegend, USA), CD8 fluorescent labeled antibody (Cat. No. 344706, Biolegend, USA), CD19 fluorescent labeled antibody (Cat. No. 302210, Biolegend, USA), CD25 fluorescent labeled antibody (Cat. No. 302614, Biolegend, USA), NKp30 fluorescent labeled antibody (Cat. No. 325214, Biolegend, USA), NKG2D fluorescent labeled antibody (Cat. No. 320812, Biolegend, USA), NKp44 fluorescent labeled antibody (Cat. No. 325116, Biolegend, USA), NKp46 fluorescent labeled antibody (Cat. No. 331916, Biolegend, USA), CD27 fluorescent labeled antibody (Cat. No. 47-0279-42, Invitrogen, USA), OX40 fluorescent labeled antibody (Cat. No. 350004, Biolegend, USA), CD107a fluorescent labeled antibody (Cat. No. 328630, Biolegend, USA), NKG2A fluorescent labeled antibody (Cat. No. FAB1059P, R&D Systems, USA), PD-1 fluorescent labeled antibody (Cat. No. 367406, Biolegend, USA), TIGIT fluorescent labeled antibody (Cat. No. 372704, Biolegend, USA), SIRPα fluorescent labeled antibody (Cat. No. 372104, Biolegend, USA), and CD158 fluorescent labeled antibody (Cat. No. FAB1848P, R&D Systems, USA).

Finally, the cell sorter was used to analyze whether the cells exhibited CD16 receptor, CD45 marker, CD4 marker, CD8 marker, CD19 marker, CD25 marker, NKp30 marker, NKG2D marker, NKp44 marker, NKp46 marker, CD27 marker, OX40 marker, CD107a marker, NKG2A marker, PD-1 marker, TIGIT marker, SIRPα marker, and CD158 marker.

Among these markers, CD16, CD25, NKp30, NKG2D, NKp44, NKp46, and CD107a are activation markers, whereas NKG2A, PD-1, TIGIT, SIRPα. CD27, OX40, and CD158 are inhibitory markers. Based on the knowledge of those skilled in the art, expression of activation markers potentiates anti-tumor activity of NK cells, whereas expression of inhibitory markers potentiates function inhibition of NK cells.

Please refer to Table 3. Table 3 shows the test result of the activation markers, inhibitory markers, and other NK cell markers of the cell suspensions obtained from embodiments 2.1.

Table 3 shows that the purified CD16⁺ populations express CD56 (98.0±0.2%), CD2 (99.5±0.2%), CD45 (99.7±0.1%), CD4 (0.8±0.3%), CD3 (0.0±0.0%), CD8 (0.0±0.0%), CD19 (0.0±0.0%), CD16 (85.7±7.0%), CD25 (42.3±13.1%), NKp30 (93.6±4.3%), NKG2D (46.1±17.4%), NKp44 (75.1±13.3%), NKp46 (46.4±16.9%), CD27 (0.62±0.08%), OX40 (0.11±0.03%), CD107a (96.1±4.3%), NKG2A (0.14±0.15%), PD-1 (27.0±19.4%), TIGIT (4.3±6.5%), SIRPα (3.2±3.0%), and CD158 (0.4±0.3%). All of the aforesaid cells expressing CD16 receptor in the purified CD16⁺ cell population have the feature of CD3⁻CD56⁺ after analysis. Preferably, the aforesaid cells expressing CD16 receptor in the purified CD16⁺ cell population is positive for CD2, CD45, and CD4 and negative for CD8 and CD19. The positiveness of CD4 is an unexpected result.

TABLE 3
the test result of the activation markers, inhibitory markers, and other
NK cell markers of the cell suspensions obtained from embodiments 2.1.

| Marker | CD56 | CD2 | CD45 | CD4 | CD3 | CD8 | CD19 |
|---|---|---|---|---|---|---|---|
| % of positive | 98.0 ± 0.2 | 99.5 ± 0.2 | 99.7 ± 0.1 | 0.8 ± 0.3 | 0.0 ± 0.0 | 0.0 ± 0.0 | 0.0 ± 0.0 |
| population | | | | | | | |

| Marker | CD16 | CD25 | NKp30 | NKG2D | NKp44 | NKp46 | CD27 | OX40 | CD107a |
|---|---|---|---|---|---|---|---|---|---|
| % of positive | 85.7 ± 7.0 | 42.3 ± 13.1 | 93.6 ± 4.3 | 46.1 ± 17.4 | 75.1 ± 13.3 | 46.4 ± 16.9 | 0.62 ± 0.08 | 0.11 ± 0.03 | 96.1 ± 4.3 |
| population | | | | | | | | | |

| Marker | NKG2A | PD-1 | TIGIT | SIRPα | CD158 |
|---|---|---|---|---|---|
| % of positive | 0.14 ± 0.15 | 27.0 ± 19.4 | 4.3 ± 6.5 | 3.2 ± 3.0 | 0.4 ± 0.3 |
| population | | | | | |

### Embodiment 4: Non-Tumorigenicity of Non-Transgenic Human CD16⁺ Natural Killer Cell Line

Six to eight-week-old female BALB/c nude mice (purchased from The Jackson Laboratory or BioLasco, Taiwan) were used in this Embodiment. 30 mice were randomly assigned into six groups, which were a SK-OV-3 group, Raji group, Daudi group, oNK group, γ-ray irradiated ACE-oNK group, and DPBS group.

A human ovarian cancer cell line “SK-OV-3” (Purchased from ATCC; The deposit number is ATCC HTB-77), human B lymphoblastoid cell lines “Raji” (Purchased from ATCC; The deposit number is ATCC CCL-86) and “Daudi” (Purchased from ATCC; The deposit number is ATCC CCL-213), a cell suspension that was obtained by culturing for 88 days with the culture method disclosed in the embodiments 2.1 (88-day cultured oNK suspension of the present invention, refer to as 88-day cultured oNK suspension), and a γ-ray irradiated ACE-oNK cell suspension were used in this Embodiment. The method for preparing γ-ray irradiated ACE-oNK cell suspension was described below.

γ-ray irradiated ACE-oNK cell suspension: the cell suspension that was obtained by culturing for 84 days with the culture method disclosed in the embodiments 2.1 (84-day cultured oNK suspension of the present invention, refer to as 84-day cultured oNK suspension) were gamma irradiated at dose 10 Gy. After binding Trastuzumab to cells in the γ-ray irradiated 84-day cultured oNK suspension using a cell linker and a Trastuzumab linker which are complementary, the γ-ray irradiated ACE-oNK cell suspension were obtained.

The procedure of binding Trastuzumab to cells (e.g., natural killer cells, cells in the 60-day cultured oNK suspension, cells in the γ-ray irradiated 60-day cultured oNK suspension) was as follows: (A) The step of preparing cell linker and binding the cell linker to the cell in order to prepare a cell-ssDNA conjugate; (B) The step of preparing Trastuzumab linker and binding the Trastuzumab linker to Trastuzumab in order to prepare the Trastuzumab-ssDNA conjugate; (C) Mixing cell-ssDNA conjugate and Trastuzumab-ssDNA conjugate to combine cell-ssDNA conjugate and Trastuzumab-ssDNA conjugate through the cell linker and its complementary sequence on the Trastuzumab linker in order to prepare Trastuzumab-conjugated cells.

Wherein the step (A) of preparing cell linker and binding the cell linker to the cell comprises the following steps (a1)˜(a4):

Step (a1) A first single strand DNA was obtained, wherein the sequence of the first single strand DNA was SEQ ID NO:5, SEQ ID NO:6, or SEQ ID NO:7.

Step (a2) The 5′ end of the first single strand DNA was modified as 5′ end thiol-modified first single strand DNA to obtain the cell linker stock. The cell linker stock is also commercially available from Integrated DNA Technologies. Actual methods of modification are known, or will be apparent, to those skilled in the art (Zimmermann, J, 2010).

Step (a3) 10-500 μL cell linker stock and 0.1-10 μL NHS-Maleimide (commercially available from Fisher Scientific) were mixed and incubated for 1-60 minute(s).

Step (a4) The mixture obtained from Step (a3) were mixed with 1×10⁶-1×10⁸ cells and incubated for 1-60 minutes to obtain cell-ssDNA conjugate.

The step (B) of preparing Trastuzumab linker and binding the Trastuzumab linker to Trastuzumab comprises the following steps (b1)˜(b4):

Step (b1) A second single strand DNA was obtained, wherein the sequence of the second single strand DNA was SEQ ID NO:8, SEQ ID NO:9, or SEQ ID NO:10, and the sequence of the second single strand DNA is the complementary strand to the first single strand DNA.

Step (b2) The 5′ end of the second single strand DNA was modified as 5′ end thiol-modified second single strand DNA to obtain a Trastuzumab linker stock. The Trastuzumab linker stock is also commercially available from Integrated DNA Technologies. Actual methods of modification are known, or will be apparent, to those skilled in the art (Zimmermann, J, 2010).

Step (b3) 10-500 μL Trastuzumab linker stock and 0.1-10 μL NHS-Maleimide (commercially available from Fisher Scientific) were mixed and incubated for 1-60 minute(s).

Step (b4) The mixture obtained from Step (b3) were mixed with 10-100 μL Trastuzumab stock (commercially available from Roche) and incubated for 10 minutes to 3 hours to obtain Trastuzumab-ssDNA conjugate.

The cell-ssDNA conjugate and the Trastuzumab-ssDNA conjugate were mixed to obtain Trastuzumab-conjugated cell such as cells in the γ-ray irradiated ACE-oNK cell suspension.

1×10⁷ SK-OV-3 cells, 1×10⁷ Raji cells, 1×10⁷ Daudi cells, 1×10⁷ cells in the 60-day cultured oNK suspension, and 1×10⁷ cells in γ-ray irradiated ACE-oNK cell suspension were suspended respectively in 100 μL, of Dulbecco's Phosphate-Buffered Saline (DPBS) to obtain different cell suspensions. The cell suspensions and 100 μL, of DPBS were subcutaneously implanted in female BALB/c nude mice in SK-OV-3 group, Raji group, Daudi group, oNK group, γ-ray irradiated ACE-oNK group, and DPBS group on Day 0 respectively. Tumor growth in each mouse was observed on Day 14, Day 21, Day 24, Day 42, and Day 59, and the mice were euthanized on Day 59.

Please refer to Table 4. Table 4 shows the results of tumor formation in nude mice xenografted with different cell lines.

Table 4 shows that there was no tumor formation in the mice of DPBS groups (negative control group) throughout the study period (0/5, 0%), while all five mice in SK-OV-3 group (positive control group) developed tumors (5/5, 100%). For mice xenografted with lymphoma cell line Daudi, 4 out of 5 mice in Daudi group developed tumors (4/5, 80%) that lasted until end of study (Day 59). For mice xenografted with lymphoma cell line Raji, 1 out of 5 (1/5) mice harbored detectable tumor before Day 42, but then returned to unmeasurable size by end of study.

For mice xenografted with oNK cells or γ-ray irradiated ACE-oNK cells of the present invention, there was no tumor formation in mice in oNK group and γ-ray irradiated ACE-oNK group throughout the study period (0/5, 0%). These study results provide evidence that non-irradiated oNK cells and the Trastuzumab-conjugated irradiated ACE-oNK cells are non-tumorigenic and safe for future clinical application and disease treatment.

TABLE 4
the results of tumor formation in nude mice
xenografted with different cell lines.

| | Tumor incidence | | | | |
|---|---|---|---|---|---|
| Cell type | Day 14 | Day 21 | Day 24 | Day 42 | Day 59 |
| SK-OV-3 suspension | 5/5 | 5/5 | 5/5 | 5/5 | 5/5 |
| Raji suspension | 1/5 | 1/5 | 1/5 | 0/5 | 0/5 |
| Daudi suspension | 0/5 | 3/5 | 4/5 | 4/5 | 4/5 |
| Non-irradiated oNK | 0/5 | 0/5 | 0/5 | 0/5 | 0/5 |
| suspension | | | | | |
| ACE-oNK-HER2 | 0/5 | 0/5 | 0/5 | 0/5 | 0/5 |
| suspension | | | | | |
| DPBS | 0/5 | 0/5 | 0/5 | 0/5 | 0/5 |

### Embodiment 5: Cytotoxicity of the Cultured Non-Transgenic Human CD16⁺ Natural Killer Cell Line Against Different Cancer Cells

The experimental method of this embodiment is almost the same as that of Embodiment 3.5, except that (1) the effector cell used in this embodiment is the cell suspension that was obtained by culturing for 104 days with the culture method disclosed in the embodiments 2.1 (104-day cultured oNK suspension of the present invention, refer to as 104-day cultured oNK suspension wherein the proportion of human CD16⁺ natural killer cell line is 82.51%), (2) the target cells used in this embodiment are SK-OV-3 (a human ovarian cancer cell line), SK-BR-3 (a human breast cancer cell line), OVCAR-3 (a human ovarian cancer cell line), MCF-7 (a human breast cancer cell line), A549 (a human lung carcinoma cell line), and T24 (a human bladder carcinoma cell line); and (3) the ratio of the number of effector cells to the number of target cells is 1:1, 2:1 and 5:1 (ET1, ET2 and ET5).

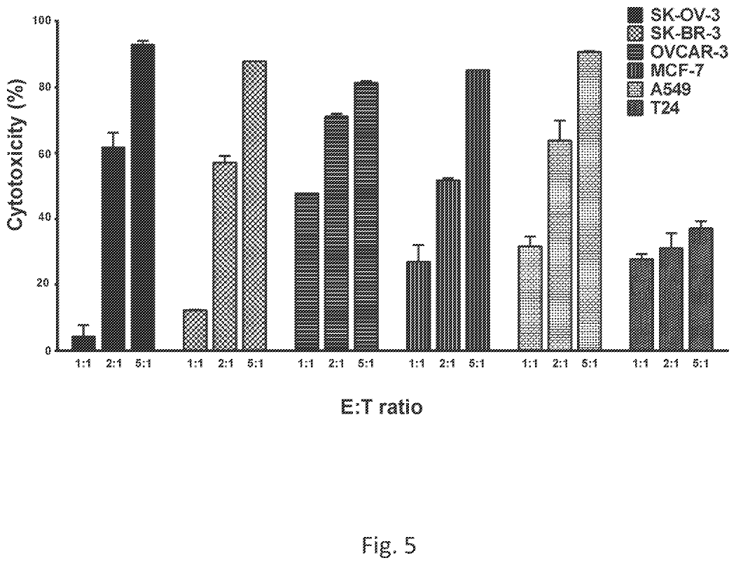

Please refer to FIG. 5. FIG. 5 is the bar chart presenting the cytotoxicity of the cultured non-transgenic human CD16⁺ natural killer cell line against different cancer cells.

These study results provide evidence that the cultured non-transgenic CD16⁺ natural killer cell line kill 4.33±3.43% to 92.98±1.06% of SK-OV-3, 12.23±0.09% to 87.88±0.01% of SK-BR-3, 47.78±0.09% to 81.30±0.52% of OVCAR-3, 27.02±5.05% to 85.15±0.01% of MCF-7, 31.68±3.00% to 90.74±0.22% of A549 and 27.77±1.57% to 37.09±2.21% of T24 at the ratio of the number of effector cells to the number of target cells being 1:1 to 5:1 (ET1 to ET5). Thus, the cultured non-transgenic CD16+ natural killer cell line harbors effective cytotoxicity against diverse types of cancer cells.

### Embodiment 6: The Comparison of the Cytotoxic Activity Between the Cultured Non-Transgenic Human CD16⁺ Natural Killer Cell Line and NK-92 Cell Line

The experimental method of this embodiment is almost the same as that of Embodiment 3.5, except that (1) the effector cell used in this embodiment is \textcircled{1} the cell suspension that was obtained by culturing for 33 days with the culture method disclosed in the embodiments 2.1 (33-day cultured oNK suspension of the present invention, refer to as 33-day cultured oNK suspension wherein the proportion of human CD16⁺ natural killer cell line is 91.74%), or \textcircled{2} the population of human peripheral blood natural killer cells having the deposit number ATCC CRL-2407 (refer to as NK-92 suspension wherein the proportion of NK-92 cell line is at least 98% as shown in FIG. 2B and the NK-92 cell line is a CD16⁻ natural killer cell line); and (2) the ratio of the number of effector cells (the total cells in the 33-day cultured oNK suspension or the total cells in the NK-92 suspension) to the number of SK-OV-3 cells (target cells) is 2:1 (ET2).

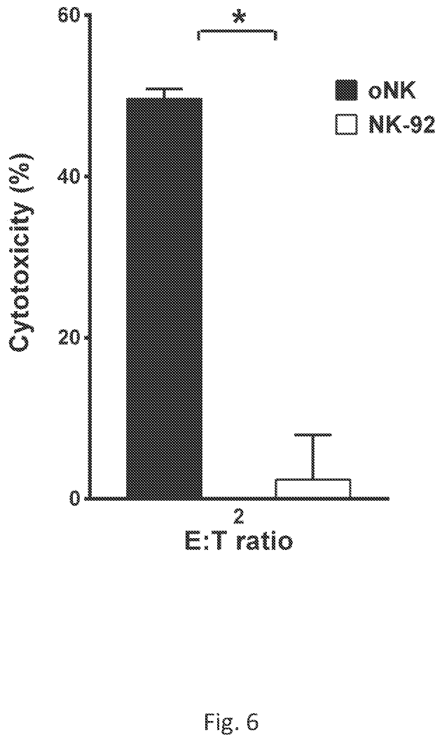

Please refer to FIG. 6. FIG. 6 is the bar chart presenting the comparison of the cytotoxic function between the cultured non-transgenic human CD16⁺ natural killer cell line and NK-92 cell line to kill cancer cells. FIG. 6 shows that NK-92 cell line (a CD16⁻ natural killer cell line and thus unable to destroy cancer cells through ADCC process) only killed 2.40±5.52% of cancer cells, whereas oNK cells (non-transgenic human CD16⁺ natural killer cells that were not linked to or co-cultured with IgG antibodies targeting the tumor-associated antigens and thus not activated to induce ADCC reaction) killed 49.68±1.19% of cancer cells.

Thus, the result shows that: as compare with NK-92 cells (NK-92 is a CD16⁻ natural killer cell line and thus unable to destroy cancer cells through ADCC process), oNK cells that were not activated to induce ADCC reaction could cause about 21-fold increase of cytotoxicity (49.68÷2.4=21). This is an unexpected result.

Moreover, based on this result, applicant believe that after isolating human CD16⁺ natural killer cell line from the 33-day cultured oNK suspension (cultured oNK) and isolating CD16⁻ natural killer cell line (NK-92) from the NK-92 suspension, similar unexpected result could be observed.

### Embodiment 7: The Comparison of the Cytotoxic Activity Between Different Amount of Non-Transgenic Human CD16⁺ Natural Killer Cell Line

The experimental method of this embodiment is almost the same as that of Embodiment 3.5, except that (1) the effector cell used in this embodiment is \textcircled{1} the cell suspension that was obtained by culturing for X days with the culture method disclosed in the embodiments 2.1 (X-day cultured oNK suspension of the present invention wherein the proportion of human CD16⁺ natural killer cell line is 8.91%, refer to as suspension with small number of oNK cells), \textcircled{2} the cell suspension that was obtained by culturing for Y days with the culture method disclosed in the embodiments 2.1 (Y-day cultured oNK of the present invention wherein the proportion of human CD16⁺ natural killer cell line is 64.15%, refer to as suspension with medium number of oNK cells), \textcircled{3} the cell suspension that was obtained by culturing for Z days with the culture method disclosed in the embodiments 2.1 (Z-day cultured oNK of the present invention wherein the proportion of human CD16⁺ natural killer cell line is 91.74%, refer to as suspension with large number of oNK cells), \textcircled{4} the population of human peripheral blood natural killer cells with the deposit number ATCC CRL-2407 (refer to as NK-92 suspension wherein the proportion of NK-92 cell line is at least 98% as shown in FIG. 2B and the NK-92 cell line is a CD16⁻ natural killer cell line), \textcircled{5} suspension with small number of ACE-oNK-HER2 cells, \textcircled{6} suspension with medium number of ACE-oNK-HER2 cells, or \textcircled{7} suspension with large number of ACE-oNK-HER2 cells; and (2) the ratio of the number of effector cells (the total cells in the suspension with small number of oNK cells, in the suspension with medium number of oNK cells, in the suspension with large number of oNK cells, in the NK-92 suspension, in the suspension with small number of ACE-oNK-HER2 cells, in the suspension with medium number of ACE-oNK-HER2 cells, or in the suspension with large number of ACE-oNK-HER2 cells) to the number of SKOV-3 cells (target cells) is 2:1 (ET2).

The method for preparing the suspension with small number of ACE-oNK-HER2 cells, the suspension with medium number of ACE-oNK-HER2 cells, and the suspension with large number of ACE-oNK-HER2 cells were described below.

The suspension with small number of ACE-oNK-HER2 cells: the total cells in “the suspension with small number of oNK cells” were linked with Trastuzumab by using a cell linker and a Trastuzumab linker that are complementary, and therefore the suspension with small number of ACE-oNK-HER2 cells were obtained wherein the proportion of ACE-oNK-HER2 cells is about 8.91%.

The suspension with medium number of ACE-oNK-HER2 cells: the total cells in “the suspension with medium number of oNK cells” were linked with Trastuzumab by using a cell linker and a Trastuzumab linker that are complementary, and therefore the suspension with medium number of ACE-oNK-HER2 cells was obtained wherein the proportion of ACE-oNK-HER2 cells is about 64.15%.

The suspension with large number of ACE-oNK-HER2 cells: the total cells in “the suspension with large number of oNK cells” were linked with Trastuzumab by using a cell linker and a Trastuzumab linker that are complementary, and therefore the suspension with large number of ACE-oNK-HER2 cells was obtained wherein the proportion of ACE-oNK-HER2 cells is about 91.74%.

The procedure of binding Trastuzumab to cells (cells in the suspension with small number of oNK cells, the suspension with medium number of oNK cells, or the suspension with large number of oNK cells) is same as that of Embodiment 4.

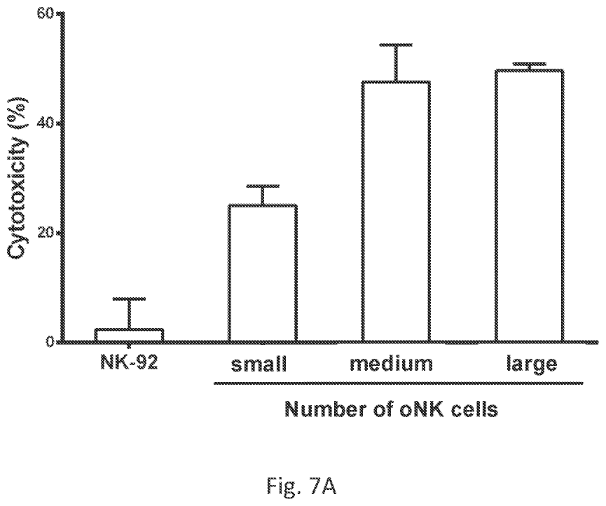

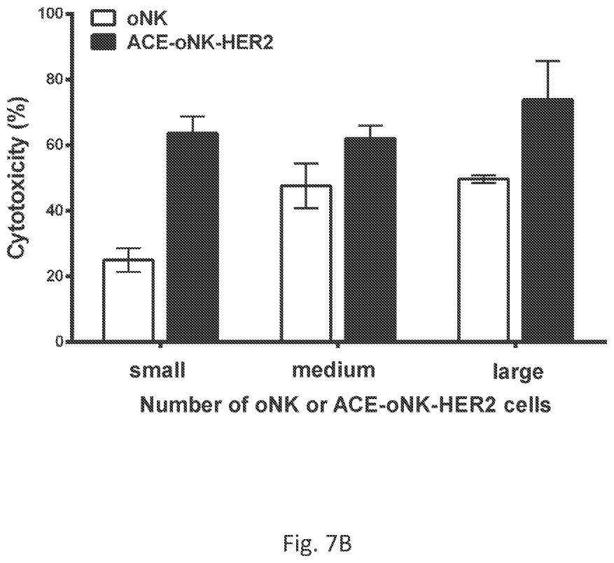

Please refer to FIG. 7A and FIG. 7B. FIG. 7A is the bar chart presenting the comparison of the cytotoxic activity between different numbers of non-transgenic human CD16⁺ natural killer cell line to kill cancer cells. FIG. 7B is the bar chart presenting the comparison of the cytotoxic activity between different numbers of anti-HER2 antibody-conjugated non-transgenic human CD16⁺ natural killer cell line to kill cancer cells through ADCC process.

FIG. 7A shows that NK-92 cell line (a CD16⁻ natural killer cell line and thus unable to destroy cancer cells through ADCC process) only killed 2.40±5.52% of cancer cells; small number of oNK cells (non-transgenic human CD16⁺ natural killer cells that were not linked to or co-cultured with IgG antibodies targeting the tumor-associated antigens and thus not activated to induce ADCC reaction) killed 25.00±3.60% of cancer cells; medium number of oNK cells (non-transgenic human CD16⁺ natural killer cells that were not linked to or co-cultured with IgG antibodies targeting the tumor-associated antigens and thus not activated to induce ADCC reaction) killed 47.60±6.80% of cancer cells; large number of oNK cells (non-transgenic human CD16⁺ natural killer cells that were not linked to or co-cultured with IgG antibodies targeting the tumor-associated antigens and thus not activated to induce ADCC reaction) killed 49.68±1.19% of cancer cells.

Thus, the result shows that: as compare with NK-92 cells (NK-92 is a CD16⁻ natural killer cell line and thus unable to destroy cancer cells through ADCC process), the suspension with small number of oNK cells (wherein the proportion of human CD16⁺ natural killer cell line is 8.91%) is enough to cause about 10-fold increase of cytotoxicity (25±2.4=10). This is an unexpected result. Therefore, it indicated that human CD16⁺ natural killer cell line in an amount equal to or more than 5% by number is enough to kill cancer cells, based on the total number of the cells in the composition as 100%. Based on this result, applicant believes that similar unexpected result could be observed in clinical trials.

The result also shows that the suspension with medium or large number of oNK cells (wherein the proportion of human CD16⁺ natural killer cell line is 64.15% or 91.74%) could cause about 20-21 fold increase of cytotoxicity (47.60±2.4=20; 49.68±2.4=21). Therefore, it indicates that the more the human CD16⁺ natural killer cell line, the more the cancer cells are killed and then reach a plateau as the human CD16⁺ natural killer cell line in an amount equal to about 60%-65% by number, based on the total number of the cells in the composition as 100%. Based on this result, applicant believes that similar result could be observed in clinical trials.

FIG. 7B shows that small number of oNK cells killed 25.00±3.60% of cancer cells; medium number of oNK cells killed 47.60±6.80% of cancer cells; large number of oNK cells killed 49.68±1.19% of cancer cells; small number of ACE-oNK-HER2 cells killed 63.70±5.00% of cancer cells; medium number of ACE-oNK-HER2 cells killed 62.00±4.00% of cancer cells; large number of ACE-oNK-HER2 cells killed 73.9±11.80% of cancer cells.

Thus, the result shows that: when the non-transgenic human CD16⁺ natural killer cell line obtained by the culture of the present invention was linked with antibodies targeting the tumor-associated antigens (such as Trastuzumab) by using a cell linker and an antibody linker (such as Trastuzumab linker) which are complementary and thus could be activated to induce ADCC reaction, the cytotoxic effect was significantly increased by 14.4%-38.7% (62.00%−47.60%=14.4%; 63.70%−25.00%=38.7%).

The result also shows that exogeneous targeting unit complexed-oNK cell (such as anti-HER2 antibody-conjugated oNK cells) in an amount equal to or more than 5% by number is enough to kill cancer cells through ADCC process; it also indicates that the more the exogeneous targeting unit complexed-oNK cell, the more the cancer cells are killed through ADCC process and reach a first plateau as the exogeneous targeting unit complexed-oNK cell in an amount equal to about 5%-10% by number, based on the total number of the cells in the composition as 100%. Based on this result, applicant believes that similar result could be observed in clinical trials.

### Embodiment 8: The Comparison of the Cytotoxic Activity Between the Anti-HER2 Antibody-Conjugated Non-Transgenic Human CD16⁺ Natural Killer Cell Line and the Anti-HER2 Antibody Co-Cultured Non-Transgenic Human CD16⁺ Natural Killer Cell Line

The experimental method of this embodiment is almost the same as that of Embodiment 3.5, except that (1) the effector cell used in this embodiment is \textcircled{1} cell suspensions obtained by culturing for 55 days with the culture method disclosed in the embodiments 2.1 (refer to as 55-day cultured oNK suspension), or \textcircled{2} cell suspension with ACE-oNK-HER2 cells (the total cells in “55-day cultured oNK suspension” were linked with Trastuzumab by using a cell linker and a Trastuzumab linker that are complementary as described in Embodiment 4); (2) the ratio of the number of effector cells (the total cells in the 55-day cultured oNK suspension or the total cells in the cell suspension with ACE-oNK-HER2 cells) to the number of SK-OV-3 cells (target cells) is 1:1 (ET1), 2:1 (ET2), or 5:1 (ET5); and (3) In the experimental wells for the 55-day cultured oNK suspension, equivalent amount of Trastuzumab as the total amount of the Trastuzumab linked to the cells in the cell suspension with ACE-oNK-HER2 cells at E:T ratio of 1 (0.55 ng), 2 (1.10 ng) and 5 (2.75 ng) was added. The detail procedure was described below.

The wells in xCELLigence E-Plate were divided into control wells, ACE-oNK-HER2 ET1 experimental well, ACE-oNK-HER2 ET2 experimental well, ACE-oNK-HER2 ET5 experimental well, oNK and Herceptin ET1 experimental well, oNK and Herceptin ET2 experimental well, oNK and Herceptin ET5 experimental well, and target cell maximum lysis control well. SK-OV-3 cells were seeded in control well, ACE-oNK-HER2 ET1 experimental well, ACE-oNK-HER2 ET2 experimental well, ACE-oNK-HER2 ET5 experimental well, oNK and Herceptin ET1 experimental well, oNK and Herceptin ET2 experimental well, oNK and Herceptin ET5 experimental well, and target cell maximum lysis control well, so that each well-contained 20000 SK-OV-3 cells, and then allowed it to sit 30 minutes.

20000, 40000, or 100000 cells in the cell suspension with ACE-oNK-HER2 cells was added to the ACE-oNK-HER2 ET1 experimental well, ACE-oNK-HER2 ET2 experimental well, and ACE-oNK-HER2 ET5 experimental well respectively; hence, the ratio of the number of effector cell (the total cells in the cell suspension with ACE-oNK-HER2 cells) to the number of SKOV-3 cells (target cells) was 1, 2 and 5.

Both of 20000, 40000, or 100000 cells in the 55-day cultured oNK suspension and 0.55, 1.10, or 2.75 ng of Trastuzumab (an antibody against HER2 protein with product name as Herceptin was purchased from Roche Swiss) were added to the “oNK and Herceptin ET1 experimental well”, “oNK and Herceptin ET2 experimental well”, and “oNK and Herceptin ET5 experimental well” respectively. Therefore, the ratio of the number of effector cell (the total cells in the 55-day cultured oNK suspension) to the number of SK-OV-3 cells (target cells) was 1, 2 and 5; the amount of Trastuzumab in the “oNK and Herceptin ET1 experimental well”, “oNK and Herceptin ET2 experimental well”, or “oNK and Herceptin ET5 experimental well” was respectively same as the total amount of the Trastuzumab linked to the cells in the ACE-oNK-HER2 ET1 experimental well, ACE-oNK-HER2 ET2 experimental well, and ACE-oNK-HER2 ET5 experimental well.

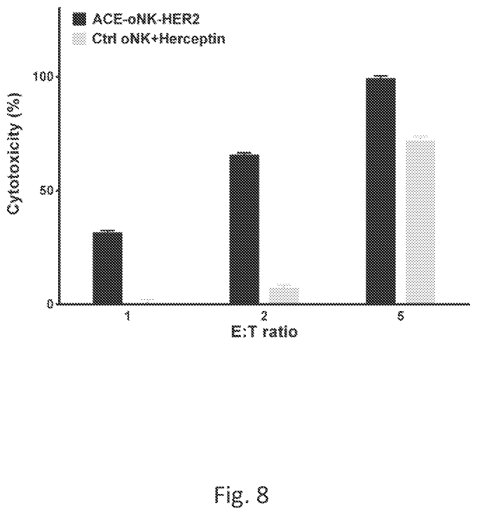

Please refer to FIG. 8. FIG. 8 is the bar chart presenting the comparison of the cytotoxic function between the anti-HER2 antibody-conjugated non-transgenic human CD16⁺ natural killer cell line and the anti-HER2 antibody co-cultured non-transgenic human CD16⁺ natural killer cell line to kill cancer cells through ADCC process. FIG. 8 shows that oNK cells that were co-cultured with IgG antibodies targeting the tumor-associated antigens (and thus activated to induce ADCC reaction) only killed 0.00±2.10%, 7.30±1.40%, or 71.8±2.10% of cancer cells at E:T ratio of 1, 2, or 5 respectively, whereas ACE-oNK-HER2 cells that were linked (conjugated) with IgG antibodies targeting the tumor-associated antigens (and thus activated to induce ADCC reaction) killed 31.40±1.10%, 65.60±1.00%, or 99.10±1.30% of cancer cells at E:T ratio of 1, 2, or 5 respectively.

Thus, the result shows that: as compare with oNK cells that were co-cultured with IgG antibodies targeting the tumor-associated antigens, ACE-oNK-HER2 cells that were linked (conjugated) with IgG antibodies targeting the tumor-associated antigens could cause 9-∞ fold increase of cytotoxicity at lower doses (ET1 with 0.55 ng Trastuzumab, or ET2 with 1.10 ng Trastuzumab; 65.60÷7.30=9; 31.40÷0.00=∞; ∞ is a symbol that represents an infinitely large number). That is, “linking CD16⁺ natural killer cells with anti-tumor antigen antibody” (e.g. linking cultured oNK with Trastuzumab) makes an unexpected result, and linking CD16⁺ natural killer cells with anti-tumor antigen antibody” make effective and safer therapy based on lower dose treatment could be achieved.

Moreover, based on this result, applicant believes that after isolating human CD16⁺ natural killer cell line from the 55-day cultured oNK suspension (cultured oNK) and isolating Trastuzumab-linked CD16⁺ natural killer cells (ACE-oNK-HER2 cells) from the cell suspension with ACE-oNK-HER2 cells, similar unexpected result could be observed.

### Embodiment 9: Detection of Genomic DNA of Non-Transgenic Human CD16⁺ Natural Killer Cell Line

### Embodiment 9.1 Detection of DNA Sequence Encoding CD16 Receptor by Droplet Digital PCR (ddPCR)

Droplet Digital PCR (ddPCR) was used in this embodiment to detect DNA sequence encoding CD16 receptor of cultured non-transgenic human CD16⁺ natural killer cell line in the present invention (oNK) or CD16-transgenic NK-92 cell line (yNK).

Cell suspensions obtained by culturing for M days with the culture method disclosed in the embodiments 2.1 (refer to as M-day cultured oNK suspension) and CD16-transgenic NK-92 cell line (Purchased from ATCC with the deposit number is ATCC PTA-6967; refer to as yNK) were used in this embodiment. Genomic DNA of yNK and cells in the M-day cultured oNK suspension were isolated by Blood & Cell Culture DNA Mini Kit (Purchased from Qiagen).

yNK sample or oNK sample: 50 ng genomic DNA isolated from yNK or M-day cultured oNK suspension was mixed with 10 μL ddPCR™ Supermix for Probes (2×) (Catalog number #1863026; Purchased from Bio-Rad), 1 μL BstXI restriction enzyme (Product name BstXI; Catalog number R0113S; Purchased form BioLabs), and 1 μL Mixture of CD16 F176F hydrolysis probe and CD16 F176V hydrolysis probe (Assay ID: C_25815666_10; Purchased form ThermoFisher; The Context Sequence

[VIC/FAM]:
TCTGAAGACACATTTTTACTCCCAA[C/A]AAGCCCCCTGCAGAAGTAGG
AGCCG;

https://www.thermofisher.com/order/genome-database/details/genotyping/C_25815666_10?CID=&ICID=&subtype=), and the final volume is 20 μL.

No-template control sample: water, 10 μL ddPCR™ Supermix for Probes (2×), 1 μL BstXI restriction enzyme, and 1 μL Mixture of CD16 F176F hydrolysis probe and CD16 F176V hydrolysis probe were mixed, and the final volume is 20 μL.

ddPCR experiments were performed using the QX100/QX200 Droplet Digital PCR (ddPCR) system (Purchased from Bio-Rad). First, samples are placed into a QX100 or QX200 Droplet Generator (a machine in the QX100/QX200 Droplet Digital PCR system) to partition each sample into 15000-20000 droplets (nanoliter-sized droplet).

Second, the wells in the 96 well plate (Product name: DG8 cartridge; Purchased from Bio-Rad) were divided into no-template control well, yNK well, and oNK well, and these wells are for no-template control group (NTC group), yNK group, and oNK group respectively. Nanolized no-template control sample, yNK sample, and oNK sample were respectively transferred into the no-template control well, yNK well, and oNK well.

Third, for the PCR amplification process, thermocycling conditions were 95° C. for 10 min, 45 cycles of 95° C. for 15 s, and 60° C. for 1 min, followed by 98° C. for 10 min then hold at 4° C. The ramp rate for each step was set to 2° C./s.

CD16 F176F hydrolysis probe is a probe labeled with FAM reporter fluorophore, and CD16 F176V hydrolysis probe is a probe labeled with VIC reporter fluorophore. The main steps in the PCR amplification process are denaturation, annealing, and extension. During annealing, the hydrolysis probe (such as CD16 F176F hydrolysis probe or CD16 F176V hydrolysis probe) binds to the target sequence; then during extension, the reporter labeled at the 5′ end of the probe is cleaved and free reporter fluoresces. The sequence of CD16 F176F hydrolysis probe is SEQ ID NO:11 and thus is expected to be able to detect DNA sequence encoding CD16 receptor located on q arm of chromosome 1 at position 1q23.3; the sequence of CD16 F176V hydrolysis probe is SEQ ID NO:12 and is expected to be able to detect the synthetic DNA sequence in yNK.

Please note that the DNA sequence encoding CD16 receptor located on q arm of chromosome 1 at position 1q23.3 in oNK would be transcribed to CD16 F176F mRNA then translated to CD16 F176F protein, wherein the sequence of the DNA encoding CD16 receptor located on q arm of chromosome 1 at position 1q23.3 in oNK comprises SEQ ID NO:1, SEQ ID NO:2, or SEQ ID NO:19; the sequence of CD16 F176F mRNA comprises SEQ ID NO:13; the sequence of CD16 F176F protein comprises SEQ ID NO:3, SEQ ID NO:4, SEQ ID NO:14, or SEQ ID NO:20. The synthetic DNA sequence encoding CD16 receptor in yNK would be transcribed to CD16 F176V mRNA then translated to CD16 F176V protein, and the sequence of CD16 F176V mRNA is SEQ ID NO:15; the sequence of CD16 F176V protein is SEQ ID NO:16.

Forth, for droplet reading process, droplets were read using a QX100/QX200 Droplet Reader (a machine in the QX100/QX200 Droplet Digital PCR system), in which droplets were spaced out individually for fluorescence reading and therefore each droplet was analyzed individually using a two-color detection system (set to detect FAM and VIC). Positive droplets, which contain at least one copy of the target DNA molecule (such as CD16 F176F hydrolysis probe detected DNA molecule or CD16 F176V hydrolysis probe detected DNA molecule), exhibit increased fluorescence compared with negative droplets.

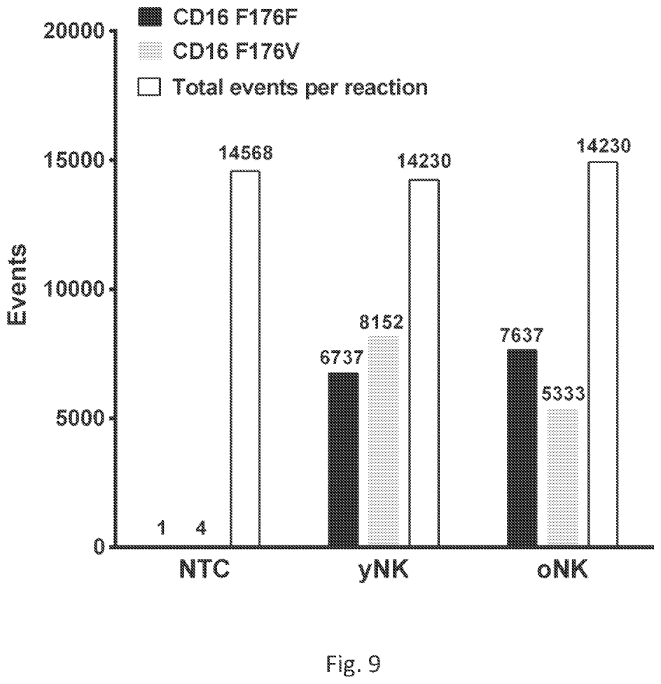

Please refer to FIG. 9. FIG. 9 is the bar chart presenting the comparison of genotype between the non-transgenic human CD16⁺ natural killer cell line and the CD16-transgenic NK-92 cell line.

In NTC group, there were only 1 positive droplet containing CD16 F176F hydrolysis probe-detectable DNA molecule and 4 positive droplets containing CD16 F176V hydrolysis probe-detectable DNA molecules in total 14568 collected droplets (events). In yNK group, there were 6737 positive droplets containing CD16 F176F hydrolysis probe-detectable DNA molecules and 8152 positive droplets containing CD16 F176V hydrolysis probe-detectable DNA molecules in total 14230 collected droplets (events). In oNK group, there were 7637 positive droplets containing CD16 F176F hydrolysis probe-detectable DNA molecules and 5333 positive droplets containing CD16 F176V hydrolysis probe-detectable DNA molecule in total 14230 collected droplets (events).

Thus, the result shows that using ddPCR system to analyze the genomic DNA of yNK cells, the ratio of CD16 F176F hydrolysis probe-detectable DNA molecule to CD16 F176V hydrolysis probe-detectable DNA molecule was 0.83 (6737÷8152=0.83), whereas using ddPCR system to analyze the genomic DNA of oNK cells, the ratio of CD16 F176F hydrolysis probe-detectable DNA molecule to CD16 F176V hydrolysis probe-detectable DNA molecule was 1.43 (7637÷5333=1.43).

That is, by using ddPCR system to analyze the genomic DNA of human CD16⁺ natural killer cell line (oNK) in the present invention, the ratio of CD16 F176F hydrolysis probe-detectable DNA molecule to CD16 F176V hydrolysis probe-detectable DNA molecule was equal to or higher than 1 (the number of CD16 F176F probe detectable DNA molecule÷the number of CD16 F176V probe detectable DNA molecule≥1).

Moreover, based on this result, applicant believes that after isolating human CD16⁺ natural killer cell line from the M-day cultured oNK suspension (cultured oNK), similar result could be observed.

Based on applicant's experience, another hydrolysis probes with sequence SEQ ID NO:17 or SEQ ID NO:18 could detect DNA sequence encoding CD16 receptor in other CD16-transgenic NK cells.

### Embodiment 9.2 Detection of DNA Sequence Encoding CD16 Receptor by Fluorescence In Situ Hybridization (FISH)

Two-color fluorescence in situ hybridization (FISH) was used in this embodiment to detect transgenic, synthetic, genetically modified, or deliberately delivered DNA sequence encoding the CD16a receptor in human natural killer cells.

The cultured non-transgenic human CD16⁺ natural killer cell line in the present invention (oNK) is used as an example to show the result of the human cell with no transgenic, synthetic, genetically modified, or deliberately delivered DNA sequence encoding the CD16 receptor, whereas the CD16-transgenic NK-92 cell line (yNK) is used as an example to show the result of the human cell with transgenic, synthetic, genetically modified, or deliberately delivered DNA sequence encoding the CD16 receptor.

For the detail, the isolated CD16⁺ NK cells (oNK cells) from the cell suspensions obtained by culturing for N days with the culture method disclosed in the embodiments 2.1 (refer to as N-day cultured oNK suspension) and CD16-transgenic NK-92 cell line (Purchased from ATCC with the deposit number is ATCC PTA-6967; refer to as yNK) were used in this embodiment.

Kallioniemi disclosed the details of the two-color fluorescence in situ hybridization (FISH) method in 1996, and a short extract is presented below.

First, nuclei from 1×10⁷ yNK cells or oNK cells (CD16⁺ NK cells) isolated form the N-day cultured oNK suspension are prepared according to protocols used in DNA flow cytometry (Kallioniemi et al., 1996; Vindelov et al., 1983). For the detail, the cell pellet is incubated in a hypotonic detergent solution and brief trypsin digestion.

Second, nuclei are dropped on microscope slides, air-dried, and fixed in methanol acetic acid.

Third, prior to hybridization, the target nuclei are treated with proteinase K or other proteolytic enzymes to improve probe penetration.

Forth, denaturation of target nuclei is usually accomplished by immersing slides in a denaturation solution (70% formamide, 2×SSC) for 2-4 min at 70° C., followed by ethanol fixation and dehydration. Denaturation time and temperature have to be optimized according to the characteristics of the target cells.

Fifth, Prior to hybridization, 20-60 ng of the first fluorescent dye-labeled FCGR3A FISH Probe (a test probe that could detect all of the human DNA sequence encoding CD16a receptor; Purchased from Empire Genomics), 20-60 ng of the second fluorescent dye-labeled Chromosome 1 Control Probe (a reference probe; Purchased from Empire Genomics), and blocking DNA (unlabeled Cot-1 or placental DNA) are added to a formamide-based hybridization buffer. It is necessary to use the blocking DNA when the probe contains repetitive sequences that will hybridize to multiple locations in the genome. Hybridization mixture is heated to 70° C. for 5 min to denature the probe fragments then applied on the target slide; a cover slip is applied and sealed with rubber cement. Hybridization is performed overnight at 37° C. in a moist chamber.

Sixth, unbound probes are washed.

Seventh, Target nuclei are counterstained with a DNA stain, typically propidium iodide or DAPI.

The hybridizations are evaluated with a regular high-quality epifluorescence microscope. Almost any recent microscope model from the major manufacturers (Zeiss, Leitz, Olympus, and Nikon) is suitable for gene-specific FISH analysis; the 60× Plan Apos or other objectives in which chromatic aberrations are carefully corrected are preferred. The number of test and reference probe signals is evaluated from a minimum of 100 randomly chosen nuclei throughout the slide. Only morphologically intact and nonoverlapping nuclei are counted. Because the nuclei are three-dimensional, it is necessary to move the focus throughout the depth of the nuclei to obtain the correct signal count.

Several formats are typically used for reporting the results of gene-specific FISH, for example: (1) the number of test probe signals per cell; (2) the number of signals per cell from the test probe divided by those from the reference probe; or (3) the percentage of cells where the test probe signal number is present at a higher or lower copy number than the reference probe.

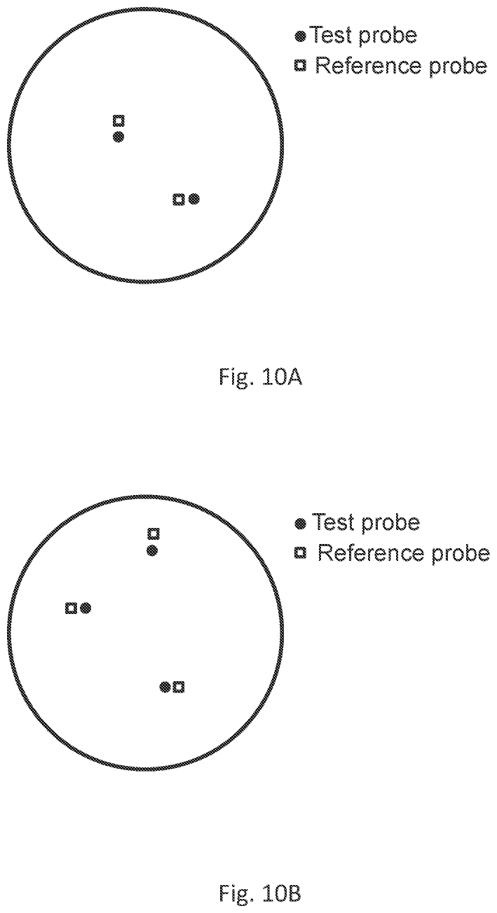

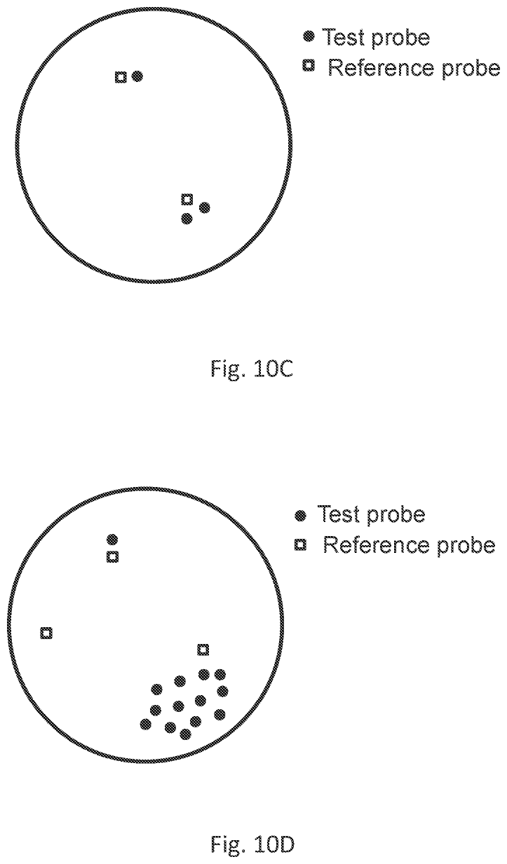

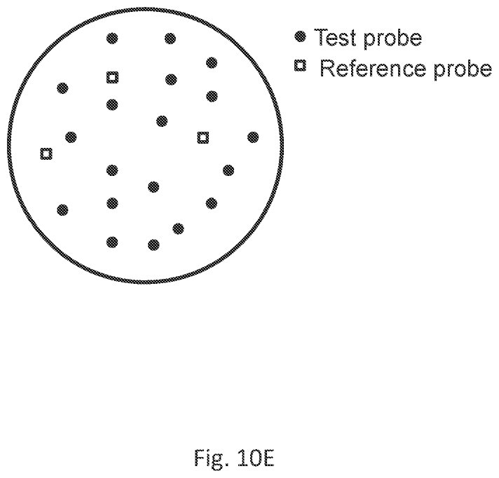

Please refer to FIG. 10A-10E. FIG. 10A-10E illustrates the principle by which two-color FISH analysis with a CD16a receptor gene-specific test probe labeled in one color and a reference probe labeled in another color can be applied to detect transgenic, synthetic, genetically modified, or deliberately delivered DNA sequence encoding the CD16a receptor in human natural killer cells.

Based on applicant's experience, the number of FCGR3A FISH Probe (a test probe which could detect all of the human DNA sequence encoding CD16a receptor) signals per oNK cell would be 2 (the actual gene copy number per cell), and two-color FISH pattern of oNK would look like FIG. 10A (normal pattern indicating the result of a human cell with no transgenic, synthetic, genetically modified, or deliberately delivered DNA sequence encoding the CD16a receptor). The number of FCGR3A FISH Probe (a test probe which could detect all of the human DNA sequence encoding CD16a receptor) signals per yNK cell may be larger than 2, and two-color FISH pattern of yNK would look like FIG. 10B-10E (CD16-transgenic pattern indicating the result of a human cell with transgenic, synthetic, genetically modified, or deliberately delivered DNA sequence encoding the CD 16a receptor).

### Embodiment 10: Effect of Freezing and Thawing on the Survival Rate of Non-Transgenic Human CD16⁺ Natural Killer Cell Line

The purified CD16⁺ cell population (the proportion of cells expressing CD16 receptor was as high as 99%) was sorted by the method of Embodiment 1.1, and then the purified CD16⁺ cell population was cultured for 21 days by the culture method of Embodiment 2.1 (purified CD16⁺ cell population was subcultured for 8 times). The sample of the cell solution was mixed with an equal volume of Trypan blue, then subjected to cell count and learned that the cell survival rate is 95%. Take a sufficient amount of the cell solution that contained 2×10⁷ viable cells, then perform the following freezing and thawing procedures.

Freezing procedure: centrifuged the cell solution containing 2×10⁷ viable cells, and removed the supernatant then resuspend the cell using 1 mL of frozen medium (CryoStor® CS10 Freeze Media, containing 10 vol % DMSO, BioLife Solutions, USA). The cell suspension was placed in a cryotube, and the cryotube was placed in the CoolCell Cell freezing container (Corning USA), then stored the CoolCell Cell freezing container in a −80° C. refrigerator overnight (which decreased 1° C. per minute). The cryotube was transferred and stored in liquid nitrogen for 17 days.

Thawing procedure: place the cryotube in a 37° C. water bath to quickly thaw the cell suspension, and mix 1 mL of cell suspension with 9 mL of cell culture medium in Embodiment 2.1. After mixing a sample of the cell mixture with an equal volume of Trypan blue, the cell number and cell viability were observed.

The experimental results showed that 1.95×10⁷ cell survived after thawing, and the Recovery rate was as high as 97.5% [(1.95×10⁷)÷(2×10⁷)×100%=97.5%], and the cell survival rate was 96% that had no significant difference from viability (95%) before freezing.

### Embodiment 11: Cytotoxic Activity of Non-Transgenic Human CD16⁺ Natural Killer Cell Line

The experimental method of this embodiment is almost the same as that of Embodiment 3.5, except that (1) the effector cell used in this embodiment is Ctrl oNK cells, Ctrl yNK cells, ACE-oNK cells, or ACE-yNK cells; and (2) the ratio of the number of effector cells to the number of SKOV-3 cells (target cells) is 2:1 (ET2) or 5:1 (ET5).

Ctrl oNK cell: Ctrl oNK cells are the cultured cell population after the purified CD16⁺ cell populations (wherein the proportion of non-transgenic human CD16⁺ natural killer cell line is as high as 99%) were cultured for 26 days by using the method of Embodiment 2.1.

Ctrl yNK cell: Ctrl yNK cells are CD16-transgenic NK-92 cell line (Purchased from ATCC; The deposit number is ATCC PTA-6967);

ACE-oNK cell: ACE-oNK cells are cells obtained by binding Trastuzumab (an antibody against HER2 protein, product name as Herceptin, purchased from Roche Swiss) to Ctrl oNK cells using a cell linker and a Trastuzumab linker that are complementary.

ACE-yNK cell: ACE-yNK cells are cells obtained by binding Trastuzumab (an antibody against HER2 protein, product name as Herceptin, purchased from Roche Swiss) to Ctrl yNK cells using a cell linker and a Trastuzumab linker that are complementary.

The procedure of binding Trastuzumab to natural killer cells (e.g., Ctrl oNK cells or Ctrl yNK cells) are as follows: (A) The step of preparing cell linker and binding the cell linker to the natural killer cell in order to prepare an NK-ssDNA conjugate; (B) The step of preparing Trastuzumab linker and binding the Trastuzumab linker to Trastuzumab in order to prepare the Trastuzumab-ssDNA conjugate; (C) Mixing NK-ssDNA conjugate and Trastuzumab-ssDNA conjugate to combine NK-ssDNA conjugate and Trastuzumab-ssDNA conjugate through the cell linker and its complementary sequence on the Trastuzumab linker in order to prepare Trastuzumab-conjugated natural killer cells (e.g., ACE-oNK cells or ACE-yNK cells).

Wherein the step (A) of preparing cell linker and binding the cell linker to the natural killer cell comprises the following steps (a1)˜(a4):

Step (a1) A first single strand DNA was obtained, wherein the sequence of the first single strand DNA was SEQ ID NO:5, SEQ ID NO:6, or SEQ ID NO:7.

Step (a2) The 5′ end of the first single strand DNA was modified as 5′ end thiol-modified first single strand DNA to obtain the cell linker stock. The cell linker stock is also commercially available from Integrated DNA Technologies. Actual methods of modification are known, or will be apparent, to those skilled in the art (Zimmermann, J, 2010).

Step (a3) 10-500 μL cell linker stock and 0.1-10 μL NHS-Maleimide (commercially available from Fisher Scientific) were mixed and incubated for 1— 60 minute(s).

Step (a4) The mixture obtained from Step (a3) were mixed with 1×10⁶-1×10⁸ natural killer cells and incubated for 1-60 minutes to obtain NK-ssDNA conjugate.

the step (B) of preparing Trastuzumab linker and binding the Trastuzumab linker to Trastuzumab comprises the following steps (b1)˜(b4):

Step (b1) A second single strand DNA was obtained, wherein the sequence of the second single strand DNA was SEQ ID NO:8, SEQ ID NO:9, or SEQ ID NO:10, and the sequence of the second single strand DNA is the complementary strand to the first single strand DNA.

Step (b2) The 5′ end of the second single strand DNA was modified as 5′ end thiol-modified second single strand DNA to obtain the Trastuzumab linker stock. The Trastuzumab linker stock is also commercially available from Integrated DNA Technologies. Actual methods of modification are known, or will be apparent, to those skilled in the art (Zimmermann, J, 2010).

Step (b3) 10-500 μL Trastuzumab linker stock and 0.1-10 μL NHS-Maleimide (commercially available from Fisher Scientific) were mixed and incubated for 1-60 minute(s).

Step (b4) The mixture obtained from Step (b3) were mixed with 10-100 μL Trastuzumab stock (commercially available from Roche) and incubated for 10 minutes to 3 hours to obtain Trastuzumab-ssDNA conjugate.

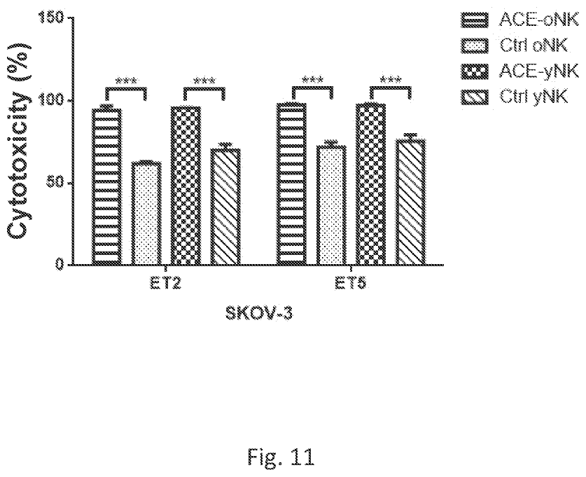

Please refer to FIG. 11. FIG. 11 is the bar chart presenting the cytotoxic function of non-transgenic human CD16⁺ natural killer cell line to kill cancer cells through ADCC process. FIG. 11 shows that regardless of the ratio of the number of effector cells to the number of SKOV-3 cells (target cells) is 2:1 (ET2) or 5:1 (ET5), non-transgenic human CD16⁺ natural killer cell lines (Ctrl oNK cells) that were not activated by Trastuzumab killed 60%-65% of cancer cells, whereas Trastuzumab-activated non-transgenic human CD16⁺ natural killer cell line (ACE-oNK cells) killed 95%˜100% of cancer cells. Thus, the non-transgenic human CD16⁺ natural killer cell line obtained by the culture of the present invention indeed have the cytotoxic function to kill cancer cells, and when the non-transgenic human CD16⁺ natural killer cell line obtained by the culture of the present invention was activated to induce ADCC reaction, the cytotoxic effect was significantly increased by at least 30% (95%−65%=30%; p<0.05).

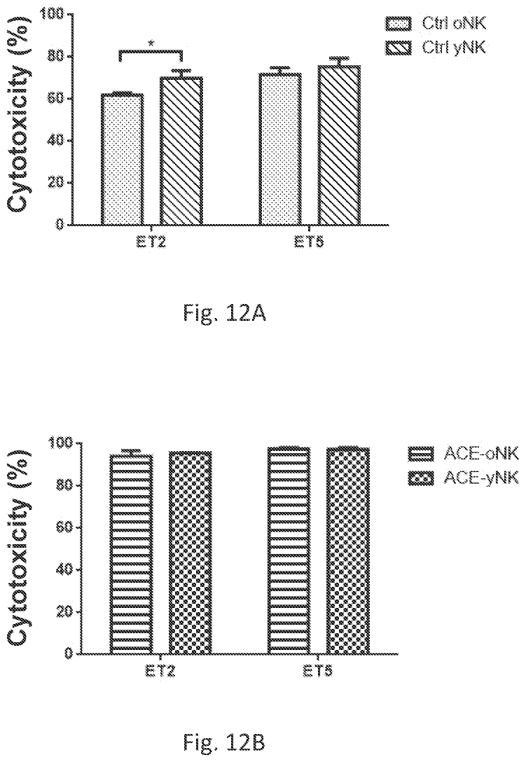

Please refer to FIGS. 12A and 12B. FIG. 12A is the bar chart presenting the comparison of the cytotoxic function between the non-transgenic human CD16⁺ natural killer cell line and CD16-transgenic NK-92 cell line to kill cancer cells at different effetor (E) to target (T) ratio; and FIG. 12B is the bar chart presenting the comparison of the cytotoxic function between the non-transgenic human CD16⁺ natural killer cell line and CD16-transgenic NK-92 cell line to kill cancer cells through ADCC process at different effetor (E) to target (T) ratio.

The results of FIG. 12A show that when the ratio of the number of effector cells to the number of SK-OV-3 cells (target cells) is 5:1 (ET5) and not activated by Trastuzumab, non-transgenic human CD16⁺ natural killer cell lines (Ctrl oNK cell) kill 70% of cancer cells, while CD16-transgenic NK-92 cell line (Ctrl yNK) kill 72% of cancer cells, there was no significant difference between the two groups (p>0.05). Thus, when the ratio of the number of effector cells to the number of SK-OV-3 cells (target cells) is 5:1 (ET5), the cytotoxic function of the non-transgenic human CD16⁺ natural killer cell line obtained by the culture of the present invention was not inferior to the CD16-transgenic NK-92 cell line. In other words, compared with the CD16-transgenic NK-92 cell line, the non-transgenic human CD16⁺ natural killer cell line obtained by the method of the present invention is not only safe but also has the same cytotoxic effect.

The results in FIG. 12B show that regardless of the ratio of the number of effector cells to the number of SK-OV-3 cells (target cells) is 2:1 (ET2) or 5:1 (ET5), Trastuzumab-activated non-transgenic human CD16⁺ natural killer cell line (ACE-oNK cells) killed 95% of cancer cells, whereas Trastuzumab-activated CD16-transgenic NK-92 cell line (ACE-yNK) also killed 95% of cancer cells, and there was no significant difference between the two groups (p>0.05). Thus, the cytotoxic function through ADCC process of the non-transgenic human CD16⁺ natural killer cell line obtained by the culture method of the present invention was not inferior to the CD16-transgenic NK-92 cell line. In other words, compared with the CD16-transgenic NK-92 cell line, the non-transgenic human CD16⁺ natural killer cell line obtained by the method of the present invention is not only safe, but also had the same cytotoxic effect in killing cancer cells through ADCC process.

### Embodiment 12: Culturing Non-Transgenic Human CD16⁺ Natural Killer Cell Line with Different Concentration of Human Platelet Lysate

The experimental method of this embodiment is almost the same as that of Embodiment 2.1, except that (1) in Step S22′, all of the cells in the cell suspensions obtained by culturing for 9 days with the culture method disclosed in the embodiments 2.1 (refer to as 9-day cultured oNK suspension) were cultured in this embodiment and the number of cells in the first container in Step S22′ was 5×10⁶; and (2) the cell culture medium comprises 500 IU/mL IL-2 and \textcircled{1} 2.5% human platelet lysate, \textcircled{2} 5.0% human platelet lysate, \textcircled{3} 10.0% human platelet lysate, or \textcircled{4} 5.0% human serum (comprising no human platelet lysate).

The experimental method of detecting cell number, cell viability, and CD16 surface marker of the cultured cells in this embodiment is the same as that of Embodiment 2.2 and 3.4.

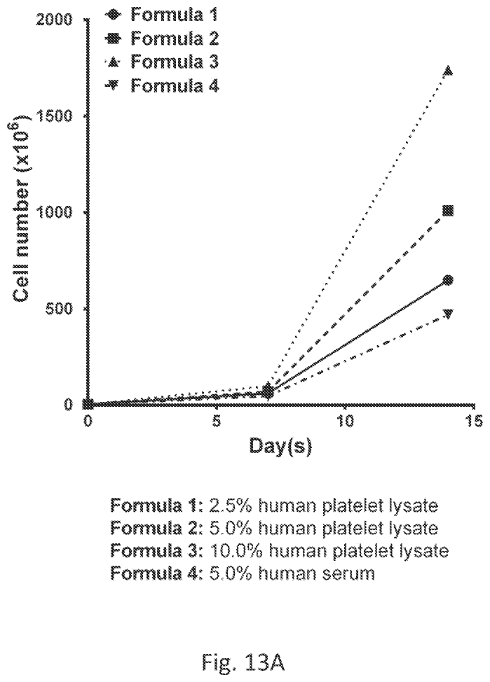

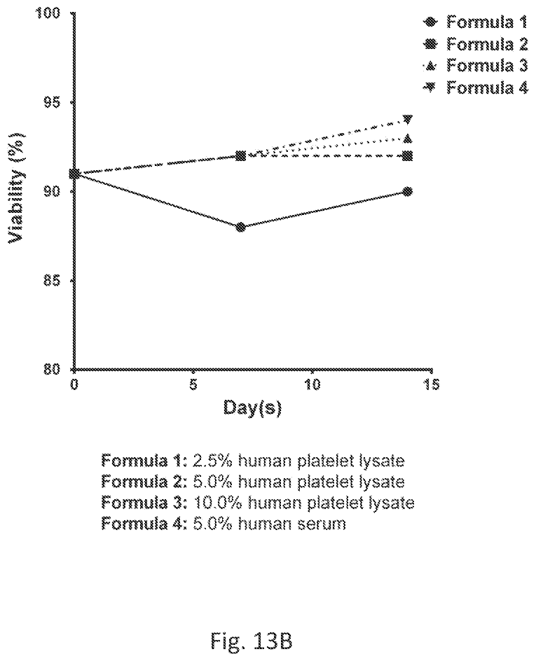

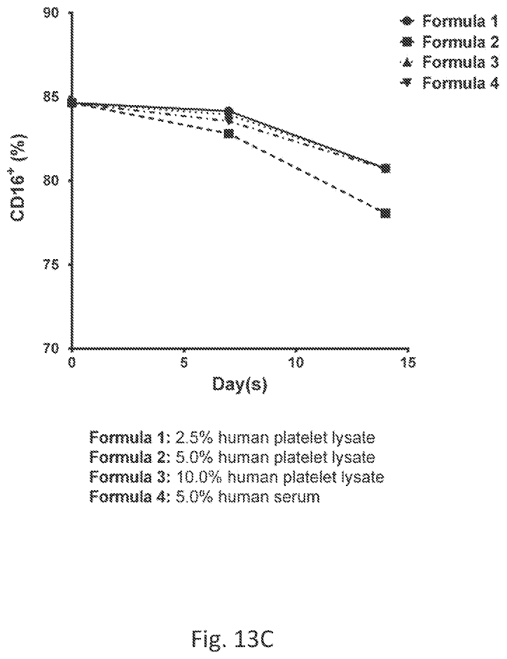

Please refer to FIG. 13A-13C. FIG. 13A-13C are the line graph presenting the effect of human platelet lysate on total cell number, cell viability, or maintaining the expression of CD16 respectively after different days of culturing human CD16⁺ natural killer cell line.

FIG. 13A showed that after culturing for 14 days, the number of the non-transgenic human CD16⁺ natural killer cells cultured in cell culture medium comprising no human platelet lysate (but comprising 5.0% human serum), 2.5% human platelet lysate, 5.0% human platelet lysate, and 10.0% human platelet lysate were 4.7×10⁸, 6.49×10⁸, 1.01×10⁹, and 1.74×10⁹ respectively. Thus, the result shows that: as compare with cell culture medium comprising no human platelet lysate (but comprising 5.0% human serum), human platelet lysate could cause 3.7 fold increase (17.4÷4.7=3.7). That is, human platelet lysate makes an unexpected result, and human platelet lysate makes non-transgenic human CD16⁺ natural killer cells expand greatly. Moreover, these results suggested that Formula 3 (comprising 10.0% human platelet lysate) was better than the rest of formulas for human CD16⁺ natural killer cells expansion.

FIG. 13B showed that after culturing for 7 days, the cell viability of the non-transgenic human CD16⁺ natural killer cells cultured in cell culture medium comprising no human platelet lysate (but comprising 5.0% human serum), 2.5% human platelet lysate, 5.0% human platelet lysate, and 10.0% human platelet lysate were maintained at 92%, 88%, 92%, and 92% respectively. After culturing for 14 days, the cell viability of the non-transgenic human CD16⁺ natural killer cells cultured in cell culture medium comprising no human platelet lysate (but comprising 5.0% human serum), 2.5% human platelet lysate, 5.0% human platelet lysate, and 10.0% human platelet lysate were maintained at 94%, 90%, 92%, and 93% respectively. Thus, the result shows that: human CD16⁺ natural killer cells that did not be treated with human platelet lysate have similar viability as human CD16⁺ natural killer cells treated with 2.5%-10.0% human platelet lysate.

FIG. 13C showed that after culturing in cell culture medium comprising no human platelet lysate (but comprising 5.0% human serum), 2.5% human platelet lysate, 5.0% human platelet lysate, or 10.0% human platelet lysate for 7 days, the percentage of CD16⁺ cells were maintained at 83.55%, 84.15%, 82.81%, and 83.95% respectively. After culturing in cell culture medium comprising no human platelet lysate (but comprising 5.0% human serum), 2.5% human platelet lysate, 5.0% human platelet lysate, or 10.0% human platelet lysate for 14 days, the percentage of CD16⁺ cells were maintained at 80.72%, 80.74%, 78.07%, and 80.76% respectively. Thus, the result shows that: 2.5%-10% human platelet lysate maintains similar CD16⁺ population as no human platelet lysate (comprising 5.0% human serum).

Moreover, based on this result, applicant believes that after isolating human CD16⁺ natural killer cell line from the 9-day cultured oNK suspension (cultured oNK), similar result could be observed.

### Embodiment 13: Culturing Non-Transgenic Human CD16⁺ Natural Killer Cell Line with Different Concentration of IL-2

The experimental method of this embodiment is almost the same as that of Embodiment 2.1, except that (1) in Step S22′, all of the cells in the cell suspensions obtained by culturing for 9 days with the culture method disclosed in the embodiments 2.1 (refer to as 9-day cultured oNK suspension) were cultured in this embodiment and the number of cells in the first container in Step S22′ was 5×10⁶; and (2) the cell culture medium comprises 5.0% human platelet lysate and \textcircled{1} 100 IU/mL IL-2, \textcircled{2} 200 IU/mL IL-2, \textcircled{3} 500 IU/mL IL-2, \textcircled{4} 750 IU/mL IL-2, or \textcircled{5} 1000 IU/mL IL-2.

Please note that both of IL-2 and human platelet lysate were required for expansion human CD16⁺ natural killer cells. In this embodiment, 1.8×10⁷ IU/mL IL-2 was equal to 1.1 mg/mL IL-2. Therefore, 100 IU/mL IL-2 was equal to 0.0612 μg/mL IL-2; 200 IU/mL IL-2 was equal to 0.1224 μg/mL IL-2; 500 IU/mL IL-2 was equal to 0.306 μg/mL IL-2; 750 IU/mL IL-2 was equal to 0.459 μg/mL IL-2; and 1000 IU/mL IL-2 was equal to 0.612 μg/mL IL-2.

The experimental method of detecting cell number, cell viability, and CD16 surface marker of the cultured cells in this embodiment is the same as that of Embodiment 2.2 and 3.4.

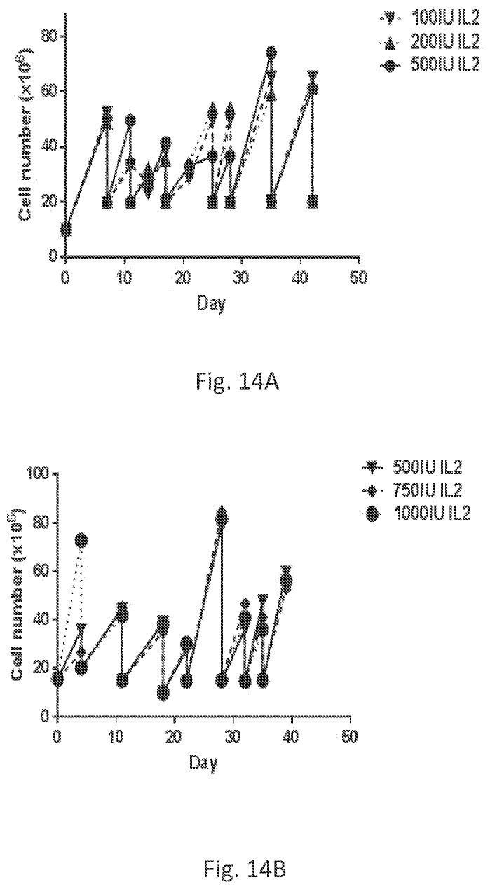

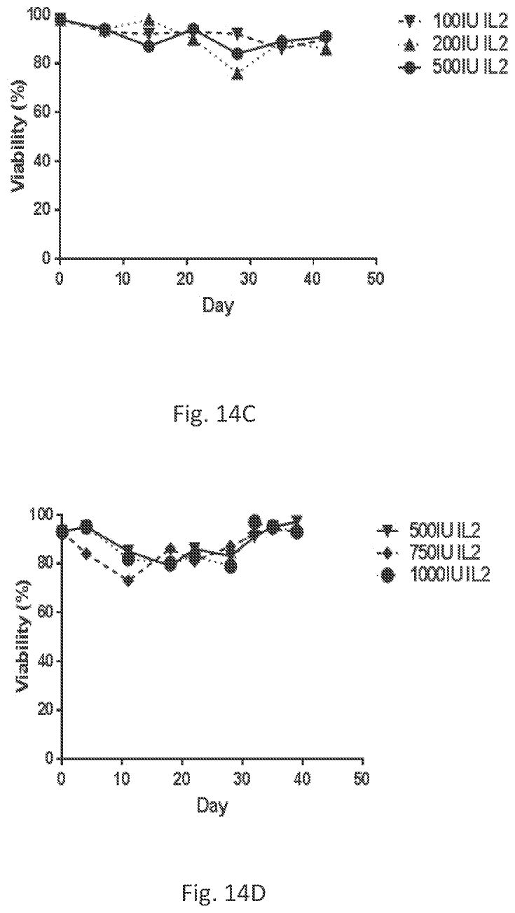

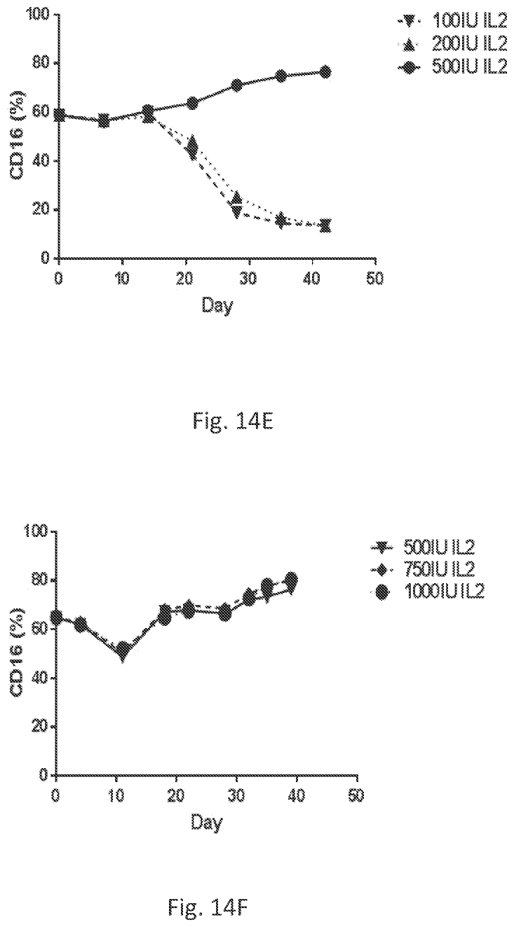

Please refer to FIG. 14A-14F. FIG. 14A-14F are the line graph presenting the effect of IL-2 on total cell number, cell viability, or maintaining the expression of CD16 respectively after different days of culturing human CD16⁺ natural killer cell line.

FIG. 14A-14B showed that IL-2 level did not influence on non-transgenic human CD16⁺ natural killer cell expansion. Please note that cells were reseeded on Day 7 and then continued to expand to Day 11; the expansion process was repeated every 11 days.

FIG. 14C-14D showed that IL-2 level did not influence on cell viability of the non-transgenic human CD16⁺ natural killer cells.

FIG. 14E-14F showed that after culturing in cell culture medium comprising 100-200 IU/mL IL-2 for 40 days, the percentage of CD16⁺ cells was dropped to less than 20%. On the other hand, after culturing in cell culture medium comprising 500-1000 IU/mL IL-2 for 40 days, the percentage of CD16⁺ cells was increased to 80%. That is, 500-1000 IU/mL IL-2 makes an unexpected result, and 500-1000 IU/mL IL-2 makes CD16⁺ population maintain greatly.

Moreover, based on this result, applicant believes that after isolating human CD16⁺ natural killer cell line from the 9-day cultured oNK suspension (cultured oNK), similar results could be observed.

### Embodiment 14: Culturing Non-Transgenic Human CD16⁺ Natural Killer Cell Line in Different Container

The experimental method of this embodiment is almost the same as that of Embodiment 2.1, except that (1) in Step S22′, all of the cells in the cell suspensions obtained by culturing for 9 days with the culture method disclosed in the embodiments 2.1 (refer to as 9-day cultured oNK suspension) were cultured in this embodiment and the number of cells in the first container in Step S22′ was 5×10⁶; (2) the cell culture medium comprises 500 IU/mL IL-2 and 5.0% human platelet lysate; and (3) the containers used in this embodiment are \textcircled{1} air-permeable container such as G-Rex 6-well culture plate or \textcircled{2} non air-permeable container such as T25 cell culture flask.

The experimental method of detecting cell number, cell viability, and CD16 surface marker of the cultured cells in this embodiment is the same as that of Embodiment 2.2 and 3.4.

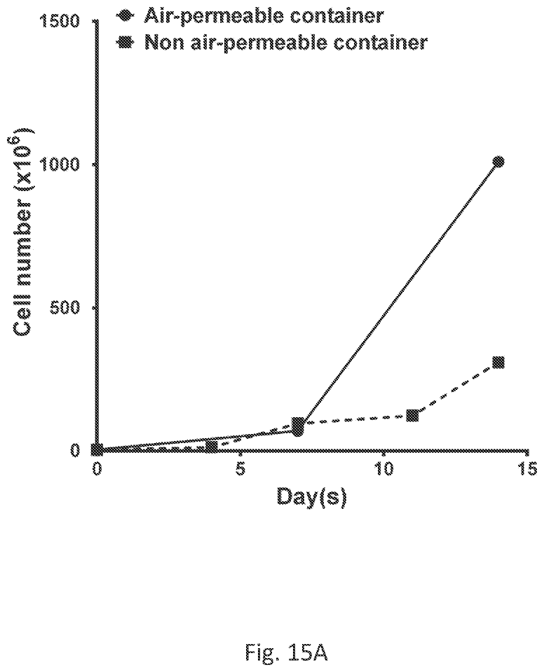

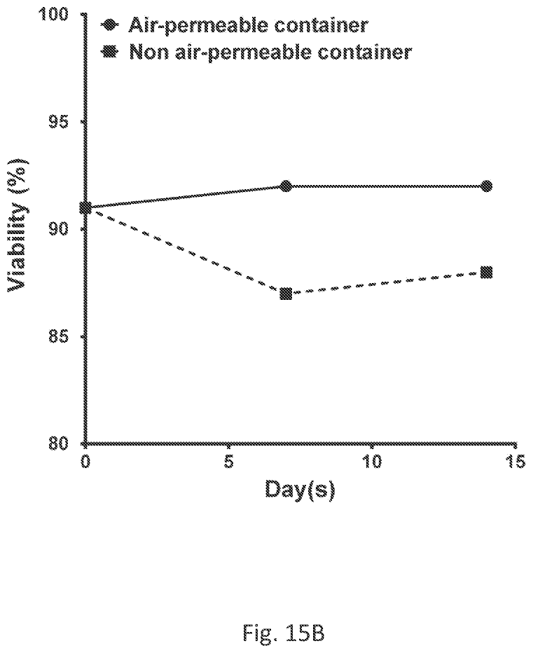

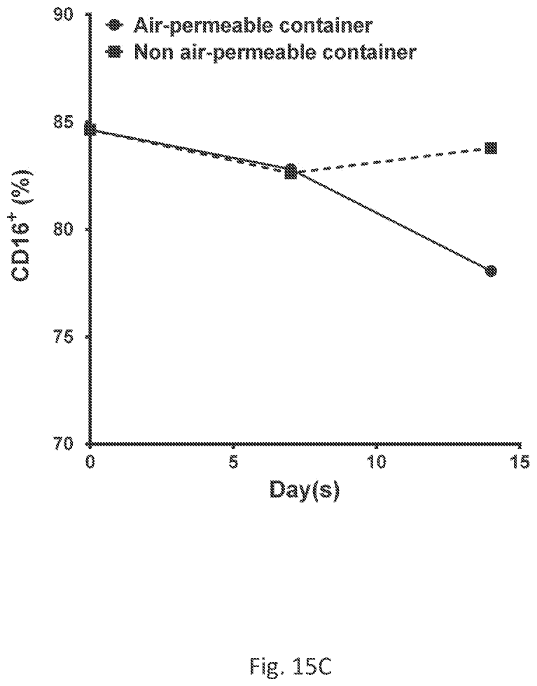

Please refer to FIG. 15A-15C. FIG. 15A-15C are the line graph presenting the effect of air-permeable container on total cell number, cell viability, or maintaining the expression of CD16 respectively after different days of culturing human CD16⁺ natural killer cell line.

FIG. 15A showed that after culturing for 14 days, the number of the non-transgenic human CD16⁺ natural killer cells cultured in non air-permeable container and air-permeable container were 3.1×10⁸ and 1.01×10⁹ respectively. Thus, the result shows that: as compare with cells cultured in non air-permeable container, air-permeable container could cause 3.26-fold increase (10.1÷3.1=3.26). That is, air-permeable container makes an unexpected result, and air-permeable container makes non-transgenic human CD16⁺ natural killer cells expand greatly.

FIG. 15B showed that after culturing for 7 days, the cell viability of the non-transgenic human CD16⁺ natural killer cells cultured in non air-permeable container and air-permeable container were maintained at 87% and 92% respectively. After culturing for 14 days, the cell viability of the non-transgenic human CD16⁺ natural killer cells cultured in non air-permeable container and air-permeable container were maintained at 88% and 92% respectively. Thus, the result shows that: human CD16⁺ natural killer cells cultured in air-permeable container better viability than human CD16⁺ natural killer cells cultured in non air-permeable container.

FIG. 15C showed that after culturing in non air-permeable container and air-permeable container for 7 days, the percentage of CD16⁺ cells were maintained at 82.63% and 82.81% respectively. After culturing in non air-permeable container and air-permeable container for 14 days, the percentage of CD16⁺ cells were maintained at 83.79% and 88.07% respectively. Thus, the result shows that: air-permeable container maintains similar CD16⁺ population as non air-permeable container does.

Moreover, based on this result, applicant believes that after isolating human CD16⁺ natural killer cell line from the 9-day cultured oNK suspension (cultured oNK), similar result could be observed.

### Embodiment 15: Prepare Exogenous Targeting Unit Complexed-oNK Cells

In this embodiment, applicant prepares an exogenous targeting unit complexed-oNK cell to which at least an exogenous targeting unit complexed. The exogenous targeting unit comprises an targeting moiety which exhibits specific binding to a biological marker on a target cell, and the targeting moiety could bind to a biological marker selected from cancer antigen, glycolipid, glycoprotein, cluster of differentiation antigen present on cells of a hematopoietic lineage, gamma-glutamyltranspeptidase, adhesion protein, hormone, growth factor, cytokine, ligand receptor, ion channel, membrane-bound form of an immunoglobulin μ. chain, alfa-fetoprotein, C-reactive protein, chromogranin A, epithelial mucin antigen, human epithelium specific antigen, Lewis(a) antigen, multidrug resistance related protein, Neu oncogene protein, neuron specific enolase, P-glycoprotein, multidrug-resistance-related antigen, p170, multidrug-resistance-related antigen, prostate specific antigen, NCAM, ganglioside molecule, MART-1, heat shock protein, sialylTn, tyrosinase, MUC-1, HER-2/neu, KSA, PSMA, p53, RAS, EGF-R, VEGF, MAGE, or other target antigen (marker) expressed by a target cell. The targeting moiety is not a nucleic acid and is not produced by the exogenous targeting unit complexed-oNK cell.

The procedure of binding a targeting moiety (such as Trastuzumab which is against HER2 protein) to oNK cells are as follows: (A) The step of preparing cell linker and binding the cell linker to the natural killer cell in order to prepare an NK-ssDNA conjugate; (B) The step of preparing targeting moiety linker (such as Trastuzumab linker) and binding the targeting moiety linker to the targeting moiety in order to prepare the targeting moiety-ssDNA conjugate; (C) Mixing NK-ssDNA conjugate and targeting moiety-ssDNA conjugate to combine NK-ssDNA conjugate and targeting moiety-ssDNA conjugate through the cell linker and its complementary sequence on the targeting moiety linker in order to prepare exogenous targeting unit complexed-conjugated natural killer cells (e.g., ACE-oNK cells or ACE-yNK cells).

Wherein the step (A) of preparing cell linker and binding the cell linker to the natural killer cell comprises the following steps (a1)˜(a4):

Step (a1) A first single strand DNA was obtained, wherein the sequence of the first single strand DNA was SEQ ID NO:5, SEQ ID NO:6, or SEQ ID NO:7.

Step (a2) The 5′ end of the first single strand DNA was modified as 5′ end thiol-modified first single strand DNA to obtain the cell linker stock. The cell linker stock is also commercially available from Integrated DNA Technologies. Actual methods of modification are known, or will be apparent, to those skilled in the art (Zimmermann, J, 2010).

Step (a3) 10-500 μL cell linker stock and 0.1-10 μL NHS-Maleimide (commercially available from Fisher Scientific) were mixed and incubated for 1-60 minute(s).

Step (a4) The mixture obtained from Step (a3) were mixed with 1×10⁶-1×10⁸ natural killer cells and incubated for 1-60 minutes to obtain NK-ssDNA conjugate.

The step (B) of preparing targeting moiety linker and binding the targeting moiety linker to targeting moiety comprises the following steps (b1)−(b4):

Step (b1) A second single strand DNA was obtained, wherein the sequence of the second single strand DNA was SEQ ID NO:8, SEQ ID NO:9, or SEQ ID NO:10, and the sequence of the second single strand DNA is the complementary strand to the first single strand DNA.

Step (b2) The 5′ end of the second single strand DNA was modified as 5′ end thiol-modified second single strand DNA to obtain the targeting moiety linker stock. The targeting moiety linker stock is also commercially available from Integrated DNA Technologies. Actual methods of modification are known, or will be apparent, to those skilled in the art (Zimmermann, J, 2010).

Step (b3) 10-500 L, targeting moiety linker stock and 0.1-10 μL NHS-Maleimide (commercially available from Fisher Scientific) were mixed and incubated for 1-60 minute(s). Step (b4) The mixture obtained from Step (b3) were mixed with 10-100 μL targeting moiety stock (commercially available from Roche) and incubated for 10 minutes to 3 hours to obtain targeting moiety-ssDNA conjugate.

The targeting moiety could be a peptide, protein, or aptamer, wherein the protein could be an antibody against a cancer antigen selected from HER2/neu (ERBB2), HER3 (ERBB3), EGFR, VEGF, VEGFR2, GD2, CTLA4, CD19, CD20, CD22, CD30, CD33 (Siglec-3), CD52 (CAMPATH-1 antigen), CD326 (EpCAM), CA-125 (MUC16), MMP9, DLL3, CD274 (PD-L1), CEA, MSLN (mesothelin), CA19-9, CD73, CD205 (DEC205), CD51, c-MET, TRAIL-R2, IGF-1R, CD3, MIF, folate receptor alpha (FOLR1), CSF1, OX-40, CD137, TfR, MUC1, CD25 (IL-2R), CD115 (CSF1R), IL1B, CD105 (Endoglin), KIR, CD47, CEA, IL-17A, DLL4, CD51, angiopoietin 2, neuropilin-1, CD37, CD223 (LAG-3), CD40, LIV-1 (SLC39A6), CD27 (TNFRSF7), CD276 (B7-H3), Trop2, Claudin1 (CLDN1), PSMA, TIM-1 (HAVcr-1), CEACAM5, CD70, LY6E, BCMA, CD135 (FLT3), APRIL, TF(F3), nectin-4, FAP, GPC3, FGFR3, a killer-cell immunoglobulin-like receptors (KIRs), a TNF receptor protein, an immunoglobulin protein, a cytokine receptor, an integrin, activating NK cell receptors, and combinations thereof.

Preferably, the targeting moiety is an antigen-binding unit or an antibody such as Trastuzumab (an antibody against HER2 protein with product name as Herceptin was purchased from Roche, Swiss).

According to the disclosures shown in the embodiments 7, 8, 11, and 15 as well as FIGS. 7B, 8, 11, and 12B, those skilled in the art would understand the chemical method of preparing antigen-binding unit-NK cell line conjugation (conjugation between antigen-binding unit and NK cell line), and also understand the application of the antigen-binding unit-NK cell line conjugation in cell therapy that specifically target abnormal cell.

### Embodiment 15.1 Cytotoxicity of Exogenous Targeting Unit Complexed-oNK Cells Against Solid Tumor

Luciferase-expressing human ovary cancer cell line SKOV3 (SKOV3-Luc, which is a Her2⁺ cell lines; catalog number AKR-232, purchased from CELL BIOLABS Inc) were intraperitoneal injection into each of the 15 female NOG mice (Jackson Laboratory) on Day 0. Five mice of each group were treated with oNK (cells in cell suspensions obtained by culturing with the culture method disclosed in the embodiments 2.1), the ACE-oNK-HER2 cells disclosed in the embodiments 7 (exogenous targeting unit complexed-oNK cells), or Vehicle (cell medium only, such as fresh growth media described in Embodiment 16.1) on Day 0, 3, 5, 11, and 18. Luminescence was detected by AMI HTX (Spectral Imaging) in the end of the experiment.

The inventors of the present invention expect that exogenous targeting unit complexed-oNK cells exerts superior potency against solid ovarian tumor, and as compare with oNK cells, exogenous targeting unit complexed-oNK cells could cause higher cytotoxicity and extend life.

### Embodiment 16: Prepare “Antigen-Binding Complex”-Expressing oNK Cells

The method for preparing oNK cells comprising a synthetic, genetically modified and/or deliberately delivered polynucleotide encoding an antigen-binding complex comprising a target-binding single-chain variable fragment (scFv) against target antigen is disclosed in this embodiment, wherein the target antigen is selected from CD2, CD3 delta, CD3 epsilon, CD3 gamma, CD4, CD7, CD8a, CD8, CD11a (ITGAL), CD11b (ITGAM), CD11c (ITGAX), CD11d (ITGAD), CD 18 (ITGB2), CD 19 (B4), CD27 (TNFRSF7), CD28, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll-like receptor, HER2, BCMA, PD-L1, VEGFR2, TCR b-chain, and combinations thereof.

Preferably, the antigen-binding complex is a chimeric antigen receptor (CAR) designed similar with a chimeric antigen receptor in a chimeric antigen receptor-T cell (CAR-T cell).

In naturally present antigen-specific T cell's membrane, there are both of (1) T cell receptors that are responsible for specifically recognizing antigen's fragments presented by HLA and (2) costimulatory molecules. Both of antigen binding signal and sufficient costimulatory signals are required for full activation of the antigen-specific T cells; wherein the costimulatory signals are induced via the costimulatory molecules being bound by their ligands that are usually shown in the target cell's membrane (Weinkove et al., 2019).

Example of costimulatory molecules expressed by T cells include CD28 subunit, ICOS (CD278) subunit, 4-1BB (CD137) subunit, OX40 (CD134) subunit, CD27 subunit, CD40 subunit, CD40L subunit, TLRs subunit, or other costimulatory molecules (Weinkove et al., 2019).

The synthetic chimeric antigen receptor (CAR) combines variable regions of an antibody with intracellular signaling components derived from the T cell receptor complex, and thus allows redirection of T cell cytotoxicity against an antigen on any HLA background (antigen processing and presentation by HLA are not required) (Weinkove et al., 2019).

The first-generation CARs incorporate only intracellular CD3 (such as CD3 zeta, also known as CD3c). The second-generation CARs further incorporate one intracellular signaling domain of costimulatory molecule (such as CD28), and the third-generation CARs incorporate more than one intracellular signaling domains of costimulatory molecules (such as combining CD28 and 4-1BB) (Weinkove et al., 2019).

Take CD19 as an example to explain the method of preparing oNK comprising a synthetic, genetically modified and/or deliberately delivered polynucleotide encoding a chimeric antigen receptor (CAR) comprising a target-binding single-chain variable fragment (scFv) against CD19 as below.

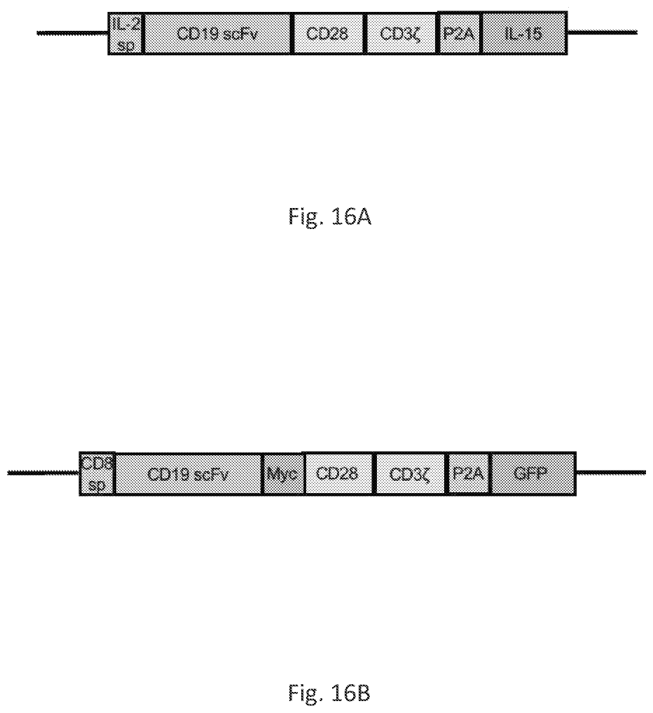

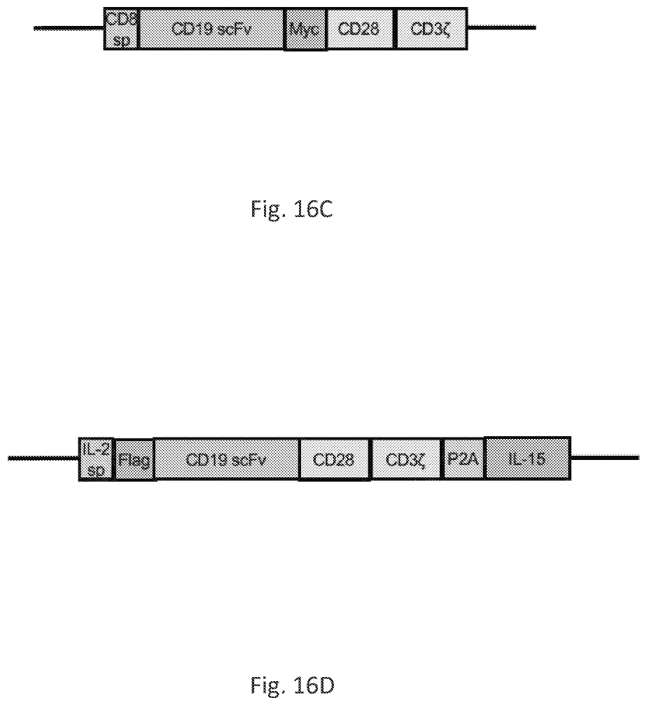

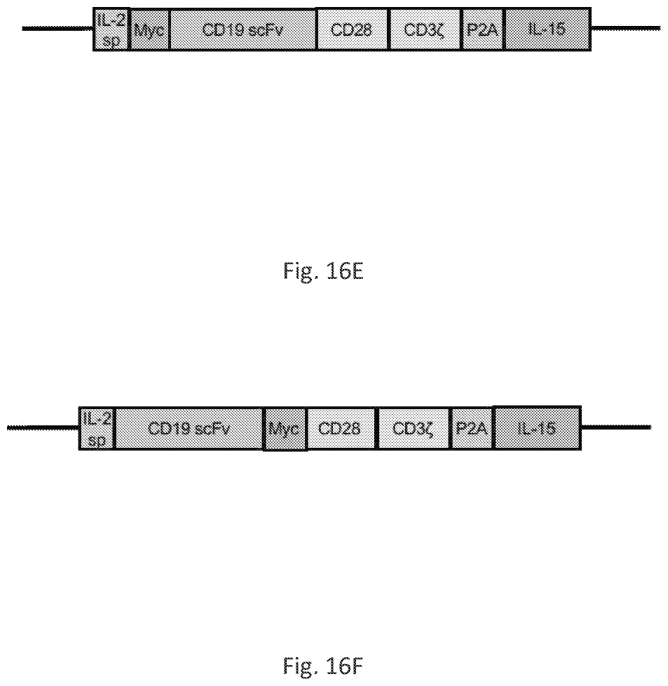

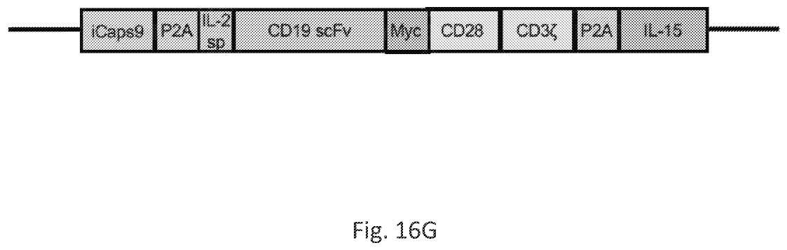

FIG. 16A-16G demonstrate the constructions of the CD19 CAR. The construct comprises a synthetic, genetically modified and/or deliberately delivered polynucleotide encoding a chimeric antigen receptor (CAR) comprising a target-binding single-chain variable fragment (scFv) against CD19.

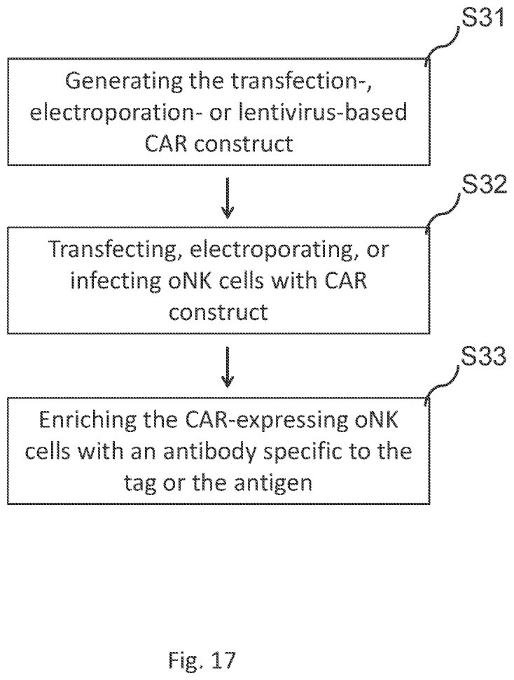

FIG. 17 illustrates the method of preparing oNK comprising a synthetic, genetically modified and/or purposely deliberately delivered polynucleotide encoding a chimeric antigen receptor (CAR) such as shown in FIG. 16A-16G. The method for establishing human CAR-expressing CD16⁺ natural killer cells comprises at least the following steps:

Step S31: Generating the transfection-, electroporation- or lentivirus-based CAR constructs;

Step S32: Transfecting, electroporating, or infecting oNK cells with CAR construct;

Step S33: Enriching the CAR-expressing oNK cells with an antibody specific to the tag or the antigen.

### Embodiment 16.1: Prepare Chimeric Antigen Receptor (CAR)-Expressing oNK Cells

The following describes a specific embodiment of establishing a CAR-expressing human CD16⁺ natural killer cell line against CD19 that does not include genetically modified polynucleotide encoding the CD16 receptor (such as anti-CD 19 CAR-expressing oNK cells) by the present invention, but the application of the invention is not limited thereto, which means the invention can also be used for establishing CAR-expressing human CD16⁺ natural killer cell lines against other CAR-target antigens that does not include genetically modified polynucleotide encoding the CD16 receptor. For example, the invention can also be used for establishing CAR-expressing human CD16⁺ natural killer cell line against CD70, GPC3, or PD-L1 that does not include genetically modified polynucleotide encoding the CD16 receptor. In the present invention, inventors expect that the higher the binding capacity of the CAR-expressing human CD16⁺ natural killer cell line against a CAR-target antigen (such as anti-CD 19 CAR-expressing oNK cells against CD19 recombinant protein), the higher the cytotoxicity of CAR-expressing human CD16⁺ natural killer cell line against the target cells that express CAR-target antigen.

In the step S31, anti-CD19 CAR nucleotide sequence of SEQ ID NO:41, SEQ ID NO:42, SEQ ID NO:43, SEQ ID NO:44, SEQ ID NO:45, SEQ ID NO:46, or SEQ ID NO:47 is chemically synthesized by Synbio Technologies. A polymerase chain reaction (PCR) Kit (M05305, New England Biolabs) is used to amplify said synthesized anti-CD19 CAR nucleotide to a large enough amount. Restriction enzyme (such as HinDIII, EcoRI, or BamHI) is used to cut the synthesized anti-CD19 CAR nucleotide and a vector such as linearized pBudCE vector, (ThermoFisher Scientific), pMAXCloning vector (Lonza), pCD810A-1 or pCD510B-1 (System Biosciences). A ligase (such as Taq DNA ligase) is mixed with the restricted enzyme digested synthesized anti-CD19 CAR nucleotide and the restricted enzyme digested vector to promote ligation reactions and “anti-CD19 CAR plasmid comprising Myc gene (or anti-CD19 CAR plasmid comprising tag)” formation. Actual methods of preparing transfection-, electroporation- or lentivirus-based CAR constructs (such as anti-CD19 CAR plasmid or anti-CD19 CAR plasmid comprising Myc gene or anti-CD19 CAR plasmid comprising tag) are known, or will be apparent, to those skilled in the art (U.S. Pat. No. 7,446,179; WO 2015157252; U.S. Pat. No. 7,446,179).

In the step S32, the anti-CD19 CAR plasmids comprising Myc gene (or anti-CD19 CAR plasmid comprising tag) are transfected, electroporated or transduced into oNK to obtain anti-CD19 CAR-expressing oNK.

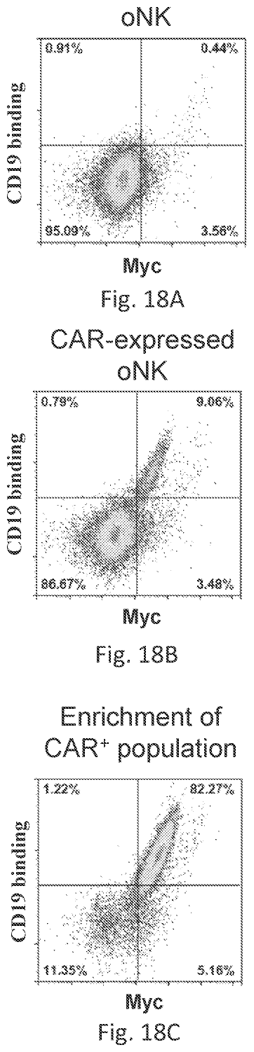

For example, cell suspensions obtained by culturing with the culture method disclosed in the embodiments 2.1 (refer to as cultured oNK suspension) were harvested (please refer to FIG. 18A) and transfected (such as Lipofectamine 2000, Lipofectamine 3000, ThermoFisher Scientific), electroporated (such as P3 Primary Cell Nucleofector Kit, SF Cell line Nucleofector Kit, Lonza) or transduced (such as lentivirus, retrovirus) with CAR construct. The CAR-expressing oNK were expanded with fresh growth media (such as DMEM culture medium, alpha modification of Eagle's minimum essential medium, or XVIVO 10 culture medium comprising 0.5%-30% (Volume percent, vol %, v/v) Human platelet lysate, and 100-3000 IU/mL Interleukin 2 (IL-2)) and cultured at 37° C. in G-Rex plate.

### Embodiment 16.2: Enrich Chimeric Antigen Receptor (CAR)-Expressing oNK Cells

In the step S33, to enrich the CAR-expressing oNK cells, the cells obtained from step S32 were stained with tagged CD19 recombinant protein (Cat No. 11880-H08H from Sino Biological, or FITC-conjugated CD 19 recombinant protein with Cat No. CD9-HF2H2 from ACROBiosystem, or APC-conjugated CD19 recombinant protein with Cat No. CD19-3309HA from Creative BioMart) and fluorescence-conjugated anti-Myc Antibody (Novus Biologicals) (please refer to FIG. 18B). The CAR-expressing oNK were further enriched by cell sorter (BD Bioscience) or anti-fluorescence MicroBeads (Miltenyl Biotec) to obtain the anti-CD19 CAR-expressing oNK cells (CAR19-oNK) (please refer to FIG. 18C), and were expanded in fresh oNK growth media (such as DMEM culture medium, alpha modification of Eagle's minimum essential medium, or XVIVO 10 culture medium comprising 0.5%-30% (Volume percent, vol %, v/v) Human platelet lysate, and 100-3000 IU/mL Interleukin 2 (IL-2)) and cultured at 37° C. in G-Rex plate.

The results for fluorescent analysis of the cultured oNK cell suspension without the transuded anti-CD19 CAR construct are shown in FIG. 18A; FIG. 18A is the two-dimensional dot plot representing the Myc⁺ cell population with CD19 binding activity in the cultured oNK cell suspension without the transuded anti-CD19 CAR construct. The results for fluorescent analysis of the cultured oNK cell suspension with the transuded anti-CD19 CAR construct are shown in FIG. 18B; FIG. 18B is the two-dimensional dot plot representing the Myc+ cell population with CD19 binding activity in the cultured oNK cell suspension with the transuded anti-CD19 CAR construct.

The results in FIG. 18A show that the cultured oNK cell suspension without the transduced anti-CD19 CAR construct exhibit no fluorescent signal of expressed Myc tag and bound human CD19 recombinant protein. There is 0.44% of double positive background signal in the cultured oNK cell suspension without transduced anti-CD19 CAR construct.

The results in FIG. 18B show that the cultured oNK cell suspension with the transuded anti-CD19 CAR construct exhibit 9.06% of double positive of fluorescent signal of expressed Myc tag and bound human CD19 recombinant protein.

Please refer to FIG. 18C. FIG. 18C is the two-dimensional dot plot representing the isolated Myc⁺ cells with CD19 binding activity that are isolated from the cell suspension as shown in FIG. 18B by the labeling of tagged CD19 recombinant protein and fluorescence-conjugated anti-Myc antibody. The results in FIG. 18C show that the isolated cultured oNK cell suspension with the transuded anti-CD19 CAR construct in FIG. 18B exhibit 82.27% of double positive of fluorescent signal of expressed Myc tag and bound human CD19 recombinant protein.

Preferably, anti-CD19 CAR nucleotide sequence comprises a sequence of SEQ ID NO:41, SEQ ID NO:42, SEQ ID NO:43, SEQ ID NO:44, SEQ ID NO:45, SEQ ID NO:46, SEQ ID NO:47, or other anti-CD19 CAR nucleotide sequence.

The method of preparing CAR plasmids and lentiviral particles comprising CAR plasmids are known, or will be apparent, to those skilled in the art.

In addition, CAR plasmids and lentiviral particles comprising CAR plasmids are commercially available. For example, lentiviral particles comprising anti-CD19 CAR plasmid could be purchased from Creative Biolabs (this anti-CD19 CAR construct comprises CD19 scFv domain, CD28 domain, and CD3 zeta domain; Cat No. VP-CAR-LC61); lentiviral particles comprising anti-BCMA CAR plasmid could be purchased from Creative Biolabs (Cat No. VP-CAR-LC534); lentiviral particles comprising anti-HER2 CAR plasmid could be purchased from Creative Biolabs (Cat No. VP-CAR-LC834); lentiviral particles comprising anti-PD-L1 CAR plasmid could be purchased from Creative Biolabs (Cat No. CAR-ZP1471); lentiviral particles comprising anti-VEGFR2 CAR plasmid could be purchased from Creative Biolabs (Cat No. VP-CAR-LC616); lentiviral particles comprising anti-TCR b-chain CAR plasmid could be purchased from Creative Biolabs (Cat No. VP-TCR-YC160); lentiviral particles comprising anti-ICAM-1 CAR plasmid could be purchased from Creative Biolabs (Cat No. CAR-ZP7800); lentiviral particles comprising anti-PD-1 CAR plasmid could be purchased from Creative Biolabs (Cat No. VP-CAR-LC412).

The cell suspensions obtained by culturing with the culture method disclosed in the embodiments 2.1 (refer to as cultured oNK suspension) could be transfected (such as Lipofectamine 2000, Lipofectamine 3000, ThermoFisher Scientific), electroporated (such as P3 Primary Cell Nucleofector Kit, SF Cell line Nucleofector Kit, Lonza) or transduced (such as lentivirus, retrovirus) with each of these CAR plasmids or lentiviral particles comprising CAR plasmids to obtain the CAR-expressing oNK cells against CD2, CD3 delta, CD3 epsilon, CD3 gamma, CD4, CD7, CD8a, CD8, CD11a (ITGAL), CD11b (ITGAM), CD11c (ITGAX), CD11d (ITGAD), CD 18 (ITGB2), CD 19 (B4), CD27 (TNFRSF7), CD28, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll-like receptor, HER2, BCMA, PD-L1, VEGFR2, TCR b-chain, or combinations thereof.

### Embodiment 17: Non-Tumorigenicity of CAR-Expressing oNK Cells

Six to eight-week-old female BALB/c nude mice (purchased from The Jackson Laboratory or BioLasco, Taiwan) were used in this Embodiment. 25 mice were randomly assigned into five groups, which were a SK-OV-3 group, Daudi group, oNK group, CAR19-oNK group, and DPBS group.

A human ovarian cancer cell line “SK-OV-3” (Purchased from ATCC; The deposit number is ATCC HTB-77), human B lymphoblastoid cell lines “Daudi” (Purchased from ATCC; The deposit number is ATCC CCL-213), a cell suspension that was obtained by culturing for 101 days with the culture method disclosed in the embodiments 2.1 (101-day cultured oNKsuspension of the present invention, refer to as 101-day cultured oNK suspension), and an anti-CD19 CAR-expressing oNK cell suspension (a cell suspension that was obtained by culturing oNK cells for 25 days with the culture method disclosed in the embodiments 2.1 and then performing the transduction process and CAR-expressing cell isolation process as disclosed in the embodiments 16.1 and 16.2, refer to as 25-day cultured CAR19-oNK cell suspension) were used in this Embodiment.

1×10⁷ SK-OV-3 cells, 1×10⁷ Daudi cells, 1×10⁷ cells in the 101-day cultured oNK suspension, and 1×10⁷ cells in 25-day cultured CAR19-oNK cell suspension were suspended respectively in 100 L of Dulbecco's Phosphate-Buffered Saline (DPBS) to obtain different cell suspensions. The cell suspensions and 100 μL of DPBS were subcutaneously implanted in female BALB/crude mice in SK-OV-3 group, Daudi group, oNK group, CAR19-oNK group, and DPBS group on Day 0 respectively. Tumor growth in each mouse was observed on Day 21, Day 24, Day 42, and Day 59, and the mice were euthanized on Day 59.

Please refer to Table 5. Table 5 shows the results of tumor formation in nude mice xenografted with different cell lines.

Table 5 shows that there was no tumor formation in the mice of DPBS groups (negative control group) throughout the study period (0/5, 0%), while all five mice in SK-OV-3 group (positive control group) developed tumors (5/5, 100%). For mice xenografted with lymphoma cell line Daudi, 4 out of 5 mice in Daudi group developed tumors (4/5, 80%) that lasted until end of study (Day 59).

For mice xenografted with oNK cells or anti-CD19 CAR-expressing oNK cells of the present invention, there was no tumor formation in mice in oNK group and CAR19-oNK group throughout the study period (0/5, 0%). These study results provide evidence that non-irradiated oNK cells and the CAR-expressed oNK cells are non-tumorigenic and safe for future clinical application and disease treatment.

TABLE 5
the results of tumor formation in nude mice
xenografted with different cell lines.

| | Tumor incidence | | | |
|---|---|---|---|---|
| Cell type | Day 21 | Day 24 | Day 42 | Day 59 |
| SK-OV-3 suspension | 5/5 | 5/5 | 5/5 | 5/5 |
| Daudi suspension | 4/5 | 4/5 | 4/5 | 4/5 |
| Non-irradiated oNK | 0/5 | 0/5 | 0/5 | 0/5 |
| suspension | | | | |
| Non-irradiated CAR19-NK | 0/5 | 0/5 | 0/5 | 0/5 |
| suspension | | | | |
| DPBS | 0/5 | 0/5 | 0/5 | 0/5 |

### Embodiment 18: Analyze Cytotoxicity of CAR-Expressing oNK Cells Against Target Cells In Vitro

### Embodiment 18.1 CD19 Binding Activity of CAR19-oNK Cells

Effector oNK (cell suspensions obtained by culturing for 70 days with the culture method disclosed in the embodiments 2.1; also refer to as 70-day cultured oNK suspension) and CAR19-oNK (70-day cultured CAR19-oNK cell suspension obtained by culturing oNK cells for 70 days with the culture method disclosed in the embodiments 2.1 and then performing the transduction process and CAR-expressing cell isolation process as disclosed in the embodiments 16.1 and 16.2; wherein the transduction process was based on the anti-CD19 CAR-expressing pseudo lentiviral particles that were prepared from pCD810A-1 and lentivirus-based CD19 CAR constructs as shown in FIG. 16A) were respectively co-incubated with fluorescence-conjugated CD19 recombinant protein (Cat No. CD9-HF2H2-25 ug from Acro Biosystems).

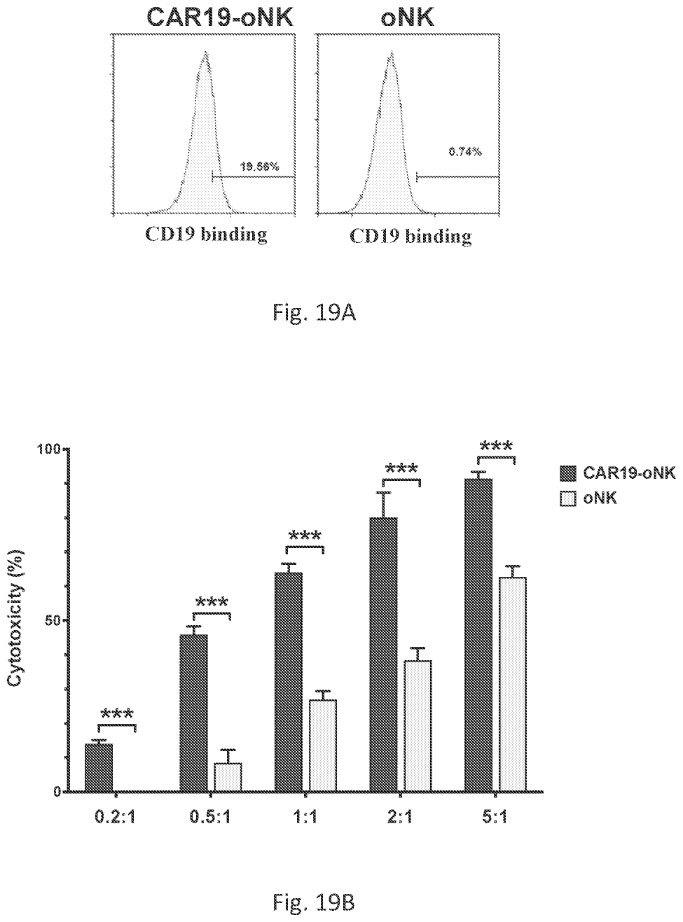

The results for CD19 binding activity of the oNK and CAR19-oNK are shown in FIG. 19A; FIG. 19A is the histogram presenting the CD19 binding activity of the oNK and CAR19-oNK.

The results in FIG. 19A show that compared with oNK cells, anti-CD19 CAR-expressing oNK cells (CAR19-oNK) elicits enhanced CD19 binding activity and thus may elicit enhanced cytotoxicity against CD19⁺ B-cell lymphoma.

### Embodiment 18.2 Cytotoxicity of CAR19-oNK Cells Against CD19⁺ B-Cell Lymphoma

Effector oNK (cell suspensions obtained by culturing for 70 days with the culture method disclosed in the embodiments 2.1; also refer to as 70-day cultured oNK suspension) and CAR19-oNK (70-day cultured CAR19-oNK cell suspension obtained by culturing oNK cells for 70 days with the culture method disclosed in the embodiments 2.1 and then performing the transduction process and CAR-expressing cell isolation process as disclosed in the embodiments 16.1 and 16.2; wherein the transduction process was based on the anti-CD19 CAR-expressing pseudo lentiviral particles that were prepared from pCD810A-1 and lentivirus-based CD19 CAR constructs as shown in FIG. 16A) were respectively co-incubated with luciferase-expressing target cell Raji (Raji-Luc, which is a CD19⁺ B cell lymphoma cell line expressing luciferase gene) at E:T ratio of 0.2:1, 0.5:1, 1:1, 2:1 and 5:1 for 1 hour. D-Luciferin (consumable substrate of luciferase, purchased form GoldBio) were added to the cells and incubated at 37° C. for 10 minutes. Luminescence of each well were detected by using HTX Multi-Mode Reader (BioTek) and calculated as percentage of cytotoxicity related to the luminescence acquired from Raji-Luc cells without treatment; wherein the Luminescence is the catalytic product of luciferase. Statistics were analyzed by student t test. **, p<0.01; ***, p<0.001; ****, p<0.0001. Actual methods of this experiment are known, or will be apparent, to those skilled in the art (Rigo V, 2017).

Please refer to 19B. FIG. 19B is the bar chart presenting the comparison of the cytotoxic function between the oNK and CAR19-oNK to kill CD19⁺ B-cell lymphoma at different effector (E) to target (T) ratio. FIG. 19B shows that the oNK kill 0.0±0.0%, 8.5±3.8%, 27.3±2.48%, 38.4±3.6%, and 62.7±3.2% of CD19⁺ B-cell lymphoma at the ratio of the number of effector cells to the number of target cells being 0.2:1, 0.5:1, 1:1, 2:1, and 5:1 (ET0.2 to ET5) respectively; the CAR-19 oNK kill 13.9±1.2%, 45.9±2.5%, 64.1±2.5%, 80.0±7.4%, and 91.4±1.9% of CD19⁺ B-cell lymphoma at the ratio of the number of effector cells to the number of target cells being 0.2:1, 0.5:1, 1:1, 2:1, and 5:1 (ET0.2 to ET5) respectively. FIG. 19B demonstrates that compared with oNK cells, anti-CD19 CAR-expressed oNK cells (CAR19-oNK) elicits enhanced cytotoxicity against CD19⁺ B-cell lymphoma at different E:T ratio.

Moreover, the result shows that: as compare with oNK cells, CAR19-oNK cells could cause about ∞-fold increase of cytotoxicity (13.1÷0=∞). This is an unexpected result.

### Embodiment 18.3 CAR19-oNK Cell has No Off-Target Cytotoxicity

Effector oNK (cell suspensions obtained by culturing for 69 days with the culture method disclosed in the embodiments 2.1; also refer to as 69-day cultured oNK suspension) and CAR19-oNK (69-day cultured CAR19-oNK cell suspension obtained by culturing oNK cells for 69 days with the culture method disclosed in the embodiments 2.1 and then performing the transduction process and CAR-expressing cell isolation process as disclosed in the embodiments 16.1 and 16.2; wherein the transduction process was based on the anti-CD19 CAR-expressing pseudo lentiviral particles that were prepared from pCD810A-1 and lentivirus-based CD19 CAR constructs as shown in FIG. 16A) were respectively co-incubated with Calcein-labeled target cell K562 (CCL-243, purchased from ATCC; K562 is a CD19⁻ cancer cell line) at E:T ratio of 0.2:1, 0.5:1, 1:1, 2:1 and 5:1 for 2.5 hours; wherein the Calcein is a fluorescent dye. Triton X-100-lysed Calcein-labeled K562 was set as 100% lysed control. The fluorescence signals of dead cells in the supernatant of each sample and 100% lysed control were detected by 490 nm excitation and 520 nm for emission by HTX Multi-Mode Reader.

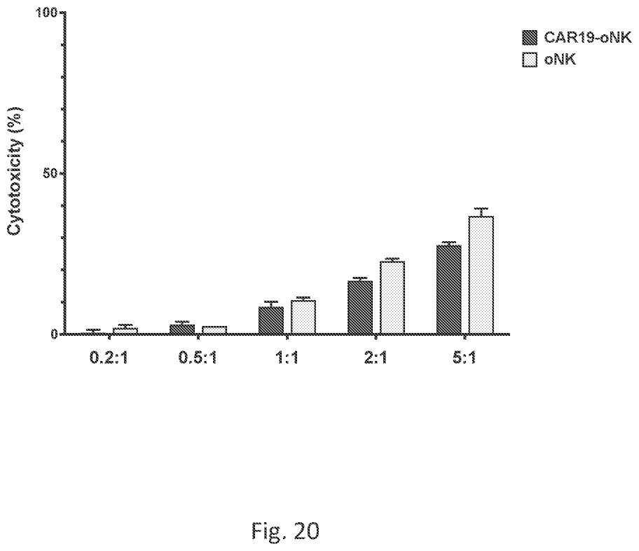

Please refer to FIG. 20. FIG. 20 is the bar chart presenting the comparison of the cytotoxic function between the oNK and CAR19-oNK to kill CD19 cancer cell at different effetor (E) to target (T) ratio.

FIG. 20 shows that CAR19-oNK does not effectively exert enhanced cytotoxicity against CD19⁻ cancer cell line compared with that of parental oNK cells. Therefore, CAR19-oNK has no off-target cytotoxicity.

### Embodiment 19: Analyze Cytotoxicity of CAR-Expressing oNK Cells Against Target Cells In Vivo

### Embodiment 19.1 Cytotoxicity of CAR-Expressing oNK Cells Against Liquid Tumor

1×10⁵ luciferase-expressing target cell Raji (Raji-Luc, which is a CD19⁺ B cell lymphoma cell lines; CCL-86, ATCC) were intravenously injected into each of the 15 female immune compromised NSG mice (Jackson Laboratory) on Day 0. Five mice of each group were treated with 5×10⁶ oNK (cells in cell suspensions obtained by culturing for 117 days with the culture method disclosed in the embodiments 2.1; also refer to as 117-day cultured oNK suspension), CAR19-oNK (117-day cultured CAR19-oNK cell suspension obtained by culturing oNK cells for 117 days with the culture method disclosed in the embodiments 2.1 and then performing the transduction process and CAR-expressing cell isolation process as disclosed in the embodiments 16.1 and 16.2; wherein the transduction process was based on the anti-CD19 CAR-expressing pseudo lentiviral particles that were prepared from pCD810A-1 and lentivirus-based CD19 CAR constructs as shown in FIG. 16A), or Vehicle (cell medium only, such as fresh growth media described in Embodiment 16.1) on Day 0, 3, 7 and 10. Luminescence was detected by AMI HTX (Spectral Imaging) on Day 0, 4, 7, 11, 14 and 18.

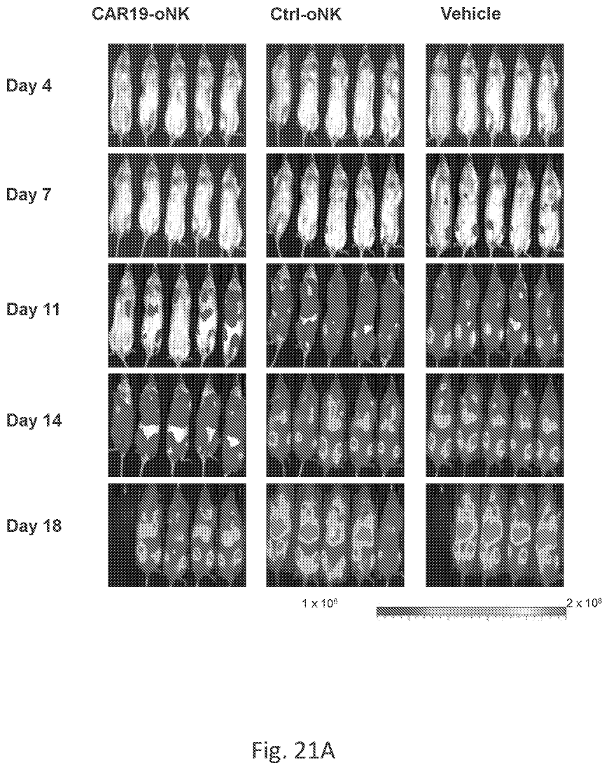

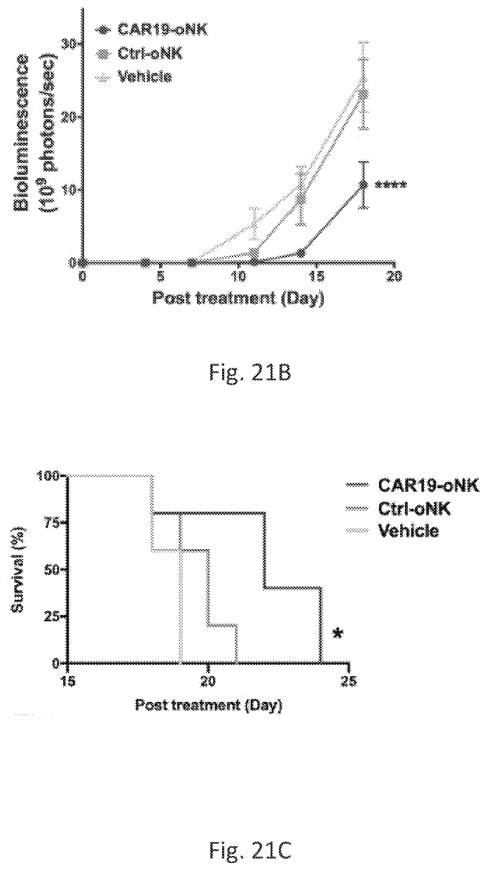

FIG. 21A is the fluorescent images of tumor cells in mice on Day 4, 7, 11, 14, and 18. FIG. 21A demonstrates that CAR19-oNK exerts superior potency against lymphoma cells.

FIG. 21B is the statistical analysis of luminescence shown in FIG. 21A by Mixed-effects model. *, p<0.05; ****, p<0.0001. FIG. 21B shows that: as compare with oNK cells, CAR19-oNK cells could cause about 11.6-fold increase of cytotoxicity (13.8×10⁸ 1.2×10⁸=11.6). This is an unexpected result.

FIG. 21C is the survival rate of mice show in FIG. 21A. FIG. 21C demonstrates significantly prolonged survival rate of mice in CAR19-oNK-treated mice. FIG. 21C shows that: by comparing with treatment of oNK cells, the treatment of CAR19-oNK could extend life as much as 20% to 80% times. This is an unexpected result.

FIGS. 21A and 21B show that compared with vehicle, CAR19-oNK cells elicits significantly enhanced potency against CD19⁺ B-cell lymphoma.

### Embodiment 19.2 Cytotoxicity of CAR-Expressing oNK Cells Against Solid Tumor

Luciferase-expressing human ovary cancer cell line SKOV3 (SKOV3-Luc, which is a HER2⁺ cell lines; catalog number AKR-232, purchased from CELL BIOLABS Inc) were intraperitoneal injection into each of the 15 female NSG mice (Jackson Laboratory) on Day 0. Five mice of each group were treated with oNK (cells in cell suspensions obtained by culturing with the culture method disclosed in the embodiments 2.1), CARHER2-oNK (CARHER2-oNK cell suspension obtained by culturing oNK cells with the culture method disclosed in the embodiments 2.1 and then performing the transduction process with the anti-HER2 CAR-expressing pseudo lentiviral particles), or Vehicle (cell medium only, such as fresh growth media described in Embodiment 16.1) on Day 0, 4, 7, 10, 14, and 17. Luminescence was detected by AMI HTX (Spectral Imaging) weekly and in the end of the experiment.

The inventors of the present invention expect that CARHER2-oNK exerts superior potency against solid ovarian tumor, and as compare with oNK cells, CARHER2-oNK cells could cause higher cytotoxicity and extend life.

### Embodiment 20: Monitor Long Term Cell Viability, Cell Proliferation, CD19 Binding Activity and Cell Surface Markers of CAR19-oNK

### Embodiment 20.1 Cell Viability and Cell Proliferation of CAR19-oNK

CAR19-oNK (cell suspensions obtained by culturing for 4-day to 83-day CAR-oNK suspension generated by the transduction of cultured oNK with anti-CD19 CAR-expressing pseudo lentiviral particles prepared from pCD810A-1 and lentivirus-based CD19 CAR constructs as shown in FIG. 16A) was used in this embodiment. The culture method for cultureing CAR-oNK is almost same as that in embodiment 2, but IL-2 in Step S22 may not be required. Therefore, the culture method for cultureing CAR-oNK comprises at least the following step:

Step S41: Obtaining CAR19-oNK;

Step S42: In the container, contacting the CAR19-oNK with a culture medium comprising human platelet lysate on Day 0; and

Step S43: Culturing the CAR19-oNK for multiple days to proliferate the CAR19-oNK.

The detail condition could be found in embodiment 2.1, embodiment 11, and embodiment 13, and could further found in embodiment 12.

Each sample of the cell suspensions, which were obtained by culturing CAR19-oNK for different days, was mixed with an equal volume of Trypan blue, and the viability and cell number were observed. The viability was determined by dividing viable cell number by total cell number.

### Embodiment 20.2 Detecting of CD56, CD3, and CD2 Surface Markers of the Cultured CAR19-oNK

Each sample of the cell suspensions, which were obtained at different time points in Embodiment 20.1, was centrifuged; the supernatant was removed, the cells were resuspended in the buffer, then mixed with 1 μL of CD56 fluorescent labeled antibody (Cat. No. 318304, Biolegend, USA), 1 μL of CD3 fluorescent labeled antibody (Cat. No. 300410, Biolegend, USA), and 1 μL of CD2 fluorescent labeled antibody (Cat. No. 300222, Biolegend, USA) to simultaneously label cells expressing CD56 molecule, CD3 molecule, and/or CD2 molecule. Finally, the cell sorter or flow cytometer was used to analyze whether the cells exhibited CD56 molecules, CD3 molecules, and/or CD2 molecules, and the percentage of cells with various cell surface makers was calculated.

### Embodiment 20.3 Detection of CD16 Expression of the Cultured CAR19-oNK

Each sample of the cell suspensions, which were obtained at different time points in Embodiment 20.1, was centrifuged; the supernatant was removed, the cells were resuspended in the buffer, then mixed with 1 μL of CD16 fluorescent labeled antibody (Cat. No. 302016, Biolegend, USA) to label cells expressing CD16. Finally, the cell sorter or flow cytometer was used to analyze whether the cells exhibited CD16, and the percentage of cells with CD16 was calculated.

### Embodiment 20.4 Detection of Binding Capacity of the Cultured CAR19-oNK to CD19 Recombinant Protein

Each sample of the cell suspensions, which were obtained at different time points in Embodiment 20.1, was centrifuged; the supernatant was removed, the cells were resuspended in the buffer, then mixed with tagged CD19 recombinant protein (Cat No. 11880-H08H from Sino Biological, or FITC-conjugated CD 19 recombinant protein with Cat No. CD9-HF2H2 from ACROBiosystem, or APC-conjugated CD19 recombinant protein with Cat No. CD19-3309HA from Creative BioMart). Finally, the cell sorter or flow cytometer was used to analyze whether the cells binding to the CD19 recombinant protein, and the percentage of cells binding to the CD19 recombinant protein was calculated.

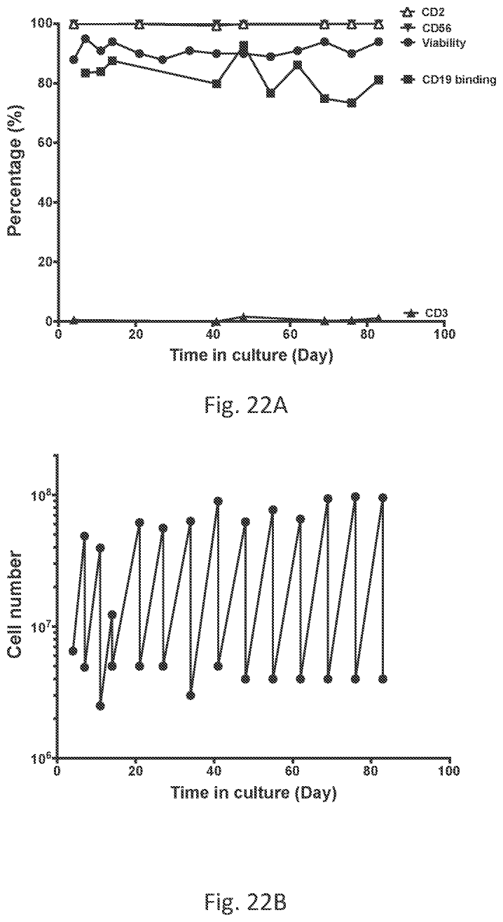

FIG. 22A is the line graph presenting the cell viability, CD19 binding activity and cell surface markers of CAR19-oNK within 83 days of culturing. FIG. 22A shows that cell viability was maintained at 88-95% after 4, 7, 11, 14, 21, 27, 34, 41, 48, 55, 62, 69, 76 and 83 days of culture of CAR19-oNK; CD2 and CD56 were maintained at >99% after 4, 21, 41, 48, 69, 76 and 83 days of culture of CAR19-oNK; CD3 was maintained at <2% after 4, 41, 48, 69, 76 and 83 days of culture of CAR19-oNK. Our data shows that CD16 were gradually decreased from 84% after 4, 7, 11, 14, 21, 27, 41, 48, 55, 62, 69, 76 and 83 days of culture of CAR19-oNK.

FIG. 22B is the line graph presenting the proliferation of CAR19-oNK within 83 days of culturing. FIG. 22B shows that culturing the CAR19-oNK with the culture method of the present invention can maintain stable expansion within 83 days. Please note that cells were reseeded on Day 7 and then continued to expand to Day 11; the expansion process was repeated every 11 days.

The cells carrying a phenotype of CD3⁻CD56⁺CD16⁺ with CD19 recombinant protein binding activity are CAR19-oNK of the present invention. These CAR19-oNK cells could be isolated by cell sorter (BD Bioscience).

### Embodiment 21 Detection of Cytokine Secretion of CAR19-oNK

CAR19-oNK cell suspensions (cell suspensions obtained by culturing for 50-day CAR-oNK suspension generated by the transduction of cultured oNK with anti-CD19 CAR-expressing pseudo lentiviral particles prepared from pCD810A-1 and lentivirus-based CD19 CAR constructs comprising IL-15 expression domain as shown in FIG. 16A) and Ctrl-oNK cell suspensions (obtained by culturing for 50 days with the culture method disclosed in the embodiments 2.1; also refer to as 50-day cultured oNK suspension) were used in this embodiment. Supernatant of CAR19-oNK cell suspensions and Ctrl-oNK cell suspensions (parental oNK cell suspensions) were detected by enzyme-linked immunosorbent assay (ELISA) (D1500, R&D Systems), and concentration of IL-15 in each sample was calculated by standard curve using intrapolation method. Actual methods of this experiment are known, or will be apparent, to those skilled in the art (Manual of D1500, R&D Systems).

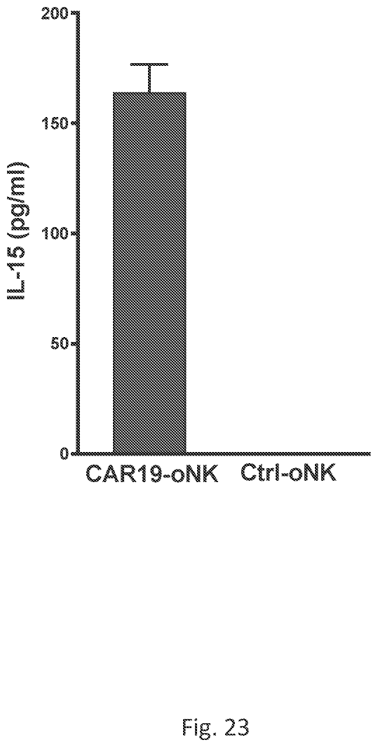

FIG. 23 is the bar chart presenting the IL-15 secretion of CAR19-oNK. FIG. 23 shows that CAR19-oNK cells secreted IL-15, whereas no IL-15 was detectable in parental oNK cells. Therefore, it demonstrated the successful production and processing of IL-15 by CAR19-oNK.

For oNK cells being transduced with the CD19 CAR construct comprises IL-18 expression domain, IL-21 expression domain, IL-2 expression domain, or other proliferation inducing cytokine expression domain, these transduced oNK cells are capable of secreting IL-18, IL-21, IL-2, or other proliferation inducing cytokine.

### Embodiment 22: Analyze Independence of Supplemented Cytokine in CAR19-oNK Culture

One million of CAR19-oNK (cell suspensions obtained by culturing for 56-day CAR-oNK suspension generated by the transduction of cultured oNK with anti-CD19 CAR-expressing pseudo lentiviral particles prepared from pCD810A-1 and lentivirus-based CD19 CAR constructs as shown in FIG. 16A) and Ctrl-oNK (cell suspensions obtained by culturing for 56 days with the culture method disclosed in the embodiments 2.1; also refer to as 56-day cultured oNK suspension) were seeded and cultured in the presence of 500, 100, 10 and 0 IU/mL IL-2. The detail condition could be found in embodiment 2.1 and embodiment 12. The cells were subcultured through replacing the culture with fresh oNK growth medium containing corresponding concentration of IL-2 every 4-7 days depending on cell concentration. The fresh oNK growth medium is the cell culture medium comprises: (1) 0.5%-30% (Volume percent, vol %, v/v) Human platelet lysate; (2) corresponding concentration of IL-2; and (3) DMEM culture medium (Dulbecco's Modified Eagle Medium), alpha modification of Eagle's minimum essential medium, or XVIVO 10 culture medium.

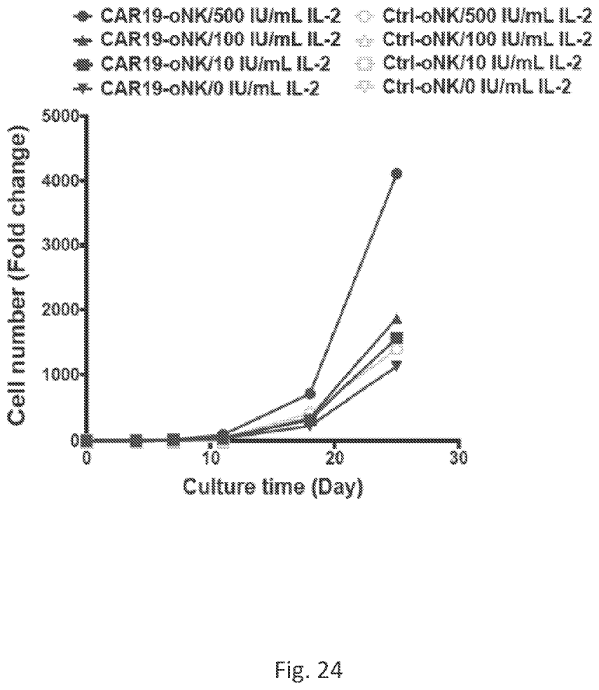

FIG. 24 is the line graph presenting the effect of IL-2 on fold increase in total cell number after different days of culturing CAR19-oNK. FIG. 24 reveals that CAR19-oNK can be cultured and maintained in the presence of IL-15 secreted by the CAR19-oNK. The results showed that CAR19-oNK could grow in the medium without IL-2, whereas Ctrl-oNK can only grow in the medium comprising IL-2.

### Embodiment 23: Detection of Binding Capacity of the CAR-Expressing oNK Cells to their Specific Recombinant Protein

CAR-expressing oNK cells comprising a target-binding single-chain variable fragment (scFv) against a target antigen (such as BCMA) was centrifuged; the supernatant was removed, the cells were resuspended in the buffer, then mixed with tagged target antigen recombinant protein (such as tagged BCMA recombinant protein). Finally, the cell sorter or flow cytometer was used to analyze whether the cells binding to the target antigen recombinant protein (such as BCMA), and the percentage of cells binding to the target antigen recombinant protein (such as BCMA) was calculated.

If the cells have the target antigen recombinant protein (such as BCMA) binding activity, the CAR-expressing oNK cells against the target antigen are successfully developed.

Based on the results shown in the embodiments 18 and 19, the inventors of the present invention believe that oNK cells being transduced with the CAR construct against CD19 or against any of the target antigens disclosed in the invention could elicit enhanced cytotoxicity against cancer cells, liquid tumor, and solid tumor expressing the target antigen without off-target cytotoxicity.

Preferably, the target antigen is CD2, CD3 delta, CD3 epsilon, CD3 gamma, CD4, CD7, CD8a, CD8, CD11a (ITGAL), CD11b (ITGAM), CD11c (ITGAX), CD11d (ITGAD), CD 18 (ITGB2), CD 19 (B4), CD27 (TNFRSF7), CD28, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll-like receptor, HER2, BCMA, PD-L1, VEGFR2, TCR b-chain, and combinations thereof.

Preferably, CAR-expressing oNK cells comprising at least an antigen-binding complex in the cell membrane, wherein the antigen-binding complex is a means for inducing the cytotoxic activity of the cell via being specifically bound by a target antigen selected from cancer antigen, glycolipid, glycoprotein, cluster of differentiation antigen present on cells of a hematopoietic lineage, antigen peptide bound by major histocompatibility complex, gamma-glutamyltranspeptidase, adhesion protein, hormone, growth factor, cytokine, ligand receptor, ion channel, membrane-bound form of an immunoglobulin μ. chain, alfa-fetoprotein, C-reactive protein, chromogranin A, epithelial mucin antigen, human epithelium specific antigen, Lewis(a) antigen, multidrug resistance related protein, Neu oncogene protein, neuron specific enolase, P-glycoprotein, multidrug-resistance-related antigen, p170, multidrug-resistance-related antigen, prostate specific antigen, NCAM, ganglioside molecule, MART-1, heat shock protein, sialylTn, tyrosinase, MUC-1, HER-2/neu, KSA, PSMA, p53, RAS, EGF-R, VEGF, MAGE, or other target antigen (marker) expressed by a target cell.

Preferably, the antigen-binding complex comprises a target-binding single-chain variable fragment (scFv) against the target antigen.

Preferably, the target antigen is a cancer antigen selected from HER2/neu (ERBB2), HER3 (ERBB3), EGFR, VEGF, VEGFR2, GD2, CTLA4, CD19, CD20, CD22, CD30, CD33 (Siglec-3), CD52 (CAMPATH-1 antigen), CD326 (EpCAM), CA-125 (MUC16), MMP9, DLL3, CD274 (PD-L1), CEA, MSLN (mesothelin), CA19-9, CD73, CD205 (DEC205), CD51, c-MET, TRAIL-R2, IGF-1R, CD3, MIF, folate receptor alpha (FOLR1), CSF1, OX-40, CD137, TfR, MUC1, CD25 (IL-2R), CD115 (CSF1R), IL1B, CD105 (Endoglin), KIR, CD47, CEA, IL-17A, DLL4, CD51, angiopoietin 2, neuropilin-1, CD37, CD223 (LAG-3), CD40, LIV-1 (SLC39A6), CD27 (TNFRSF7), CD276 (B7-H3), Trop2, Claudin1 (CLDN1), PSMA, TIM-1 (HAVcr-1), CEACAM5, CD70, LY6E, BCMA, CD135 (FLT3), APRIL, TF(F3), nectin-4, FAP, GPC3, FGFR3, ICAM-1 (CD54), ROBO1, NKG2D ligands, CD123, CS1/SLAMF7/CD319/CRACC, CD7, CD142 (platelet tissue factor, factor III, tissue factor), CD38, CD138, EGFR VIII, EGFR, EGFR806, EGFR family member, PD-1, ROR1, CSPG4, CLL-1 (CLEC12A), CD147, PSCA, EPHA2, GPRC5D, CD133, B7H6, DSC2, AE1 (SLC4A1), GUCY2C, CDH17, HPSE, CD24, MUC4, AFP-L3, SP17, DCLK1, CAIX (CA9), IL13RA2, IL13Ra, CD56, CD44v6, TCR beta-chain, ligands of chlorotoxin, claudin-6, claudin-18.2, EIIIB (fibronectin), Glypican-1 (GPC1), PLAP (Placental alkaline phosphatase), uPAR, HCMV glycoprotein B (gB), HLA-DR (Lym1 antibody target), tumor-associated αvβ6 integrin, LunX, integrin αvβ3, folate receptor beta (FRβ), LILRB4, MISIIR (Müllerian inhibiting substance type 2 receptor), 5T4, CD83 ligand, HBsAg, CD171 (L1-CAM), TAG72 (TAG72 (Tumour-associated glycoprotein 72)), B7-H4, CD166 (ALCAM), AC133 (PROM1), LeY, CD13 (TIM1), CD117, TEM8 (ANTXR1), CD26, IL13Ra2, IGF1R, Muc3a, IL1RAP, TSLPR (CRLF2), LMP1, Siglec7, Siglec9, Epstein-Barr Virus gp350, CD1a, CLEC14A, MAGE-A1, MAGE-A4, Neurofilament M (NEFM), HERV-K env protein, HLA-A*0201/NY-ESO-1(157-165) peptide, 2B4, TACI (TNFRSF13B), CD32A(131R), AXL, Lewis Y, CD80, CD86, ROR2, a killer-cell immunoglobulin-like receptors (KIRs), a T cell receptor, a major histocompatibility complex protein, a TNF receptor protein, an immunoglobulin protein, a cytokine receptor, an integrin, activating NK cell receptors, and combinations thereof.

Preferably, the antigen-binding complex is a chimeric antigen receptor (CAR).

Preferably, a chromosome DNA sequence of the cell is at least 90% or 95% similar to the corresponding chromosome DNA sequence of the natural killer cell deposited at NPMD having the deposit number NITE BP-03017.

Preferably, a chromosome DNA sequence of the cell is at least 99%, 99.99%, or 99.995% similar to the corresponding chromosome DNA sequence of the natural killer cell deposited at NPMD having the deposit number NITE BP-03017.

Preferably, the chromosome DNA sequence is a DNA of chromosome 17, a DNA sequence of chromosome 19, a DNA sequence of chromosome 22, a DNA sequence of chromosome 4, a DNA sequence of chromosome 18, a DNA sequence of chromosome Y, or a DNA sequence of chromosome X. Preferably, the chromosome DNA sequence is a DNA sequence of chromosome 1, a DNA sequence of chromosome 2, a DNA sequence of chromosome 5, a DNA sequence of chromosome 6, a DNA sequence of chromosome 7, a DNA sequence of chromosome 8, a DNA sequence of chromosome 9, a DNA sequence of chromosome 10, a DNA sequence of chromosome 11, a DNA sequence of chromosome 12, a DNA sequence of chromosome 13, a DNA sequence of chromosome 14, a DNA sequence of chromosome 15, a DNA sequence of chromosome 16, a DNA sequence of chromosome 20, a DNA sequence of chromosome 21, or a DNA sequence of chromosome 3.

Preferably, a whole genome of the cell is at least 99.995% similar to the whole genome of the natural killer cell deposited at NPMD having the deposit number NITE BP-03017.

From the embodiments of the present invention, it demonstrated that all of the non-transgenic human CD16⁺ natural killer cell line obtained by the method of the present invention, the exogenous targeting unit complexed-natural killer cell of the present invention, and the chimeric antigen receptor (CAR)-expressing oNK cells of the invention can indeed kill the target cell (e.g., cancer cells) though ADCC-like process. Therefore, the applicable fields of these cells of the present invention, include but not limited to cancer treatment, autoimmune disease treatment, neuronal disease treatment, human immunodeficiency virus (HIV) eradication, hematopoietic cell-related diseases, metabolic syndrome treatment, pathogenic disease treatment, treatment of viral infection, and treatment of bacterial infection.
Reference 1—Eileen Scully and Galit Alter, 2016. NK cells in HIV disease. Curr HIV/AIDS Rep. 13 (2):85-94.
Reference 2—Jordan S. Orange, 2013. Natural killer cell deficiency. J Allergy Clin Immunol 132(3):515-525.
Reference 3—Kallioniemi A, Visakorpi T, Karhu R, Pinkel D, and Kallioniemi OP, 1996. Gene Copy number analysis by fluorescence in situ hybridization and comparative genomic hybridization. Methods. 9(1):113-121.
Reference 4—Littwitz-Salomon E, Dittmer U, Sutter K, 2016. Insufficient natural killer cell responses against retroviruses: how to improve NK cell killing of retrovirus-infected cells. Retrovirology. 13(1):77.
Reference 5—Pernick, N, 2018.

http://www.pathologyoutlines.com/topic/cdmarkerscd56.html

http://www.pathologyoutlines.com/topic/cdmarkerscd3.html

http://www.pathologyoutlines.com/topic/cdmarkerscd2.html

http://www.pathologyoutlines.com/topic/cdmarkerscd16.html
Reference 6—Rezvani K and Rouce RH, 2015. The application of natural killer cell immunotherapy for the treatment of cancer. Front Immunol. 6:578.
Reference 7—Vindeløv, L. L., Christensen, I. J., and Nissen, N. I., 1983. A Detergent-trypsin method for the preparation of nuclei for flow cytometric DNA analysis. Cytometry. 3(5), 323-327.
Reference 8—Zimmermann, J, Nicolaus, T, Neuert, G. and Blank, K. 2010. Thiol-based, site-specific and covalent immobilization of biomolecules for single-molecule experiments. Nat. Protoc. 5(6):975-985.
Reference 9—Rigo V, Emionite L, Daga A, Astigiano S, Comas MV, Quintarelli C, Locatelli F, Ferrini S, Croce M. 2017. Combined immunotherapy with anti-PDL-1/PD-1 and anti-CD4 antibodies cures syngeneic disseminated neuroblastoma. Sci. Rep. 7(1):14049.
Reference 10—Manual of Human IL-15 Immunoassay (DL1500, R&D Systems) https://resources.rndsystems.com/pdfs/datasheets/d1500.pdf
Reference 11—Robert Weinkove, Philip George, Nathaniel Dasyam, Alexander D McLellan, 2019. Selecting costimulatory domains for chimeric antigen receptors: functional and clinical considerations. Clin Transl Immunology. 8(5): e1049.

The foregoing descriptions are merely the preferred embodiments of the present invention and are not intended to limit the scope of the patent application of the present invention. Therefore, any alteration or modification that does not depart from the spirits disclosed herein should be included within the scope of the patent application of the present invention.

1-67. (canceled)

68. A composition comprising:
at least a human cell with cytotoxic capability, wherein the human cell with cytotoxic capability has the following characteristics:
i) carrying a phenotype of CD3⁻CD56⁺ and expressing a CD16 receptor; and
ii) comprising at least an antigen-binding complex in the cell membrane, wherein the antigen-binding complex is a means for inducing the cytotoxic activity of the cell via being specifically bound by an antigen selected from cancer antigen, glycolipid, glycoprotein, cluster of differentiation antigen present on cells of a hematopoietic lineage, antigen peptide bound by major histocompatibility complex, gamma-glutamyltranspeptidase, adhesion protein, hormone, growth factor, cytokine, ligand receptor, ion channel, membrane-bound form of an immunoglobulin μ. chain, alfa-fetoprotein, C-reactive protein, chromogranin A, epithelial mucin antigen, human epithelium specific antigen, Lewis(a) antigen, multidrug resistance related protein, Neu oncogene protein, neuron specific enolase, P-glycoprotein, multidrug-resistance-related antigen, p170, multidrug-resistance-related antigen, prostate specific antigen, NCAM, ganglioside molecule, MART-1, heat shock protein, sialylTn, tyrosinase, MUC-1, HER-2/neu, KSA, PSMA, p53, RAS, EGF-R, VEGF, MAGE, or other target antigen (marker) expressed by a target cell;
wherein the cell is not genetically modified from the natural killer cell having the deposit number ATCC CRL-2407.

69. The composition according to claim 68,
wherein the cell is non-tumorigenic in an immune compromised mouse; or
wherein, after being irradiated with γ-ray, the cell is non-tumorigenic in an allogeneic subject.

70. The composition according to claim 68, wherein the cell is capable of mediating an antibody-dependent cell cytotoxicity (ADCC) response, and the cell is a male cell.

71. The composition according to claim 68, wherein the cell is a natural killer cell genetically modified to express the antigen-binding complex.

72. The composition according to claim 68, wherein the cell and the natural killer cell line NK3.3 are derived from different subjects.

73. The composition according to claim 68, wherein the cell is derived from a subject with a cancer.

74. The composition according to claim 68, wherein the cell is derived from a Caucasian male.

75. The composition according to claim 68, wherein the cell and the natural killer cell having the deposit number ATCC CRL-2407 are derived from the same subject.

76. The composition according to claim 68, wherein the cell retains its capability to proliferate after subculture for at least 1 month, 2 months, 3 months, 4 months, 5 months or 6 months.

77. The composition according to claim 68, wherein the antigen-binding complex is produced by the cell.

78. The composition according to claim 68, wherein the cell further exhibits IL-15 secretion capability, IL-18 secretion capability, IL-21 secretion capability, IL-2 secretion capability, or other proliferation-inducing cytokine secretion capability, or the combination thereof; or
wherein the cell further carries a phenotype of CD2⁺; or
wherein the cell further carries a phenotype of CD45⁺; or
wherein the cell further carries a phenotype selected from CD4⁺, CD25⁺, NKp30⁺, NKG2D⁺, NKp44⁺, NKp46⁺, CD27⁺, OX40⁺, CD107a⁺, NKG2A⁺, PD-1⁺, SIRPα⁺, CD158⁺ or the combination thereof.

79. The composition according to claim 68, wherein the antigen-binding complex comprises CD3 zeta (CD3) subunit.

80. The composition according to claim 79, wherein the antigen-binding complex further comprises CD28 subunit, ICOS (CD278) subunit, 4-1BB (CD137) subunit, OX40 (CD134) subunit, CD27 subunit, CD40 subunit, CD40L subunit, TLRs subunit, or other costimulatory molecule expressed by at least one of effector cells, or the combination thereof.

81. The composition according to claim 68, wherein the cell further comprises a synthetic, genetically modified and/or deliberately delivered polynucleotide encoding a target-binding single-chain variable fragment (scFv) against the antigen, and the target-binding single-chain variable fragment is at least a subunit of the antigen-binding complex.

82. The composition according to claim 68, wherein a chromosome DNA sequence of the cell is at least 90% or 95% similar to the corresponding chromosome DNA sequence of the natural killer cell deposited at NPMD having the deposit number NITE BP-03017.

83. The composition according to claim 68, wherein the cell does not include synthetic, genetically modified and/or deliberately delivered polynucleotide encoding the CD16 receptor.

84. The composition according to claim 68, wherein the number of the human cells in the composition is at least 5×10⁵ and the human cells are in an amount equal to or more than 5% by number, based on the total number of the cells in the composition as 100%.

85. A method of obtaining a composition substantially enriched in human cells according to claim 68; the method comprising:
(a) obtaining a population of human CD16⁺ natural killer cells; and
(b) delivering a polynucleotide encoding the antigen-binding complex comprising a target-binding single-chain variable fragment (scFv) against the antigen into the human CD16⁺ natural killer cells thereby obtaining the composition substantially enriched in human cells;
wherein the human CD16⁺ natural killer cell has the following characteristics:
i) a chromosome DNA sequence of the human CD16⁺ natural killer cells is at least 90% or 95% similar to the corresponding chromosome DNA sequence of the natural killer cell deposited at NPMD having the deposit number NITE BP-03017, and
ii) not genetically modified from the natural killer cell having the deposit number ATCC CRL-2407.

86. The method according to claim 85, wherein the antigen-binding complex comprises a CD3 zeta (CD3ζ) peptide.

87. The method according to claim 86, wherein the antigen-binding complex further comprises CD28 peptide, ICOS (CD278) peptide, 4-1BB (CD137) peptide, OX40 (CD134) peptide, CD27 peptide, CD40 peptide, CD40L peptide, TLRs peptide, or other peptide of costimulatory molecule expressed by at least one of effector cells, or the combination thereof.

88. The method according to claim 86, the method further comprising a step:
(c) in a container, contacting the human cells with a culture medium comprising 0.5-10 vol % human platelet lysate and 100-3000 IU/mL IL-2; and culturing the cells for multiple days.

89. A method of treating cancer, tumor, autoimmune disease, neuronal disease, human immunodeficiency virus (HIV) infection, hematopoietic cell-related diseases, metabolic syndrome, pathogenic disease, viral infection, or bacterial infection, comprising administering a composition comprising an effective amount of the cell selected form claim 68 to a subject in need thereof.

90. The method of claim 89, wherein the antigen is a cancer antigen.

91. The method according to claim 89, wherein the method is for treating cancer or tumor.

92. The method according to claim 89, wherein the method is for treating solid tumor or liquid tumor.


SEQUENCE LISTING (57 sequences)

SEQ ID NO: 1 - DNA, 2137 bp, Homo sapiens
cttgtccactccagtgtggcatcatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagttgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttgtcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatgacccccatcccatgggggtaataagagcagtagcagcagcatctctgaacatttctctggatttgcaaccccatcatcctcaggcctctctacaagcagcaggaaacatagaactcagagccagatcccttatccaactctcgacttttccttggtctccagtggaagggaaaagcccatgatcttcaagcagggaagccccagtgagtagctgcattcctagaaattgaagtttcagagctacacaaacactttttctgtcccaaccgttccctcacagcaaagcaacaatacaggctagggatggtaatcctttaaacatacaaaaattgctcgtgttataaattacccagtttagaggggaaaaaaaaacaattattcctaaataaatggataagtagaattaatggttgaggcaggaccatacagagtgtgggaactgctggggatctagggaattcagtgggaccaatgaaagcatggctgagaaatagcaggtagtccaggatagtctaagggaggtgttcccatctgagcccagagataagggtgtcttcctagaacattagccgtagtggaattaacaggaaatcatgagggtgacgtagaattgagtcttccaggggactctatcagaactggaccatctccaagtatataacgatgagtcctcttaatgctaggagtagaaaatggtcctaggaaggggactgaggattgcggtggggggtggggtggaaaagaaagtacagaacaaaccctgtgtcactgtcccaagttgctaagtgaacagaactatctcagcatcagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatatctaattcctgtgtagctttgttcattgcatttattaaacaaatgttgtataaccaatactaaatgtactactgagcttcgctgagttaagttatgaaactttcaaatccttcatcatgtcagttccaatgaggtggggatggagaagacaattgttgcttatgaaagaaagctttagctgtctctgttttgtaagctttaagcgcaacatttcttggttccaataaagcattttacaagatcttgcatgctactcttagatagaagatgggaaaaccatggtaataaaatatgaatgataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - DNA, 820 bp, Homo sapiens
cactccagtgtggcatcatgtggcagctgctcctcccaactgctctgctacttctagtttcagctggcatgcggactgaagatctcccaaaggctgtggtgttcctggagcctcaatggtacagcgtgcttgagaaggacagtgtgactctgaagtgccagggagcctactcccctgaggacaattccacacagtggtttcacaatgagagcctcatctcaagccaggcctcgagctacttcattgacgctgccacagtcaacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaagacaggaagtattttcatcataattctgacttccacattccaaaagccacactcaaagatagcggctcctacttctgcagggggcttgttgggagtaaaaatgtgtcttcagagactgtgaacatcaccatcactcaaggtttggcagtgtcaaccatctcatcattctctccacctgggtaccaagtctctttctgcttggtgatggtactcctttttgcagtggacacaggactatatttctctgtgaagacaaacatttgaagctcaacaagagactggaaggaccataaacttaaatggagaaaggaccctcaagacaaatgacccccatcccatgggagtaataagagcagtggcagcag

SEQ ID NO: 3 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 4 - protein, 233 aa, Homo sapiens
MWQLLLPTALLLLVSAGMRTEDLPKAVVFLEPQWYSVLEKDSVTLKCQGAYSPEDNSTQWFHSESLISSQASSYFIDAATVNESGEYRCQTNLSTLSDPVQLEVHIGWLLLRAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 5 - DNA, 20 bp, Artificial sequence
  synthetic
cacacacacacacacacaca

SEQ ID NO: 6 - DNA, 20 bp, Artificial sequence
  synthetic
tcatacgactcactctaggg

SEQ ID NO: 7 - DNA, 23 bp, Artificial sequence
  synthetic
agttaccatgacgtcaatttcag

SEQ ID NO: 8 - DNA, 20 bp, Artificial sequence
  synthetic
tgtgtgtgtgtgtgtgtgtg

SEQ ID NO: 9 - DNA, 20 bp, Artificial sequence
  synthetic
ccctagagtgagtcgtatga

SEQ ID NO: 10 - DNA, 23 bp, Artificial sequence
  synthetic
ctgaaattgacgtcatggtaact

SEQ ID NO: 11 - DNA, 51 bp, Artificial sequence
  synthetic
tctgaagacacatttttactcccaaaaagccccctgcagaagtaggagccg

SEQ ID NO: 12 - DNA, 51 bp, Artificial sequence
  synthetic
tctgaagacacatttttactcccaacaagccccctgcagaagtaggagccg

SEQ ID NO: 13 - DNA, 765 bp, Homo sapiens
  misc_feature: cDNA
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 14 - protein, 254 aa, Homo sapiens; identical to SEQ ID NO: 3

SEQ ID NO: 15 - DNA, 765 bp, Homo sapiens
  misc_feature: mRNA
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 16 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 17 - DNA, 51 bp, Artificial sequence
  synthetic
tccttcacaatttctgcagccactctgtggccaccgtcaccttattcctga

SEQ ID NO: 18 - DNA, 51 bp, Artificial sequence
  synthetic
tccttcacaatttctgcagccactccgtggccaccgtcaccttattcctga

SEQ ID NO: 19 - DNA, 765 bp, Homo sapiens; identical to SEQ ID NO: 13

SEQ ID NO: 20 - protein, 80 aa, Homo sapiens
HSWKNTALHKVTYLQNGKGRKYFHHNSDFYIPKATLKDSGSYFCRGLFGSKNVSSETVNITITQGLAVSTISSFFPPGYQ

SEQ ID NO: 21 - DNA, 20 bp, Artificial sequence
  synthetic
aaaaaaaaaaaaaaaaaaaa

SEQ ID NO: 22 - DNA, 20 bp, Artificial sequence
  synthetic
tttttttttttttttttttt

SEQ ID NO: 23 - DNA, 20 bp, Artificial sequence
  synthetic
actgactgactgactgactg

SEQ ID NO: 24 - DNA, 20 bp, Artificial sequence
  synthetic
cagtcagtcagtcagtcagt

SEQ ID NO: 25 - DNA, 20 bp, Artificial sequence
  synthetic
gtaacgatccagctgtcact

SEQ ID NO: 26 - DNA, 20 bp, Artificial sequence
  synthetic
agtgacagctggatcgttac

SEQ ID NO: 27 - DNA, 20 bp, Artificial sequence
  synthetic
actgatggtaatctgcacct

SEQ ID NO: 28 - DNA, 20 bp, Artificial sequence
  synthetic
aggtgcagattaccatcagt

SEQ ID NO: 29 - DNA, 40 bp, Artificial sequence
  synthetic
aaaaaaaaaaaaaaaaaaaaaaaaaaaaaaaaaaaaaaaa

SEQ ID NO: 30 - DNA, 40 bp, Artificial sequence
  synthetic
tttttttttttttttttttttttttttttttttttttttt

SEQ ID NO: 31 - DNA, 40 bp, Artificial sequence
  synthetic
actgactgactgactgactgactgactgactgactgactg

SEQ ID NO: 32 - DNA, 40 bp, Artificial sequence
  synthetic
cagtcagtcagtcagtcagtcagtcagtcagtcagtcagt

SEQ ID NO: 33 - DNA, 40 bp, Artificial sequence
  synthetic
tgtgtgtgtgtgtgtgtgtgtgtgtgtgtgtgtgtgtgtg

SEQ ID NO: 34 - DNA, 40 bp, Artificial sequence
  synthetic
cacacacacacacacacacacacacacacacacacacaca

SEQ ID NO: 35 - DNA, 40 bp, Artificial sequence
  synthetic
gtaacgatccagctgtcactgtaacgatccagctgtcact

SEQ ID NO: 36 - DNA, 40 bp, Artificial sequence
  synthetic
agtgacagctggatcgttacagtgacagctggatcgttac

SEQ ID NO: 37 - DNA, 40 bp, Artificial sequence
  synthetic
tcatacgactcactctagggtcatacgactcactctaggg

SEQ ID NO: 38 - DNA, 40 bp, Artificial sequence
  synthetic
ccctagagtgagtcgtatgaccctagagtgagtcgtatga

SEQ ID NO: 39 - DNA, 40 bp, Artificial sequence
  synthetic
actgatggtaatctgcacctactgatggtaatctgcacct

SEQ ID NO: 40 - DNA, 40 bp, Artificial sequence
  synthetic
aggtgcagattaccatcagtaggtgcagattaccatcagt

SEQ ID NO: 41 - DNA, 2025 bp, Artificial sequence
  synthetic
atgtatcggatgcagttgctttcctgcatcgctttgtctcttgctttggtaaccaactcagacatccagatgacccagacgacaagtagcctgtctgccagtcttggtgatagagtgacaatttcttgtagagcgagccaggacatatctaagtacctgaactggtatcaacaaaagccggatggtacagtgaagctcctcatatatcatacgagtcggcttcatagcggggtgccaagtcgattttctgggagtggttccggtacagactactcattgacaatatcaaacttggaacaggaagacattgctacatacttttgccaacaaggcaataccctcccgtatacatttggcggaggcacaaaactcgagattacaggcggcggtggtagcggaggaggcgggtccggcggcggtggcagtgaggttaagctccaggagtctggccccggactcgtggccccttcacagagtctgagtgttacatgtacggtgtccggtgtgagcttgcctgattacggagtgtcctggatcaggcaaccgccacgaaaaggattggagtggcttggtgtgatctggggctctgaaacaacttattataactccgctttgaaaagtaggcttaccatcataaaagataacagtaagtcccaggtctttcttaaaatgaactccctgcaaaccgatgacaccgccatttactattgtgctaagcattattactacggcggtagctacgcaatggactactggggccagggaacctctgtgaccgtgtcatcagaattcgcatcatttgagattgaagttatgtatccaccgccgtatcttgacaatgagaagagtaacggaacaatcatacacgttaagggtaaacatttgtgtccttctccactgtttcccggtccctcaaagcccttttgggtcctggttgtcgttggaggtgtattggcatgctatagtctgcttgtcacggtagcgtttatcattttttgggtcaggtccaaacgatctagactgctccattcagattatatgaacatgacgccccgccgaccaggaccgacgcgaaaacattaccagccgtacgcgccaccacgagactttgccgcgtaccggagtagagtcaaattctctaggagtgcagacgccccggcatatcaacagggacaaaaccaactttataatgagcttaatctggggcgaagggaagagtatgacgttctcgataagcggagggggcgggatccagagatgggtggtaagccgcagaggcgcaagaatccgcaagaaggtttgtacaatgagctccaaaaggacaagatggcagaagcctacagtgagattggaatgaagggtgaaaggcggcgaggtaagggacatgatgggttgtatcaaggtcttagtacagctaccaaggacacttatgatgccctccatatgcaggccttgccgccaagaaagcttgggagcggcgccacaaacttttctctcctcaaacaggcaggagacgtcgaggaaaatcccgggcctatgcgaatttctaagcctcaccttagaagtatctctatccaatgttacctttgtctcttgctcaacagtcactttttgacggaagccgggatacacgtcttcattctcgggtgtttctccgccgggctcccgaagaccgaggccaattgggtgaacgtaatcagtgaccttaagaaaatagaagatcttattcaatctatgcatatcgacgcaaccctctacacagaaagcgatgtgcatccttcttgcaaggttacggcaatgaagtgtttcctgctggaattgcaggtaataagtctcgaatccggtgacgccagtatacatgatacagtagaaaaccttatcatcttggcaaataacagcctgtcttcaaatggaaatgtaacagagagtggctgcaaagagtgcgaagaacttgaagagaaaaacatcaaagagtttctgcagtcatttgtgcacattgtccagatgttcattaataccagttga

SEQ ID NO: 42 - DNA, 2265 bp, Artificial sequence
  synthetic
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcagaattcgaacaaaaactcatctcagaagaggatctggcatcatttgagattgaagttatgtatccaccgccgtatcttgacaatgagaagagtaacggaacaatcatacacgttaagggtaaacatttgtgtccttctccactgtttcccggtccctcaaagcccttttgggtcctggttgtcgttggaggtgtattggcatgctatagtctgcttgtcacggtagcgtttatcattttttgggtcaggtccaaacgatctagactgctccattcagattatatgaacatgacgccccgccgaccaggaccgacgcgaaaacattaccagccgtacgcgccaccacgagactttgccgcgtaccggagtagagtcaaattctctaggagtgcagacgccccggcatatcaacagggacaaaaccaactttataatgagcttaatctggggcgaagggaagagtatgacgttctcgataagcggagggggcgggatccagagatgggtggtaagccgcagaggcgcaagaatccgcaagaaggtttgtacaatgagctccaaaaggacaagatggcagaagcctacagtgagattggaatgaagggtgaaaggcggcgaggtaagggacatgatgggttgtatcaaggtcttagtacagctaccaaggacacttatgatgccctccatatgcaggccttgccgccaagaagaggaagcggagctactaacttcagcctgctgaagcaggctggagacgtggaggagaaccctggacctatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaatga

SEQ ID NO: 43 - DNA, 1500 bp, Artificial sequence
  synthetic
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcagaattcgaacaaaaactcatctcagaagaggatctggcatcatttgagattgaagttatgtatccaccgccgtatcttgacaatgagaagagtaacggaacaatcatacacgttaagggtaaacatttgtgtccttctccactgtttcccggtccctcaaagcccttttgggtcctggttgtcgttggaggtgtattggcatgctatagtctgcttgtcacggtagcgtttatcattttttgggtcaggtccaaacgatctagactgctccattcagattatatgaacatgacgccccgccgaccaggaccgacgcgaaaacattaccagccgtacgcgccaccacgagactttgccgcgtaccggagtagagtcaaattctctaggagtgcagacgccccggcatatcaacagggacaaaaccaactttataatgagcttaatctggggcgaagggaagagtatgacgttctcgataagcggagggggcgggatccagagatgggtggtaagccgcagaggcgcaagaatccgcaagaaggtttgtacaatgagctccaaaaggacaagatggcagaagcctacagtgagattggaatgaagggtgaaaggcggcgaggtaagggacatgatgggttgtatcaaggtcttagtacagctaccaaggacacttatgatgccctccatatgcaggccttgccgccaagatga

SEQ ID NO: 44 - DNA, 2049 bp, Artificial sequence
  synthetic
atgtatcggatgcagttgctttcctgcatcgctttgtctcttgctttggtaaccaactcagactacaaggacgatgatgacaaagacatccagatgacccagacgacaagtagcctgtctgccagtcttggtgatagagtgacaatttcttgtagagcgagccaggacatatctaagtacctgaactggtatcaacaaaagccggatggtacagtgaagctcctcatatatcatacgagtcggcttcatagcggggtgccaagtcgattttctgggagtggttccggtacagactactcattgacaatatcaaacttggaacaggaagacattgctacatacttttgccaacaaggcaataccctcccgtatacatttggcggaggcacaaaactcgagattacaggcggcggtggtagcggaggaggcgggtccggcggcggtggcagtgaggttaagctccaggagtctggccccggactcgtggccccttcacagagtctgagtgttacatgtacggtgtccggtgtgagcttgcctgattacggagtgtcctggatcaggcaaccgccacgaaaaggattggagtggcttggtgtgatctggggctctgaaacaacttattataactccgctttgaaaagtaggcttaccatcataaaagataacagtaagtcccaggtctttcttaaaatgaactccctgcaaaccgatgacaccgccatttactattgtgctaagcattattactacggcggtagctacgcaatggactactggggccagggaacctctgtgaccgtgtcatcagaattcgcatcatttgagattgaagttatgtatccaccgccgtatcttgacaatgagaagagtaacggaacaatcatacacgttaagggtaaacatttgtgtccttctccactgtttcccggtccctcaaagcccttttgggtcctggttgtcgttggaggtgtattggcatgctatagtctgcttgtcacggtagcgtttatcattttttgggtcaggtccaaacgatctagactgctccattcagattatatgaacatgacgccccgccgaccaggaccgacgcgaaaacattaccagccgtacgcgccaccacgagactttgccgcgtaccggagtagagtcaaattctctaggagtgcagacgccccggcatatcaacagggacaaaaccaactttataatgagcttaatctggggcgaagggaagagtatgacgttctcgataagcggagggggcgggatccagagatgggtggtaagccgcagaggcgcaagaatccgcaagaaggtttgtacaatgagctccaaaaggacaagatggcagaagcctacagtgagattggaatgaagggtgaaaggcggcgaggtaagggacatgatgggttgtatcaaggtcttagtacagctaccaaggacacttatgatgccctccatatgcaggccttgccgccaagaaagcttgggagcggcgccacaaacttttctctcctcaaacaggcaggagacgtcgaggaaaatcccgggcctatgcgaatttctaagcctcaccttagaagtatctctatccaatgttacctttgtctcttgctcaacagtcactttttgacggaagccgggatacacgtcttcattctcgggtgtttctccgccgggctcccgaagaccgaggccaattgggtgaacgtaatcagtgaccttaagaaaatagaagatcttattcaatctatgcatatcgacgcaaccctctacacagaaagcgatgtgcatccttcttgcaaggttacggcaatgaagtgtttcctgctggaattgcaggtaataagtctcgaatccggtgacgccagtatacatgatacagtagaaaaccttatcatcttggcaaataacagcctgtcttcaaatggaaatgtaacagagagtggctgcaaagagtgcgaagaacttgaagagaaaaacatcaaagagtttctgcagtcatttgtgcacattgtccagatgttcattaataccagttga

SEQ ID NO: 45 - DNA, 2055 bp, Artificial sequence
  synthetic
atgtatcggatgcagttgctttcctgcatcgctttgtctcttgctttggtaaccaactcagaacaaaaactcatctcagaagaggatctggacatccagatgacccagacgacaagtagcctgtctgccagtcttggtgatagagtgacaatttcttgtagagcgagccaggacatatctaagtacctgaactggtatcaacaaaagccggatggtacagtgaagctcctcatatatcatacgagtcggcttcatagcggggtgccaagtcgattttctgggagtggttccggtacagactactcattgacaatatcaaacttggaacaggaagacattgctacatacttttgccaacaaggcaataccctcccgtatacatttggcggaggcacaaaactcgagattacaggcggcggtggtagcggaggaggcgggtccggcggcggtggcagtgaggttaagctccaggagtctggccccggactcgtggccccttcacagagtctgagtgttacatgtacggtgtccggtgtgagcttgcctgattacggagtgtcctggatcaggcaaccgccacgaaaaggattggagtggcttggtgtgatctggggctctgaaacaacttattataactccgctttgaaaagtaggcttaccatcataaaagataacagtaagtcccaggtctttcttaaaatgaactccctgcaaaccgatgacaccgccatttactattgtgctaagcattattactacggcggtagctacgcaatggactactggggccagggaacctctgtgaccgtgtcatcagaattcgcatcatttgagattgaagttatgtatccaccgccgtatcttgacaatgagaagagtaacggaacaatcatacacgttaagggtaaacatttgtgtccttctccactgtttcccggtccctcaaagcccttttgggtcctggttgtcgttggaggtgtattggcatgctatagtctgcttgtcacggtagcgtttatcattttttgggtcaggtccaaacgatctagactgctccattcagattatatgaacatgacgccccgccgaccaggaccgacgcgaaaacattaccagccgtacgcgccaccacgagactttgccgcgtaccggagtagagtcaaattctctaggagtgcagacgccccggcatatcaacagggacaaaaccaactttataatgagcttaatctggggcgaagggaagagtatgacgttctcgataagcggagggggcgggatccagagatgggtggtaagccgcagaggcgcaagaatccgcaagaaggtttgtacaatgagctccaaaaggacaagatggcagaagcctacagtgagattggaatgaagggtgaaaggcggcgaggtaagggacatgatgggttgtatcaaggtcttagtacagctaccaaggacacttatgatgccctccatatgcaggccttgccgccaagaaagcttgggagcggcgccacaaacttttctctcctcaaacaggcaggagacgtcgaggaaaatcccgggcctatgcgaatttctaagcctcaccttagaagtatctctatccaatgttacctttgtctcttgctcaacagtcactttttgacggaagccgggatacacgtcttcattctcgggtgtttctccgccgggctcccgaagaccgaggccaattgggtgaacgtaatcagtgaccttaagaaaatagaagatcttattcaatctatgcatatcgacgcaaccctctacacagaaagcgatgtgcatccttcttgcaaggttacggcaatgaagtgtttcctgctggaattgcaggtaataagtctcgaatccggtgacgccagtatacatgatacagtagaaaaccttatcatcttggcaaataacagcctgtcttcaaatggaaatgtaacagagagtggctgcaaagagtgcgaagaacttgaagagaaaaacatcaaagagtttctgcagtcatttgtgcacattgtccagatgttcattaataccagttga

SEQ ID NO: 46 - DNA, 2055 bp, Artificial sequence
  synthetic
atgtatcggatgcagttgctttcctgcatcgctttgtctcttgctttggtaaccaactcagacatccagatgacccagacgacaagtagcctgtctgccagtcttggtgatagagtgacaatttcttgtagagcgagccaggacatatctaagtacctgaactggtatcaacaaaagccggatggtacagtgaagctcctcatatatcatacgagtcggcttcatagcggggtgccaagtcgattttctgggagtggttccggtacagactactcattgacaatatcaaacttggaacaggaagacattgctacatacttttgccaacaaggcaataccctcccgtatacatttggcggaggcacaaaactcgagattacaggcggcggtggtagcggaggaggcgggtccggcggcggtggcagtgaggttaagctccaggagtctggccccggactcgtggccccttcacagagtctgagtgttacatgtacggtgtccggtgtgagcttgcctgattacggagtgtcctggatcaggcaaccgccacgaaaaggattggagtggcttggtgtgatctggggctctgaaacaacttattataactccgctttgaaaagtaggcttaccatcataaaagataacagtaagtcccaggtctttcttaaaatgaactccctgcaaaccgatgacaccgccatttactattgtgctaagcattattactacggcggtagctacgcaatggactactggggccagggaacctctgtgaccgtgtcatcagaattcgaacaaaaactcatctcagaagaggatctggcatcatttgagattgaagttatgtatccaccgccgtatcttgacaatgagaagagtaacggaacaatcatacacgttaagggtaaacatttgtgtccttctccactgtttcccggtccctcaaagcccttttgggtcctggttgtcgttggaggtgtattggcatgctatagtctgcttgtcacggtagcgtttatcattttttgggtcaggtccaaacgatctagactgctccattcagattatatgaacatgacgccccgccgaccaggaccgacgcgaaaacattaccagccgtacgcgccaccacgagactttgccgcgtaccggagtagagtcaaattctctaggagtgcagacgccccggcatatcaacagggacaaaaccaactttataatgagcttaatctggggcgaagggaagagtatgacgttctcgataagcggagggggcgggatccagagatgggtggtaagccgcagaggcgcaagaatccgcaagaaggtttgtacaatgagctccaaaaggacaagatggcagaagcctacagtgagattggaatgaagggtgaaaggcggcgaggtaagggacatgatgggttgtatcaaggtcttagtacagctaccaaggacacttatgatgccctccatatgcaggccttgccgccaagaaagcttgggagcggcgccacaaacttttctctcctcaaacaggcaggagacgtcgaggaaaatcccgggcctatgcgaatttctaagcctcaccttagaagtatctctatccaatgttacctttgtctcttgctcaacagtcactttttgacggaagccgggatacacgtcttcattctcgggtgtttctccgccgggctcccgaagaccgaggccaattgggtgaacgtaatcagtgaccttaagaaaatagaagatcttattcaatctatgcatatcgacgcaaccctctacacagaaagcgatgtgcatccttcttgcaaggttacggcaatgaagtgtttcctgctggaattgcaggtaataagtctcgaatccggtgacgccagtatacatgatacagtagaaaaccttatcatcttggcaaataacagcctgtcttcaaatggaaatgtaacagagagtggctgcaaagagtgcgaagaacttgaagagaaaaacatcaaagagtttctgcagtcatttgtgcacattgtccagatgttcattaataccagttga

SEQ ID NO: 47 - DNA, 3327 bp, Artificial Sequence
  Synthetic
atgctcgaaggagtgcaggttgagacgataagccctggagatggccggacgttcccaaagagggggcaaacttgtgtagtacattacaccgggatgttggaggatggtaagaaggttgactcatctagggatagaaacaaaccttttaagtttatgctcgggaagcaagaggtcattaggggctgggaagaaggcgtggcgcagatgtccgtgggtcaacgggctaaactcacaataagtcccgattatgcctatggtgctacggggcaccctggcattatcccgccacatgcaacacttgtgttcgacgtcgagttgcttaaactcgaatccggcgggggatccggggtggatggtttcggggacgtcggtgcgctggagtctttgcgcggaaatgccgatcttgcatacattcttagtatggaaccatgcggtcactgcctgataatcaacaatgtaaacttctgccgagagtccggcttgaggacacggacagggagtaatatagattgcgaaaaacttagaagaaggttcagctcattgcactttatggtcgaggtaaagggggatttgaccgctaaaaagatggtgctggcgctcctcgaactcgctcgacaagatcatggtgccttggattgctgtgttgttgtaatactgagtcatggttgccaagcgagccacctgcagttcccgggcgctgtttacggtactgacgggtgccctgtgagcgtcgaaaaaatcgttaacatatttaacggtacctcatgtccttctctcggtgggaaacctaagctgtttttcattcaagcatgtgggggagaacagaaagatcatggtttcgaagttgcgtctacttcacctgaggatgaatcaccgggttcaaatccagaaccagacgctactccattccaagaaggacttagaacgttcgatcagttggacgcgataagtagtcttccaacaccttctgacatctttgtttcatattcaaccttcccgggatttgtctcatggcgggacccaaagtccggtagttggtacgttgaaaccttggatgatattttcgagcagtgggcacattcagaggacctgcaatctttgctgcttcgcgtggcaaacgcggtaagcgtaaagggtatatataagcaaatgcccggatgcttcaacttcctgcggaaaaaactcttctttaagacgtccgttgacgggagcggcgccacaaacttttctctcctcaaacaggcaggagacgtcgaggaaaatcccgggcctatgtatcggatgcagttgctttcctgcatcgctttgtctcttgctttggtaaccaactcagacatccagatgacccagacgacaagtagcctgtctgccagtcttggtgatagagtgacaatttcttgtagagcgagccaggacatatctaagtacctgaactggtatcaacaaaagccggatggtacagtgaagctcctcatatatcatacgagtcggcttcatagcggggtgccaagtcgattttctgggagtggttccggtacagactactcattgacaatatcaaacttggaacaggaagacattgctacatacttttgccaacaaggcaataccctcccgtatacatttggcggaggcacaaaactcgagattacaggcggcggtggtagcggaggaggcgggtccggcggcggtggcagtgaggttaagctccaggagtctggccccggactcgtggccccttcacagagtctgagtgttacatgtacggtgtccggtgtgagcttgcctgattacggagtgtcctggatcaggcaaccgccacgaaaaggattggagtggcttggtgtgatctggggctctgaaacaacttattataactccgctttgaaaagtaggcttaccatcataaaagataacagtaagtcccaggtctttcttaaaatgaactccctgcaaaccgatgacaccgccatttactattgtgctaagcattattactacggcggtagctacgcaatggactactggggccagggaacctctgtgaccgtgtcatcagaattcgaacaaaaactcatctcagaagaggatctggcatcatttgagattgaagttatgtatccaccgccgtatcttgacaatgagaagagtaacggaacaatcatacacgttaagggtaaacatttgtgtccttctccactgtttcccggtccctcaaagcccttttgggtcctggttgtcgttggaggtgtattggcatgctatagtctgcttgtcacggtagcgtttatcattttttgggtcaggtccaaacgatctagactgctccattcagattatatgaacatgacgccccgccgaccaggaccgacgcgaaaacattaccagccgtacgcgccaccacgagactttgccgcgtaccggagtagagtcaaattctctaggagtgcagacgccccggcatatcaacagggacaaaaccaactttataatgagcttaatctggggcgaagggaagagtatgacgttctcgataagcggagggggcgggatccagagatgggtggtaagccgcagaggcgcaagaatccgcaagaaggtttgtacaatgagctccaaaaggacaagatggcagaagcctacagtgagattggaatgaagggtgaaaggcggcgaggtaagggacatgatgggttgtatcaaggtcttagtacagctaccaaggacacttatgatgccctccatatgcaggccttgccgccaagaaagcttgggagcggcgccacaaacttttctctcctcaaacaggcaggagacgtcgaggaaaatcccgggcctatgcgaatttctaagcctcaccttagaagtatctctatccaatgttacctttgtctcttgctcaacagtcactttttgacggaagccgggatacacgtcttcattctcgggtgtttctccgccgggctcccgaagaccgaggccaattgggtgaacgtaatcagtgaccttaagaaaatagaagatcttattcaatctatgcatatcgacgcaaccctctacacagaaagcgatgtgcatccttcttgcaaggttacggcaatgaagtgtttcctgctggaattgcaggtaataagtctcgaatccggtgacgccagtatacatgatacagtagaaaaccttatcatcttggcaaataacagcctgtcttcaaatggaaatgtaacagagagtggctgcaaagagtgcgaagaacttgaagagaaaaacatcaaagagtttctgcagtcatttgtgcacattgtccagatgttcattaataccagttga

SEQ ID NO: 48 - DNA, 729 bp, Artificial Sequence
  Synthetic
cagattcagcttgtgcagagcggaccggagttgaaaaaacccggagaaacagtgaaaataagctgtaaggccagcggctacacattcacagattactctatcaattgggtaaagcgggcgccgggaaaaggtctcaagtggatggggtggatcaacaccgagacacgggagcccgcatacgcgtatgattttcggggacggtttgcatttagcctggaaacgtcagcaagcacagcgtatctgcagataaataacttgaagtacgaagacacagcaacgtacttttgcgctctggactatagctacgccatggattattggggacaagggacatcagtcacggtatctagcggcggaggtggaagcggcgggggagggtccggcggcgggggaagcgacatcgttcttaccggctcccctccctcacttgccatgagtctgggtaagagggccacaatatcttgccgagcctcagagagtgttacaattctgggatcccacctcattcactggtatcaacaaaaaccaggacaaccgccaacactcttgatacagcttgcttcaaatgttcaaactggtgtcccagcaaggtttagtggatccggttcccggacggacttcacgttgacgattgacccggttgaggaggatgatgttgctgtatattattgtctgcaaagccggacaattcctcggaccttcggcgggggcacaaaactggaaattaaa

SEQ ID NO: 49 - protein, 243 aa, Artificial sequence
  synthetic
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTGSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 50 - DNA, 798 bp, Artificial sequence
  synthetic
atggattttcaggtgcagatttttagctttctgctgattagcgcgagcgtgattatgagccgcggcgatattcagatgacccagagcccgagcagcctgagcgcgagcgtgggcgatcgcgtgaccattacctgccgcgcgagccaggatgtgaacaccgcggtggcgtggtatcagcagaaaccgggcaaagcgccgaaactgctgatttatagcgcgagctttctgtatagcggcgtgccgagccgctttagcggcagccgcagcggcaccgattttaccctgaccattagcagcctgcagccggaagattttgcgacctattattgccagcagcattataccaccccgccgacctttggccagggcaccaaagtggaaattaaacgcaccggcagcaccagcggcagcggcaaaccgggcagcggcgaaggcagcgaagtgcagctggtggaaagcggcggcggcctggtgcagccgggcggcagcctgcgcctgagctgcgcggcgagcggctttaacattaaagatacctatattcattgggtgcgccaggcgccgggcaaaggcctggaatgggtggcgcgcatttatccgaccaacggctatacccgctatgcggatagcgtgaaaggccgctttaccattagcgcggataccagcaaaaacaccgcgtatctgcagatgaacagcctgcgcgcggaagataccgcggtgtattattgcagccgctggggcggcgatggcttttatgcgatggatgtgtggggccagggcaccctggtgaccgtgagcagc

SEQ ID NO: 51 - protein, 266 aa, Artificial sequence
  synthetic
MDFQVQIFSFLLISASVIMSRGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 52 - DNA, 549 bp, Artificial sequence
  synthetic
gatattcagatgacccagagcccgagcagcctgagcgcgagcgtgggcgatcgcgtgaccattacctgctggtatcagcagaaaccgggcaaagcgccgaaactgctgatttatggcgtgccgagccgctttagcggcagcggcagcggcaccgattttaccctgaccattagcagcctgcagccggaagattttgcgacctattattgctttggccagggcaccaaagtggaaattaaacgcggcggcggcggcagcggcggcggcggcagcggcggcggcggcagcggcggcggcggcagcgaagtgcagctggtggaaagcggcggcggcctggtgcagccgggcggcagcctgcgcctgagctgcgcggcgagctgggtgcgccaggcgccgggcaaaggcctggaatgggtggcgcgctttaccattagccgcgatgatagcaaaaacaccgcgtatctgcagatgaacagcctgcgcgcggaagataccgcggtgtattattgcaccacctggggccagggcaccctggtgaccgtgagcagc

SEQ ID NO: 53 - protein, 183 aa, Artificial sequence
  synthetic
DIQMTQSPSSLSASVGDRVTITCWYQQKPGKAPKLLIYGVPSRFSGSGSGTDFTLTISSLQPEDFATYYCFGQGTKVEIKRGGGGSGGGGSGGGGSGGGGSEVQLVESGGGLVQPGGSLRLSCAASWVRQAPGKGLEWVARFTISRDDSKNTAYLQMNSLRAEDTAVYYCTTWGQGTLVTVSS

SEQ ID NO: 54 - DNA, 723 bp, Artificial sequence
  synthetic
gatattcagatgacccagagcccgagcagcctgagcgcgagcgtgggcgatcgcgtgaccattacctgccgcgcgagccagagcattagcagctatctgaactggtatcagcagaaaccgggcaaagcgccgaaactgctgatttatgcggcgagcagcctgcagagcggcgtgccgagccgctttagcggcagcggcagcggcaccgattttaccctgaccattagcagcctgcagccggaagattttgcgacctattattgccagcagagctatagcaccccgctgacctttggcggcggcaccaaagtggaaattaaacgcagccgcggcggcggcggcagcggcggcggcggcagcggcggcggcggcagcgaagtgcagctggtggaaagcggcggcggcctggtgcagccgggcggcagcctgcgcctgagctgcgcggcgagcggctttaccagcagcagctattggatgagctgggtgcgccaggcgccgggccgcggcctggaatgggtggcgaacattaaacaggatggcagcgaaaaatattatgtggatagcgtgaaaggccgctttaccattagccgcgataacgcgaaaaacagcctgtatctgcagatgaacagcctgcgcgcggaagataccgcggtgtattattgcgcgcgcggcggctggagctatgatatgtggggccagggcaccctggtgaccgtgagcagc

SEQ ID NO: 55 - protein, 241 aa, Artificial sequence
  synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRSRGGGGSGGGGSGGGGSEVQLVESGGGLVQPGGSLRLSCAASGFTSSSYWMSWVRQAPGRGLEWVANIKQDGSEKYYVDSVKGRFTISRDNAKNSLYLQMNSLRAEDTAVYYCARGGWSYDMWGQGTLVTVSS

SEQ ID NO: 56 - DNA, 780 bp, Artificial sequence
  synthetic
atggattttcaggtgcagatttttagctttctgctgattagcgcgagcgtgattatgagccgcggcgaaattgtgatgacccagagcccggcgaccctgagcctgagcccgggcgaacgcgcgaccctgagctgccgcgcgagccagagcgtgagcagctatctggcgtggtatcagcagaaaccgggccaggcgccgcgcctgctgatttatgatagcagcaaccgcgcgaccggcattccggcgcgctttagcggcagcggcagcggcaccgattttaccctgaccattagcagcctggaaccggaagattttgcgacctattattgcctgcagcataacacctttccgagcacctttggccagggcaccaaagtggaaattaaaggcagcaccagcggcagcggcaaaccgggcagcggcgaaggcagcgaagtgcagctggtgcagagcggcggcggcctggtgaaaccgggcggcagcctgcgcctgagctgcgcggcgagcggctttacctttagcagctatagcatgaactgggtgcgccaggcgccgggcaaaggcctggaatgggtgagcagcattagcagcagcagcagctatatttattatgcggatagcgtgaaaggccgctttaccattagccgcgataacgcgaaaaacagcctgtatctgcagatgaacagcctgcgcgcggaagataccgcggtgtattattgcgcgcgcgtgaccgatgcgtttgatatttggggccagggcaccatggtgaccgtgagcagc

SEQ ID NO: 57 - protein, 260 aa, Artificial sequence
  synthetic
MDFQVQIFSFLLISASVIMSRGEIVMTQSPATLSLSPGERATLSCRASQSVSSYLAWYQQKPGQAPRLLIYDSSNRATGIPARFSGSGSGTDFTLTISSLEPEDFATYYCLQHNTFPSTFGQGTKVEIKGSTSGSGKPGSGEGSEVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWGQGTMVTVSS